SEQUENCE LISTING (13 sequences)

SEQ ID NO: 1 - DNA, 34 bp, Artificial sequence
  Synthetic sequence
atggacaaagactgcgaaatgaagcgcaccaccc

SEQ ID NO: 2 - DNA, 20 bp, Artificial sequence
  synthetic sequence
acccagcccaggcttgccca

SEQ ID NO: 3 - DNA, 59 bp, Artificial sequence
  synthetic sequence
ggcaagcctgggctgggtgactacaaagaccatgacggtgattataaagatcatgacat

SEQ ID NO: 4 - DNA, 59 bp, Artificial sequence
  synthetic sequence
gtgcgcttcatttcgcagtctttgtccatgcttccgccgccgcggccgccacaggtaca

SEQ ID NO: 5 - DNA, 24 bp, Artificial sequence
  synthetic sequence
atgcagccagatcccaggcctagc

SEQ ID NO: 6 - DNA, 39 bp, Artificial sequence
  synthetic sequence
gatatctgcagaattccaccacactggactagtggatcc

SEQ ID NO: 7 - DNA, 59 bp, Artificial sequence
  synthetic sequence
ccagtgtggtggaattctgcagatatcatggacaaagactgcgaaatgaagcgcaccac

SEQ ID NO: 8 - DNA, 53 bp, Artificial sequence
  synthetic sequence
gcctgggatctggctgcatgctccctccgccgccacccagcccaggcttgccc

SEQ ID NO: 9 - DNA, 47 bp, Artificial sequence
  synthetic sequence
tagtaatgagtttggaattaattctgtggaatgtgtgtcagttaggg

SEQ ID NO: 10 - DNA, 28 bp, Artificial sequence
  synthetic sequence
ggtgaagggatcaattccaccacactgg

SEQ ID NO: 11 - DNA, 39 bp, Artificial sequence
  synthetic sequence
ggtggaattgatcccttcaccatgcagccagatcccagg

SEQ ID NO: 12 - DNA, 60 bp, Artificial sequence
  synthetic sequence
cacattccacagaattaattccaaactcattactacttgtcatcgtcatccttgtagtcg

SEQ ID NO: 13 - DNA, 46 bp, Artificial sequence
  synthetic sequence
ggtggaattgatcccttcaccatggacaaagactgcgaaatgaagc

(19) United States
(12) Patent Application Publication
(10) Pub. No.: US 2023/0137943 A1
(43) Pub. Date: May 4, 2023

(54) PHOTOPROXIMITY PROFILING OF PROTEIN-PROTEIN INTERACTIONS IN CELLS

(71) Applicant: The University of Chicago, Chicago, IL (US)

(72) Inventors: Raymond E. Moellering, Chicago (US); David C. McCutcheon, Chicago (US); Anthony Carlos, Chicago, IL (US)

(21) Appl. No.: 17/762,180

(22) PCT Filed: Sep. 21, 2020
(86) PCT No.: PCT/US2020/051834

§ 371 Date: Mar. 21, 2022

Related U.S. Application Data
(60) Provisional application No. 62/903,621, filed on Sep. 20, 2019.

(51) Int. Cl. G01N 33/58; G01N 33/68; C07D 519/00

(52) U.S. Cl. CPC G01N 33/582; G01N 33/6845; C07D 519/00

(57) ABSTRACT

Photoactive probes and probe systems for detecting biological interactions are described. The photoactive probes include probes that combine both photocleavable and photoreactive moieties. The photoactive probe systems can include a first probe comprising a photocatalytic group and a second probe comprising a group that can act as a substrate for the reaction catalyzed by the photocatalytic group. The probes and probe systems can also include groups that can specifically bind to a binding partner on a biological entity of interest and a detectable group or a precursor thereof. The probes and probe systems can detect spatiotemporal interactions of proteins or cells. In some embodiments, the interactions can be detected in live cells. Also described are methods of detecting the biological interactions.

### RELATED APPLICATIONS

The presently disclosed subject matter claims the benefit of U.S. Provisional Patent Application Ser. No. 62/903,621, filed Sep. 20, 2019, the disclosure of which is incorporated herein by reference in its entirety.

### STATEMENT OF GOVERNMENT SUPPORT

This invention was made with government support under grant numbers GM008720, CA175399, and GM128199 awarded by the National Institutes of Health. The government has certain rights in the invention.

### TECHNICAL FIELD

The presently disclosed subject matter relates to methods of detecting spatiotemporal interactions in biological systems, such as protein-protein interactions and cell-cell interactions, as well to photoactive probes for use in detecting the interactions.
Abbreviations
° C.=degrees Celsius
%=percentage
μL=microliter
μm=microns
μM=micromolar
AP=affinity purification
BnG=benzylguanine
Boc=tert-butoxycarbonyl
BSA=bovine serum albumin
BTOI=biological target of interest
DMSO=dimethylsulfoxide
FITC=fluorescein isothiocyanate
g=grams
h (or hr)=hours
kDa=kilodaltons
KEAP1=Kelch Like ECH Associated Protein 1
LC-MS=liquid chromatography-mass spectroscopy
min=minutes
mL=milliliter
mM=millimolar
mmol=millomoles
MS=mass spectrometry
nm=nanometer
nM=nanomolar
NMR=nuclear magnetic resonance
POI=protein of interest
PP1=photoproximity probe 1
PPI=protein-protein interaction
s=seconds
SILAC=stable isotope labeling by amino acids in cell culture

### BACKGROUND

Building high-fidelity and dynamic maps of molecular interaction networks inside of living cells is currently a challenge. The ability to elucidate the so-called “social network” for a biomolecule of interest under a wide range of biological contexts can provide the basis for understanding how cellular machinery is organized in space and time.¹ This information can provide better understanding of cellular signaling events, as well as the potential to perturb these events for therapeutic purposes. Methods of drafting high-quality interactome maps that can detect transient binding interactions ideally would preserve the spatial, mechanical, and chemical environment found in living systems (i.e. cells or tissues). Affinity purification-mass spectrometry (AP-MS) pull-down approaches have historically been used to enrich a tagged protein-of-interest (POI), with the intention that binding partners will co-elute during iterative rounds of enrichment and washing, followed by protein detection by mass spectrometry.² While powerful, the steps involved in cellular lysis and dilution through iterative washes biases against weaker binding interactions that dominate many protein-protein interactions, and also has the propensity to introduce non-specific interactions between partners that were compartmentalized in the cell. There are also many classes of interactions and proteins that are not appropriate for this approach, including membrane proteins, chromatin-associated complexes, and redox-sensitive complexes, among others. Therefore, while many of the protein interactome maps drafted to date have been developed with AP-MS methods, new methods to augment or replace these maps are needed.

To circumvent the need to lyse cells and perform in vitro enrichments, technologies have been developed that enable chemical or enzymatic tagging of proximal protein binding partners within live cells, which are then retrieved after cellular lysis and identified by LC-MS/MS. For instance, the BioID method fuses an engineered biotin ligase BirA to a target POI, which then converts intracellular ATP and exogenous biotin into an amine-reactive biotinoyl-5′-AMP that can diffuse from the POI and covalently label proximal proteins.³⁻⁵ Proximal enzymatic labeling with similarly reactive thioesters, for example with the NEDDylator⁶, PUP-IT⁷, and EXCEL⁸ systems, has been shown to label proximal proteins with small protein or peptide tags, providing for subsequent profiling. Genetic fusions of horseradish peroxidase⁹ and engineered ascorbic acid peroxidase enzymes can convert exogenous chemical probes into reactive phenoxyl radicals^{10,11} to label proximal proteins in cells. Indeed, this method has been used to map the sub-cellular proteome in organelles,^{11,12} as well as to identify protein complex members in diverse conditions.¹³ But, even with the success of these approaches, there are significant limitations imposed by the requirement for co-factors, an exogenous biotin-phenol probe, and high levels of H₂O₂ to initiate labeling, which could bias efficient labeling in specific cellular and chemical environments. Additionally, the requirement for peroxide limits application of this approach with proteins or pathways that involve redox regulation, which likely includes a large percentage of the proteome.¹⁴⁻¹⁶

Accordingly, there is an ongoing need for new methods and probes for proximity profiling in biological systems, e.g., for diagnostic and/or research purposes. In particular, new methods and probes that can label proximal proteins in live cells or label other biological targets of interest with high spatial and temporal control, ideally without significant perturbation to the cellular or other biological environment, would be beneficial for mapping spatiotemporal biological interactions, including PPIs, cell-cell interactions, protein-metabolite interactions, cell-protein interactions, and protein-drug interactions.

### SUMMARY

In some embodiments, the presently disclosed subject matter provides a photoactive chemical probe or probe system for proximity profiling of biological interactions, wherein the photoactive chemical probe or probe system comprises a target recognition moiety capable of specifically binding a first binding partner associated with a biological target of interest (BTOI), optionally wherein the first binding partner is a peptide or protein tag attached to the BTOI; a detectable moiety or precursor thereof; and at least two photoactive moieties, wherein one of said photoactive moieties is a photocleavable or photocatalytic moiety. In some embodiments, the photoactive chemical probe or probe system comprises a photoactive probe having a structure of Formula (I):

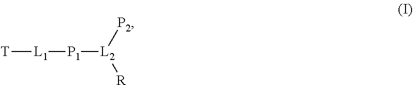

wherein: T is a target recognition moiety capable of specifically binding a first binding partner, optionally wherein the first binding partner is a peptide or protein tag attached to a biological target of interest; L₁ is a bivalent linker; P₁ is a photocleavable moiety; L₂ is a trivalent linker moiety; P₂ is a photoreactive moiety; and R is a detectable moiety or a precursor thereof capable of specifically binding a second binding partner, subject to the proviso that the first and second binding partners are different. In some embodiments, the photoactive probe or probe system comprises a probe system comprising: a photocatalytic probe having a structure of Formula (VII): T-L₁₀-P_{c}; and a probe substrate having a structure of Formula (VIII): P₃-L₁₁-R; wherein: T is a target recognition moiety capable of specifically binding a first binding partner, optionally wherein the first binding partner is a peptide or protein tag attached to a biological target of interest; L₁₀ and L₁₁ are bivalent linkers; P_{c} is a photocatalytic moiety. P₃ is a photoreactive moiety that is capable of undergoing a reaction catalyzed by P_{c}; and R is a detectable moiety or a precursor thereof capable of specifically binding a second binding partner, subject to the proviso that the first and second binding partners are different.

In some embodiments, R comprises biotin, a biotin analog, or an alkyne. In some embodiments, R is selected from:

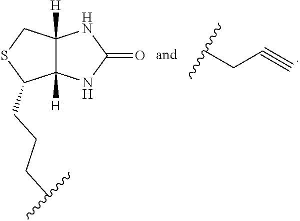

In some embodiments, T comprises a moiety selected from the group comprising a benzylguanine group, a chloroalkane group, a benzylcytosine group, an azide, biotin, desthiobiotin, AP1867 or an orthogonal FK506 analog, and a methotrexate derivative. In some embodiments, T is selected from:

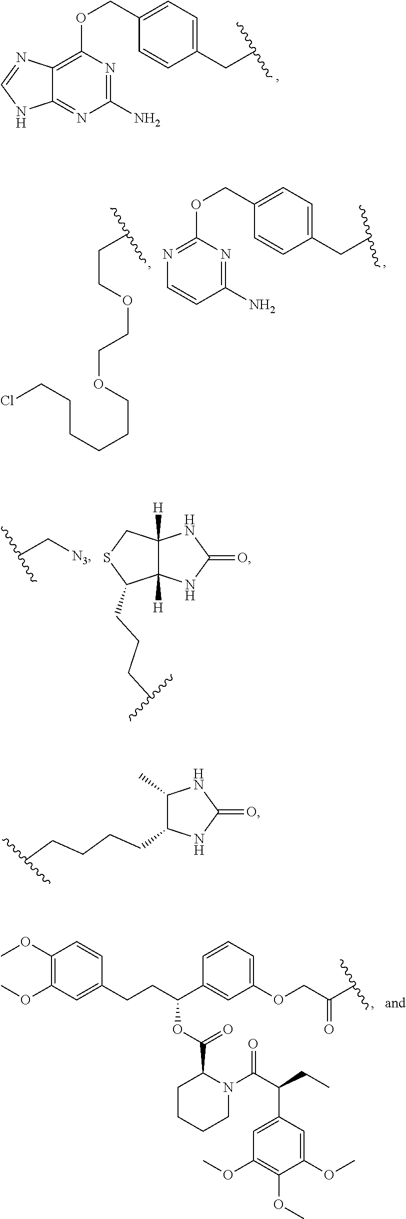

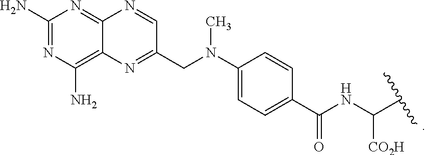 [SMILES: *C(NC(=O)c1ccc(N(C)Cc2cnc3nc(N)nc(N)c3n2)cc1)C(=O)O]

In some embodiments, P₂ comprises a diazirine derivative, a benzophenone derivative, or an aryl azide derivative. In some embodiments, P₂ is selected from:

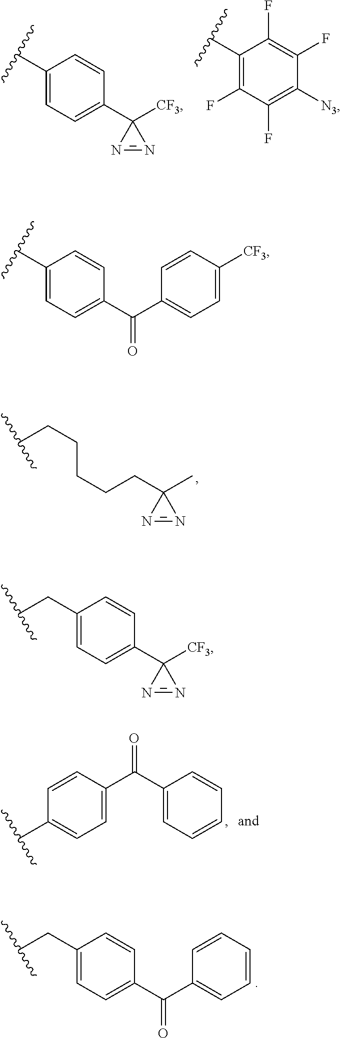

In some embodiments, L₁ is selected from —NH—C(═O)-alkylene-; —NH—C(═O)—O—CH₂CH₂—O—; and —NH—C(═O)—O—CH₂CH₂—NH—C(═O)-alkylene-, wherein said alkylene is substituted or unsubstituted, optionally wherein said alkylene is propylene. In some embodiments, L₂ is selected from the group comprising:

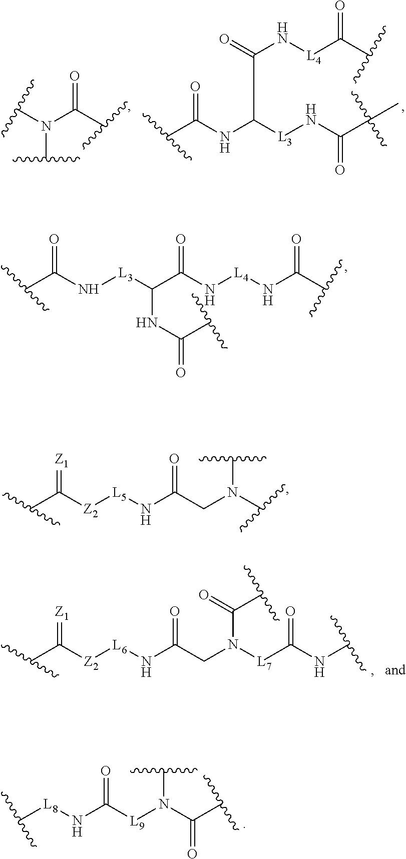

wherein each L₃, L₄, L₅, L₆, L₇, L₈, and L₉ is alkylene, wherein said alkylene is substituted or unsubstituted, optionally wherein said alkylene comprises one or more oxygen atoms inserted along the alkylene group; wherein Z₁ and Z₃ are selected from O and S; and wherein Z₂ and Z₄ are selected from O, S, and NH; optionally wherein L₂ is selected from:

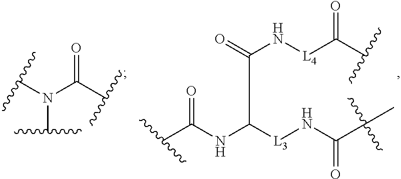

wherein L₃ is butylene and L₄ is pentylene; and

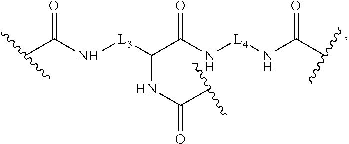

wherein L₃ is butylene and L₄ is ethylene.

In some embodiments, P₁ comprises a divalent nitroaryl derivative, a divalent coumarin derivative, or a divalent hydroxyaryl derivative. In some embodiments, P₁ comprises a divalent ortho-nitrobenzyl derivative, a divalent coumarin derivative, a divalent nitroindoline derivative, a divalent nitrobenzopiperidine derivative, a divalent ortho-hydroxybenzyl derivative, or a divalent ortho-hydroxynaphthyl derivative.

In some embodiments, the compound of Formula (I) has a structure of Formula (II):

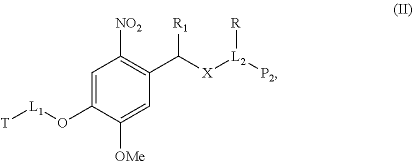

wherein: T, L₁, L₂, R, and P₂ are as defined for the compound of Formula (I); and X is selected from O, NR′, and S, wherein R′ is selected from H and alkyl; and R₁ is selected from H, alkyl, perhaloalkyl, and cyano. In some embodiments, X and L₂ together form a group comprising a carbamate, a urea, a thiourea, an amide, an ester, an ether, an amine, or a sulfide. In some embodiments, the probe is selected from:

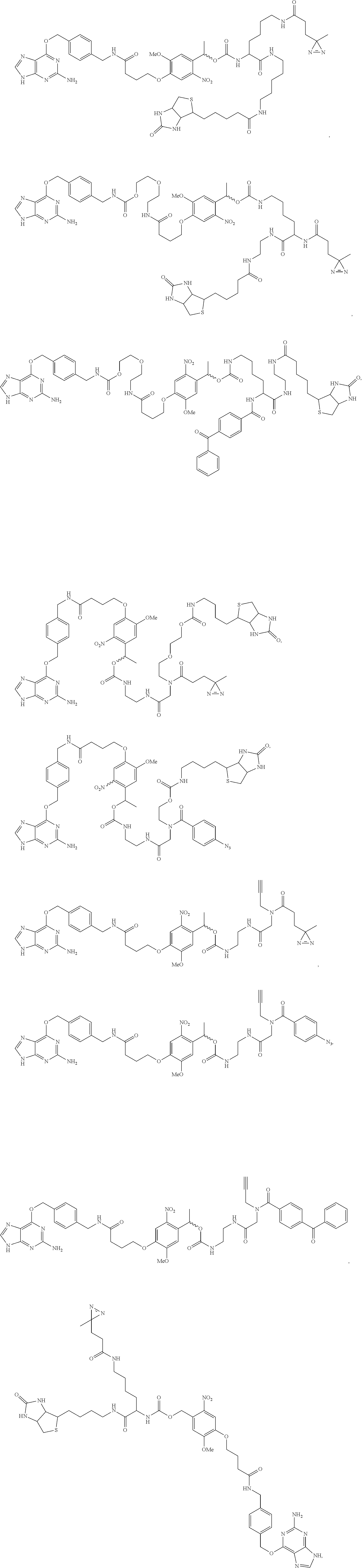

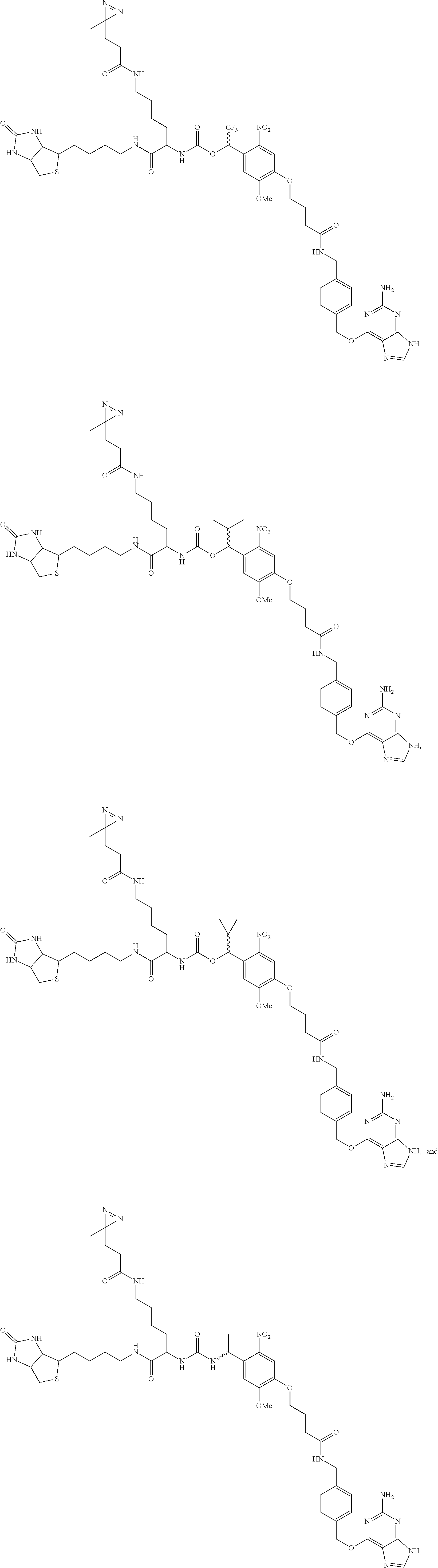

In some embodiments, L₂ is —N—C(═O)—, and the compound of Formula (I) has a structure of Formula (IIIa) or Formula (IIIb):

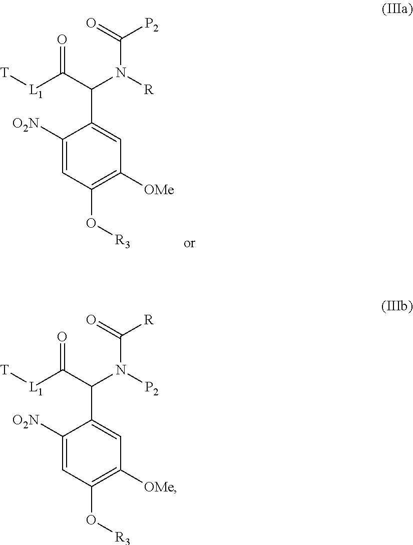

wherein: T, L₁, R, and P₂ are as defined for the compound of Formula (I); and R₃ is alkyl, optionally methyl. In some embodiments, the compound of Formula (I) has the structure:

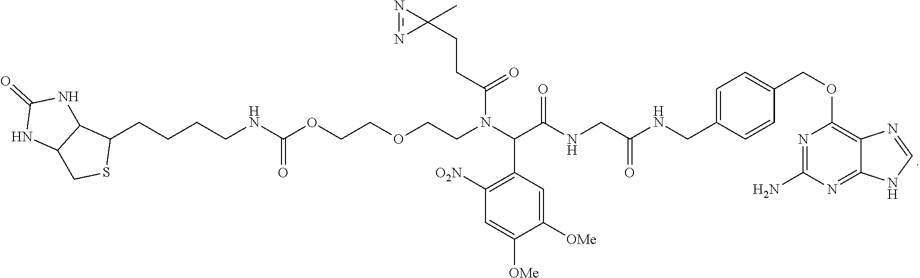 [SMILES: COc1cc(C(C(=O)NCC(=O)NCc2ccc(COc3nc(N)nc4[nH]cnc34)cc2)N(CCOCCOC(=O)NCCCCC2SCC3NC(=O)NC32)C(=O)CCC2(C)N=N2)c([N+](=O)[O-])cc1OC]

In some embodiments, the compound of Formula (I) has a structure of Formula (IVa) or (IVb):

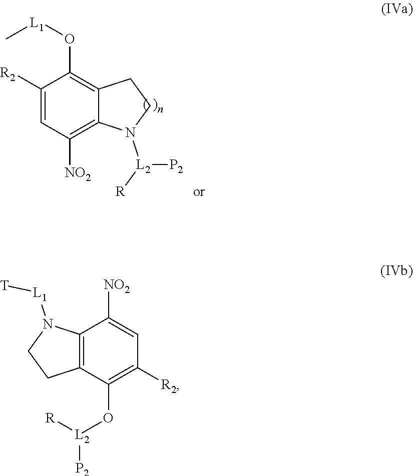

wherein: T, L₁, L₂, R and P₂ are as defined for Formula (I), n is 1 or 2; and R₂ is selected from NO₂ and H. In some embodiments, the probe is a compound of Formula (IVa) and L₂ and the nitrogen atom to which L₂ is attached together form a carbamate, a urea, a thiourea, an amide, or a sulfonamide; or wherein the probe is a compound of Formula (IVb) and L₁ and the nitrogen atom to which L₁ is attached together form a carbamate, a urea, a thiourea, an amide, or a sulfonamide.

In some embodiments, the compound of Formula (I) has a structure of Formula (Va) or (Vb):

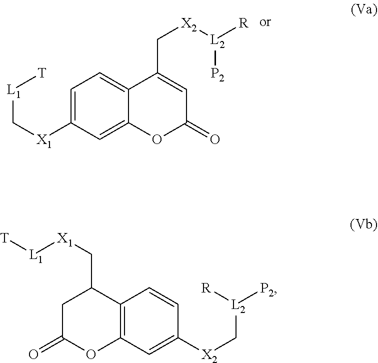

wherein: T, L₁, L₂, R, and P₂ are as defined for the compound of Formula (I), and X₁ and X₂ are independently selected from O, NR′, and S, wherein R′ is H or alkyl. In some embodiments, the compound has a structure of Formula (Va) and X₂ and L₂ together form a carbamate, a urea, an amide, an ester, an ether, an amine, a sulfide, or a thiourea group; or wherein the compound has a structure of Formula (Vb) and X₁ and L₁ together form a carbamate, a urea, an amide, an ester, an ether, an amine, a sulfide, or a thiourea group.

In some embodiments, the compound of Formula (I) has a structure of one of Formula (VIa) and (VIb):

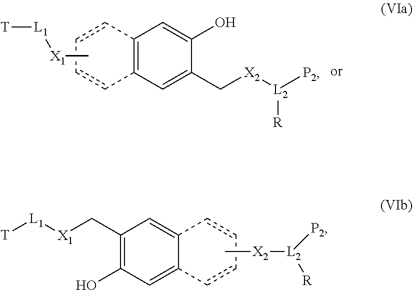

Wherein: T, L₁, L₂, P₂, and R are as defined for the compound of Formula (I); the dotted lines can be present or absent, and when absent, X₁ or X₂ is substituted on the remaining aryl ring; and X₁ and X₂ are independently selected from O, NR′, and S, wherein R′ is selected from H and alkyl. In some embodiments, L₁ and X₁ together and L₂ and X₂ together each independently form a group selected from a carbamate, a urea, an amide, an ester, an ether, an amine, a sulfide, or a thiourea group.

In some embodiments, P_{c} is a monovalent isoalloxazine moiety, optionally having the structure:

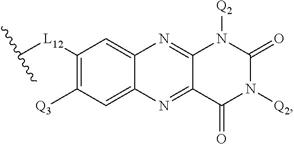

wherein: L₁₂ is present or absent and when present is a bivalent moiety selected from the group comprising —O-alkylene, —S-alkylene, —NQ₄-alkylene, and alkylene, wherein said alkylene is substituted or unsubstituted; and each of Q₁, Q₂, Q₃ and Q₄ are independently selected from H, alkyl, and cycloalkyl. In some embodiments, L₁₂ is absent or is —O-alkylene, optionally wherein the alkylene is methylene. In some embodiments, Q₃ is methyl and Q₁ and Q₂ are each H, methyl or cyclopropyl. In some embodiments, the compound of Formula (VII) is selected from:

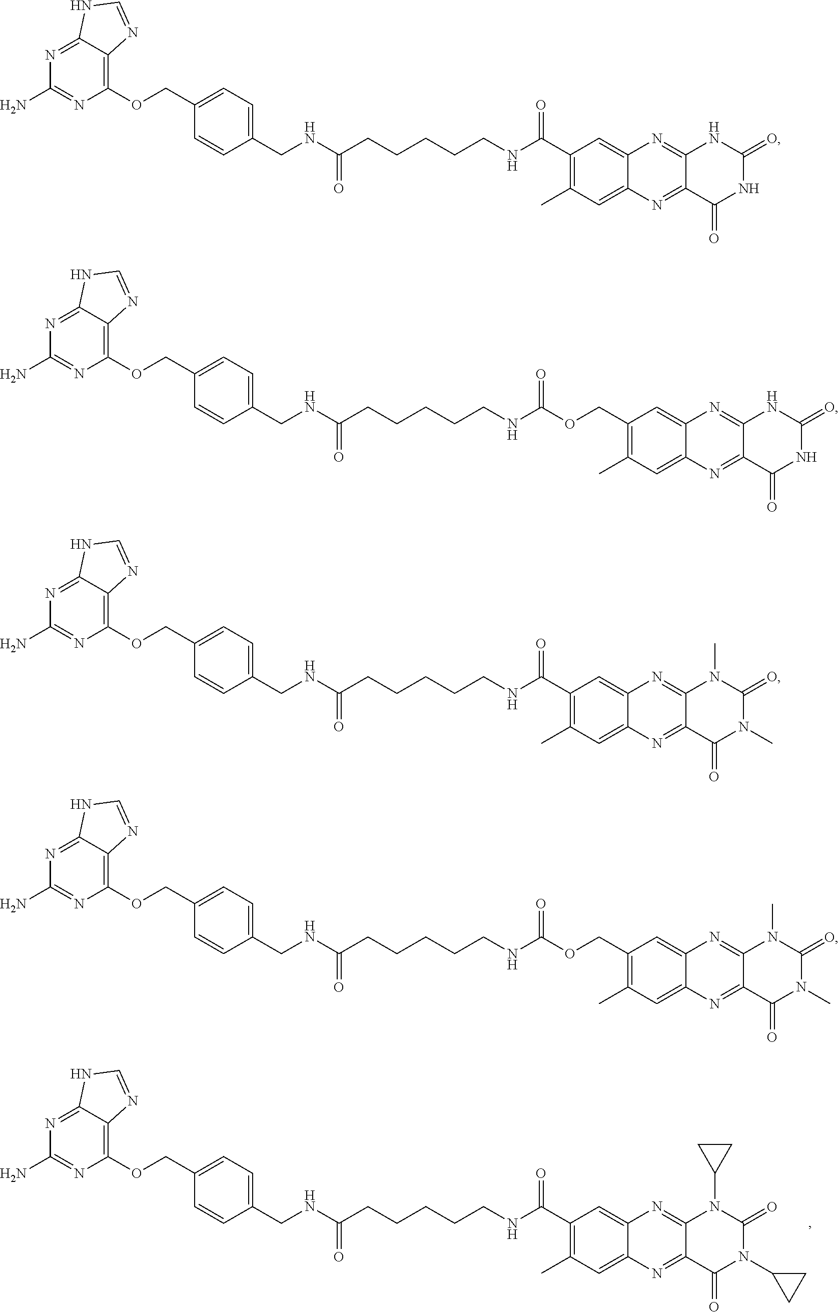 [SMILES: Cc1cc2nc3c(=O)[nH]c(=O)[nH]c3nc2cc1C(=O)NCCCCCC(=O)NCc1ccc(COc2nc(N)nc3[nH]cnc23)cc1 ; Cc1cc2nc3c(=O)[nH]c(=O)[nH]c3nc2cc1COC(=O)NCCCCCC(=O)NCc1ccc(COc2nc(N)nc3[nH]cnc23)cc1 ; Cc1cc2nc3c(=O)n(C)c(=O)n(C)c3nc2cc1C(=O)NCCCCCC(=O)NCc1ccc(COc2nc(N)nc3[nH]cnc23)cc1 ; Cc1cc2nc3c(=O)n(C)c(=O)n(C)c3nc2cc1COC(=O)NCCCCCC(=O)NCc1ccc(COc2nc(N)nc3[nH]cnc23)cc1 ; Cc1cc2nc3c(=O)n(C4CC4)c(=O)n(C4CC4)c3nc2cc1C(=O)NCCCCCC(=O)NCc1ccc(COc2nc(N)nc3[nH]cnc23)cc1]

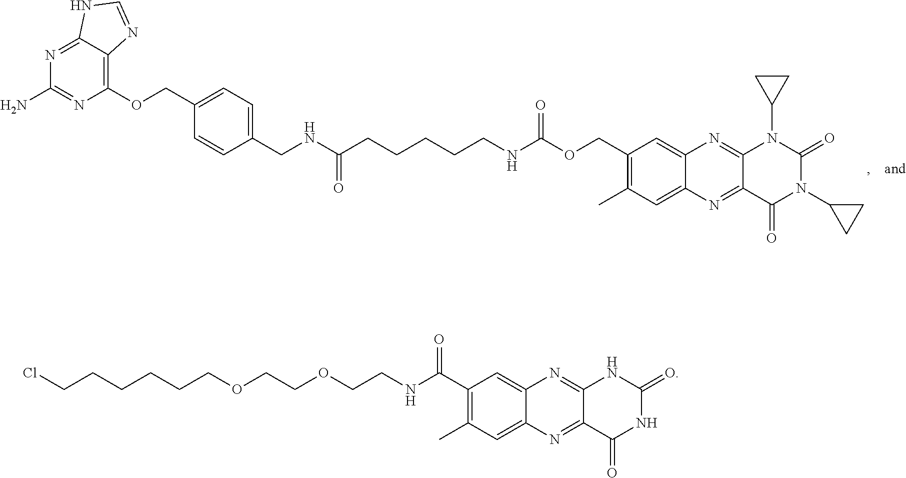 [SMILES: Cc1cc2nc3c(=O)n(C4CC4)c(=O)n(C4CC4)c3nc2cc1COC(=O)NCCCCCC(=O)NCc1ccc(COc2nc(N)nc3[nH]cnc23)cc1 ; Cc1cc2nc3c(=O)[nH]c(=O)[nH]c3nc2cc1C(=O)NCCOCCOCCCCCCCl]

In some embodiments, P₃ is selected from a phenol, an aniline, and a diazirine. In some embodiments, the robe substrate has a structure selected from the group consisting of:

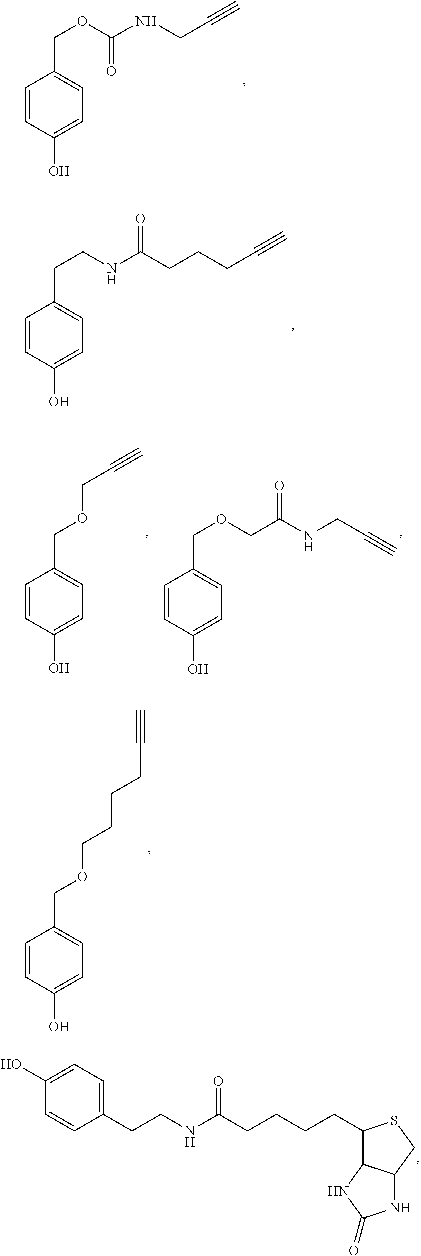

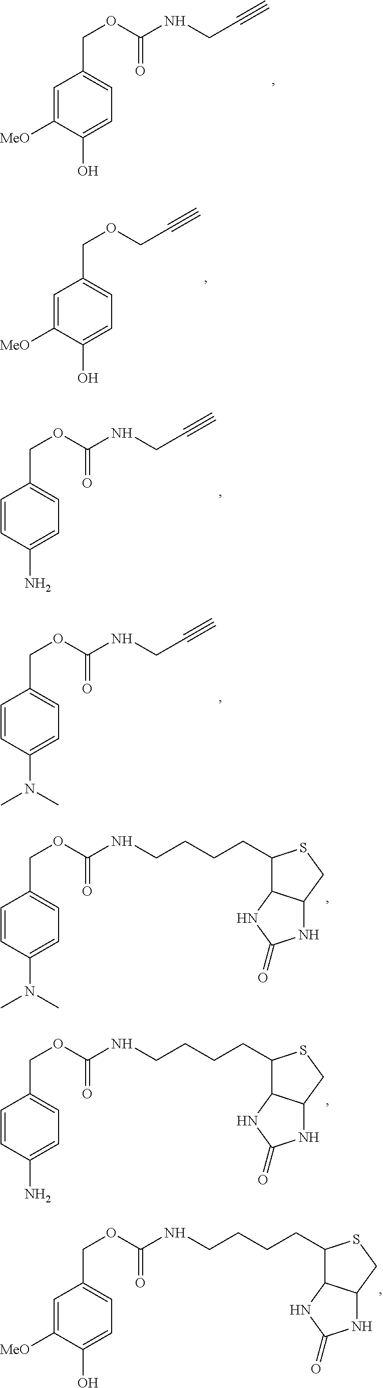

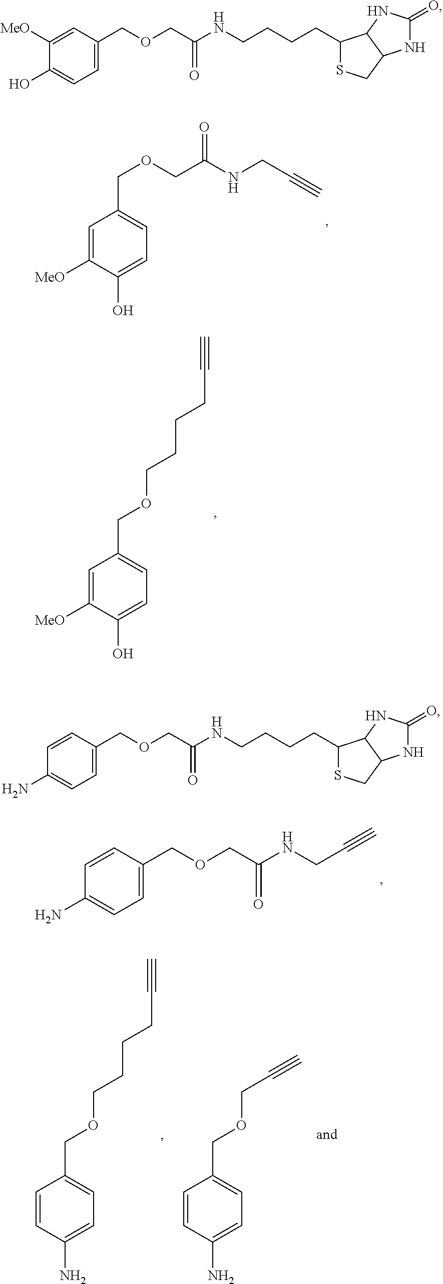

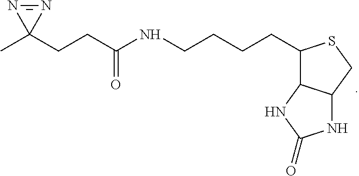 [SMILES: CC1(CCC(=O)NCCCCC2SCC3NC(=O)NC32)N=N1]

In some embodiments, the presently disclosed subject matter provides for the use of a photoactive probe or probe system of the presently disclosed subject matter in detecting one or more biological interactions, optionally one or more transient biological interactions, between a biological target of interest (BTOI) and one or more second entities, optionally wherein said one or more interactions are selected from the group comprising a protein-protein interaction; a protein-metabolite interaction; a cell-cell interaction; a protein-nucleic acid interaction, optionally a protein-RNA interaction or a protein-DNA interaction; a protein-drug interaction, and a nucleic acid-drug interaction. In some embodiments, the detecting comprises detecting one or more interactions between a BTOI and one or more second entities, wherein the detecting is performed in an organ, tissue, live cell, or bodily fluid.

In some embodiments, the BTOI is a protein and the detecting is performed in a live cell transiently or stably expressing a fusion protein comprising the BTOI and a detectable protein or peptide tag. In some embodiments, the BTOI is a cell and the detecting is performed in a cell culture, tissue, organ or bodily fluid comprising the cell BTOI wherein said cell BTOI expresses a detectable protein or peptide tag on a luminal surface of said cell.

In some embodiments, the presently disclosed subject matter provides a method for detecting a spatiotemporal interaction of a biological target of interest (BTOI), optionally a cell or protein of interest, wherein the method comprises: (a) labeling the BTOI with a moiety comprising a first binding partner; (b) contacting the BTOI with a photoactive probe comprising: (i) a moiety that binds the first binding partner, (ii) a photoreactive moiety attached to a moiety that binds a second binding partner, and (iii) a photocleavable moiety attaching (i) and (ii); and (c) exposing the probe to light, thereby cleaving the photocleavable moiety and causing the photoreactive moiety to diffuse from the BTOI and react covalently or non-covalently with one or more biological entities in proximity to the BTOI and within a diffusion radius associated with the chemical probe, thereby labeling said one or more biological entities with the moiety that binds a second binding partner. In some embodiments, a diffusion radius of the photoactive probe and a radius of interrogation of spatiotemporal interactions of the BTOI is adjustable based on the reactivity of the photoreactive moiety and/or the reactivity of the photocleavable moiety. In some embodiments, the method comprises contacting the BTOI with two or more chemical probes, wherein each of said two or more chemical probes has a different diffusion radius and the moiety that binds a second binding partner of each of said two or more chemical probes binds a different second binding partner.

In some embodiments, the contacting is performed in a live cell, a cell culture, a tissue sample, a bodily fluid sample, or an organ sample. In some embodiments, the method is free of a chemical co-factor to activate the photoreactive group. In some embodiments, the method comprises detecting one or more cell-cell interactions, one or more cell-protein interactions, and/or one or more cell-drug interactions. In some embodiments, the method comprises detecting one or more protein-protein interactions; one or more protein-metabolite interactions; one or more protein-nucleic acid interactions, optionally one or more protein-RNA or protein-DNA interactions; and/or one or more protein-drug interactions.

In some embodiments, the presently disclosed subject matter provides a method for detecting a spatiotemporal interaction of a biological target of interest (BTOI), optionally a cell or protein of interest, wherein the method comprises: (a) providing a sample comprising a BTOI labeled with a moiety comprising a first binding partner; (b) contacting the BTOI with a photocatalytic probe comprising: (i) a moiety that binds the first binding partner and (ii) a photocatalytic moiety; (c) contacting the sample with one or more probe substrates, wherein each probe substrate comprises: (iii) a photoreactive moiety that is capable of undergoing a reaction catalyzed by the photocatalytic moiety and (iv) a detectable moiety or precursor thereof that is capable of specifically binding a second binding partner; and (d) exposing the sample to light, thereby exciting said photocatalytic moiety and causing the photocatalytic moiety to catalyze a reaction where the photoreactive moiety is transformed into a moiety that can react covalently or non-covalently with one or more biological entities in proximity to the BTOI, thereby labeling said one or more biological entities with the moiety that binds a second binding partner. In some embodiments, the sample is a live cell, a cell culture, a tissue sample, a bodily fluid sample, or an organ sample. In some embodiments, the method comprises detecting one or more cell-cell interactions, one or more cell-protein interactions, and/or one or more cell-drug interactions. In some embodiments, the method comprises detecting one or more protein-protein interactions; one or more protein-metabolite interactions; one or more protein-nucleic acid interactions, optionally one or more protein-RNA or protein-DNA interactions; and/or one or more protein-drug interactions In some embodiments, a radius of interrogation of spatiotemporal interactions of the BTOI is adjustable based on one or more of reactivity of the photocatalytic moiety, distance between the photocatalytic moiety and the moiety that binds the first binding partner, and reactivity and/or half-life of the moiety resulting from the reaction of the photoreactive moiety catalyzed by the photocatalytic moiety.

In some embodiments, the presently disclosed subject matter provides a method of detecting interactions of a biological target of interest (BTOI), the method comprising: (a) providing a sample comprising a labelled BTOI, wherein said labelled BTOI comprises the BTOI and a detectable tag; optionally wherein said BTOI is a cell or a protein, further optionally wherein the detectable tag is protein or peptide; (b) contacting the sample with a photoactive probe of Formula (I) or a photoactive probe system comprising a photocatalytic probe of Formula (VII) and a probe substrate of Formula (VIII), wherein the target recognition moiety T specifically binds to the detectable tag of the labelled BTOI; (c) exposing the sample to light, thereby (i) triggering the cleavage of the photocleavable moiety P₁ and the activation of the photoreactive moiety P₂, wherein the photoreactive moiety P₂ reacts to form a covalent linkage with a second entity in proximity to the POI, thereby tagging said second entity with the detectable moiety R; or (ii) activating the photocatalytic moiety Pc, thereby catalyzing a reaction of the photoreactive moiety P3, transforming said photoreactive moiety P3 into a moiety that can react to form a covalent linkage with a second entity in proximity to the POI, thereby tagging said second entity with the detectable moiety R; and (d) detecting the detectable moiety R, thereby detecting the second entity interacting with or in proximity to the BTOI.

In some embodiments, the BTOI is a protein of interest (POI) and providing a sample comprising a labelled BTOI comprises providing a sample comprising a labelled POI, wherein said labelled POI comprises the POI and a detectable tag; optionally wherein the detectable tag is protein or peptide, further optionally wherein the detectable tag is selected from a SNAP-tag, a Halo-Tag, a Clip-Tag, a receptor engineered with strained cyclooctyne, monomeric streptavidin, neutravidin, avidin, FKBP12 or a mutant thereof, and DHFR; wherein the target recognition moiety T of the chemical probe specifically binds to the detectable tag of the labelled POI; and wherein detecting the detectable moiety R of the chemical probe, thereby detecting the protein in proximity to the POI. In some embodiments, the sample comprises a live cell comprising the labelled POI. In some embodiments, the method further comprises lysing the cells prior to the detecting of step (d).

In some embodiments, the method comprises enriching the sample for the detectable moiety R, optionally wherein the enriching comprises contacting the sample with a solid support comprising a binding partner of the detectable moiety R. In some embodiments, the detectable moiety R is biotin or an analog thereof, and wherein the enriching comprises contacting the sample with streptavidin-coated beads, further optionally wherein the streptavidin-coated beads are streptavidin-coated magnetic beads. In some embodiments, the detecting comprises performing liquid chromatography-tandem mass spectrometry (LC-MS/MS) on the digested sample.

In some embodiments, the sample comprises a live cell that stably or transiently expresses the labelled POI, wherein the labelled POI is a fusion protein comprising the POI and a detectable protein or peptide tag. In some embodiments, the method further comprises culturing the live cell in a cell culture medium comprising heavy isotopes prior to the contacting of step (b), thereby providing a “heavy” cell sample, optionally wherein the cell culture medium comprises ¹³C- and/or ¹⁵N-labeled amino acids, further optionally wherein the cell culture medium comprises ¹³C-, ¹⁵N-labeled lysine and arginine. In some embodiments, after steps (b) and (c) and prior to the detecting of step (d), the heavy cells of the heavy cell sample are lysed to provide a lysed sample, and the detecting comprises: (d1) enriching the lysed sample for the detectable moiety R to provide an enriched sample; (d2) combining the enriched sample with an enriched sample prepared from a lysed sample of “light” live cells, wherein said light live cells are cells that (i) stably or transiently express the labelled POI, (ii) were cultured in a culture medium free of heavy isotopes, and (iii) were not contacted with the chemical probe, thereby providing a combined enriched sample; (d3) performing liquid chromatography-tandem mass spectrometry (LC-MS/MS) on the combined enriched sample; and (d4) analyzing the data obtained in step (d3) to determine the identity of one or more proteins that interact with the POI.

In some embodiments, the presently disclosed subject matter provides a kit comprising: (a) a photoactive probe or probe system of the presently disclosed subject matter; and (b) one or more of: a cell culture medium, optionally containing one or more heavy isotopes; a buffer; and a solid support material comprising a binding partner of the detectable moiety, optionally wherein said solid support material comprises streptavidin-coated beads.

Accordingly, it is an object of the presently disclosed subject matter to provide photoactive chemical probes and probe systems and methods of detecting biological interactions.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1A is a schematic drawing showing the general connectivity and composition of an exemplary photoactive chemical probe of the presently disclosed subject matter. The probe includes a target (i.e. protein target) recognition moiety T, a photocleavable moiety P₁, a photoreactive moiety P₂, and a detectable moiety R, wherein the photocleavable moiety P₁ connects the target recognition moiety T to the photoreactive and detectable moieties P₂ and R. Additional linking groups (represented by the lines) can also be included.

FIG. 1B is a schematic drawing showing the generally connectivity and composition of an exemplary photoactive chemical probe system of the presently disclosed subject matter. The system includes at least two separate probe molecules. At the top is a probe catalyst that includes a target recognition moiety T and a photocatalytic group P_{c}. At the bottom is a probe substrate that includes a detectable moiety R and a photoreactive group P₃ that, when the system is irradiated and the probe substrate is in proximity to the probe catalyst, can undergo a reaction catalyzed by P_{c}. Additional linking groups (represented by the lines) can also be included.

FIG. 2A is a schematic drawing showing the chemical structure of an exemplary chemical probe of the presently disclosed subject matter referred to as photoproximity probe 1 (PP1). The triangle represents the target recognition moiety T, the star represents the photoreactive moiety P₂, and the oval represents the detectable moiety R. Probe PP1 also includes a nitroveratryl group as photocleavable moiety P₁.

FIG. 2B is a schematic drawing showing steps of an exemplary method of detecting protein-protein interactions using photoproximity probe 1 (PP1) shown in FIG. 2A.

FIG. 3A is a composite image of a fluorescence gel of a recombinant SNAP protein labeled with a model photoproximity probe, PF-BnG, exposed to ultraviolet (UV) light for the time indicated at the top of the gel in vitro.

FIG. 3B is a composite image of a Western blot of total and biotin-labeled SNAP-FLAG protein expressed in human embryonic kidney cells (HEK293T cells) and treated with the indicated amount of the exemplary photoproximity probe 1 (PP1, shown in FIG. 2A) for two hours. Cells and lysates were not irradiated prior to gel and Western blot.

FIG. 3C is a graph showing the quantification of SNAP-FLAG protein labeling (as a percentage (%)) and relative human embryonic kidney cell (HEK293T cell) viability (as determined by relative ATP content) at the doses of exemplary photoproximity probe 1 (PP1) indicated in the x-axis. Points and error bars represent the mean and standard error of the mean from two or more biological replicates.

FIG. 4A is a schematic drawing showing a model protein-protein complex formed between SNAP-FLAG and α-FLAG antibody and the theoretical photoproximity labeling of individual proteins, i.e., light chain (LC) and heavy chain (HC).

FIG. 4B is a composite image of the anti-biotin (streptavidin-800) and anti-mouse Western blot analysis of the photoproximity probe 1 (PP1)-labeled SNAP and SNAP-FLAG protein incubated with α-FLAG antibody prior to ultraviolet (UV) irradiation. “SNAP” label represents SNAP-Tag protein without the FLAG epitope.

FIG. 4C is a composite image of anti-biotin (streptavidin-800) and anti-mouse Western blot analysis of photoproximity probe 1 (PP1)- and photoproximity probe 2 (PP2)-labeled SNAP-FLAG/α-FLAG antibody complex with and without ultraviolet (UV) irradiation prior to analysis. Labels for individual proteins are included at appropriate molecular weights: LC—light chain, HC—heavy chain, “SNAP” label represents SNAP-Tag protein without the FLAG epitope.

FIG. 5A is an image showing anti-biotin (streptavidin-800) and anti-FLAG Western blot analysis of PP1 labeled KEAP1-SNAP protein from HEK293T cells treated with the indicated PP1 doses for 2 hr. Cells and lysates were not irradiated prior to gel analysis.

FIG. 5B is a schematic drawing showing an exemplary method of determining protein-protein interactions using an exemplary probe, i.e., photoproximity probe 1 (PP1) and using stable isotope labeling by amino acids in cell culture (SILAC)-labeled cells expressing SNAP-KEAP1 constructs. Both bulk and anti-biotin enriched proteome profiles are integrated to identify KEAP1 binders in cells.

FIG. 5C is a volcano plot graph of the bulk protein abundance SILAC ratios and P-values for both SNAP-KEAP1 and KEAP1-SNAP expressing cells treated with photoproximity probe 1 (PP1) probe shown in FIG. 2A.

FIG. 5D is a volcano plot graph of streptavidin-enriched protein SILAC ratios and BH-corrected P-values for both SNAP-KEAP1 and KEAP1-SNAP expressing cells treated with the exemplary photoproximity probe 1 (PP1) probe shown in FIG. 2A, irradiated, and enriched using streptavidin beads (SA beads) prior to on-bead trypsinolysis and liquid chromatograph-tandem mass spectrometry (LC-MS/MS) analysis (heavy cells).

FIG. 6 is a schematic drawing showing the chemical structures of exemplary photoproximity probes of the presently disclosed subject matter used in the Examples described hereinbelow.

FIG. 7 is a schematic drawing showing the C-terminal (KEAP1-SNAP) and N-terminal (SNAP-KEAP1) genetic fusions used to study photoproximity profiling of KEAP1 in cells. GₓS represents a glycine-serine spacer, with X indicating the number of glycines.

FIG. 8A is a schematic diagram showing the metascape network analysis of protein ontology categories in the KEAP1-P3-enriched profile.

FIG. 8B is a list of the significantly enriched proteins used to develop the network analysis shown in FIG. 8A.

FIG. 9A is a composite image showing the validation of a novel KEAP1 interacting protein-hexokinase 2 (HK2). The images show anti-FLAG immunoprecipitation of FLAG-SNAP protein (control) and FLAT-KEAP1 protein after bead pulldown, washing elute complexes with 3×FLAG peptide, and Western blot detection of co-immunoprecipitation partners.

FIG. 9B is a schematic drawing of a model of KEAP1 localization to the mitochondrial membrane, with either direct contact to hexokinase 2 (HK2), which is also known to localize to the mitochondrial surface, or interaction through other protein mediators. The model suggests a possible dual metabolic sensing function at the mitochondrial surface.

FIG. 10 is a volcano plot graph for detecting altered interactions in response to dynamic cellular stimuli. The plot shows similar interaction partners and several enriched interacting partners in response to CBR-470-1 pretreatment (10 micromolar (μM) for 14 hours) of SNAP-KEAP1 and KEAP1-SNAP expressing 293T cells treated with the exemplary photoproximity probe 2 (PP2) probe (15 μM), irradiated, and enriched using streptavidin beads (SA beads), lysed, and analyzed via liquid chromatography-tandem mass spectrometry (LC-MS/MS) analysis. PGAM5=serine/threonine-protein phosphatase, mitochondrial.

FIG. 11A is a graph showing the cleavage of diazirine (0.2 millimolar (mM)) in methanol at 4° C. versus time (in seconds (see)). The appearance of products was monitored via liquid chromatography-mass spectrometry (LC/MS) in response to 365 nanometer (nm) light to determine the relative kinetics of diazirine reaction in response to light. Half-life (t½)=55.8 seconds.

FIG. 11B is a graph showing the cleavage of nitroveratryl (0.2 millimolar (mM)) in methanol at 4° C. versus time (in seconds (sec)). The disappearance of starting material was monitored via liquid chromatography-mass spectrometry (LC/MS) in response to 365 nanometer (nm) light to determine the relative kinetics of nitroveratryl reaction in response to light. Half-life (t½)=45.2 seconds.

FIG. 12A is a schematic drawing showing the chemical structure of an exemplary photoreactive photoproximity probe (referred to herein as AC1) of the presently disclosed subject matter.

FIG. 12B is a composite image of anti-biotin (streptavidin-800) and anti-mouse Western blot analysis of photoproximity probe AC1-labeled SNAP-FLAG/α-FLAG antibody complex with (+UV) and without (−UV) ultraviolet (UV) irradiation prior to analysis. Labels for individual proteins are included at appropriate molecular weights: LC—light chain, HC—heavy chain.

FIG. 12C is a graph showing the results of a cell viability test of HEK293T cells treated with the indicated AC1 doses (0 to 50 micromolar (μM) for 2 hours. Cell viability is reported as relative cell viability compared to untreated cells.

FIG. 12D is a composite image showing anti-biotin (streptavidin-800) and anti-FLAG Western blot analysis of AC1-labeled KEAP1-SNAP protein from HEK293T cells treated with the indicated AC1 doses (0, 5, 15, 0r 50 micromolar (μM) for 1 hour (left) or 2 hours (right). Cells and lysates were not irradiated prior to gel analysis.

FIG. 12E is a graph showing the normalized fluorescence intensity from the Western blot analysis shown in FIG. 12D of the AC1-labeled KEAP1-SNAP protein from HEK293T cells treated with AC1 at 0 to 50 micromolar (μM) doses for one hour.

FIG. 12F is a graph showing the normalized fluorescence intensity from the Western blot analysis shown in FIG. 12D of the AC1-labeled KEAP1-SNAP protein from HEK293T cells treated with AC1 at 0 to 50 micromolar (μM) doses for two hours.

FIG. 12G is a composite image showing anti-biotin (streptavidin-800) and anti-FLAG Western blot analysis of AC1-labeled KEAP1-SNAP protein from HEK293T cells treated with the indicated AC1 doses (0, 0.5, 1, 5, 10, 20, 30, or 50 micromolar (μM) for 1 hour. Cells and lysates were not irradiated prior to gel analysis.

FIG. 13A is a schematic drawing showing the chemical structure of a model probe compound (referred to herein as AC-M3) that contains an isopropyl-substituted nitroveratryl group.

FIG. 13B is a graph showing the photodegradation (normalized intensity of the starting material versus time (in seconds (sec)) of the model probe compound shown in FIG. 13A. n=3.

FIG. 14 a schematic drawing showing steps of an exemplary method of detecting protein-protein interactions using a catalytic photoproximity probe system of the presently disclosed subject matter. The catalytic photoproximity probe system includes a probe molecule comprising a photocatalytic group (e.g., a flavin derivative, oval) and a binding moiety (triangle) that can interact with a binding partner of a labelled protein of interest (POI). Upon irradiation, the photocatalytic group can catalyze a reaction of a probe substrate (e.g., a biotin-phenol probe substrate) that comprises a group (e.g., a phenol, shown as a hexagon) that can undergo a photocatalyzed reaction to from a group (e.g., a phenoxy radical) that can covalently bond to molecules (e.g., ProtX and ProtY) in proximity to the POI and a detectable moiety (e.g., a biotin, shown as a star). One probe molecule can catalyze the reaction of multiple probe substrates, resulting in an increased number of labelled molecules in proximity to a POI.

FIG. 15A is a schematic drawing showing the chemical structure of an exemplary photocatalytic probe molecule of the presently disclosed subject matter, referred to herein as FBG or FBG-1, comprising a benzylguanine moiety attached via a linker to a flavin derivative.

FIG. 15B is a schematic drawing of two exemplary probe substrates for the photocatalytic probe molecule shown in FIG. 15A. Both probe substrates include a phenol moiety. The probe substrate at the top includes an alkyne that can be further elaborated to form a detectable group, while the probe substrate at the bottom include a biotin moiety and is referred to herein as biotin-phenol (BP) or phenyl-biotin probe (PBP)

FIG. 16A is a schematic drawing of a model photocatalytic probe system used in proof-of-concept studies described in the Examples. The model system includes the phenol biotin probe substrate (PBP) shown in FIG. 15B and flavin carboxylic acid (FC) as a model of the catalytic probe.

FIG. 16B is a graph showing the high-performance liquid chromatograph (HPLC) analysis of the photocatalytic activity of the probe system shown in FIG. 16A, n=1. In the absence of light (−hν) or of light (−hν) and catalyst (−FC), no change occurs to the HPLC trace of the phenol biotin probe substrate. However, when all three of light (+hν), catalyst (+FC) and probe substrate (+BPB) are present/used, the peak corresponding to BPB disappears and two new peaks appear, indicating the oxidation of the probe substrate.

FIG. 16C is a graph showing the high-performance liquid chromatograph (HPLC) analysis of the photocatalytic activity of the probe system shown in FIG. 16A, n=2. In the absence of light (−hν) or of light (−hν) and catalyst (−FC), no change occurs to the HPLC trace of the phenol biotin probe substrate. However, when all three of light (+hν), catalyst (+FC) and probe substrate (+BPB) are present/used, the peak corresponding to BPB disappears and two new peaks appear, indicating the oxidation of the probe substrate.

FIG. 17A is a graph showing the high-performance liquid chromatograph (HPLC) analysis of the photocatalytic activity of the probe system comprising the phenol biotin probe substrate (PBP) shown in FIGS. 16A and 15B and the benzylguanine-derivatized flavin probe catalyst (FBG-1) shown in FIG. 15A, n=1. In the absence of one or both of light (−UV) or catalyst (−FP), no change occurs to the HPLC trace of the phenol biotin probe substrate. However, when all three of light (+UV), catalyst (+FB) and probe substrate (+BPB) are present/used, the peak corresponding to BPB is reduced and new peaks appear, corresponding to the formation of a dimer of BPB.

FIG. 17B is a schematic drawing showing the chemical structure of the dimer of BPB. The molecular weight of the dimer is 724.94 daltons.

FIG. 18 is an image of a fluorescence assay showing the in vitro labeling of bovine serum albumin (BSA) using flavin carboxylic acid (FCA) as a probe catalyst and biotin-phenol (BP) as a probe substrate. Biotinylation (as indicated by the fluorescence in the lane on the right, from the binding of an anti-biotin antibody) only occurs when light, FCA, and BP are all used (+UV, +FCA, +BP). BP does not label BSA in the presence of BP alone (−UV, −FCA, +BP) or in the presence of light when the catalyst is absent (+UV, −FCA, +BP). Fluorescence in the lane on the left is from labeling with an anti-mouse control antibody.

FIG. 19 is an image of a fluorescence assay showing the in vitro labeling of bovine serum albumin (BSA) using the benzylguanine-derivatized flavin probe catalyst (FBG) as a probe catalyst and biotin-phenol (BP) as a probe substrate. Biotinylation (as indicated by the fluorescence in the lane on the right, from the binding of an anti-biotin antibody) only occurs when light. FBG and BP are all used (+UV, +FBG, +BP). BP does not label BSA in the presence of BP alone (−UV, −FGB, +BP) or in the presence of light when the catalyst is absent (+UV, −FBG, +BP). Fluorescence in the lane on the left is from labeling with an anti-mouse control antibody.

FIG. 20 is a composite image of anti-biotin (streptavidin-800) and anti-mouse Western blot analysis of the SNAP-FLAG/α-FLAG antibody complex with (+FBG1) or without (−FBG1) benzylguanine-derivatized flavin catalyst (FBG1), with (+PBP1) or without (−PBP1) phenyl biotin probe substrate (PBP1) and with (+UV) and without (−UV) ultraviolet (UV) irradiation prior to analysis. In the presence of both FBG1 and PBP1 irradiated samples show biotin labeling of the α-FLAB antibody. Labels for individual proteins are included at appropriate molecular weights: LC—light chain, HC—heavy chain.

FIG. 21 is a composite image of anti-biotin (streptavidin-800) and anti-mouse Western blot analysis of the SNAP-FLAG/α-FLAG antibody complex with (+FBG1) or without (−FBG1) benzylguanine-derivatized flavin catalyst (FBG1), with (+PBP1) or without (−PBP1) phenyl biotin probe substrate (PBP1) and with (+UV) and without (−UV) ultraviolet (UV) irradiation prior to analysis. For comparison, some samples of the same complex were exposed to an exemplary noncatalytic probe (+AC1). Labels for individual proteins are included at appropriate molecular weights: LC—light chain, HC—heavy chain.

FIG. 22A is an image of a fluorescence gel showing the results of an in vitro SNAP-labelling competition assay in HEK293T cells expressing KEAP1-SNAP. The cells were exposed to varying concentrations (0, 1, 2, 5, 10, 15, 25, 50, or 75 micromolar (μM) of benzylguanine-derivatized flavin catalyst (FBG1, “Flavin-BG”) for two hours and lysed in the presence of 20 μM fluorescein isothiocyanate (FITC)-labeled flavin.

FIG. 22B is a graph of the data (normalized intensity of fluorescein isothiocyanate-labeled flavin versus benzylguanine-derivatives flavin catalyst probe concentration (in micromolar (μM)) from the assay described for FIG. 22A.

FIG. 23 is a graph of a cell viability assay (measured as normalized luminescence intensity) of HEK293T cells stably expressing SNAP-Flag exposed to varying concentrations (0 to 50 micromolar (μM)) of benzylguanine-derivatized flavin probe catalyst (FBG-1).

FIG. 24 is a composite image of the gel analysis of an in situ photolabeling study of the presently disclosed photocatalytic probe system in live cells. HEK293T cells stably expressing KEAP1-SNAP were treated with (+FBG) or without (−FBG) a benzylguanine-derivatized flavin catalyst probe, with (+BP) or without (−BP) a biotin-phenol probe substrate, and with (+UV) or without (−UV) light.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

Throughout the specification and claims, a given chemical formula or name shall encompass all optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising.” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of size, temperature, time, weight, volume, concentration, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.10/% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

As used herein the term “alkyl” can refer to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and alkenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms or having up to about 5 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds.

The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc.) in the backbone of a ring structure. Nitrogen-containing heteroaryl moieties include, but are not limited to, pyridine, imidazole, benzimidazole, pyrazole, pyrazine, triazine, pyrimidine, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Arylene” refers to a bivalent aromatic group.

“Aralkylene” refers to a bivalent group including both arylene and alkylene moieties.

As used herein, the term “acyl” refers to a carboxylic acid group wherein the —OH of the carboxylic acid group has been replaced with another substituent. Thus, an acyl group can be represented by RC(═O)—, wherein R is an H, alkyl, substituted alkyl, aralkyl, substituted aralkyl, aryl, or substituted aryl group as defined herein. Specific examples of acyl groups include formyl (i.e., —C(═O)H), acetyl, and benzoyl.

The term “keto” as used herein refers to the group R—C(═O)—, wherein R is alkyl, substituted alkyl, aralkyl, substituted aralkyl, aryl, and substituted aryl.

“Aralkyl” refers to an aryl-alkyl-group wherein aryl and alkyl are as previously described and can include substituted aryl and substituted alkyl. Exemplary aralkyl groups include benzyl, phenylethyl, and naphthylmethyl.

The term “thioether” refers to the —SR group, wherein R is alkyl, substituted alkyl, aralkyl, substituted aralkyl, aryl or substituted aryl.

The term “thioester” refers to the —S—C(═O)R group, wherein R is alkyl, substituted alkyl, aralkyl, substituted aralkyl, aryl, or substituted aryl.

The terms “hydroxyl” and “hydroxyl” refer to the —OH group.

The term “phenol” as used herein can refer to a compound of the formula R—OH group, wherein R is aryl or substituted aryl.

The term “phenolic” refers to a hydroxyl group that is directly attached to an aromatic group, e.g., a phenyl ring, a napthyl ring, etc.

The terms “mercapto” or “thiol” refer to the —SH group.

The term “carboxyl” refers to the —C(═O)— group.

The terms “carboxylate” and “carboxylic acid” can refer to the groups —C(═O)O⁻ and —C(═O)OH, respectively. In some embodiments, “carboxylate” can refer to either the —C(═O)⁻ or —C(═O)OH group.

The terms “amide” and “amido” refer to the group —C—C(═O)—NR₁R₂, wherein R₁ and R₂ are independently H, alkyl, substituted alkyl, aralkyl, substituted aralkyl, aryl or substituted aryl.

The term “carbamate” refers to the group —O—C(═O)—NR—, wherein R is H, alkyl, substituted alkyl, aralkyl, substituted aralkyl, aryl, or substituted aryl.

The term “aliphatic” as used herein refers to a hydrocarbon compound or moiety that is not aromatic. The compound or moiety can be saturated or partially or fully unsaturated (i.e., can include alkenyl and/or alkynyl groups). In some embodiments, the term “aliphatic” refers to a chemical moiety wherein the main chain of the chemical moiety does not comprise an arylene group.

A line crossed or terminated by a wavy line, e.g., in the structure:

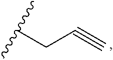 [SMILES: *CC#C]

indicates the site where a chemical moiety can bond to another group.

The term “peptide” as used herein refers to a polymer of amino acid residues, wherein the polymer can optionally further contain a moiety or moieties that do not consist of amino acid residues (e.g., an alkyl group, an aralkyl group, an aryl group, a protecting group, or a synthetic polymer, such as, but not limited to a biocompatible polymer). The term applies to amino acid polymers in which one or more amino acid residue is an artificial chemical mimetic of a corresponding naturally occurring amino acid, as well as to naturally occurring amino acid polymers and non-naturally occurring amino acid polymer.

The terms “peptidyl” and “peptidyl moiety” refer to a monovalent peptide or peptide derivative (e.g., a peptide comprising one or more terminal or side chain protecting or other moieties to mask a reactive functional group).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs are compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics are chemical compounds that have a structure that is different from the general chemical structure of an amino acid, but that functions in a manner similar to a naturally occurring amino acid.

The term “amino acid residue” as used herein refers to a monovalent amino acid or derivative thereof. In some embodiments, the term “amino acid residue” refers to the group —NHC(R′)C(═O)OR″, wherein R′ is an amino acid side chain or protected derivative thereof and wherein R″ is H or a carboxylic acid protecting group, e.g., methyl.

An “affinity” label is a moiety that can specifically bind to its molecular binding partner. The binding can be through covalent or non-covalent (e.g., ionic, hydrogen, etc.) bonds. Thus, an affinity label refers to a moiety that can be used to isolate or purify the affinity label and compositions to which it is bound, from a complex mixture. One example of an affinity label is a member of a specific binding pair (e.g., biotin:avidin, antibody:antigen). In some embodiments, an affinity label, such as biotin, can selectively bind to an affinity matrix, such as streptavidin-coated beads or particles.

In some embodiments, the affinity label is a peptide tag. In some embodiments, the affinity label is a covalent peptide tag (i.e., a peptide tag that is covalently attached to the labeled moiety). In some embodiments, the affinity label is a protein tag. Other affinity tags include, but are not limited to, chitin binding protein-tag, maltose binding protein-tag, glutathione-S-transferase-tag, polyhistidine (His-tag), FLAG-tag, V5 tag, VSV-tag, Myc-tag, c-Myc-tag, HA-tag, E-tag, S-tag, SBP-tag, Softag 1, Softag 3, Strep-tag, TC tag, calmodulin-tag, Avi-tag, Xpress tag, isopeptag, Spy-tag, biotin carboxyl carrier protein (BCCP), green fluorescent protein-tag, HaloT-tag, Nus-tag, Fc-tag, Ty tag, thioredoxin-tag, or poly(NANP). In some embodiments, the affinity label is biotin or desthiobiotin. In some embodiments, the affinity label is selected from the group consisting of: biotin or an analogue thereof; digoxigenin; fluorescein; dinitrophenol; and an immunotag.

As used herein the term “selectively hybridizing”, and grammatical variations thereof, refers to the associative interaction between two complementary nucleic acid sequences. In some embodiments, selective hybridization refers to the association of two nucleic acids that are hybridize under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., greater than about 30° C., or greater than about 37° C. Longer DNA fragments can require higher hybridization temperatures for specific hybridization. Since the stringency of hybridization can be affected by other factors such as probe composition, the presence of organic solvents and the extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context. In some embodiments, intact protein molecules can be ionized by the above techniques, and then introduced to a mass analyzer. Alternatively, protein molecules can be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers can be employed in the methods of the presently disclosed subject matter. For example, whole protein mass spectroscopy analysis can be conducted using time-of-flight (TOF) or Fourier transform ion cyclotron resonance (FT-ICR) instruments. For peptide mass analysis, MALDI time-of-flight instruments can be employed, as they permit the acquisition of peptide mass fingerprints (PMFs) at high pace. Multiple stage quadrupole-time-of-flight and the quadrupole ion trap instruments can also be used.

The terms “high throughput protein identification,” “proteomics” and other related terms are used herein to refer to the processes of identification of a large number or (in some cases, all) proteins in a certain protein complement. Post-translational protein modifications and quantitative information can also be assessed by such methods. One example of “high throughput protein identification” is a gel-based process that includes the pre-fractionation and purification of proteins by one-dimensional protein gel electrophoresis. The gel can then be fractionated into several molecular weight fractions to reduce sample complexity, and proteins can be in-gel digested with trypsin. The tryptic peptides are extracted from the gel, further fractionated by liquid chromatography and analyzed by mass spectrometry. In another approach, a sample can be fractionated without using the gels, for example, by protein extraction followed by liquid chromatography. The proteins can then be digested in-solution, and the proteolytic fragments further fractionated by liquid chromatography and analyzed by mass spectrometry.

As used herein, the term “Western blot,” which can be also referred to as “immunoblot”, and related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

The expression “stable isotope labeling by amino acids in cell culture” (SILAC) is used herein to refer to an approach for incorporation of a label into proteins for mass spectrometry (MS)-based quantitative proteomics. SILAC comprises metabolic incorporation of a given “light” or “heavy” form of the amino acid into the proteins. For example, SILAC comprises the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

### II. Photoproximity Profiling Probes and Probe Systems

In some embodiments, the presently disclosed subject matter provides a photoactive chemical probe or probe system for use in photoproximity profiling of biological interactions, e.g., protein-protein interactions, protein-metabolite interactions, protein-drug interactions, cell-cell interactions, nucleic acid-drug interactions, etc. The probe or probe system can be used to profile such interactions in or on live cells, thereby eliminating biases against low concentration biological entities, weak interaction affinities, and false-positive interactions caused when studying biological interactions in lysed cells.

Generally, the presently disclosed photoactive probes or probe systems include, as parts of a single probe molecule or as parts of more than one different probe molecules (e.g., two different probe molecules) designed to be used in combination, a target recognition moiety capable of specifically binding (covalently or non-covalently) a first binding partner, a detectable moiety or a precursor thereof, and at least two photoactive moieties. The target recognition moiety can be a moiety that specifically binds a moiety associated with a biological target of interest (BTOI). For example, the target recognition moiety can be capable of binding a peptide or protein tag attached to a BTOI, such as a protein of interest (POI). Alternatively, the target recognition moiety can be capable of specifically binding a moiety naturally present as part of the BTOI. For example, the target recognition moiety can be a nucleic acid or nucleic acid analog capable of binding (e.g., selectively hybridizing) a sequence in a nucleic acid of interest (NAOI). In some embodiments, the target recognition moiety is a nucleic acid, a nucleic acid analog, a peptide, or a peptide analog that is capable of specific binding to a small molecule BTOI, such as a drug or drug metabolite. In some embodiments, the target recognition moiety does not directly interact with the BTOI, but instead is capable of binding to an entity known to be in a proximal network to the BTOI. For example, the target recognition moiety could specifically bind to a protein (e.g., a receptor or enzyme) known to interact with a drug or metabolite of interest.

In some embodiments, the detectable moiety is a moiety that is capable of bonding to a second binding partner (e.g., which is different than the first binding partner that the target recognition moiety binds to). The detectable moiety can also be referred to as an “affinity identification handle” or a “recognition handle.” In some embodiments, the detectable moiety is a group that can undergo one or more chemical transformations to form a moiety that is capable of bonding to a second binding partner. In some embodiments, one of said photoactive moieties is a photocleavable moiety or a photocatalytic moiety. In some embodiments, at least one of the photoactive moieties is a photoreactive moiety capable of forming a covalent or non-covalent bond with another molecule (e.g., a protein, a drug metabolite, a lipid, etc.) when activated by light or upon undergoing a reaction or activation catalyzed by a photocatalytic moiety in the presence of light. This photoactive moiety can also be referred to as a “photoaffinity moiety” or a “photocapture group.”

II.A. Photocleavable Probes

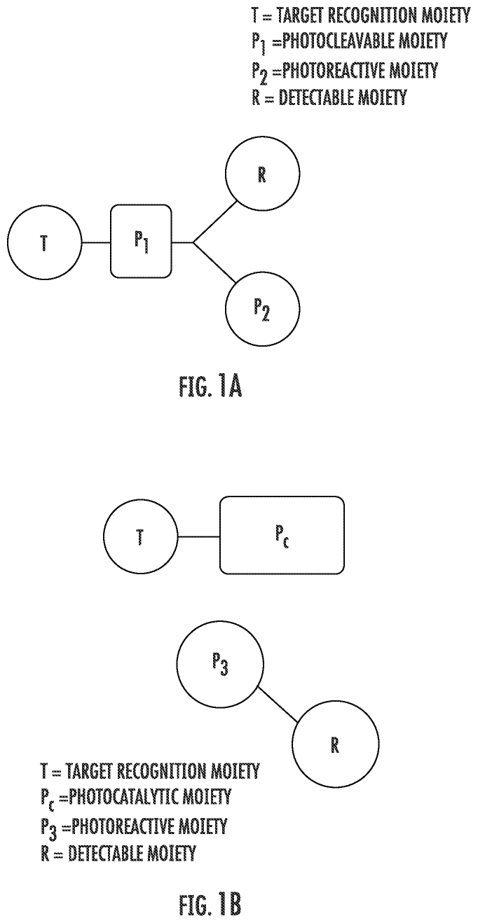

In some embodiments, the presently disclosed subject matter provides a photoactive probe comprising a photocleavable group. FIG. 1A shows a schematic drawing showing the main components of photoproximity probes that include a photocleavable group. More particularly, in some embodiments, the presently disclosed probe includes a target recognition moiety T that is capable of specifically binding a modular first binding partner protein in cells; a photocleavable moiety P₁, a photoreactive moiety P₂, and a detectable moiety R capable of specifically binding a second binding partner. Photoreactive moiety P₂ can act as a “photoaffinity moiety” or a “photocapture group.” Detectable moiety R can act as a “affinity identification handle” or a “recognition handle”. When the probe is contacted with a sample comprising a biological target of interest (e.g., a labelled BTOI, such as a BTOI genetically labeled with a first binding partner, such as a protein or peptide tag), the target recognition moiety specifically binds to the BTOI (i.e., the label on the BTOI or other moiety already present on the BTOI that forms the first binding partner). In some embodiments, the interaction between the recognition moiety and the first binding partner is covalent in nature, such that the photoproximity probe is irreversibly localized to the BTOI in or on live cells. In some embodiments, the interaction between the recognition moiety and the first binding partner is non-covalent, resulting in reversible localization of the photoproximity probe to the BTOI. When the cellular sample is exposed to light (e.g., 365 nm light), photocleavable moiety P₁ undergoes a photochemical cleavage reaction, resulting in diffusion of the photoreactive moiety P₂ and the detectable moiety R (which are still attached to one another) from the BTOI. Simultaneously, the photoreactive moiety P₂ is activated or unmasked via exposure to the light and can covalently react with or bond to an entity (e.g., a small molecule such as a drug or metabolite, a nucleic acid, a peptide, a protein, or a cell) near the BTOI, within a diffusion radius related to the probe (e.g., the rate of cleavage of P₁ and the reactivity of P₂). In some embodiments, P₁ and P₂ can exhibit similar cleavage/activation rates, resulting in simultaneous cleavage and covalent labeling of proteins or biomolecules in the radius of the BTOI.

All portions of the probe are modular, providing for a combinatorial library of probes that can study a variety of interactions of diverse BTOIs. The mode of BTOI targeting of the probe can be varied to include both reversible and irreversible binding to the BTOI. As noted above, the particular combination of photocleavable and photoreactive groups can provide tuning of the labeling radius of the probe, responsive light wavelengths, and the timing of the probe-detectable interactions. The combination of modular elements can retain activity of each individual element, as well as suitable pharmacologic properties like aqueous solubility and cell permeability.

The simultaneous cleavage and unmasking of P₁ and P₂ provides exquisite spatiotemporal control for proximity profiling using the presently disclosed probes, e.g., as no secondary reagent or endogenous co-factors are needed to label entities in proximity of a BTOI. In particular, the lack of the use of a peroxide-based secondary reagent provides for the use of the probes to study redox-related interactions in biological systems. Further, the presently disclosed probes are amenable for use in all cell compartments.

Thus, in some embodiments, the presently disclosed subject matter provides a chemical probe having a structure of Formula (I):

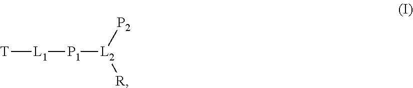

wherein: T is a target recognition moiety capable of specifically binding a first binding partner; L₁ is a bivalent linker; P₁ is a photocleavable moiety; L₂ is a trivalent linker moiety; P₂ is a photoreactive moiety; and R is a detectable moiety or a precursor thereof capable of specifically binding a second binding partner; subject to the proviso that the first and second binding partner are different. Stated another way, if R is a moiety capable of specifically binding to biotin, then T is a moiety that specifically binds a binding partner other than biotin.

R can be any suitable group that selectively binds to a binding partner, such as any suitable affinity label known in the art. In some embodiments, R can be a monovalent moiety derived from a small molecule, a peptide (e.g., an antigenic peptide), a peptide analog (e.g., a peptoid), or a nucleic acid or nucleic acid analog. In some embodiments, R can comprise biotin, a biotin analog (e.g., desthiobiotin) or a precursor thereof (e.g., an alkyne that can react with a biotin-azide reagent via Cu-catalyzed Click cycloaddition). In some embodiments, R is selected from:

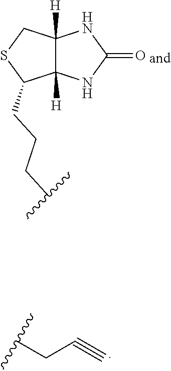

Target recognition moiety T can also be any suitable group that selectively binds to a binding partner, so long as it selectively binds to a different binding partner than the R moiety used in the same probe. In some embodiments, T is other than a protein or peptidyl moiety. For example, in some embodiments, T is a monovalent derivative of a small molecule or a nucleic acid sequence. In some embodiments, T is suitable for selective binding to a protein or peptide tag. In some embodiments, T comprises a moiety selected from the group comprising a benzylguanine group (which can specifically bind to MGMT-fusion or “SNAP-tag” fusion proteins), a chloroalkane group (which can specifically bind to “Halo-Tag” fusion proteins), a benzylcytosine group (which can specifically bind to “Clip-Tag” fusion proteins), an azide (which can specifically bind to a receptor engineered with a strained cyclooctyne or other group for [3+2] Huisgen chemistry), biotin or a biotin analog (e.g., desthiobiotin)(which can specifically bind to monomeric streptavidin, neutravidin, or avidin fusion proteins and can be used as long as R does not specifically bind to streptavidin, neutravidin, or avidin as well) AP1867 or an orthogonal FK506 analog (i.e., a tacrolimus analog) (which can specifically bind to FK binding protein (e.g., FKBP12 or mutant FKBP12) fusion proteins) and a methotrexate derivative (which can specifically bind to fusion proteins of wild-type or engineered dihydrofolate reductase (DHFR)). In some embodiments, T is selected from:

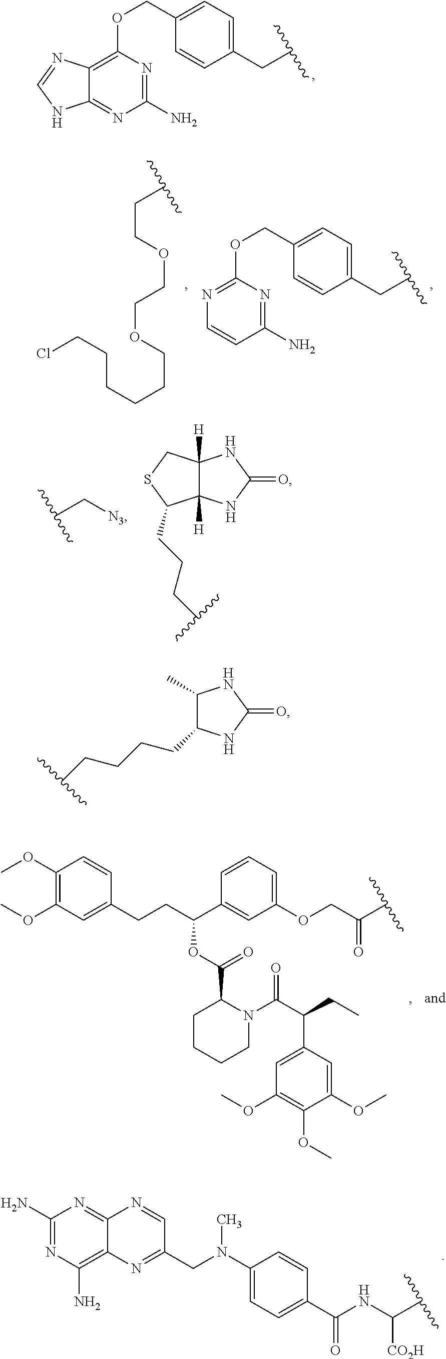

The photoreactive moiety P₂ can be any suitable photoreactive moiety that can be activated by light to form a reactive species capable of bonding (covalently or non-covalently) to a protein, peptide, small molecule, or nucleic acid. In some embodiments, P₂ is activated by light at the same wavelength that causes photocleavage of P₁. In some embodiments. P₂ comprises a group selected from a diazirine derivative, a benzophenone derivative, or an aryl azide derivative. In some embodiments, P₂ is selected from:

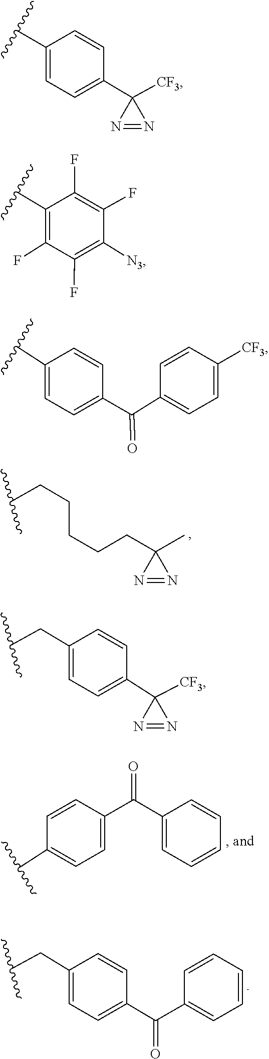

Any suitable bivalent and trivalent linkers can be used as L₁ and L₂. In some embodiments, L₁ includes an amide and/or carbamate group. In some embodiments, L₁ further includes an alkylene group, optionally wherein the alkylene group includes one or more oxygen atoms inserted in the alkylene chain and/or one or more alkyl group substituents. In some embodiments, L₁ is selected from —NH—C(═O)-alkylene-; —NH—C(═O)—O—CH₂CH₂—O—; and —NH—C(═O)—O—CH₂CH₂—NH—C(═O)-alkylene-; wherein alkylene is a C₁-C₆ alkylene. In some embodiments, the alkylene is propylene.

In some embodiments, L₂ can comprise one or more amide, thioamide, thioester, or thioether groups as well as one or more alkylene groups. In some embodiments, L₂ is selected from the group comprising:

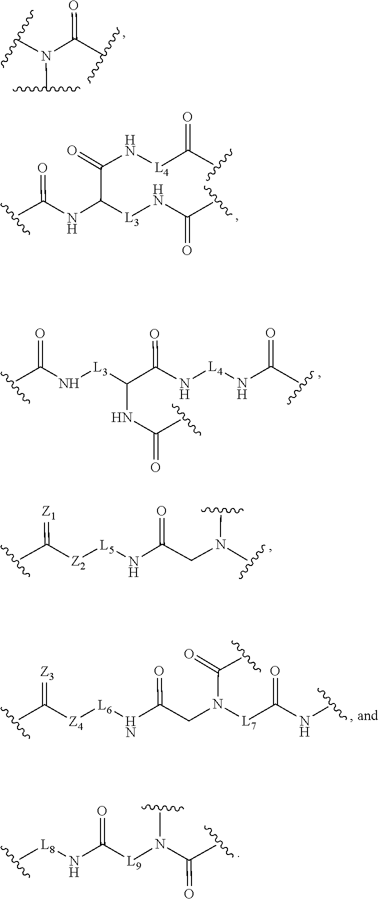

wherein each L₃, L₄, L₅, L₆, L₇, L₈, and L₉ is alkylene, which can be substituted or unsubstituted (e.g., C₁-C₆alkylene); Z₁ and Z₃ are selected from O and S; and Z₂ and Z₄ are selected from O, S, and NH. In some embodiments, one or more oxygen atoms can be inserted along one or more of the alkylene groups thereby forming an ether. In some embodiments, L₂ is selected from:

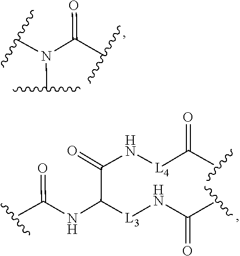

wherein L₃ is butylene and L₄ is pentylene; and

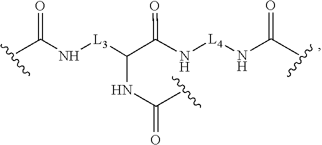

wherein L₃ is butylene and L₄ is ethylene.

P₁ can be any suitable photocleavable group. P₁ can be based, for instance, on moieties used in photocleavable protecting groups known for use in organic synthesis. See Klán et al., Chem. Rev., 2013, 113, 119-191. In some embodiments, P₁ comprises a divalent nitroaryl derivative, a divalent coumarin derivative, or a divalent hydroxyaryl derivative. In some embodiments, the divalent nitroaryl derivative is a divalent ortho-nitrobenzyl derivative, a divalent nitroindoline derivative, or a divalent nitrobenzopiperidine derivative. In some embodiments, the divalent hydroxyaryl derivative is a divalent ortho-hydroxybenzyl derivative or a divalent ortho-hydroxynaphthyl derivative. Thus, in some embodiments, P₁ comprises a divalent ortho-nitrobenzyl derivative, a divalent coumarin derivative, a divalent nitroindoline derivative, a divalent nitrobenzopiperidine derivative, a divalent ortho-hydroxybenzyl derivative, or a divalent ortho-hydroxynaphthyl derivative. Variation of P₁ can be used to tune the balance of the photo reactivity kinetics for cleavage (and diffusion) of the probe versus the activation of the photoaffinity group.

In some embodiments. P₁ is a divalent ortho-nitrobenzyl derivative. e.g., a derivative of nitroveratryl alcohol. In some embodiments, the compound of Formula (I) has a structure of Formula (II):

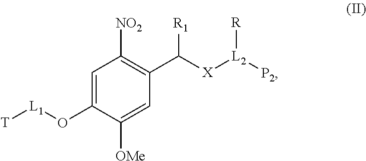

wherein: T, L₁, L₂, R, and P₂ are as defined for the compound of Formula (I); X is selected from O, NR′, and S, wherein R′ is selected from H and alkyl (e.g., C₁-C₆ alkyl); and R₁ is selected from H, alkyl (e.g., C₁-C₆ alkyl), perhaloalkyl (e.g., perfluoralkyl, such as —CF₃), and cyano. In some embodiments, R₁ is selected from H, methyl, isopropyl, —CF₃, and cyano. In some embodiments, the identity of R₁ can affect the rate of photocleavable of the photocleavable moiety. For example, when R₁ is isopropyl, the photocleavable group has a faster rate of cleavage than when R₁ is methyl. In some embodiments, X and L₂ together form a group comprising a carbamate, a urea, a thiourea, an amide, an ester, an ether, an amine, a sulfonamide, or a sulfide (i.e., a thioether).

In some embodiments, the compound is selected from the group comprising:

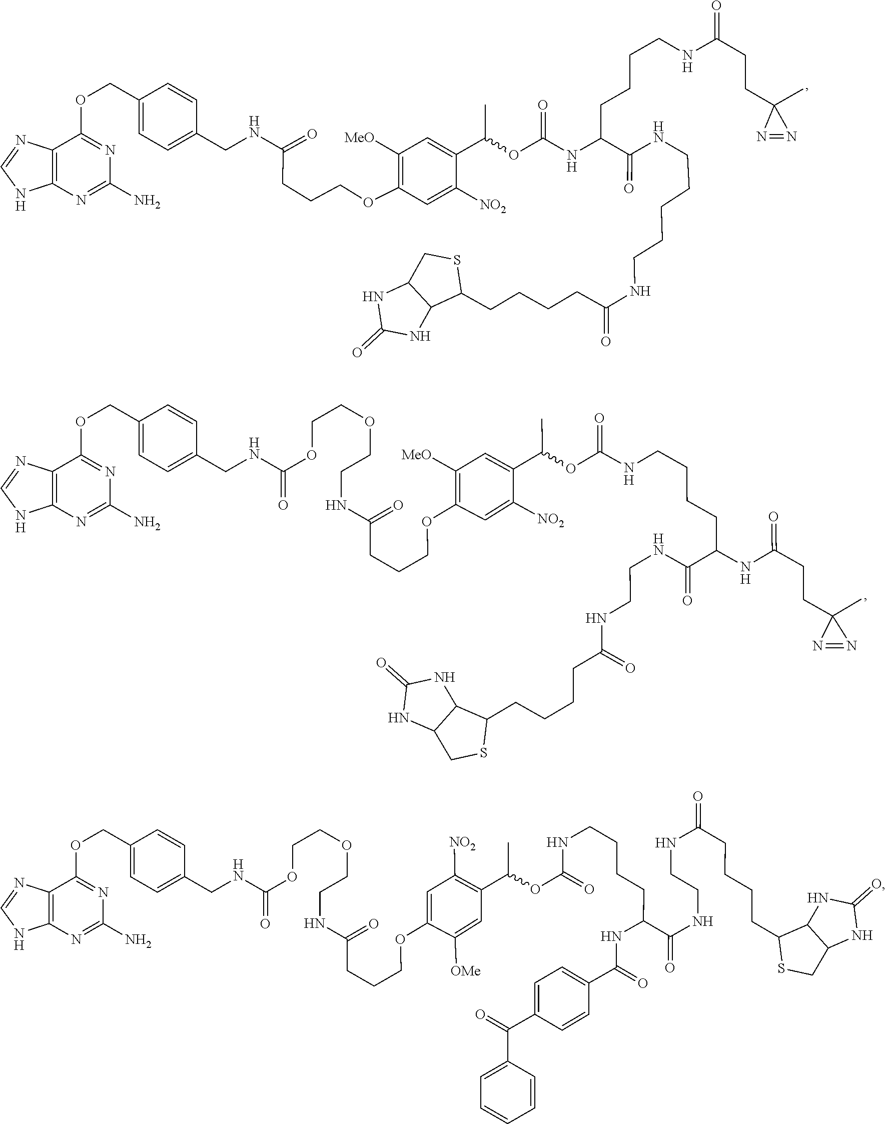 [SMILES: COc1cc(C(C)OC(=O)NC(CCCCNC(=O)CCC2(C)N=N2)C(=O)NCCCCCNC(=O)CCCCC2SCC3NC(=O)NC32)c([N+](=O)[O-])cc1OCCCC(=O)NCc1ccc(COc2nc(N)nc3[nH]cnc23)cc1 ; COc1cc(C(C)OC(=O)NCCCCC(NC(=O)CCC2(C)N=N2)C(=O)NCCNC(=O)CCCCC2SCC3NC(=O)NC32)c([N+](=O)[O-])cc1OCCCC(=O)NCCOCCOC(=O)NCc1ccc(COc2nc(N)nc3[nH]cnc23)cc1 ; COc1cc(C(C)OC(=O)NCCCCC(NC(=O)c2ccc(C(=O)c3ccccc3)cc2)C(=O)NCCNC(=O)CCCCC2SCC3NC(=O)NC32)c([N+](=O)[O-])cc1OCCCC(=O)NCCOCCOC(=O)NCc1ccc(COc2nc(N)nc3[nH]cnc23)cc1]

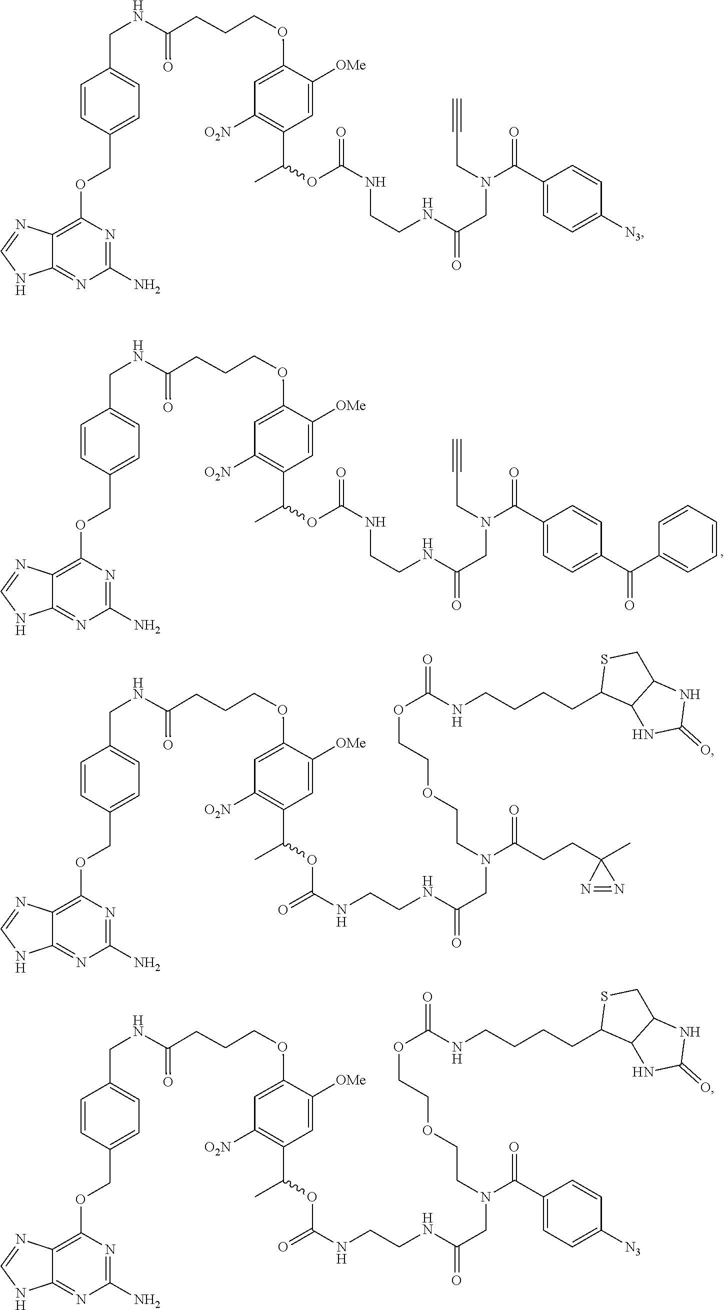

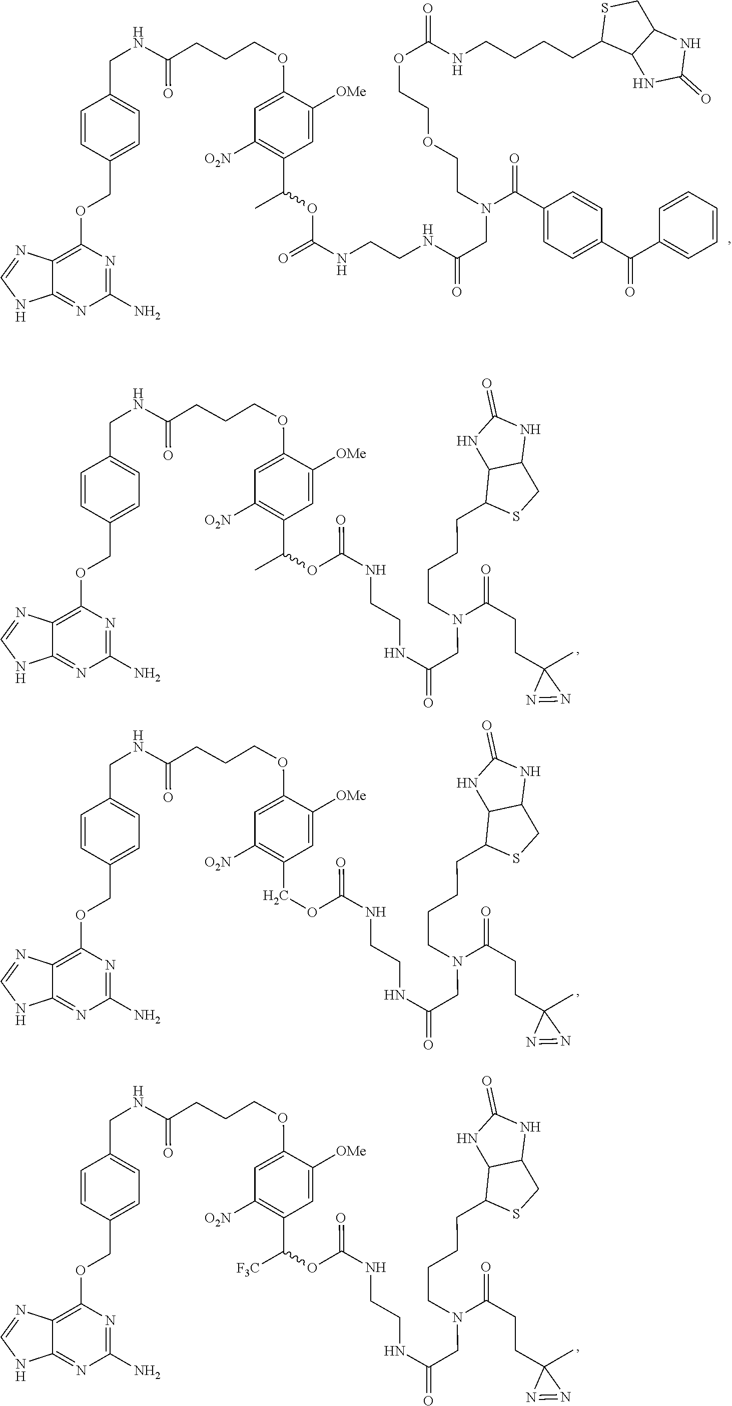

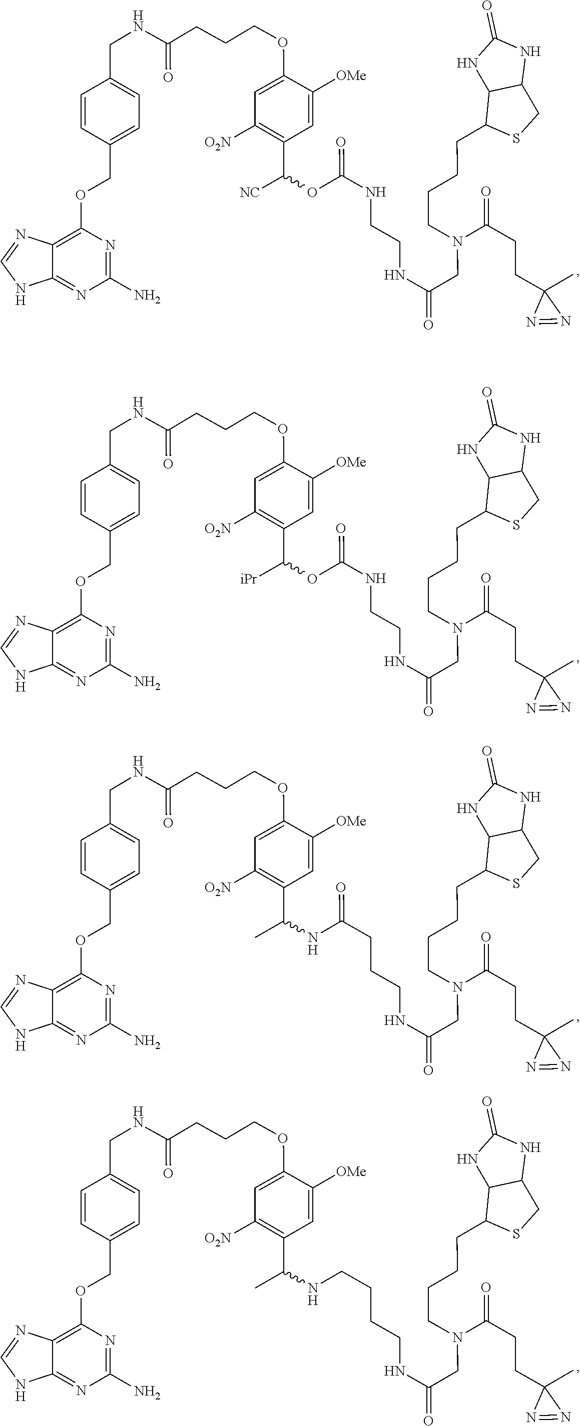

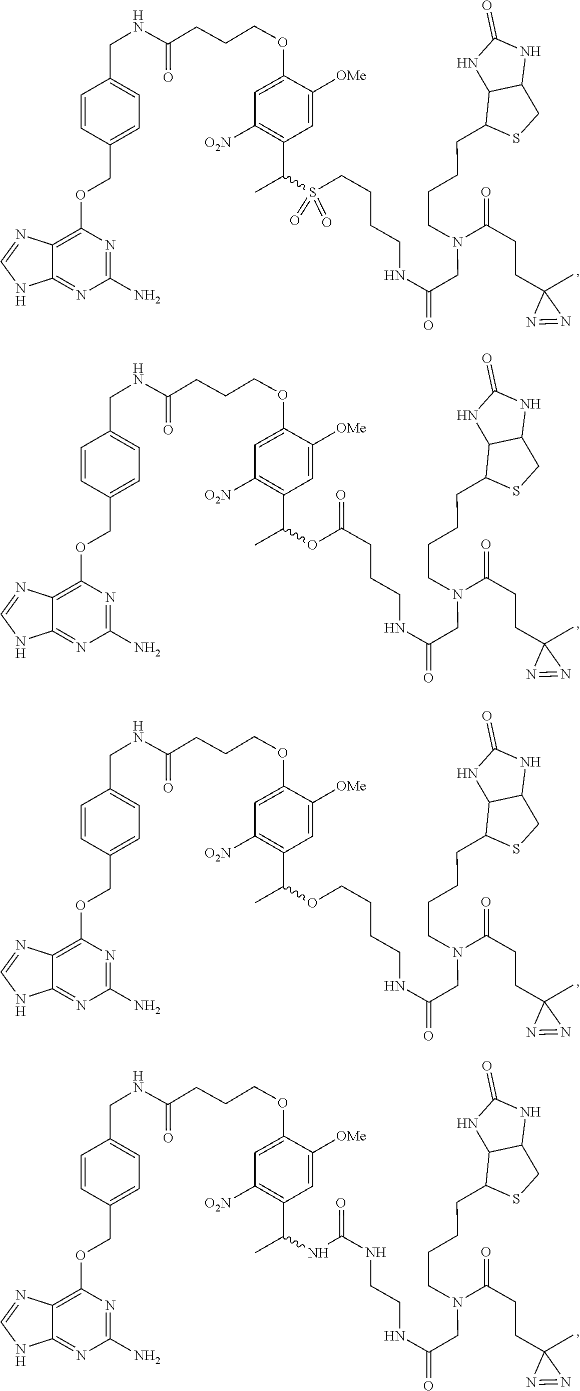

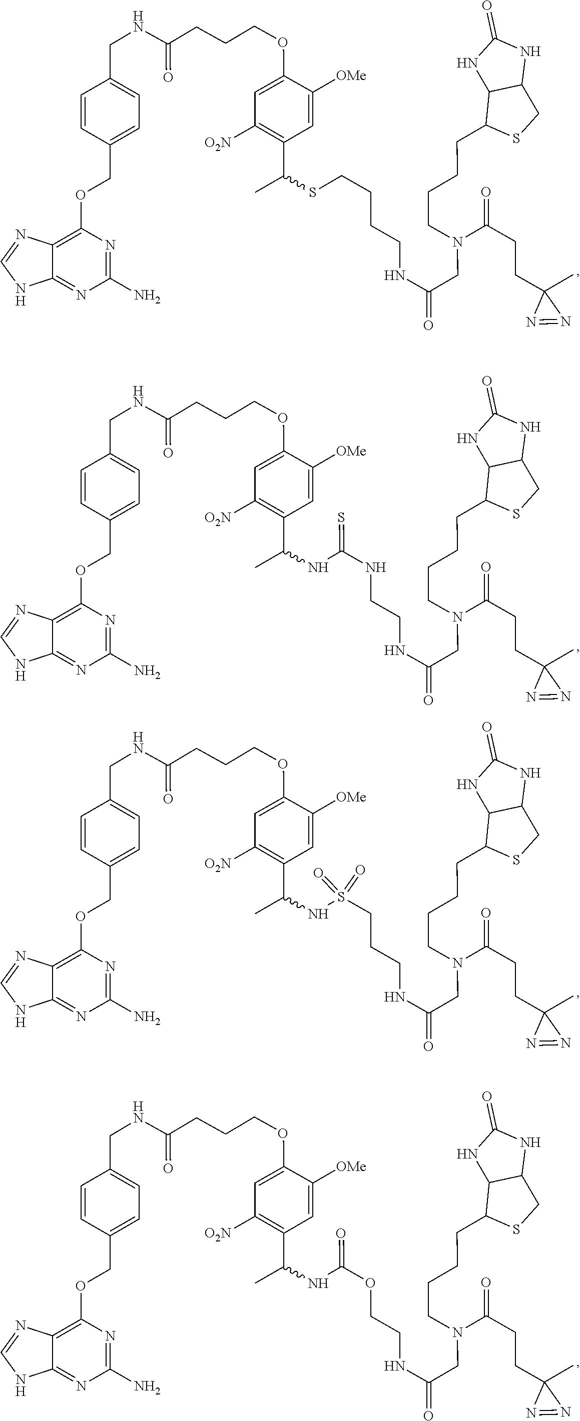

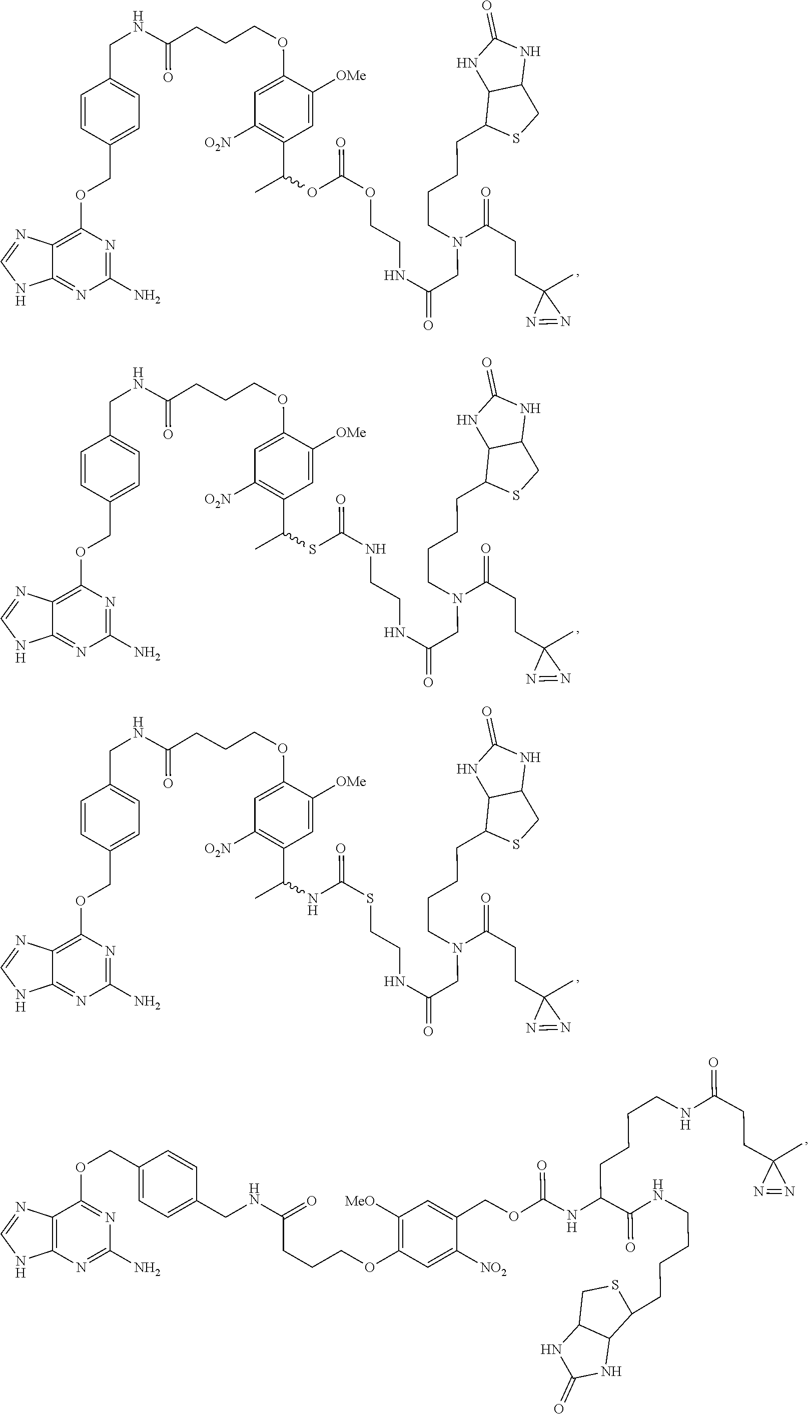

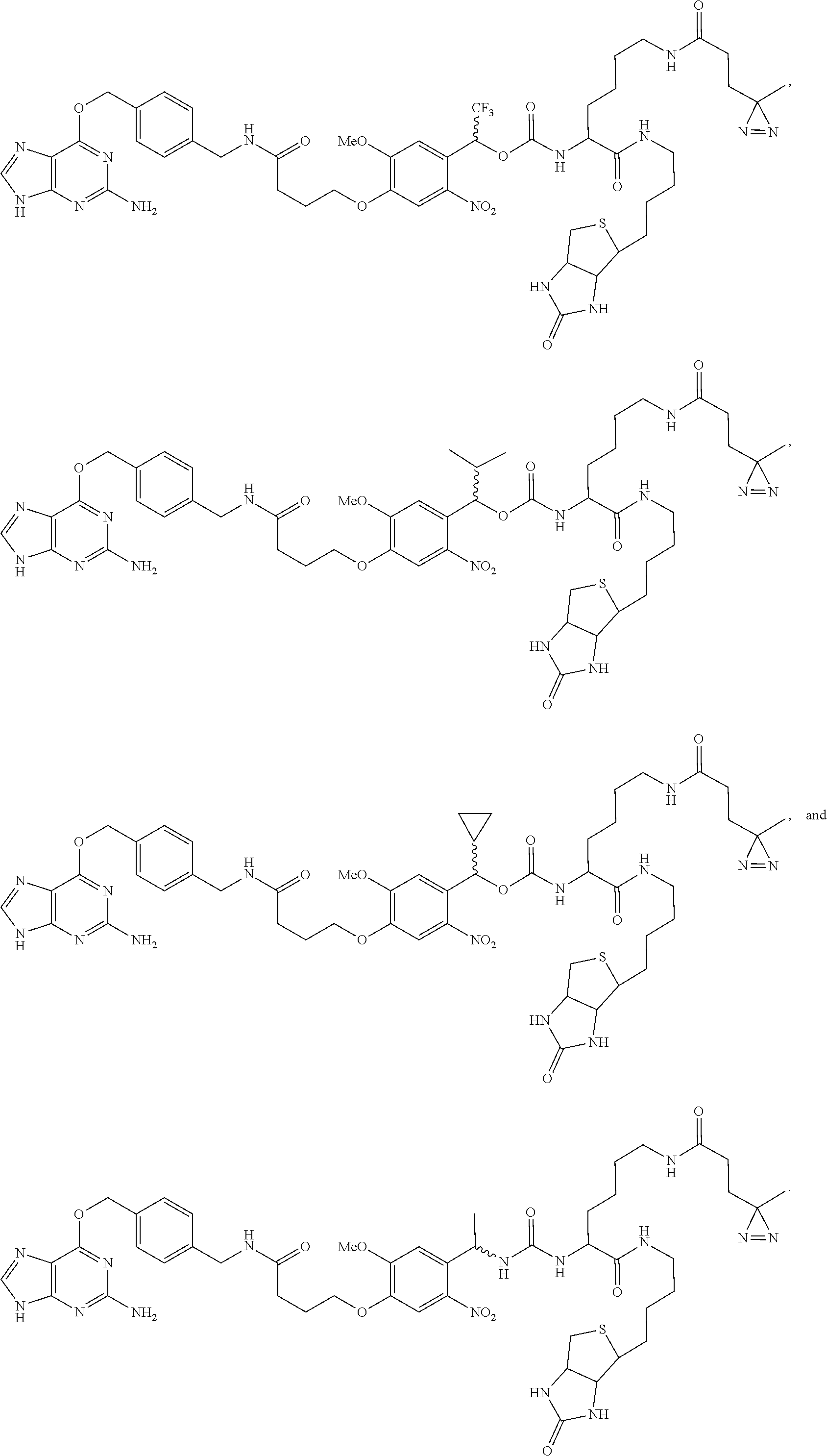

In some embodiments, the compound is selected from:

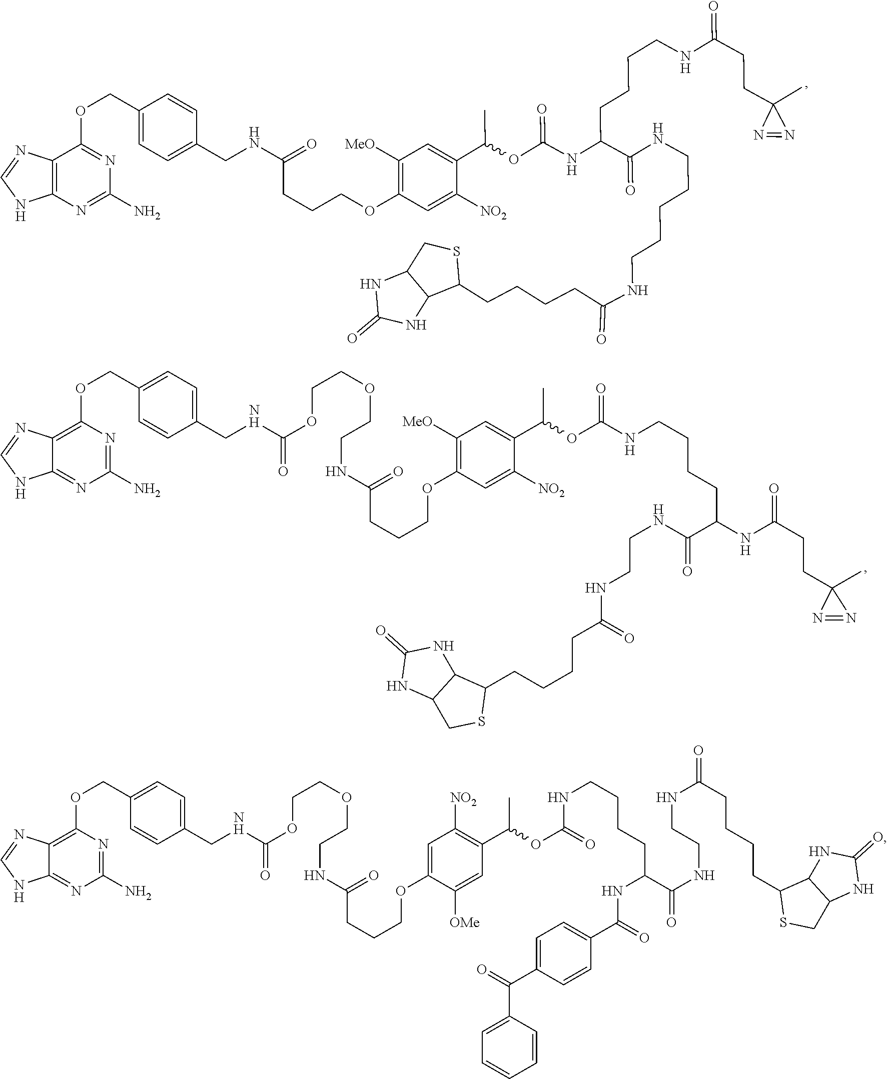

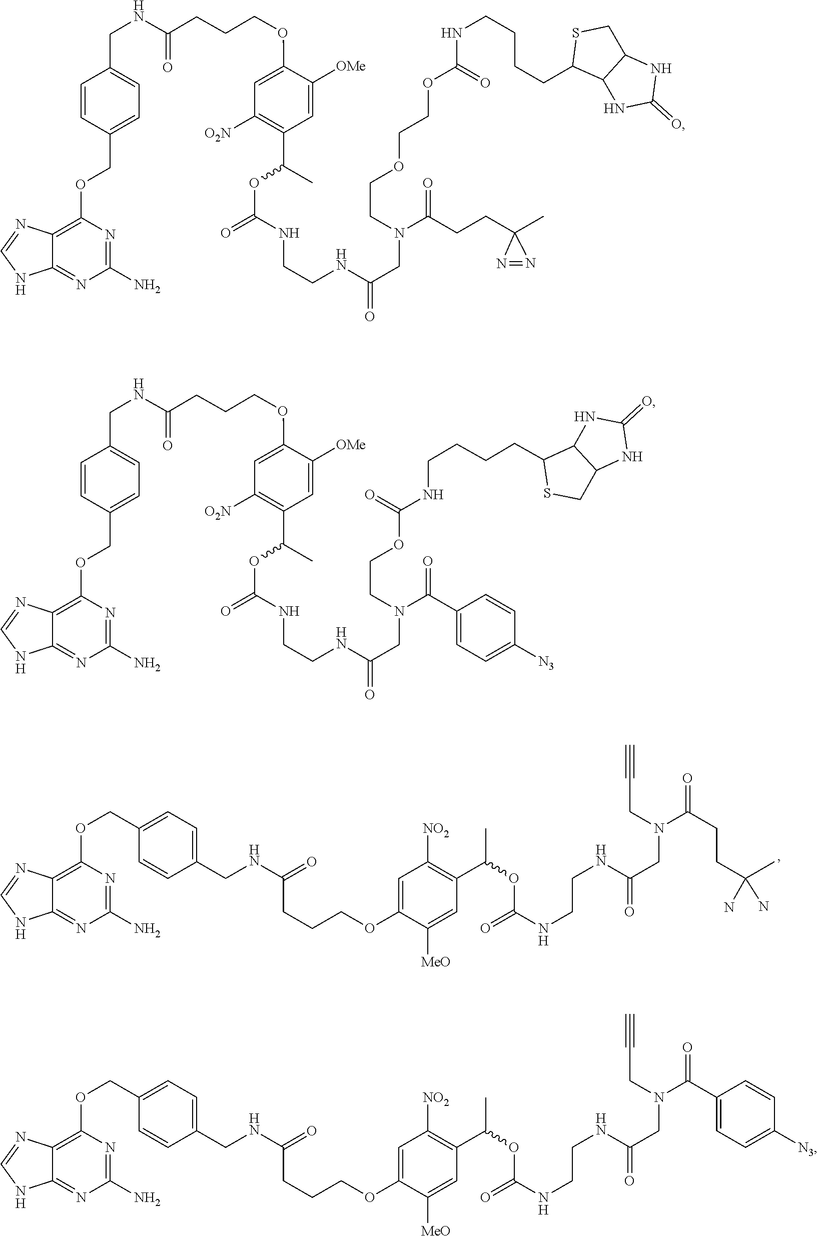

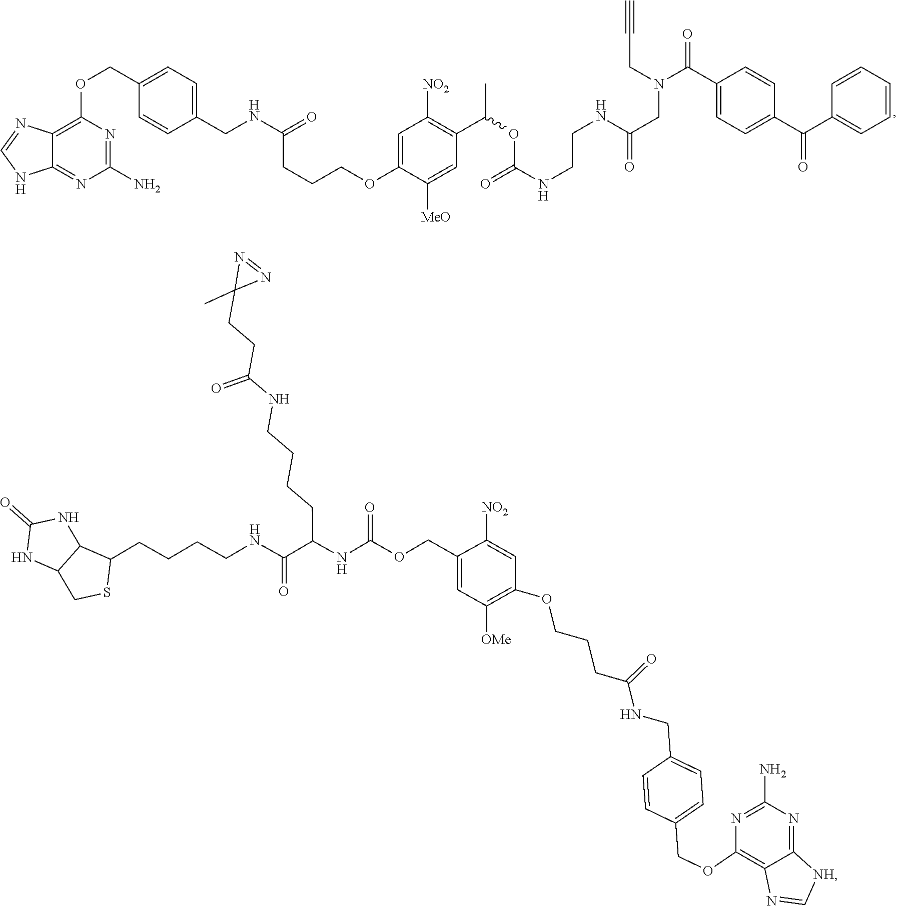 [SMILES: C#CCN(CC(=O)NCCNC(=O)OC(C)c1cc(OC)c(OCCCC(=O)NCc2ccc(COc3nc(N)nc4[nH]cnc34)cc2)cc1[N+](=O)[O-])C(=O)c1ccc(C(=O)c2ccccc2)cc1 ; COc1cc(COC(=O)NC(CCCCNC(=O)CCC2(C)N=N2)C(=O)NCCCCC2SCC3NC(=O)NC32)c([N+](=O)[O-])cc1OCCCC(=O)NCc1ccc(COc2nc(N)nc3[nH]cnc23)cc1]

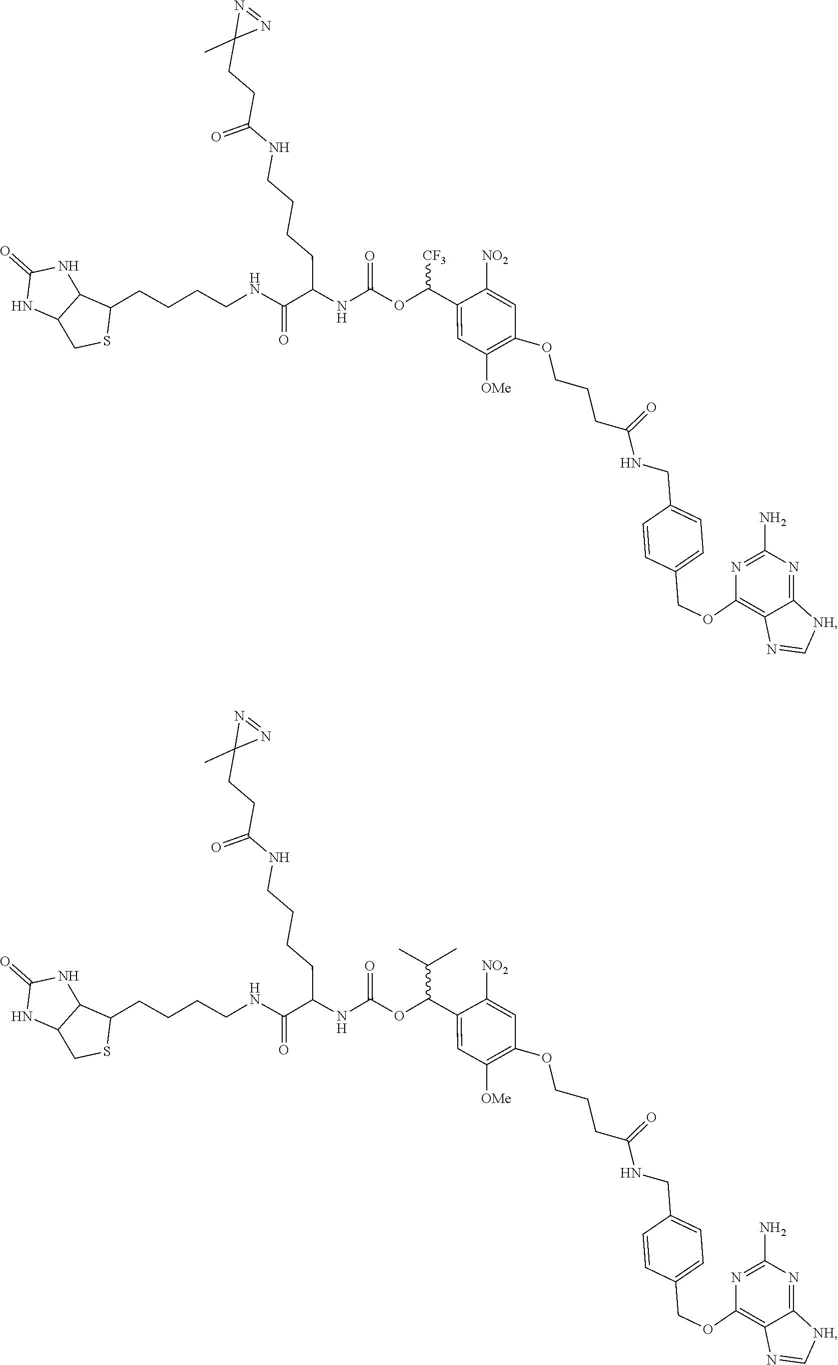 [SMILES: COc1cc(C(OC(=O)NC(CCCCNC(=O)CCC2(C)N=N2)C(=O)NCCCCC2SCC3NC(=O)NC32)C(F)(F)F)c([N+](=O)[O-])cc1OCCCC(=O)NCc1ccc(COc2nc(N)nc3[nH]cnc23)cc1 ; COc1cc(C(OC(=O)NC(CCCCNC(=O)CCC2(C)N=N2)C(=O)NCCCCC2SCC3NC(=O)NC32)C(C)C)c([N+](=O)[O-])cc1OCCCC(=O)NCc1ccc(COc2nc(N)nc3[nH]cnc23)cc1]

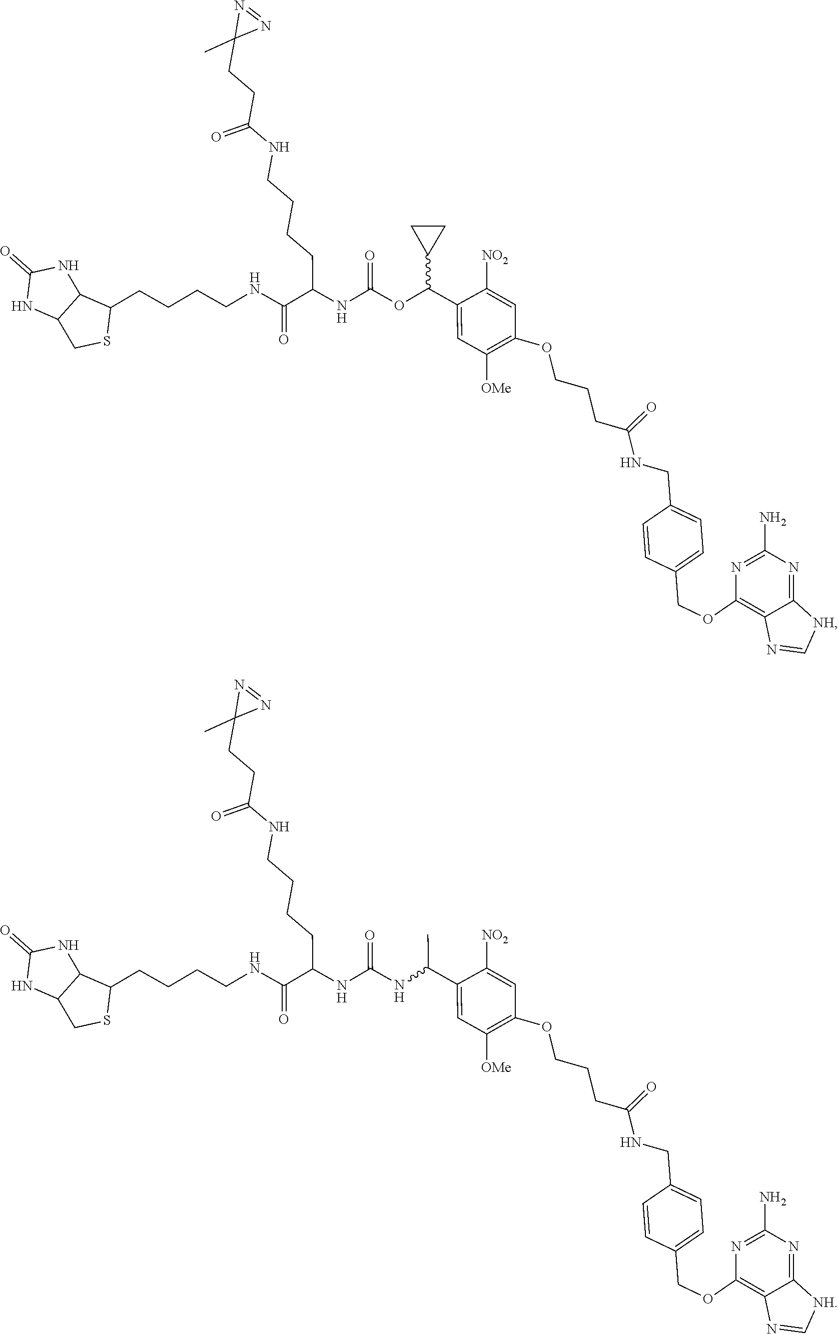 [SMILES: COc1cc(C(OC(=O)NC(CCCCNC(=O)CCC2(C)N=N2)C(=O)NCCCCC2SCC3NC(=O)NC32)C2CC2)c([N+](=O)[O-])cc1OCCCC(=O)NCc1ccc(COc2nc(N)nc3[nH]cnc23)cc1 ; COc1cc(C(C)NC(=O)NC(CCCCNC(=O)CCC2(C)N=N2)C(=O)NCCCCC2SCC3NC(=O)NC32)c([N+](=O)[O-])cc1OCCCC(=O)NCc1ccc(COc2nc(N)nc3[nH]cnc23)cc1]

In some embodiments, P₁ is a divalent derivative of an ortho-nitrobenzyl compound and L₂ is —N—C(═O)—. In some embodiments, the compound of Formula (I) has a structure of Formula (IIIa) or Formula (IIIb):

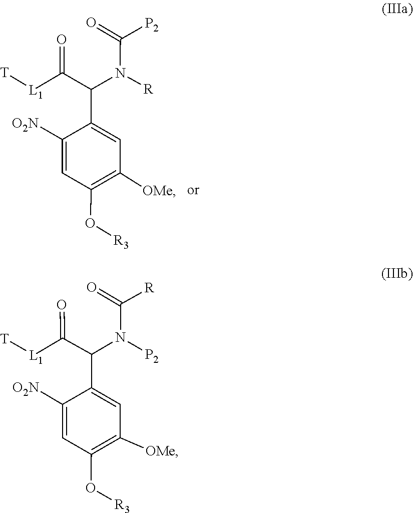

wherein: T, L₁, R, and P₂ are as defined for the compound of Formula (I); and R₃ is alkyl (e.g., C₁-C₆ alkyl). In some embodiments, R₃ is methyl.

In some embodiments, the compound is selected from:

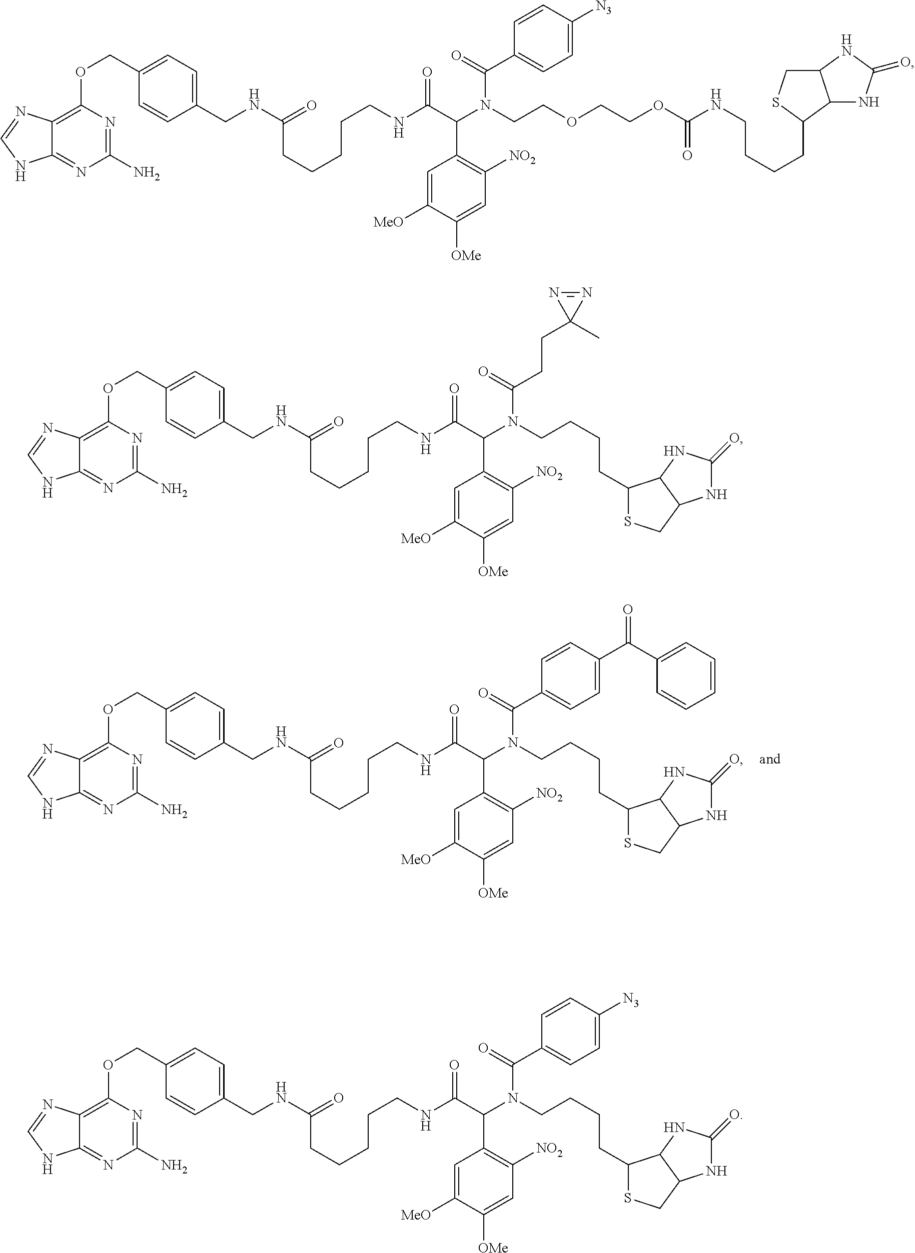

In some embodiments, the compound is:

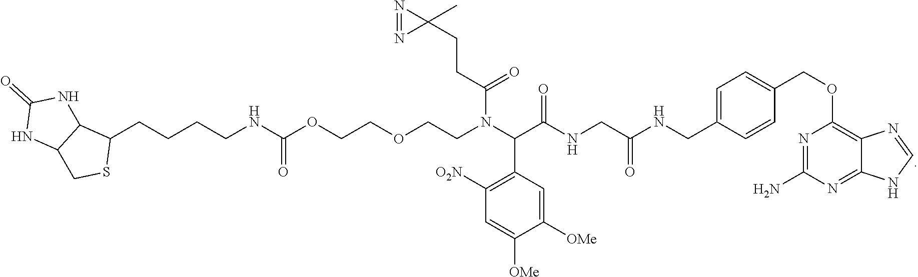 [SMILES: COc1cc(C(C(=O)NCC(=O)NCc2ccc(COc3nc(N)nc4[nH]cnc34)cc2)N(CCOCCOC(=O)NCCCCC2SCC3NC(=O)NC32)C(=O)CCC2(C)N=N2)c([N+](=O)[O-])cc1OC]

In some embodiments, P₁ comprises a divalent nitroindoline derivative or a divalent nitrobenzopiperidine derivative. In some embodiments, the compound of Formula (I) has a structure of Formula (IVa) or (IVb):

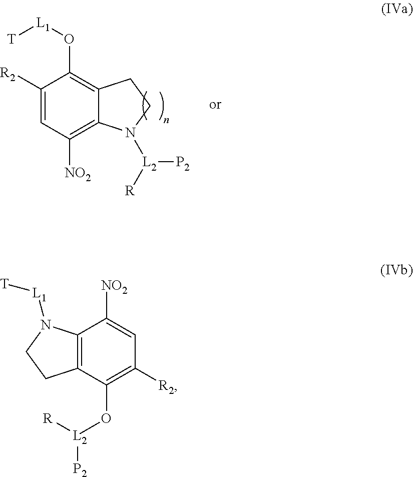

wherein: T, L₁, L₂, R and P₂ are as defined for Formula (I); n is 1 or 2; and R₂ is selected from NO₂ and H. In some embodiments, the probe is a compound of Formula (IVa) and L₂ and the nitrogen atom to which L₂ is attached together form a carbamate, a urea, a thiourea, an amide, or a sulfonamide. In some embodiments, the probe is a compound of Formula (IVb) and L₁ and the nitrogen atom to which L₁ is attached together form a carbamate, a urea, a thiourea, an amide, or a sulfonamide.

In some embodiments, the compound has a structure selected from:

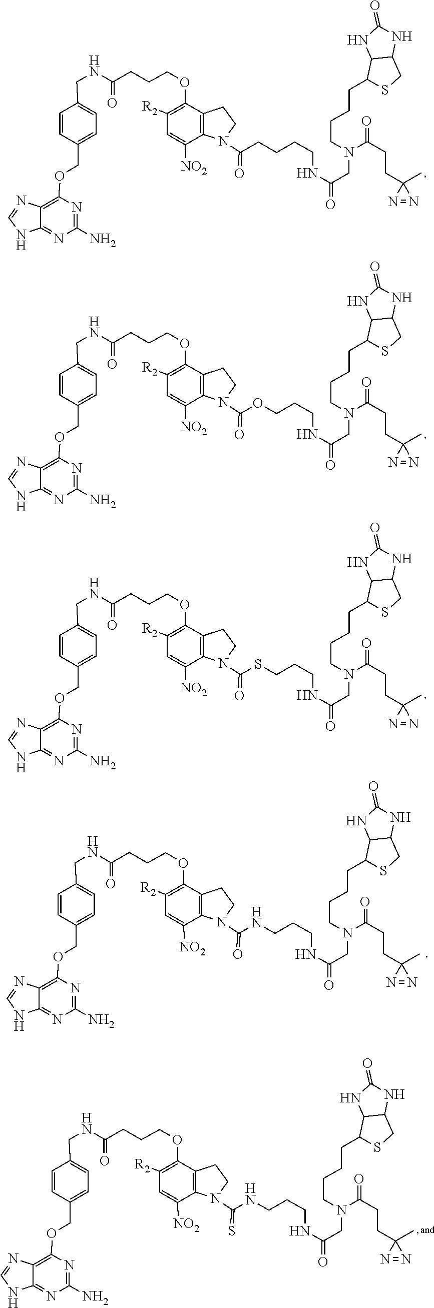

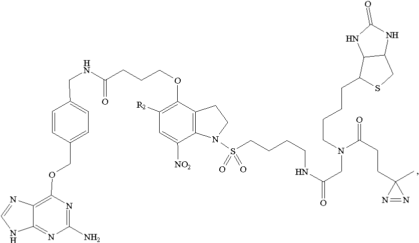

wherein R₂ is NO₂ or H.

In some embodiments, P₁ is a divalent coumarin derivative. In some embodiments, the compound of Formula (I) has a structure of Formula (Va) or (Vb):

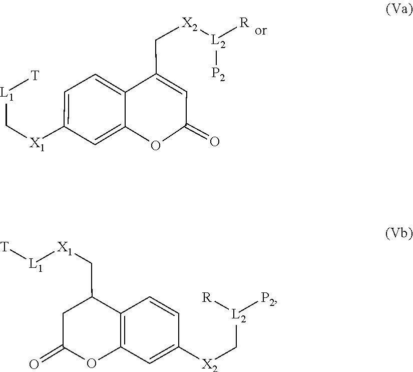

wherein: T, L₁, L₂, R, and P₂ are as defined for the compound of Formula (I); and X₁ and X₂ are independently selected from O, NR′, and S, wherein R′ is H or alkyl (e.g., C₁-C₆ alkyl). In some embodiments, the compound has a structure of Formula (Va) and X₂ and L₂ together form a carbamate, a urea, an amide, an ester, an ether, an amine, a sulfide, or a thiourea group. In some embodiments, the compound has a structure of Formula (Vb) and X₁ and L₁ together form a carbamate, a urea, an amide, an ester, an ether, an amine, a sulfide, or a thiourea group.

In some embodiments, the compound is selected from the group comprising:

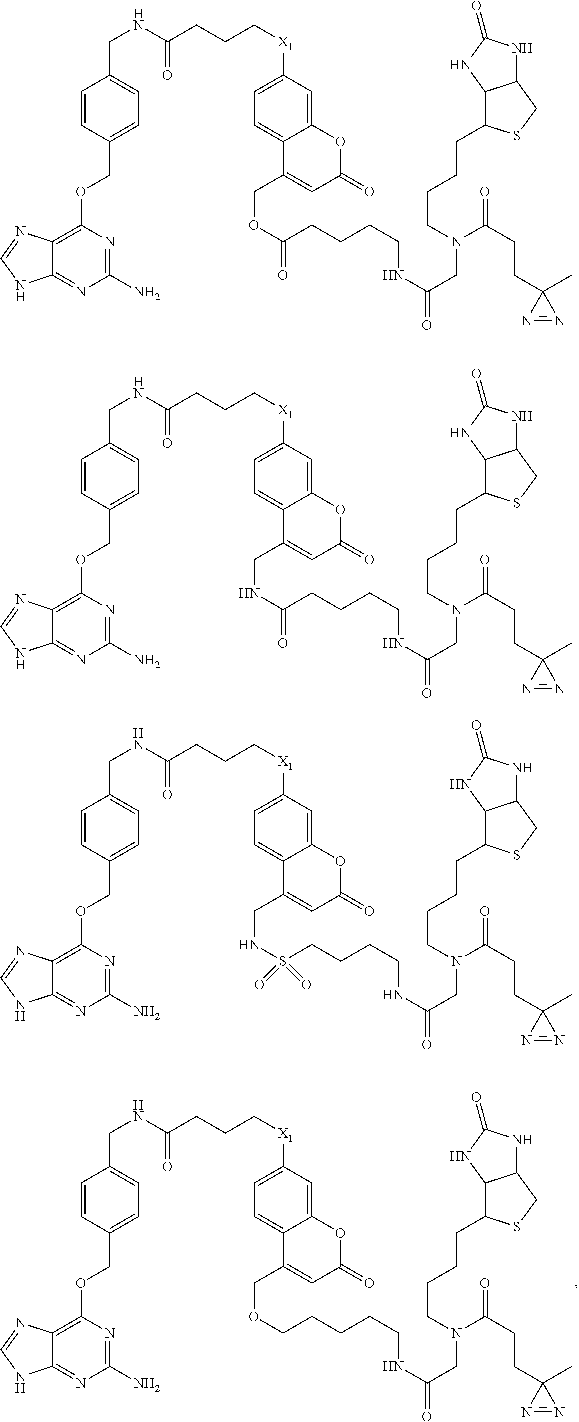

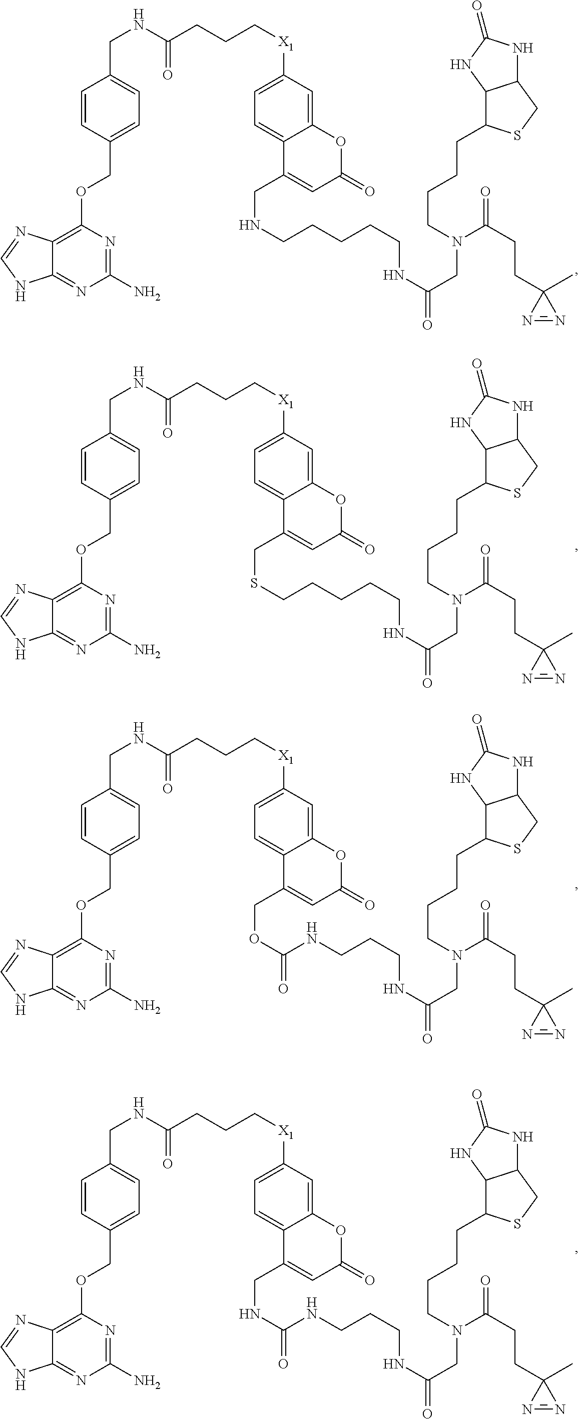

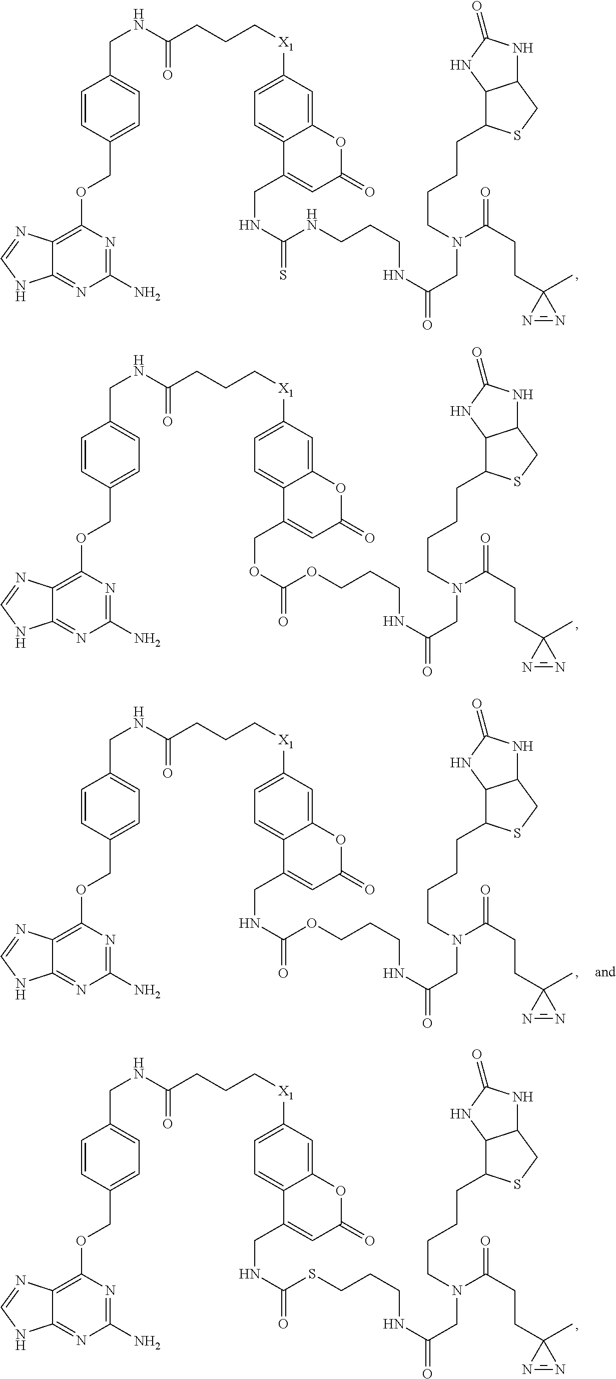

where X₁ is O, NR′ (e.g., C₁-C₆ alkyl) or S.

In some embodiments, P₁ is a divalent ortho-hydroxybenzyl derivative or an ortho-hydroxynaphthyl derivative. In some embodiments, the compound of Formula (I) has a structure of one of Formula (VIa) and (VIb):

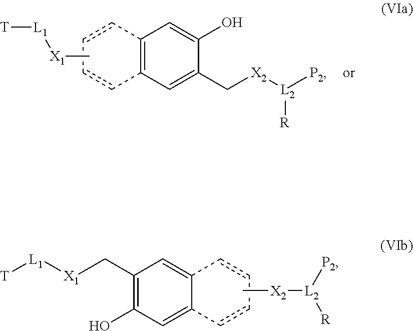

wherein: T, L₁, L₂, P₂, and R are as defined for the compound of Formula (I); the dotted lines can be present or absent, and when absent, X₁ or X₂ is substituted on the remaining aryl ring; and X₁ and X₂ are independently selected from O, NR′, and S, wherein R′ is selected from H and alkyl (e.g., C₁-C₆ alkyl). In some embodiments. L₁ and X₁ together form a carbamate, a urea, an amide, an ester, an amine, a sulfide, or a thiourea. In some embodiments, L₂ and X₂ together form a carbamate, a urea, an amide, an ester, an amine, a sulfide or a thiourea.

In some embodiments, the compound is selected from the group comprising:

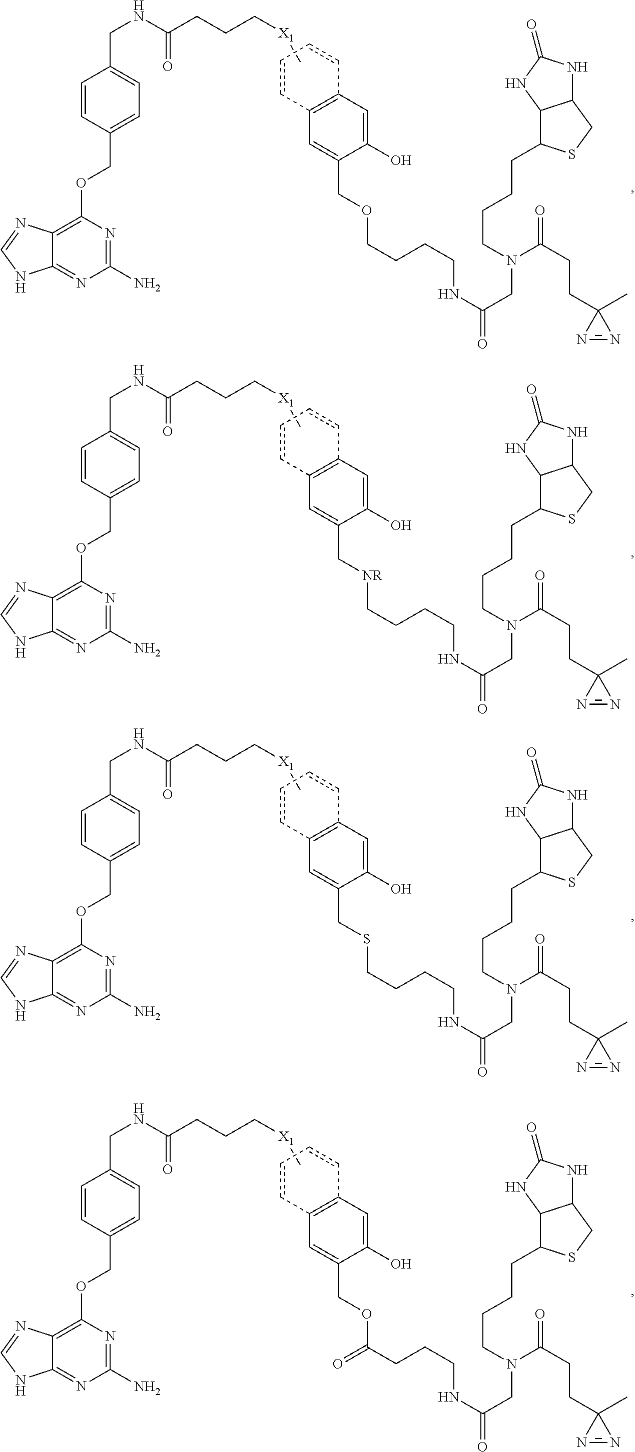

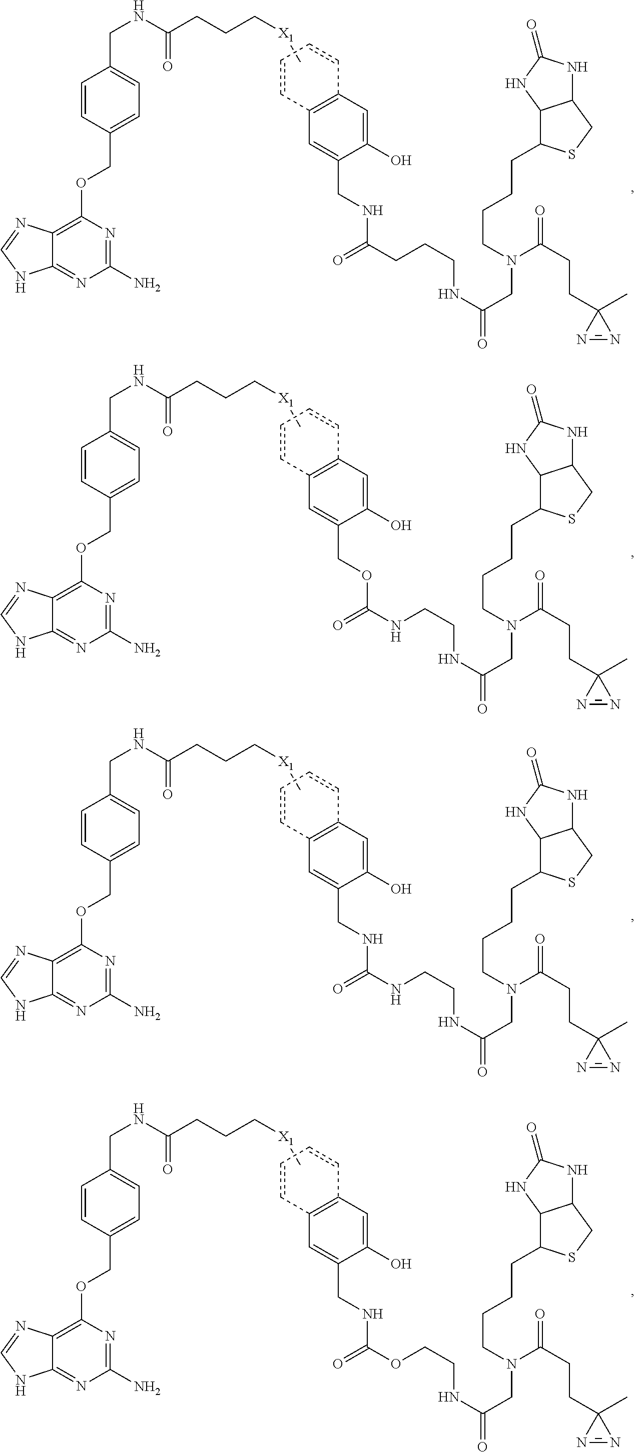

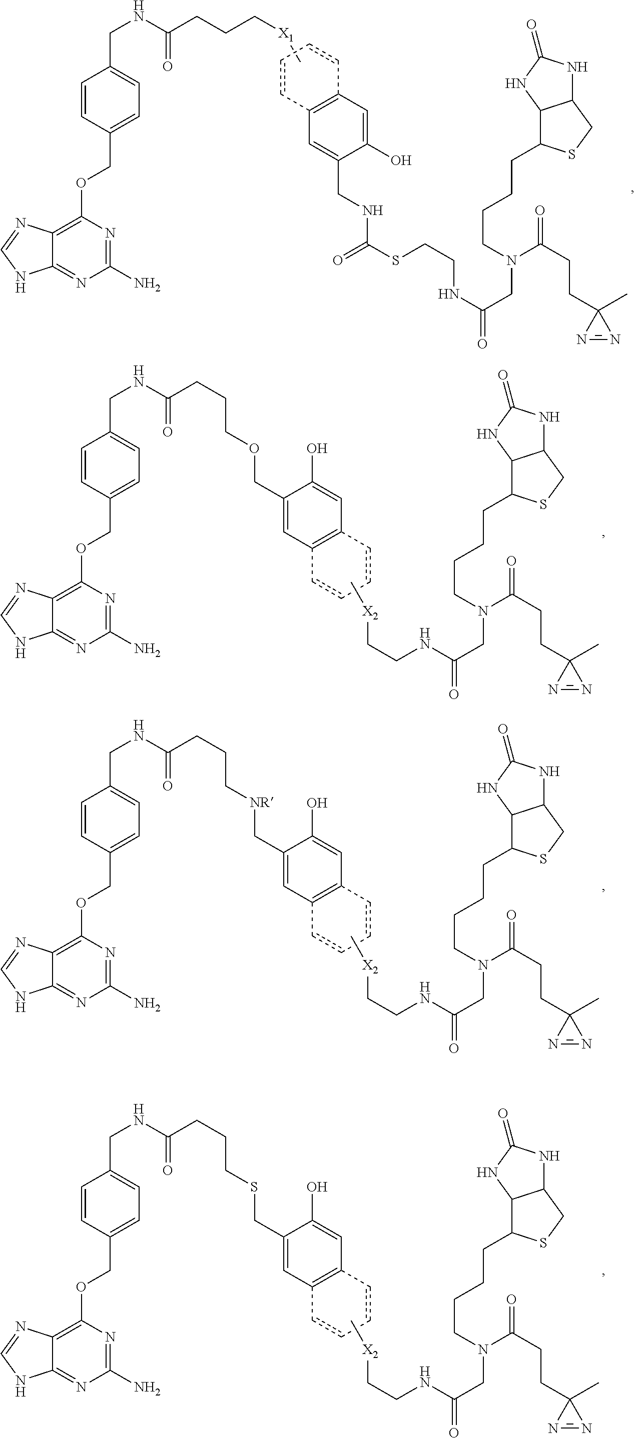

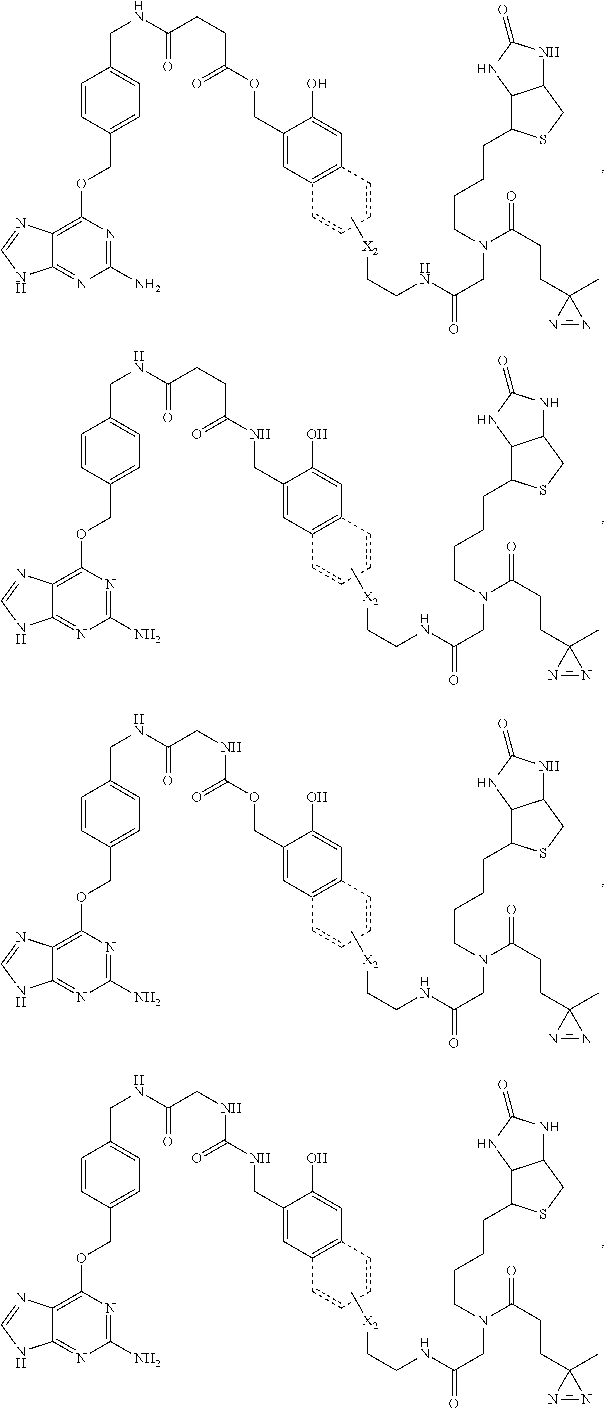

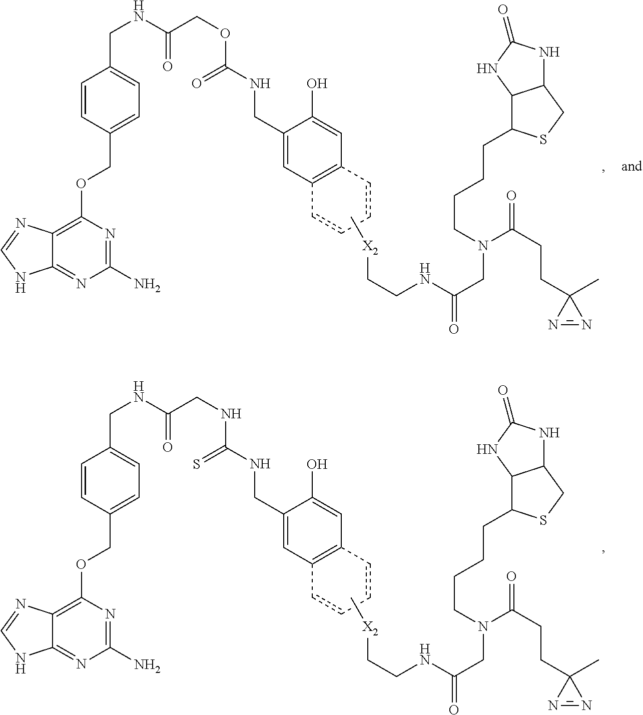

wherein X₁ and X₂ are selected from O, NR′ and S and wherein R′ is H or C₁-C₆ alkyl. In some embodiments, R′ is C₁-C₆ alkyl. In some embodiments, R′ is methyl.

II.B. Catalytic Photoactive Probe Systems

In some embodiments, the presently disclosed subject matter provides a photoactive probe system comprising at least two different probe molecules, wherein one of the probe molecules comprises a photocatalytic moiety. FIG. 1B shows a schematic drawing showing the main components of a photoactive photoproximity probe system that includes a probe molecule that comprises a photocatalytic group. More particularly, in some embodiments, the presently disclosed probe system includes a first probe molecule which includes a target recognition moiety T that is capable of specifically binding a modular first binding partner (e.g. a first binding partner protein) and a photocatalytic moiety P_{c}. This probe molecule can also be referred to as a “photocatalytic probe” or a “probe catalyst.” P_{c} is a photoreactive group that can be excited by light to form a moiety (e.g. an excited triplet state) capable of catalyzing the chemical transformation or activation of another moiety, such as another moiety on a separate probe molecule. In some embodiments, P_{c} is a moiety comprising a flavin scaffold (i.e., a monovalent isoalloxazine moiety). The presently disclosed probe system further includes at least one additional probe molecule that comprises a detectable moiety R (e.g., a group capable of specifically binding a second binding partner (i.e., a recognition handle” or “affinity identification handle”) and a photoreactive group P₃ that can undergo a chemical transformation or activation catalyzed by the photocatalytic moiety P_{c} to become more reactive to other entities. This additional probe molecule can also be referred to as a “probe substrate” or a “tagging probe” and the photoreactive moiety P₃ can act as a “photoaffinity moiety” or a “photocapture group” following the photocatalyzed chemical transformation or activation catalyzed by P_{c}.

When the probe system is contacted with a sample comprising a labeled BTOI (e.g., a protein or cell genetically labeled with a first binding partner, such as a protein or peptide tag), or labelled moiety known to be in the proximal network of the BTOI, the target recognition moiety T specifically binds to the first binding partner on the BTOI (e.g., the protein or peptide tag). In some embodiments, the interaction between the target recognition moiety and the first binding partner is covalent in nature, such that the probe catalyst is irreversibly localized to the BTOI (e.g., in or on a live cell). In some embodiments, the interaction between the target recognition moiety and the first binding partner is non-covalent, resulting in reversible localization of the photocatalytic probe to the BTOI. For example, the interaction can involve selective hybridization between nucleic acid sequences. When the sample comprising the BTOI and the probe system is exposed to light (e.g., 365 nm light), photocatalytic moiety P_{c} catalyzes a chemical transformation (e.g., an oxidation) or activation of a P₃ moiety on at least one, and typically more than one, nearby probe substrates (probe substrates near enough to the photocatalytic probe to be involved in an interaction where a complex forms between the P_{c} group and the P₁ group). This catalysis results in the transformation or activation of the photoreactive moiety P₃ from a chemical group (e.g., a phenol group) that is relatively unreactive with other nearby entities to a group (e.g., a phenoxy radical) that is relatively reactive with nearby entities. Thus, the transformed P₃ group can bond to an entity (e.g., a peptide, a protein, a small molecule such as a drug or metabolite, a nucleic acid (e.g., a RNA or DNA), or a cell) near to the BTOI, thereby labeling that molecule with the detectable moiety R. The entities labeled with detectable moiety R are near to the BTOI, within a diffusion radius that can be varied, e.g., based on the length of the moiety linking T and P_{c} and the lifetime of the transformed photoreactive moiety P₃. In some embodiments, the system can include two different photocatalytic probes, e.g., comprising different length linker moieties and/or different catalytic moieties. In some embodiments, the system can include two different probe substrates. e.g., comprising different P₃ groups.

In contrast to the probe molecules shown in FIG. 1A, where one molecule of probe labels a single entity in proximity to the BTOI, a photocatalytic probe of the system of FIG. 1B can catalyze the transformation of the P₃ group on multiple probe substrates, resulting in the labeling of more than one entity near the BTOI and/or higher labeling signal from a single photocatalytic probe. Accordingly, in some embodiments the probe molecule shown in FIG. 1A is referred to here as a stoichiometric probe, in comparison to the photocatalytic probe of the probe system of FIG. 1B. In some embodiments, the combination of photocatalytic probe and probe substrate comprises a molar excess of the probe substrate compared to the photocatalytic probe.

However, like the probe of FIG. 1A, all portions of the probe molecules of the probe system (i.e., the photocatalytic probe and the probe substrate) are modular, providing for a combinatorial library of photocatalytic probes and probe substrates that can study a variety of interactions of diverse BTOIs. The mode of BTOI targeting of the probe can be varied to include both reversible and irreversible binding to the BTOI. As noted above, the particular combination of photocatalytic and photoreactive groups can provide tuning of the labeling radius of the probe system, responsive light wavelengths, and the timing of the probe-detectable interactions. The combination of modular elements can retain activity of each individual element, as well as suitable pharmacologic properties like aqueous solubility and cell permeability.

In some embodiments, the presently disclosed subject matter provides a probe system comprising: a photocatalytic probe having a structure of Formula (VII): T-L₁₀-P_{c}, and a probe substrate having a structure of Formula (VIII): P₃-L₁₁-R, wherein: T is a target recognition moiety capable of specifically binding a first binding partner, optionally wherein the first binding partner is a peptide or protein tag attached to a biological target of interest; L₁₀ and L₁₁ are bivalent linkers; P_{c} is a photocatalytic moiety; P₃ is a photoreactive moiety that is capable of undergoing a reaction catalyzed by P_{c}; and R is a detectable moiety or a precursor thereof capable of specifically binding a second binding partner, subject to the proviso that the first and second binding partners are different.

R can be any suitable group that selectively binds to a binding partner, such as any suitable affinity label known in the art, or a precursor thereof. For example, R of the probe substrate of Formula (VIII) can be selected from the same R used for the probe of Formula (I), described hereinabove. In some embodiments, R can be a monovalent moiety derived from a small molecule, a peptide (e.g., an antigenic peptide), a peptide analog (e.g., a peptoid), or a nucleic acid or nucleic acid analog. In some embodiments. R can comprise biotin, a biotin analog (e.g., desthiobiotin) or a precursor thereof (e.g., an alkyne that can react with a biotin-azide reagent via Cu-catalyzed Click cycloaddition). In some embodiments. R is selected from:

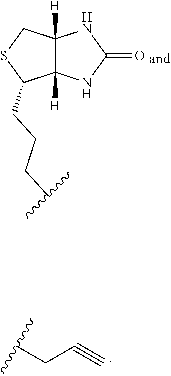

Target recognition moiety T can also be any suitable group that selectively binds to a binding partner, so long as it selectively binds to a different binding partner than the R moiety used in the probe substrate used with the photocatalytic probe comprising T. For example, T of the photocatalytic probe of Formula (VII) can be selected from the same T used for the probe of Formula (I), described hereinabove. In some embodiments, T is other than a protein or peptidyl moiety. In some embodiments, T is suitable for selective binding to a protein or peptide tag. In some embodiments, T comprises a moiety selected from the group comprising a benzylguanine group (which can specifically bind to MGMT-fusion or “SNAP-tag” fusion proteins), a chloroalkane group (which can specifically bind to “Halo-Tag” fusion proteins), a benzylcytosine group (which can specifically bind to “Clip-Tag” fusion proteins), an azide (which can specifically bind to a receptor engineered with a strained cyclooctyne or other group for [3+2] Huisgen chemistry), biotin or a biotin analog (e.g., desthiobiotin)(which can specifically bind to monomeric streptavidin, neutravidin, or avidin fusion proteins and can be used as long as R does not specifically bind to streptavidin, neutravidin, or avidin as well) AP1867 or an orthogonal FK506 analog (i.e., a tacrolimus analog)(which can specifically bind to FK binding protein (e.g., FKBP12 or mutant FKBP12) fusion proteins) and a methotrexate derivative (which can specifically bind to fusion proteins of wild-type or engineered dihydrofolate reductase (DHFR)). In some embodiments, T is selected from:

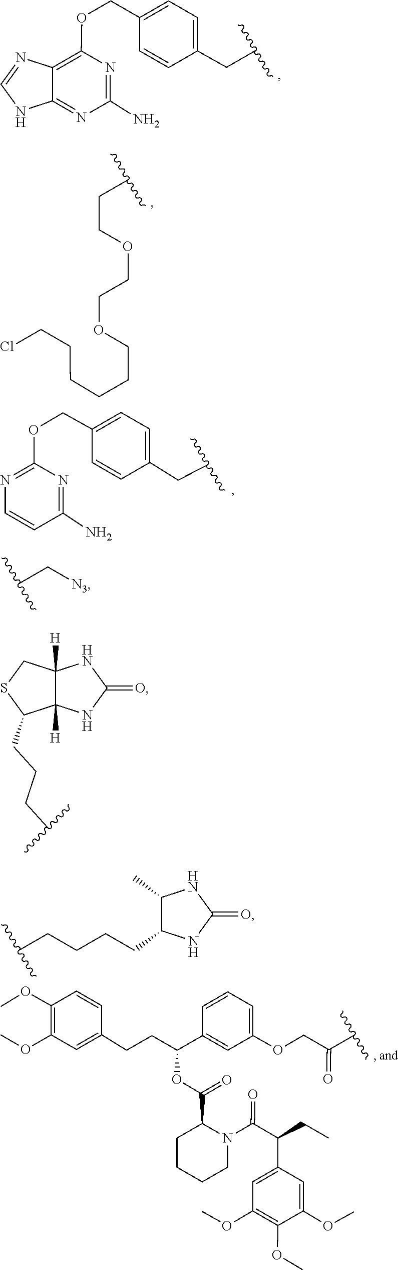

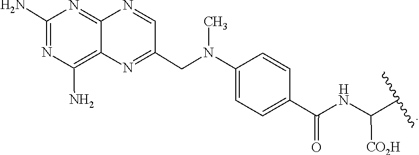 [SMILES: *C(NC(=O)c1ccc(N(C)Cc2cnc3nc(N)nc(N)c3n2)cc1)C(=O)O]

The photocatalytic group P_{c} can be any suitable photocatalytic group. The photocatalytic group should be a group that retains photoactivity when attached via a linker moiety to a targeting group capable of specific delivery and localization of the photocatalytic group to a biological target of interest; that is capable of retaining the photoactivity after the targeting group is covalently or non-covalently bonded to a group on the BTOI; and that provides light-dependent activation of one or more substrates. In some embodiments, the photocatalytic group can retain photoactivity either inside or outside of a cell. In some embodiments, the photocatalytic group is free of (or significantly free of) toxicity to living cells. In some embodiments, P_{c} is a group based on the structure of flavin. Thus, in some embodiments, P_{c} is a monovalent isoalloxazine moiety. In some embodiments, P_{c} has the structure:

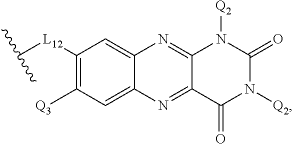

where L₁₂ is present or absent and, when present, is a bivalent moiety selected from the group comprising —O-alkylene, —S-alkylene, —NQ₄-alkylene, and alkylene, wherein said alkylene is substituted or unsubstituted; and each of Q₁, Q₂, Q₃ and Q₄ are independently selected from H, alkyl (e.g., C₁-C₆ alkyl), and cycloalkyl (e.g., C₃-C₇ cycloalkyl). In some embodiments, L₁₂ is absent and the linker group L₁₀ is directly attached to an aryl ring of the isoalloxazine group. In some embodiments, —O-alkylene (e.g., C₁-C₆ alkylene). In some embodiments, the alkylene group is a methylene group. Thus, in some embodiments, L₁₂ is —O—CH₂—. In some embodiments, Q₃ is methyl. In some embodiments, Q₁ and Q₂ are each H. In some embodiments, Q₁ and Q₂ are each methyl. In some embodiments, Q₁ and Q₂ are each cyclopropyl. In some embodiments, L₁₀ is a group selected from —NH—C(═O)— and —NH—C(═O)-alkylene-NH—C(═O)—. The alkylene can be substituted or unsubstituted. In some embodiments, the alkylene is pentylene.

In some embodiments, T is a benzylguanine group or a chloroalkane group. In some embodiments, the photocatalytic probe of Formula (VII) is selected from the group comprising:

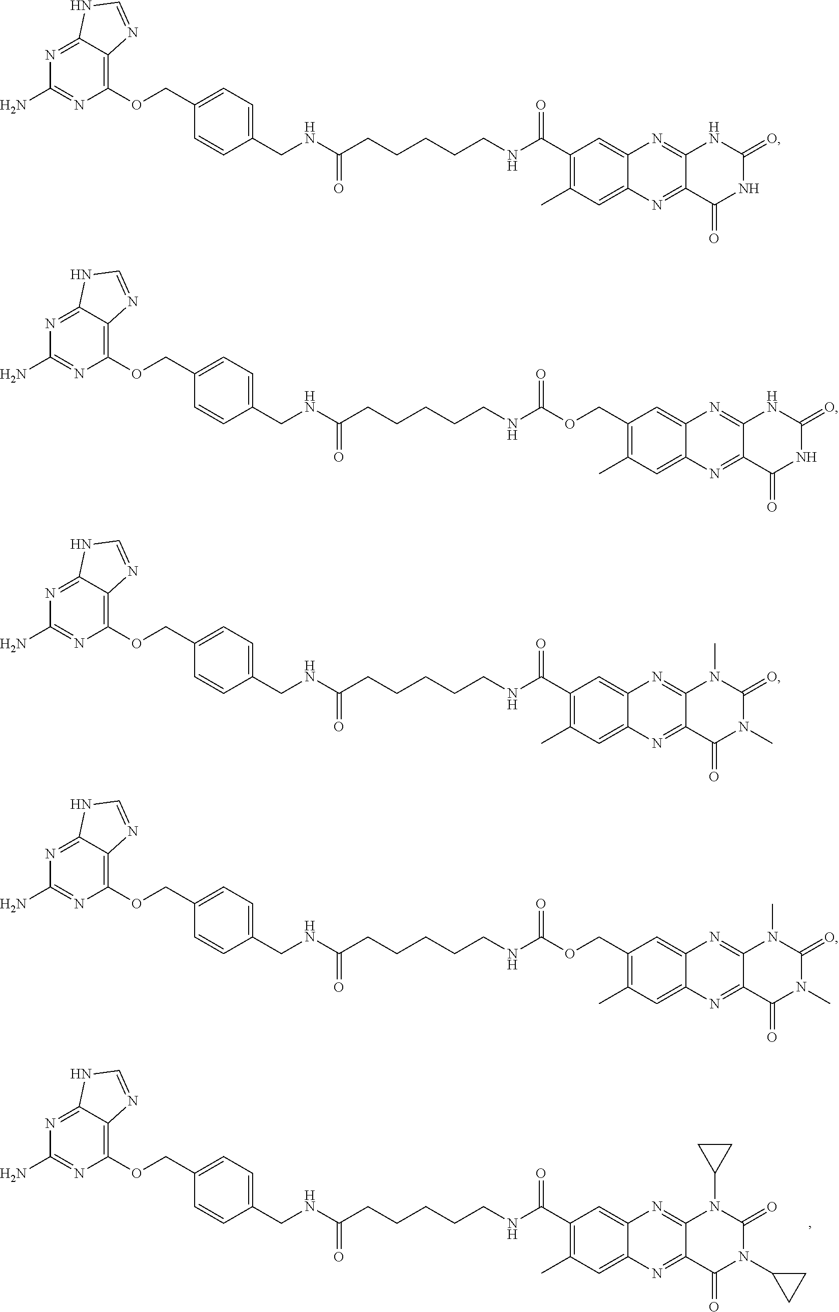 [SMILES: Cc1cc2nc3c(=O)[nH]c(=O)[nH]c3nc2cc1C(=O)NCCCCCC(=O)NCc1ccc(COc2nc(N)nc3[nH]cnc23)cc1 ; Cc1cc2nc3c(=O)[nH]c(=O)[nH]c3nc2cc1COC(=O)NCCCCCC(=O)NCc1ccc(COc2nc(N)nc3[nH]cnc23)cc1 ; Cc1cc2nc3c(=O)n(C)c(=O)n(C)c3nc2cc1C(=O)NCCCCCC(=O)NCc1ccc(COc2nc(N)nc3[nH]cnc23)cc1 ; Cc1cc2nc3c(=O)n(C)c(=O)n(C)c3nc2cc1COC(=O)NCCCCCC(=O)NCc1ccc(COc2nc(N)nc3[nH]cnc23)cc1 ; Cc1cc2nc3c(=O)n(C4CC4)c(=O)n(C4CC4)c3nc2cc1C(=O)NCCCCCC(=O)NCc1ccc(COc2nc(N)nc3[nH]cnc23)cc1]

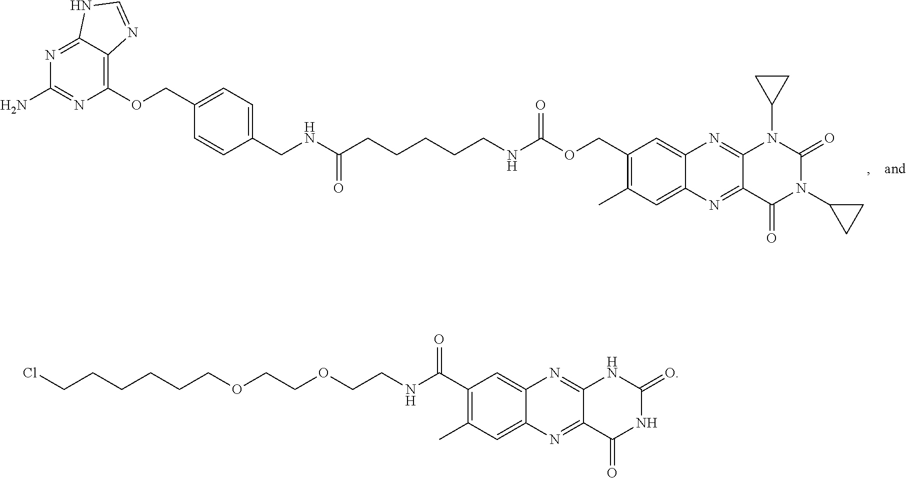 [SMILES: Cc1cc2nc3c(=O)n(C4CC4)c(=O)n(C4CC4)c3nc2cc1COC(=O)NCCCCCC(=O)NCc1ccc(COc2nc(N)nc3[nH]cnc23)cc1 ; Cc1cc2nc3c(=O)[nH]c(=O)[nH]c3nc2cc1C(=O)NCCOCCOCCCCCCCl]

P₃ can be any photoreactive group that can be activated by or undergo a chemical transformation catalyzed by the P_{c} moiety to form a more reactive group (i.e., a group having higher chemical reactivity with biological entities). In some embodiments, P₃ is comprises a phenol, an aniline, or a diazirine. The aniline or phenol of P₃ can optionally include one or more aryl group substituents. For example, in some embodiments, P₃ is a phenol or an aniline group having the structure:

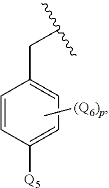

where p is an integer between 0 and 4; Q₅ is OH or N(Q₇)₂, wherein each Q₇ is independently H or alkyl (e.g., C₁-C₆ alkyl) and where each Q₆ is an aryl group substituent, such as alkyl or alkoxy (e.g., C₁-C₆alkyl or C₁-C₆ alkoxy). In some embodiments, Q₅ is OH, p is 1, and Q₆ is alkoxy (e.g., C₁-C₆ alkoxy). In some embodiments, the Q₆ group is attached at a carbon atom adjacent to the carbon atom attached to the Q₅ moiety. In some embodiments, Q₆ is methoxy. In some embodiments, P₃ is a diazirine. In some embodiments, L₁₁ is selected from —O—, —O-alkylene-, —O—C(═O)—NH—, —O—C(═O)—NH-alkylene, —O—CH₂—C(═O)—NH—, —O—CH₂—C(═O)—NH-alkylene, —CH₂—C(═O)—, —CH₂—NH—C(═O)—, —CH₂—NH—C(═O)-alkylene, and —C(═O)—NH—. In some embodiments, the probe substrate of Formula (VIII) has a structure selected from the group comprising:

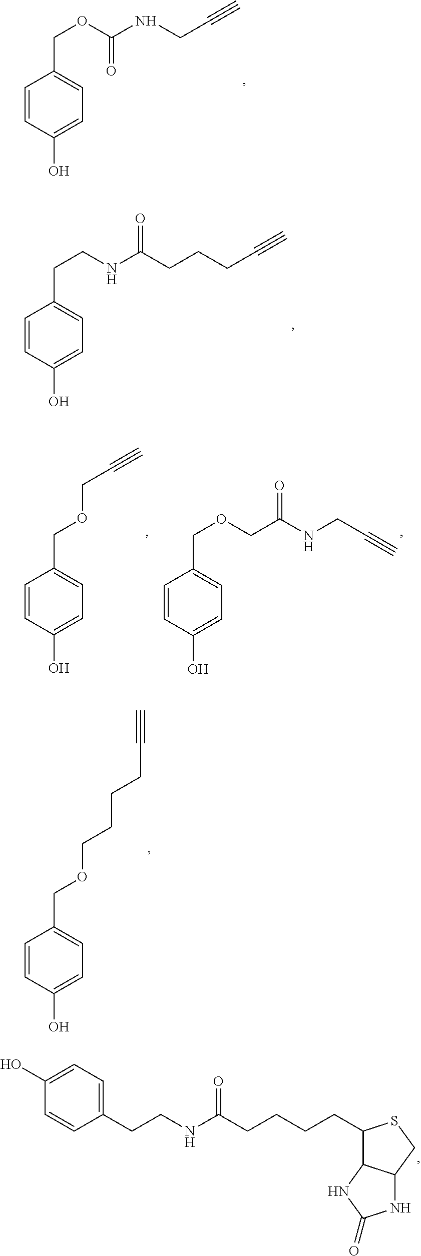

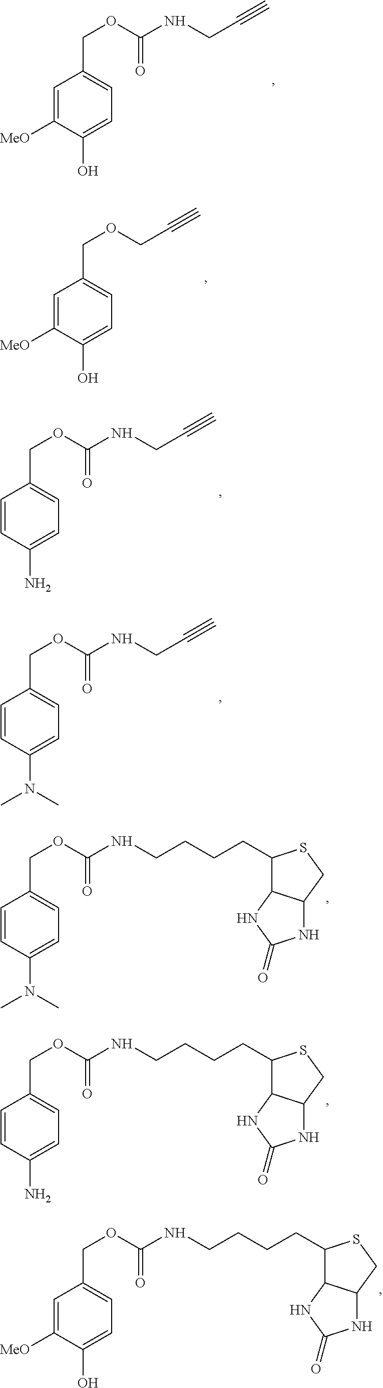

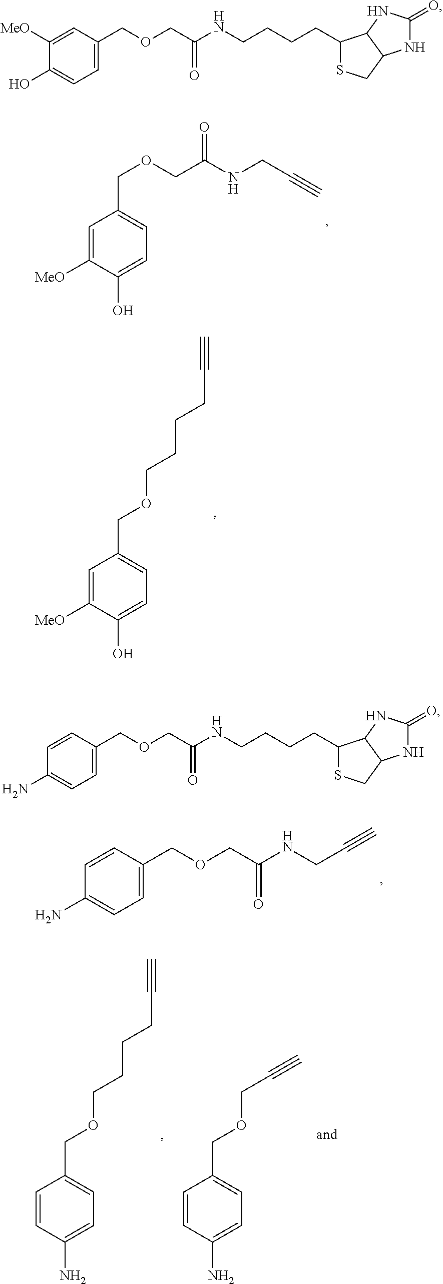

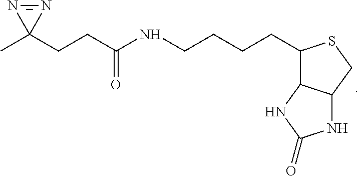 [SMILES: CC1(CCC(=O)NCCCCC2SCC3NC(=O)NC32)N=N1]

### III. Methods of Detecting Biological Interactions

In some embodiments, the presently disclosed probes and probe systems can be used in detecting one or more biological interactions between a biological target of interest (BTOI) and one or more second entities. In some embodiments, the BTOI is a protein (e.g., an enzyme, a cytokine, or a receptor), a peptide, a nucleic acid (e.g., a RNA or DNA), a drug or drug metabolite, or a cell (e.g., a particular type of cell, a particular type of cancer cell, or a unicellular microorganism (e.g., a bacteria)). The one or more second entities can include one or more classes of molecules or macromolecules expected or know to be present in a particular biological environment of interest, e.g., in a cell or cell compartment or in an extracellular environment. Thus, the detected biological interactions, which can include transient biological interactions, can include, but are not limited to protein-protein interactions, protein-metabolite interactions, cell-cell interactions, protein-nucleic acid interactions (e.g., protein-RNA or protein-DNA interactions), nucleic acid-drug interactions, and protein-drug interactions. In some embodiments, the detecting is performed in an organ, tissue, bodily fluid, or a live cell. In some embodiments, the detecting is performed in a cell culture or cell extract.

In some embodiments, the BTOI is a protein and the detecting is performed in a live cell transiently or stably expressing a fusion protein comprising the BTOI and a detectable protein or peptide tag (e.g., a SNAP-tag, a HALO-tag, or a CLIP-tag). In some embodiments, the BTOI is a cell and the detecting is performed in a cell culture, tissue, bodily fluid or organ comprising the BTOI wherein said BTOI expresses a detectable protein or peptide tag on a luminal surface of said BTOI. In some embodiments, the BTOI is a nucleic acid. In some embodiments, the BTOI is a drug compound or a metabolite thereof. In some embodiments, the same BTOI can be studied using two or more different probes and/or probe systems of the presently disclosed subject matter and/or under two or more different conditions.

In some embodiments, the presently disclosed subject matter provides a method for detecting spatiotemporal interactions of a BTOI, wherein the method comprises: (a) optionally labeling the BTOI with a moiety comprising a first binding partner (e.g., if such a moiety is not already present on the BTOI); (b) contacting the BTOI with a photoactive probe comprising: (i) a moiety that binds the first binding partner, (ii) a photoreactive moiety attached to a moiety that binds a second binding partner, and (iii) a photocleavable moiety attaching (i) and (ii); and (c) exposing the probe to light, thereby cleaving the photocleavable moiety and causing the photoreactive moiety to diffuse from the BTOI and react covalently or non-covalently with one or more biological entities in proximity to the BTOI and within a diffusion radius associated with the photoactive probe, thereby labeling said one or more biological entities with the moiety that binds a second binding partner. In some embodiments, the BTOI is a protein or a cell. In some embodiments, the method comprises detecting one or more cell-cell interactions, one or more cell-protein interactions, and/or one or more cell-drug interactions. In some embodiments, the method comprises detecting one or more protein-protein interactions, one or more protein-metabolite interactions, one or more protein-nucleic acid interactions, and/or one or more protein-drug interactions. In some embodiments, the method comprises detecting one or more protein-RNA interactions. In some embodiments, the method comprises detecting one or more protein-DNA interactions. In some embodiments, the method comprises detecting one or more nucleic acid-drug interactions.

Due to the modular nature of the presently disclosed stoichiometric photoproximity probes, the diffusion radius of the probe (and thus the radius of interrogation of the spatiotemporal interactions of the BTOI) is adjustable, e.g., based on the reactivity of the photoreactive moiety and/or the reactivity of the photocleavable moiety, such that the interactions of the BTOI over longer or shorter time periods can be determined and/or such that the entities within shorter or longer distances from the BTOI under particular conditions can be determined. The modular nature also provides for the detection of a wider variety of types of interactions of the BTOI, e.g., by adjusting the chemistry of the photoreactive moiety such that it can interact with different types of molecules or macromolecules. The “social network” of a BTOI can be determined with two or more different probes sequentially or simultaneously. For example, in some embodiments, the method can comprise contacting the BTOI with two or more stoichiometric probes, wherein each of said two or more stoichiometric probes has a different diffusion radius and the moiety that binds a second binding partner of each of said two or more stoichiometric probes binds a different second binding partner.

In some embodiments, the contacting the BTOI with the stoichiometric probe is performed in a live cell, a cell culture, a tissue sample, bodily fluid or an organ sample. The cleavage of the photocleavable group and activation of the photoreactive group are both triggered by light. Thus, in some embodiments, the method is free of a chemical or biological co-factor to activate the photoreactive group.

In some embodiments, the presently disclosed subject matter provides a method for detecting a spatiotemporal interaction of a BTOI wherein the method comprises: (a) providing a sample comprising a BTOI comprising a first binding partner (optionally a BTOI labeled with a moiety comprising the first binding partner); (b) contacting the BTOI with a photocatalytic probe comprising: (i) a moiety that binds the first binding partner and (ii) a photocatalytic moiety; (c) contacting the sample with one or more probe substrates, wherein each probe substrate comprises: (iii) a photoreactive moiety that is capable of undergoing a reaction catalyzed by the photocatalytic moiety and (iv) a detectable moiety or precursor thereof that is capable of specifically binding a second binding partner; and (d) exposing the sample to light, thereby exciting said photocatalytic moiety and causing the photocatalytic moiety to catalyze a reaction where the photoreactive moiety is transformed into a moiety that can react covalently or non-covalently with one or more biological entities in proximity to the BTOI, thereby labeling said one or more biological entities with the moiety that binds a second binding partner. In some embodiments, the sample is a live cell, a cell culture, a tissue sample, bodily fluid, or an organ sample. In some embodiments, the BTOI is a protein, a cell, a nucleic acid, a drug, or a drug metabolite. In some embodiments, the BTOI is a protein or a cell. In some embodiments, the method comprises detecting one or more cell-cell interactions, one or more cell-protein interactions, and/or one or more cell-drug interactions. In some embodiments, the method comprises detecting one or more protein-protein interactions, one or more protein-metabolite interactions, one or more protein-nucleic acid interactions (e.g., protein-DNA or protein-RNA interactions), and/or one or more protein-drug interactions.

Due to the modular nature of the presently disclosed catalytic photoproximity probe systems, the diffusion radius of the probe substrate (and thus the radius of interrogation of the spatiotemporal interactions of the BTOI) is adjustable, e.g., based on the reactivity and/or half-life of the moiety that is formed by the catalytic interaction between the photocatalytic group and the photoreactive group and/or the reactivity of the photocatalytic group and/or the length of the linker moiety of the photocatalytic probe, such that the interactions of the BTOI over longer or shorter time periods can be determined and/or such that the entities within shorter or longer distances from the BTOI under particular conditions can be determined. For example, if the photocatalytic group catalyzes a reaction of the photoreactive group to form a relatively longer-lived reactive moiety, the radius of interrogation will be larger than if the catalysis produces a reactive moiety with a shorter half-life. Furthermore, the rate of substrate conversion via the photocatalyst can affect the rate at which a reactive species is generated, further providing for fine tuning of the reactivity of the catalyst/catalyst substrate pair to interrogate biomolecular interactions at specified radii within the cell. The modular nature also provides for the detection of a wider variety of types of interactions of the BTOI, e.g., by adjusting the chemistry of the photoreactive moiety such that it can interact with different types of molecules or macromolecules. The “social network” of a BTOI can be determined with two or more different probe catalysts and/or probe substrates sequentially or simultaneously. For example, in some embodiments, the method can comprise contacting the BTOI with two or more probe substrates wherein each of said two or more probe substrates has a different diffusion radius and the moiety that binds a second binding partner of each of said two or more probe substrates binds a different second binding partner. In some embodiments, the method is free of a chemical or biological co-factor to activate the photoreactive group.

In some embodiments, the presently disclosed subject matter provides a method of detecting interactions of a BTOI, the method comprising: (a) providing a sample comprising a BTOI comprising a detectable tag, optionally a labelled BTOI wherein said labelled BTOI comprises the BTOI and a detectable tag; optionally wherein said BTOI is a cell or a protein, further optionally wherein the detectable tag is protein or peptide; (b) contacting the sample with a photoactive probe or probe system of the presently disclosed subject matter (e.g., with a probe of Formula (I) or a combination of a photocatalytic probe of Formula (VII) and a probe substrate of Formula (VIII)), wherein the target recognition moiety T specifically binds to the detectable tag of the labelled BTOI; (c) exposing the sample to light, thereby (i) triggering the cleavage of the photocleavable moiety P₁ and the activation of the photoreactive moiety P₂, wherein the photoreactive moiety P₂ reacts to form a covalent linkage with a second entity in proximity to the POI, thereby tagging said second entity with the detectable moiety R; or (ii) activating the photocatalytic moiety P_{c}, thereby catalyzing a reaction of the photoreactive moiety P₃, transforming said photoreactive moiety P₃ into a moiety that can react to form a covalent linkage with a second entity in proximity to the POI, thereby tagging said second entity with the detectable moiety R; and (d) detecting the detectable moiety R, thereby detecting the second entity interacting with or in proximity to the BTOI.

For example, in some embodiments, the method comprises: (a) providing a sample comprising a labelled BTOI, wherein said labelled BTOI comprises the BTOI and a detectable tag; (b) contacting the sample with a probe of one of Formulae (I), (II), (IIIa), (IIIb), (IVa), (IVb), (Va), (Vb), (VIa), or (VIb), wherein the target recognition moiety T of the probe specifically binds to the detectable tag of the labelled BTOI; (c) exposing the sample to light, thereby triggering the cleavage of the photocleavable moiety P₁ of the probe and the activation of the photoreactive moiety P₂ of the probe, wherein the photoreactive moiety P₂ reacts to form a covalent linkage with a second entity in proximity to the POI, thereby tagging said second entity with the detectable moiety R of the probe; and (d) detecting the detectable moiety R of the probe, thereby detecting the second entity interacting with or in proximity to the BTOI. In some embodiments, the BTOI is a protein or a cell. In some embodiments, the detectable tag is a protein or a peptide.

In some embodiments, the method comprises: (a) providing a sample comprising a labelled BTOI, wherein said labelled BTOI comprises the BTOI and a detectable tag; (b) contacting the sample with a photocatalytic probe of Formula (VII) and a probe substrate of Formula (VIII) (e.g., wherein the probe substrate of Formula (VIII) is present in a molar excess (e.g., a two, three, four, five, six, seven, eight, nine, or ten-fold excess or more) compared to the probe catalyst of Formula (VII); (c) exposing the sample to light, thereby activating the photocatalytic moiety P_{c} and catalyzing a reaction of the photoreactive moiety P₃, transforming said photoreactive moiety P₃ into a moiety that can react to form a covalent linkage with a second entity in proximity to the POI, thereby tagging said second entity with the detectable moiety R; and (d) detecting the detectable moiety R, thereby detecting the second entity interacting with or in proximity to the BTOI. In some embodiments, the BTOI is a protein or a cell. In some embodiments, the detectable tag is a protein or a peptide.

In some embodiments, the presently disclosed subject matter provides a method of detecting interactions (e.g., protein-protein interactions) of a protein of interest (POT), the method comprising: (a) providing a sample comprising a labelled POI, wherein said labelled POI comprises the POI and a detectable tag; (b) contacting the sample with a probe of one of Formula (I), (II), (IIIa), (IIIb), (IVa), (IVb), (Va), (Vb), (VIa), or VIb), wherein the target recognition moiety T of the probe specifically binds to the detectable tag of the labelled POI; (c) exposing the sample to light, thereby triggering the cleavage of the photocleavable moiety P₁ of the probe and the activation of the photoreactive moiety P₂ of the probe, wherein the photoreactive moiety P₂ reacts to form a covalent linkage with an entity (e.g., a protein) in proximity to the POI, thereby tagging said protein with the detectable moiety R of the probe; and (d) detecting the detectable moiety R of the probe, thereby detecting an entity (e.g., a protein) in proximity to the POI. In some embodiments, the detectable tag is protein or peptide. In some embodiments, the detectable tag is selected from the group including, but not limited to, a SNAP-tag, a Halo-Tag, a Clip-Tag, a receptor engineered with strained cyclooctyne, monomeric streptavidin, neutravidin, avidin, FKBP12 or a mutant thereof, and DHFR.

In some embodiments, the presently disclosed subject matter provides a method of detecting interactions (e.g., protein-protein interactions) of a protein of interest (POI), the method comprising: (a) providing a sample comprising a labelled POI, wherein said labelled POI comprises the POI and a detectable tag; (b) contacting the sample with a photocatalytic probe of Formula (VII) and a probe substrate of Formula (VIII) (e.g., an excess of the probe substrate compared to the photocatalytic probe), wherein the target recognition moiety T of the probe catalyst specifically binds to the detectable tag of the labelled POI; (c) exposing the sample to light, thereby activating the photocatalytic group of the photocatalytic probe to catalyze a reaction of the photoreactive group of the probe substrate, transforming the photoreactive group into a moiety that reacts to form a covalent linkage with an entity (e.g., a protein) in proximity to the POI, thereby tagging said protein with the detectable moiety R of the probe substrate; and (d) detecting the detectable moiety R of the probe, thereby detecting an entity (e.g., a protein) in proximity to the POI. In some embodiments, the detectable tag is protein or peptide. In some embodiments, the detectable tag is selected from the group including, but not limited to, a SNAP-tag, a Halo-Tag, a Clip-Tag, a receptor engineered with strained cyclooctyne, monomeric streptavidin, neutravidin, avidin, FKBP12 or a mutant thereof, and DHFR.

In some embodiments of these methods, the sample comprises a live cell comprising the labelled POI (e.g., a cell stably or transiently transformed to express a fusion protein comprising the POI and a peptide or protein tag).

In some embodiments, the method further comprises lysing the cells prior to the detecting of step (d). In some embodiments, the method further comprises enriching the sample for the detectable moiety R of the probe. For example, the enriching can comprise contacting the lysed cell sample with a solid support (e.g., polymeric beads or particles) comprising a binding partner of the detectable moiety R. In some embodiments, the detectable moiety R is biotin or an analog thereof, and the enriching comprises contacting the sample with streptavidin-coated beads. In some embodiments, the streptavidin-coated beads are streptavidin-coated magnetic beads. In some embodiments, the enriching can comprise affinity chromatography using a matrix attached to the binding partner of detectable moiety R. In some embodiments, the method further comprises contacting the enriched sample with trypsin or another enzyme to partially digest the proteins present in the enriched sample.

In some embodiments, the detecting comprises performing liquid chromatography-tandem mass spectrometry (LC-MS/MS) on the digested sample. In some embodiments, the detecting comprises immunoblotting.

In some embodiments, the method further comprises isotopic labeling of the sample. For instance, in some embodiments, the method further comprises culturing the live cell in a cell culture medium comprising heavy isotopes prior to the contacting of step (b), thereby providing a “heavy” cell sample. In some embodiments, the cell culture medium comprises ¹³C- and/or ¹⁵N-labeled amino acids. For example, in some embodiments, the cell culture medium comprises ¹³C-, ¹⁵N-labeled lysine and arginine. In some embodiments, after steps (b) and (c) and prior to the detecting of step (d), the heavy cells of the heavy cell sample are lysed to provide a lysed sample, and the detecting comprises: (d1) enriching the lysed sample for the detectable moiety R of the probe or probe substrate to provide an enriched sample; (d2) combining the enriched sample with an enriched sample prepared from a lysed sample of “light” live cells, wherein said light live cells are cells that (i) stably or transiently express the labelled POI, (ii) were cultured in a culture medium free of heavy isotopes, and (iii) were not contacted with the probe or probe system, thereby providing a combined enriched sample; (d3) performing liquid chromatography-tandem mass spectrometry (LC-MS/MS) on the combined enriched sample; and (d4) analyzing the data obtained in step (d3) to determine the identity of one or more proteins or other entities that interact with the POI.

In some embodiments, the presently disclosed subject matter further provides a kit for detecting one or more biological interactions of a biological target of interest (e.g., a cell or protein of interest). In some embodiments, the kit comprises: one or more probe of Formula (I) or a probe system comprising a probe catalyst of Formula (VII) and a probe substrate of Formula (VIII); and, optionally one or more additional components, such as one or more of: a cell culture medium, (optionally including a cell culture medium containing one or more heavy isotopes); a buffer; and a solid support material comprising a binding partner of the detectable moiety. In some embodiments, e.g., when the detectable moiety is biotin, the solid support material of the kit can comprise streptavidin-coated beads. In some embodiments, the kit can include at least two probes of Formula (I) or at least two probe catalysts of Formula (VII) and/or two probe substrates of Formula (VIII). In some embodiments, the at least two chemical probes or at least two different probe catalysts and/or probe substrates can have different diffusion radii. In some embodiments, the at least two probes or probe substrates comprise photoreactive moieties that react or undergo a catalytic transformation to react with different reactivity (e.g., to react with different types biological molecules or with different chemical groups on a biological molecule). In some embodiments, the kit comprises instructions for employing the components of the kit.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

General Synthetic Methods: Reagents purchased from commercial suppliers were analytical grade and used without further purification. All reactions were carried out in oven dried flasks using anhydrous solvents (Acros Organics, Thermo Fisher Scientific, Waltham, Mass., United States of America) unless otherwise specified. Reaction progress was monitored by thin-layer chromatography (TLC) on MACHEREY-NAGEL™ SIL G-25 UV254 TLC plates (Macherey, Nagel GmbH & Co., KG, Düren, Germany), visualized with UV light, ceric ammonium molybdate (CAM), p-anisidine, bromophenol blue, 2,4-dinitrophenyl hydrazine (DNP), or KMnO₄ TLC stains. Nuclear magnetic resonance spectra were acquired using either a Bruker AVANCE II+ 500; 11.7 Tesla NMR or Bruker DRX 400; 9.3 Tesla NMR instrument (Bruker, Billerica, Mass., United States of America). Accurate mass measurements were obtained using an Agilent 6224 TOF-MS instrument (Agilent Technologies, Santa Clara, Calif., United States of America). When necessary, compounds were purified via flash column chromatography using Siliaflash F60 60 Å, 230-400 mesh silica gel (Silicycle Inc., Quebec City, Canada)

### Chemical Synthesis of Photoproximity Profiling (P3)-Probes

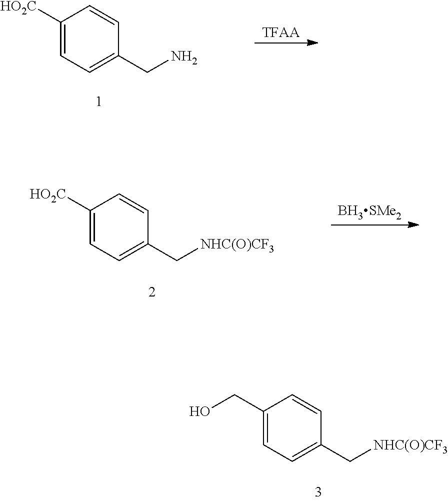

### 4-[[(2,2,2-trifluoroacetyl)amino]methyl]benzoic acid (2)

Solid 4-(aminomethyl)benzoic acid 1 (15.1 g, 100 mmol) was dissolved in TFAA (42 mL) cooled to 0° C. Once dissolved, the ice bath was removed and the reaction was allowed to stir at room temperature (rt) until starting material was consumed, about 2 hours. Upon completion, the reaction was quenched with H₂O (100 mL) and precipitate collected via vacuum filtration. The product was dried under suction then collected to afford benzoic acid 2 (24.0 g 97%) as a white solid.

¹H NMR (500 MHz, DMSO-d⁶) δ 7.93 (d, J=8.3 Hz, 2H), 7.39 (d, J=8.3 Hz, 2H), 4.47 (d, J=6.0 Hz, 2H). ¹³C NMR (125 MHz, DMSO-d⁶) δ 167.12, 156.58 (q, J=36.3 Hz), 142.50, 129.89, 129.65, 127.44, 116.05 (q, J=228.1 Hz), 42.41.

### 2,2,2-trifluoro-N-[[4-(hydroxymethyl)phenyl]methyl]acetamide (3)

Borane dimethylsulfide (13.8 mL, 145 mmol) was added dropwise to an anhydrous THF (483 mL) solution of benzoic acid 2 (11.9 g, 48.3 mmol) while maintaining an internal temperature of 0° C. After complete addition the ice bath was removed and the mixture was stirred at rt overnight. The reaction was quenched with MeOH (100 mL) and stirred at rt for an additional 1 hr. The volatiles were removed and the residue taken up in EtOAc. Impurities were removed by successive washes with 1M NaOH, H₂O, and brine. The organics were then dried over Na₂SO₄, filtered, and concentrated in vacuo. Subsequent column chromatographic purification of the residue, eluting with 20:1 (CH₂Cl₂:MeOH), provided acetamide 3 (10.4 g, 92%) as a white solid.

¹H NMR (500 MHz, CDCl₃-d) δ 7.35 (d, J=8.2 Hz, 1H), 7.27 (d, J=8.0 Hz, 1H), 4.67 (s, 1H), 4.50 (d, J=5.8 Hz, 1H). ¹³C NMR (125 MHz, CDCl3-d) δ 157.32 (q, J=36.7 Hz), 141.11, 135.33, 128.29, 127.67, 115.97 (q, J=287.8 Hz), 64.90, 43.75.

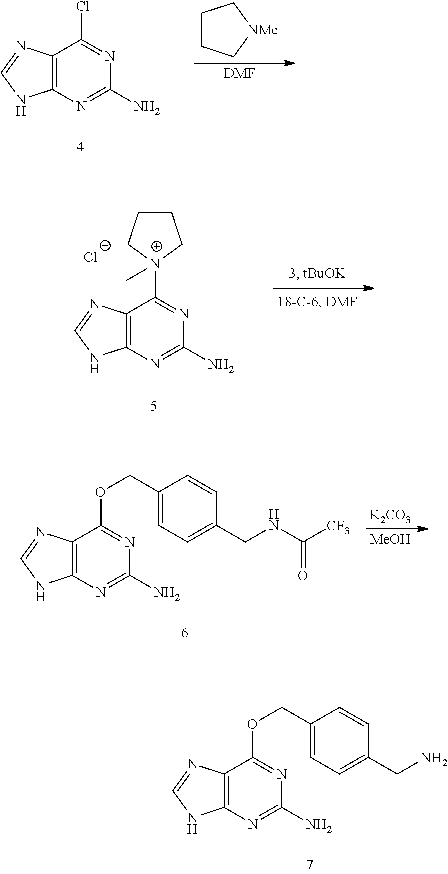

### 6-(1-methylpyrrolidin-1-ium-1-yl)-7H-purin-2-amine chloride (5)

Neat N-methylpyrollidine (7.80 mL, 73.7 mmol) was added to an anhydrous DMF (144 mL) solution of 6-chloro-7H-purin-2-amine 4 (5.00 g, 29.5 mmol) and stirred at 40° C. overnight. The resultant chloride salt 5 (5.45 g, 73%) was collected via vacuum filtration, dried under suction, and used without further purification.

### N-[[4-[(2-amino-7H-purin-6-yl)oxymethyl]phenyl]methyl]-2,2,2-trifluoroacetamide (6)

An oven dried round-bottom flask containing methylpyrrolidinium chloride 4 (6.90 g, 27.1 mmol), acetamide 3 (12.6 g, 54.2 mmol), potassium tert-butoxide (12.2 g, 108 mmol), and 18-c-6 (1.07 g, 4.07 mmol) in 54 mL DMF was stirred for 6 hrs at 50° C. Upon completion the solvent was evaporated and the crude residue absorbed to silica. Purification was achieved using column chromatography eluting with a gradient of MeOH in CH₂Cl₂ (2-10%) to give the trifluoroacetamide protected amine 6 (8.94 g, 90%) as a white solid.

¹H NMR (500 MHz, DMSO-d⁶) δ 7.81 (s, 1H), 7.49 (d, J=7.9 Hz, 2H), 7.30 (d, J=8.0 Hz, 2H), 5.46 (s, 2H), 4.4 (d, J=5.9 Hz, 2H).

### 6-[[4-(aminomethyl)phenyl]methoxy]-7H-purin-2-amine (7)

Trifluoroacetamide 6 (2.59 g, 7.06 mmol) was added to a suspension of K₂CO₃ (4.84 g, 35.0 mmol) in 21 mL of MeOH:H₂O (20:1) and stirred vigorously overnight at 50° C. Upon consumption of the starting material the mixture was filtered through a pad of celite, washing with MeOH. The filtrate was concentrated in vacuo and the residue taken up in 10 mL H₂O. While cooling, the pH was adjusted to about 7 with HCl. The resultant precipitate was isolated via suction filtration and washed with cold water to yield amine 7 (1.77 g, 93%) as a white solid. ¹H NMR (500 MHz, DMSO-d⁶) δ 7.82 (s, 1H), 7.43 (d, J=7.9 Hz, 2H), 7.34 (d, J=7.8 Hz, 2H), 5.45 (s, 2H), 3.71 (s, 2H). ¹³C NMR (125 MHz, DMSO-d⁶) δ 159.65, 157.73, 143.72, 140.45, 135.00, 134.67, 128.47, 127.14, 126.98, 66.66, 45.27.

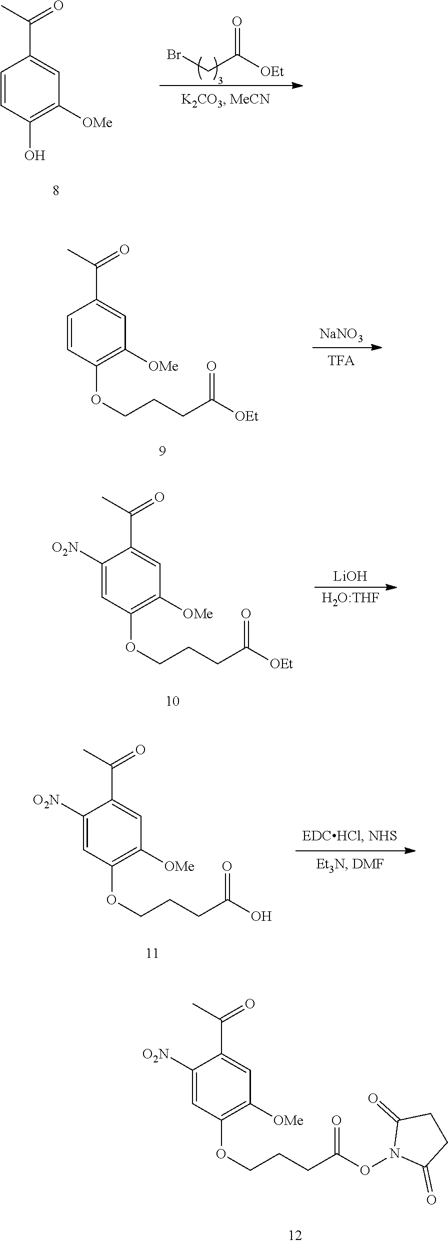

### ethyl 4-(4-acetyl-2-methoxyphenoxy)butanoate (9)

Solid 1-(4-hydroxy-3-methoxyphenyl)ethanone 8 (8.31 g, 50 mmol) was added to a suspension of K₂CO₃ (69.1 g, 500 mmol) in 100 mL of anhydrous MeCN. Ethyl 4-bromobutanoate (14.3 mL, 100 mmol) was added and the mixture stirred overnight at 60° C. Upon consumption of starting material the mixture was filtered over a pad of celite, washing with cold MeCN. Volatiles were evaporated and the residue recrystallized from Et₂O to provide ester 9 (13.2 g, 94%) as a white powder. ¹H NMR (400 MHz, CDCl3-d) δ 7.57 (dd, J=8.3, 2.1 Hz, 1H), 7.55 (d, J=2.0 Hz, 1H), 6.92 (d, J=8.3 Hz, 1H), 4.21-4.13 (m, 4H), 3.94 (s, 3H), 2.59 (s, 3H), 2.56 (t, J=7.2 Hz, 2H), 2.26-2.17 (m, 2H), 1.28 (t, J=7.1 Hz, 3H).

### ethyl 4-(4-acetyl-2-methoxy-5-nitrophenoxy)butanoate (10)

Using an ice bath, 50 mL of trifluoroacetic acid (50 mL) was cooled to 0° C. prior to the addition of ester 9 (12.8 g, 45.7 mmol). Solid NaNO₃ (11.6 g, 136 mmol) was added in portions to the stirred mixture maintaining 0° C. Upon completion the reaction was quenched with 200 mL of H₂O, the resulting precipitate filtered, then dried under suction to afford the nitro arene 10 (13.8 g, 93%) as a yellow powder. ¹H NMR (400 MHz, CDCl3) δ 7.61 (s, 1H), 6.74 (s, 1H), 4.21-4.12 (m, 4H), 3.95 (s, 3H), 2.54 (t, J=7.2 Hz, 2H), 2.49 (s, 3H), 2.25-2.16 (m, 2H), 1.27 (t, J=7.2 Hz, 3H).

### 4-(4-acetyl-2-methoxy-5-nitrophenoxy)butanoic acid (11)

A solution of nitro arene 10 (2.52 g, 7.76 mmol) in THF (76 mL) was combined with 39 mL of 2M LiOH. The reaction was allowed to stir vigorously for 3 hrs at rt. Upon starting material consumption, the reaction was quenched with 1M NaHSO₄ (100 mL) and extracted with EtOAc. The combined organic extracts were dried over NaSO₄ and filtered. Precipitate that formed upon solvent removal in vacuo was filtered and dried under suction to provide the title acid 11 (1.86 g, 81%) as a pale yellow powder. ¹H NMR (400 MHz, CDCl₃) δ 7.62 (s, 1H), 6.75 (s, 1H), 4.17 (t, J=6.2 Hz, 2H), 3.95 (s, 3H), 2.63 (t, J=7.1 Hz, 2H), 2.49 (s, 3H), 2.27-2.13 (m, 2H).

### (2,5-dioxopyrrolidin-1-yl) 4-(4-acetyl-2-methoxyphenoxy)butanoate (12)

Acid 11 (4.65 g, 15.6 mmol) was added to an anhydrous DMF (30 mL) solution containing EDC.HCl (4.48 g, 23.4 mmol) and NHS (2.69 g, 23.4 mmol). The mixture was stirred overnight at rt. Addition of chilled Et₂O to the bulk solution resulted in precipitate, which was collected via vacuum filtration and dried under suction to yield NHS-ester 12 (5.47 g, 89%) as a yellow powder.

¹H NMR (500 MHz, CDCl₃) δ 7.63 (s, 1H), 6.75 (s, 1H), 4.21 (t, J=6.0 Hz, 2H), 3.96 (s, 3H), 2.89 (t, J=7.3 Hz, 2H), 2.85 (bs, 4H), 2.49 (s, 3H), 2.35-2.28 (m, 2H). ¹³C NMR (125 MHz, CDCl₃) δ 200.28, 169.20, 168.18, 154.54, 148.71, 133.29, 108.88, 108.44, 67.66, 56.72, 30.58, 27.62, 25.73, 24.21.

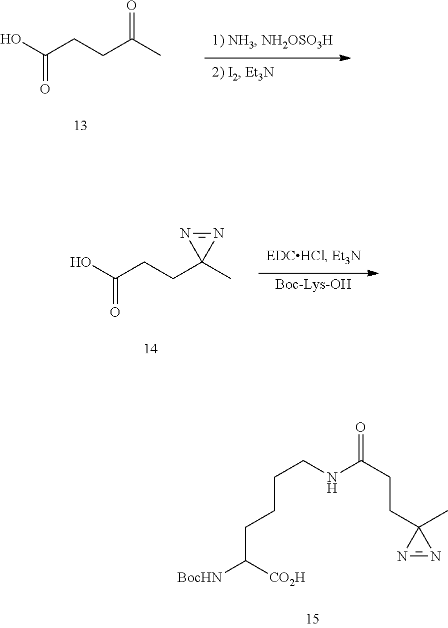

### 3-(3-methyldiazirin-3-yl)propanoic acid (14)

Gaseous ammonia (about 200 mL) was condensed at −78° C. into an oven dried two-neck round-bottom flask, 4-oxopentanoic acid 13 (11.6 g, 100 mmol) added, then the mixture refluxed at 0° C. for 4 hrs. A suspension of amino hydrogen sulfate (14.0 g, 123 mmol) in anhydrous MeOH (150 mL) was added via addition funnel over a 45 min period maintaining 0° C. The heterogenous reaction was then vigorously stirred overnight, allowing the ammonia to slowly evaporate as the temperature rose to rt. The resulting slurry was filtered over a pad of celite, washing the solids with MeOH. The solvent was reduced under vacuum (50 mL) to ensure removal of residual ammonia. The crude residue was then diluted with MeOH (100 mL) and cooled to 0° C. before the addition of Et₃N (20.8 mL, 150 mmol). Solid I₂ (25.5 g, 100 mmol) was added in portions until the color of iodine persisted and the reaction stirred for 2 hrs allowing the temperature to rise to rt. Upon completion, the volatiles were removed in vacuo and the residue diluted with EtOAc (150 mL). The organics were successively washed with 1 M NaHSO₄ (50 mL×2), 0.5 M Na₂S₂O₃ (50 mL), brine (50 mL) then dried over NaSO₄. The combined organics were filtered and absorbed to silica then subjected to chromatographic purification eluting with a gradient of MeOH in CH₂Cl₂ (2-5%). The title acid 14 (7.29 g, 57%) was isolated as a thin red oil.

¹H NMR (500 MHz, CDCl₃) δ 11.18 (bs, 1H), 2.18-2.14 (m, 2H), 1.68-1.60 (m, 2H), 0.95 (s, 3H). ¹³C NMR (125 MHz, CDCl₃) δ 178.66, 29.24, 28.48, 25.05, 19.55.

### (2S)-6-[[3-(3-methyldiazirin-3-yl)propanoyl]amino]-2-[(2-methylpropan-2-yl)oxycarbonylamino]hexanoic acid (15)

HATU (910 mg, 2.39 mmol) was added to a 20 mL vial charged with acid 14 (323 mg, 2.52 mmol), iPr₂NEt (1.30 mL, 7.87 mmol) in anhydrous DMF (12 mL). The mixture was stirred for 1 hr at rt after which Boc-Lys-OH (621 mg, 2.52 mmol) was added in one portion and stirring continued overnight. Upon completion, the reaction was diluted with EtOAc and washed successively with 1 M NaHSO₄ (20 mL×2), H₂O (20 mL×2), and brine (20 mL). The organics were filtered and concentrated before being purified by reverse phase HPLC, eluting with a gradient of MeOH in H₂O (0-95%). The desired acid 15 (381 mg, 42%) was obtained in modest yield as a beige solid.

¹H NMR (400 MHz, DMSO-d⁶) δ 12.4 (s, 1H), 7.83 (t, J=5.6 Hz, 1H), 7.01 (d, J=8.0 Hz, 1H), 3.81 (ddd, J=9.5, 7.9, 4.7 Hz, 1H), 3.00 (q, J=6.4 Hz, 2H), 1.93 (dd, J=8.5, 6.9 Hz, 2H), 1.67-1.59 (m, 1H), 1.59-1.52 (m, 4H), 1.37-1.27 (m, 12H), 0.97 (s, 3H). ¹³C NMR (125 MHz, DMSO-d⁶) δ 174.28, 170.50, 155.61, 77.95, 53.43, 38.24, 30.41, 29.88, 29.79, 28.68, 28.23, 25.85, 23.07, 19.34.

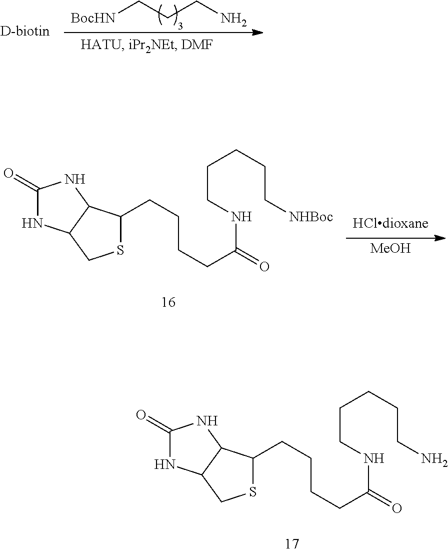

### (3aS,4S,6aR)-N-[5-[(1,1-dimethylethoxy)carbonyl]aminopentyl]hexahydro-2-oxo-1H-thieno[3,4-d]imidazole-4-pentanamide (16)

A solution of D-biotin (1.95 g, 7.98 mmol), iPr₂NEt (4.20 mL, 24.1 mmol) and HATU (6.08 g, 16.0 mmol) in anhydrous DMF (20 mL) was stirred for 1 hr at rt. Neat tert-butyl N-(5-aminopentyl)carbamate (1.78 g, 8.78 mmol) was added and the reaction stirred overnight. Upon completion Et₂O was flowed in and the resultant precipitate filtered under vacuum. The crude solid was purified by iterative recrystallization from acetone and hexanes then H₂O and acetone. Amide 16 (2.95 g, 80%) was isolated by vacuum filtration as a white powder. ¹H NMR (500 MHz, DMSO-d⁶) δ 7.73 (t, J=5.6 Hz, 1H), 6.76 (t, J=5.7 Hz, 1H), 6.43 (s, 1H), 6.36 (s, 1H), 4.30 (dd, J=7.6, 5.0 Hz, 1H), 4.12 (ddd, J=7.7, 4.4, 1.9 Hz, 1H), 3.09 (ddd, J=8.7, 6.1, 4.4 Hz, 1H), 2.99 (q, J=6.6 Hz, 2H), 2.87 (q, J=6.7 Hz, 2H), 2.82 (dd, J=12.4, 5.1 Hz, 1H), 2.57 (d, J=12.4 Hz, 1H), 2.03 (t, J=7.4 Hz, 2H), 1.60-1.57 (m, 1H), 1.54-1.41 (m, 3H), 1.36-1.17 (m, 20H). ¹³C NMR (125 MHz, DMSO-d⁶) δ 171.78, 162.71, 155.58, 77.32, 61.05, 59.19, 55.46, 38.35, 35.23, 29.20, 28.89, 28.30 (4C), 28.25, 28.06, 25.36, 23.74.

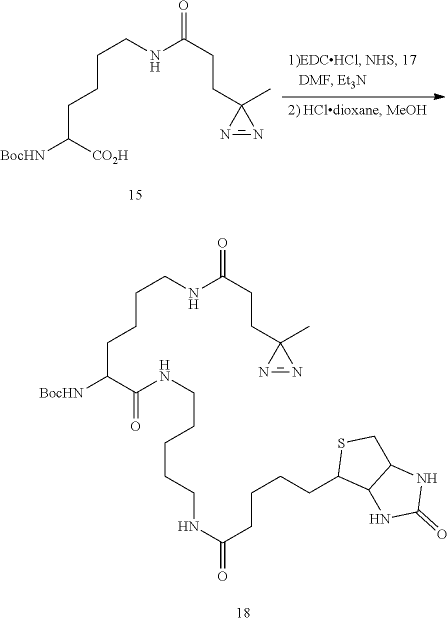 [SMILES: CC1(CCC(=O)NCCCCC(NC(=O)OC(C)(C)C)C(=O)O)N=N1 ; CC1(CCC(=O)NCCCCC(NC(=O)OC(C)(C)C)C(=O)NCCCCCNC(=O)CCCCC2SCC3NC(=O)NC32)N=N1]

### tert-butyl N-[6-[[3-(3-methyldiazirin-3-yl)propanoyl]amino]-1-oxo-1-[5-[5-(2-oxo-1,3,3a,4,6,6a-hexahydrothieno[3,4-d]imidazol-4-yl)pentanoylamino]pentylamino]hexan-2-yl]carbamate (18)

Amide 16 (1.80 g, 4.20 mmol) was dissolved with 4 M HCl in 1,4-dioxane (21 mL) and MeOH (1 mL). The mixture stirred at rt for 1 hr after which the volatiles were evaporated. The crude residue was taken up in 20 mL of MeOH and chilled with an ice bath before the addition of 7 M ammonia in MeOH (6 mL). The organics were reduced under vacuum and filtered to remove solids. The residue resulting from filtrate concentration was recrystallized from MeOH in Et₂O to afford amine 17 in quantitative yield and was used without further purification. A separate round-bottom flask containing acid 15 (712 mg, 2.00 mmol), HATU (912 mg, 2.40 mmol), and Et₃N (417 μL, 3.0 mmol) in anhydrous MeCN (10 mL) was stirred at it for 4 hrs. Afterward, an anhydrous DMSO (7 mL) solution of amine 17 (788 mg, 2.40 mmol) was flowed in and the mixture stirred overnight. Upon completion, the reaction was diluted with EtOAc and washed successively with 1 M NaHSO₄ (20 mL), H₂O (20 mL×2), brine (20 mL) then dried over NaSO₄. The crude residue was purified by preparatory reverse phase HPLC, eluting with a gradient of MeOH in H₂O (50-95%). Carbamate 18 (453 mg, 34%) was isolated as a waxy solid. ¹H NMR (500 MHz, CD₃OD) δ 4.50 (dd, J=7.9, 4.8 Hz, 1H), 4.31 (dd, J=7.9, 4.4 Hz, 1H), 3.95 (dd, J=8.9, 5.2 Hz, 1H), 3.24-3.13 (m, 7H), 2.93 (dd, J=12.7, 5.0 Hz, 1H), 2.71 (d, J=12.7 Hz, 1H), 2.20 (t, J=7.4 Hz, 2H), 2.07 (dd, J=8.5, 6.9 Hz, 2H), 1.79-1.30 (m, 29H), 1.01 (s, 3H). ¹³C NMR (125 MHz, CD₃OD) δ 175.80, 175.03, 174.29, 165.98, 157.68, 80.47, 63.31, 61.54, 56.99, 56.13, 41.05, 40.17, 40.13, 40.06, 36.78, 33.06, 31.49, 31.31, 29.97, 29.93, 29.76, 29.46, 28.74, 26.89, 26.33, 25.12, 24.26, 19.76.

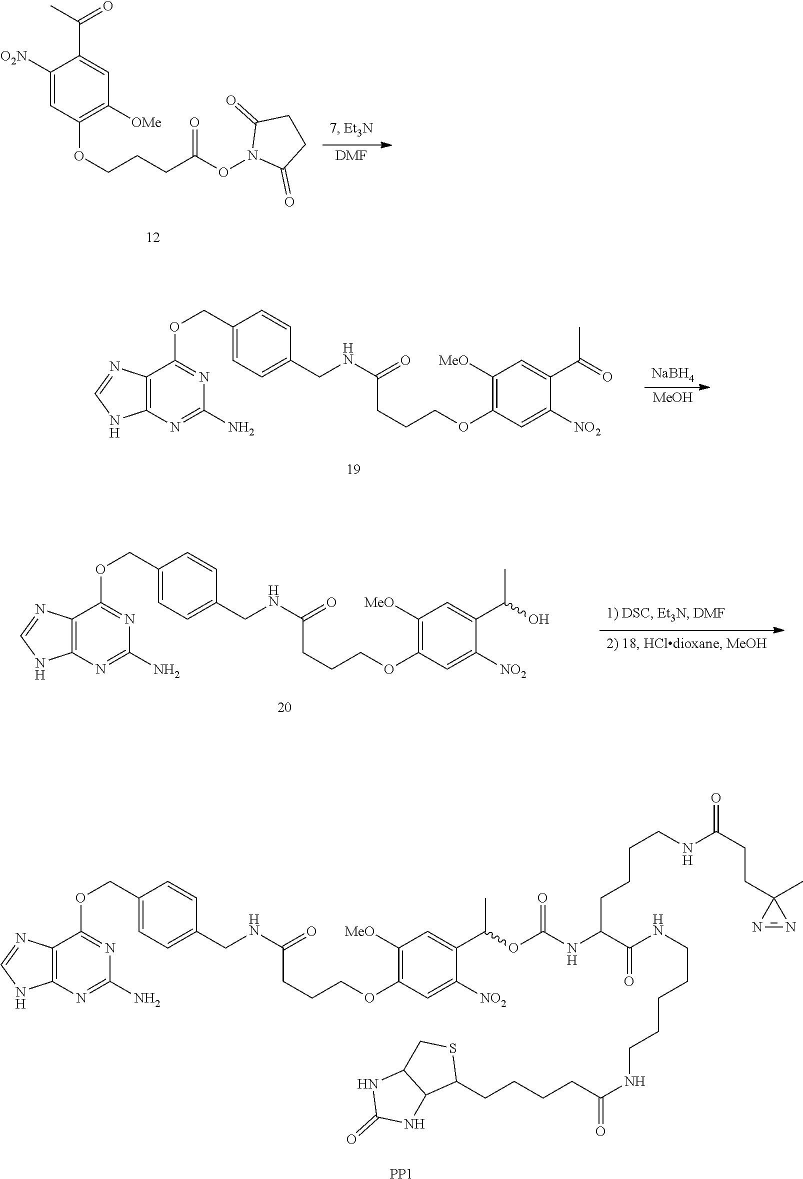

### Ketone (19)

A DMF (16 mL) solution of NHS-ester 12 (2.30 g, 5.83 mmol) was added to a suspension of amine 7 (1.50 g, 5.55 mmol) and iPr₂NEt (2.75 mL, 16.6 mmol) in anhydrous DMF (25 mL). The resulting homogenous mixture was stirred at rt for 5 hrs. Upon consumption of starting material, chilled Et₂O was flowed into the reaction and supernatant decanted from resultant oil. The crude residue was taken up in CH₂Cl₂ and sonicated until formation of precipitate, which was isolated by filtration. The solids were dried under suction to yield pure ketone 19 (1.32 g, 43%) as a beige powder. ¹H NMR (500 MHz, DMSO-d⁶) δ 8.42 (t, J=5.9 Hz, 1H), 8.19 (s, 1H), 7.62 (s, 1H), 7.46 (d, J=7.7 Hz, 2H), 7.27 (d, J=7.8 Hz, 2H), 7.23 (s, 1H), 5.48 (s, 2H), 4.28 (d, J=5.9 Hz, 2H), 4.12 (t, J=6.4 Hz, 2H), 3.92 (s, 3H), 2.51 (s, 3H), 2.33 (t, J=7.4 Hz, 2H), 2.00 (p, J=6.9 Hz, 2H).

¹³C NMR (125 MHz, DMSO-d⁶) δ 199.35, 171.42, 159.13, 158.88, 154.76, 154.19, 153.28, 148.58, 139.83, 138.36, 134.59, 131.11, 128.77, 127.31, 109.84, 107.96, 68.55, 67.38, 56.67, 41.89, 31.47, 30.73, 30.05, 24.58.

### Alcohol (20)

Solid NaBH₄ (113 mg, 3.00 mmol) was added in portions to a rt methanolic solution (10 mL) of ketone 19 (550 mg, 1.00 mmol) with vigorous stirring. Upon completion, the reaction was passed through a plug of silica to remove inorganics. The filtrate was concentrated in vacuo and the residue sonicated in Et₂O. The resultant precipitate was isolated via vacuum filtration then dried under suction to yield alcohol 20 (395 mg, 72%) as a yellow powder. ¹H NMR (400 MHz, CD₃OD) δ 7.84 (bs, 11H), 7.52 (s, 11H), 7.42 (d, J=7.9 Hz, 2H), 7.37 (s, 11H), 7.26 (d, J=8.0 Hz, 2H), 5.49 (s, 2H), 5.44 (q, J=6.2 Hz, 1H), 4.36 (s, 2H), 4.04 (t, J=6.1 Hz, 2H), 3.91 (s, 3H), 2.46 (t, J=7.3 Hz, 2H), 2.12 (p, J=6.6 Hz, 2H), 1.45 (d, J=6.3 Hz, 3H).

### Photoproximity Probe One (PP1)

A solution of carbamate 18 (270 mg, 405 μmol) in MeOH (1 mL) was added to 4 M HCl.dioxane (2 mL) at 0° C. The temperature was allowed to come to rt while stirring for 2 hrs. Upon disappearance of starting material the volatiles were evaporated and the residue left to dry under vacuum overnight. The crude hydrochloride salt was used without further purification.

In a separate flask, alcohol 20 (57.9 mg, 105 μmol) and Et₃N (41.0 μL, 294 μmol) was stirred at rt in anhydrous MeCN (10 mL) while solid DSC (75.3 mg, 294 μmol) was added. The mixture was stirred overnight, after which additional Et₃N (22 μL, 157 μmol) was added. A solution of the amine in DMSO (1 mL) was added and the mixture stirred until consumption of starting material. The volatiles were removed under vacuum and the residue purified by reverse phase HPLC eluting with a gradient of MeOH in H₂O (50-95%). The proximity probe PP1 (7.60 mg, 6%) was isolated as a yellow solid and characterized as mixture of diastereomers. ¹H NMR (500 MHz, CD₃OD) δ ¹H NMR (500 MHz, CD₃OD) δ 7.78 (s, 2H), 7.58 (s, 2H), 7.44 (d, J=7.7 Hz, 4H), 7.29-7.23 (m, 4H), 7.17 (d, J=1.7 Hz, 1H), 6.25 (q, J=6.3 Hz, 1H), 5.51 (s, 414), 4.51-4.41 (m, 3H), 4.37 (s, 4H), 4.31-4.21 (m, 3H), 4.06 (q, J=6.0 Hz, 4H), 4.00-3.88 (m, 8H), 3.23-2.96 (m, 19H), 2.94-2.84 (m, 3H), 2.73-2.63 (m, 3H), 2.46 (t, J=7.3 Hz, 4H), 2.22-2.09 (m, 8H), 2.10-1.98 (m, 4H), 1.78-1.16 (m, 35H), 0.98 (d, J=20.3 Hz, 7H). ¹³C NMR (125 MHz, CD₃OD) δ 175.97, 175.96, 175.90, 175.89, 175.14, 175.13, 174.61, 174.61, 174.44, 174.37, 170.32, 166.08, 166.07, 161.11, 160.78, 157.44, 157.42, 155.72, 155.62, 148.67, 148.58, 141.02, 140.73, 139.89, 139.89, 139.87, 137.22, 135.31, 135.15, 135.15, 129.63, 129.59, 128.60, 114.45, 110.16, 110.13, 109.45, 109.40, 70.23, 70.12, 69.62, 68.47, 63.35, 63.34, 61.60, 61.59, 57.00, 56.95, 56.39, 49.85, 49.51, 49.34, 49.17, 49.00, 48.83, 48.66, 48.49, 43.88, 41.05, 40.21, 40.18, 40.11, 40.09, 36.82, 36.80, 33.41, 33.02, 32.88, 31.54, 31.48, 31.36, 31.32, 29.96, 29.85, 29.77, 29.48, 26.91, 26.88, 26.41, 26.36, 25.13, 24.99, 24.31, 24.27, 22.45, 22.40, 19.74, 19.73. HRMS (ESI⁻) calculated for C₅₃H₇₃N₁₅O₁₂SH [M+H]⁺ 1144.5362, found 1144.5371.

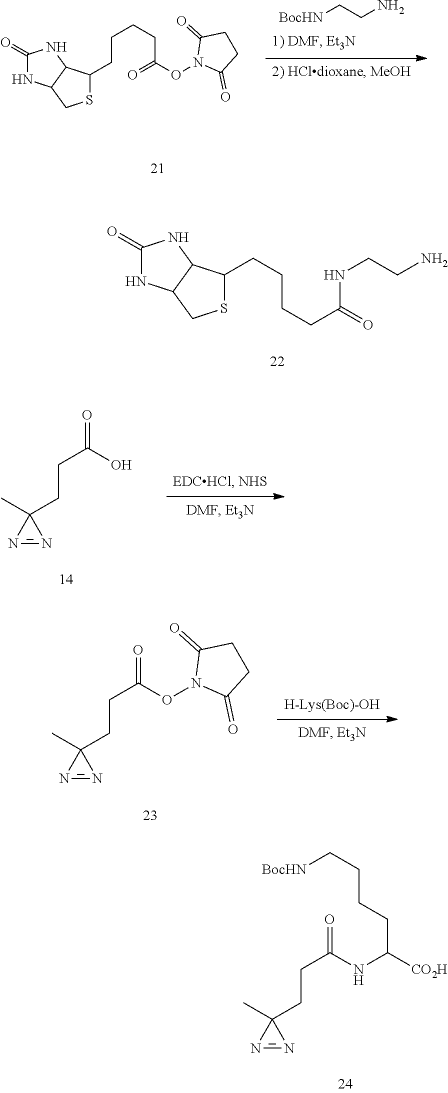

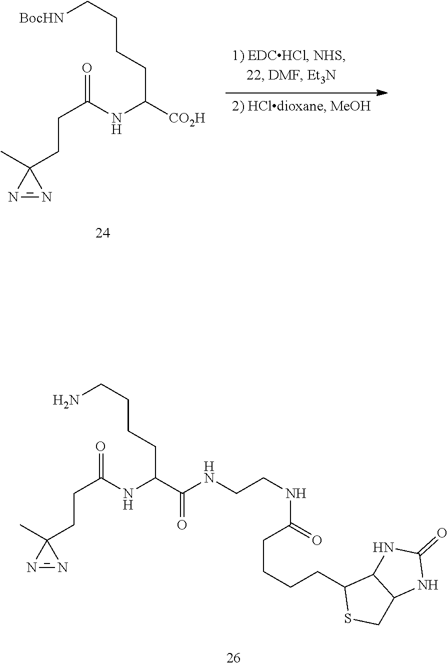 [SMILES: CC1(CCC(=O)NC(CCCCNC(=O)OC(C)(C)C)C(=O)O)N=N1 ; CC1(CCC(=O)NC(CCCCN)C(=O)NCCNC(=O)CCCCC2SCC3NC(=O)NC32)N=N1]

### (2,5-dioxopyrrolidin-1-yl)5-(2-oxo-1,3,3a,4,6,6a-hexahydrothieno[3,4-d]imidazol-4-yl)pentanoate (21)

An oven dried 100 mL round-bottom containing D-biotin (2.44 g, 10 mmol), EDC.HCl (2.87 g, 15 mmol), and NHS (1.73 g, 15 mmol) in 40 mL of anhydrous DMF was stirred overnight at rt. Upon consumption of starting material, cold Et₂O was flowed in and supernatant decanted from the resultant oil. The residue was sonicated in fresh Et₂O to provide pure NHS-ester 21 (5.73 g, 83%) as a fine white powder that was isolated by vacuum filtration and dried under suction. ¹H NMR (500 MHz, DMSO-d⁶) δ 6.45 (s, 1H), 6.38 (s, 1H), 4.31 (dd, J=7.6, 5.0 Hz, 1H), 4.15 (ddd, J=7.7, 4.5, 1.8 Hz, 1H₁), 3.10 (ddd, J=8.3, 6.3, 4.3 Hz, 1H), 2.87-2.79 (m, 5H), 2.67 (t, J=7.4 Hz, 2H), 2.58 (d, J=12.4 Hz, 1H), 1.71-1.58 (m, 3H), 1.55-1.34 (m, 3H). ¹³C NMR (125 MHz, DMSO-d⁶) δ 170.35, 169.01, 162.80, 61.07, 59.24, 55.31, 30.06, 27.89, 27.65, 25.50, 24.37.

### N-(2-aminoethyl)-5-(2-oxo-1,3,3a,4,6,6a-hexahydrothieno[3,4-d]imidazol-4-yl)pentanamide (22)

Neat tert-butyl N-(2-aminoethyl)carbamate was added to a suspension of NHS-ester 21 in anhydrous DMF (8.0 mL) and the reaction stirred at rt overnight. Upon consumption of starting material Et₂O was flowed in the mixture chilled to −20° C. overnight. The resultant precipitate was collected via vacuum filtration and dried under suction to yield Boc-protected amine (1.18 g, 78%) as a white powder. The intervening carbamate was stirred in MeOH containing 4M HCl to provide the amine after evaporation of the solvent, which was taken on without further purification. ¹H NMR (500 MHz, DMSO-d⁶) δ 6.45 (s, 1H), 6.39 (s, 1H), 4.30 (dd, J=7.7, 5.1 Hz, 1H), 4.13 (ddd, J=7.8, 4.4, 1.9 Hz, 1H), 3.28 (q, J=6.2 Hz, 2H), 3.10 (ddd, J=8.6, 6.1, 4.4 Hz, 1H), 2.86-2.78 (m, 3H), 2.57 (d, J=12.4 Hz, 1H), 2.10 (t, J=7.5 Hz, 2H), 1.65-1.41 (m, 4H), 1.39-1.22 (m, 2H). ¹³C NMR (125 MHz, DMSO-d⁶) δ 172.78, 162.75, 61.05, 59.22, 55.42, 38.59, 36.39, 35.16, 28.26, 28.08, 25.03.

### (2,5-dioxopyrrolidin-1-yl) 3-(3-methyldiazirin-3-yl)propanoate (23)

To a solution of crude acid 14 (2.40 g, 18.7 mmol) in anhydrous DMF (37 mL) was added EDC.HCl (4.48 g, 23.4 mmol) and NHS (2.69 g, 23.4 mmol). This mixture was stirred at room temperature overnight. Upon completion the reaction was diluted with H₂O and extracted with Et₂O (50 mL×3). The combined extracts were dried over Na₂SO₄, filtered, and concentrated under vacuum. Precipitate that formed during evaporation of the volatiles was filtered and dried under suction to provide NHS ester 23 (2.27 g, 54%) as a white solid. ¹H NMR (500 MHz, CDCl₃) δ 2.83 (s, 4H), 2.54-2.47 (m, 2H), 1.86-1.75 (m, 2H), 1.06 (s, 3H). ¹³C NMR (125 MHz, CDCl₃) δ 169.08, 167.71, 29.58, 25.82, 25.68, 24.86, 19.59.

### Biotinyl N-Boc-Photo Lysine (26)

N₆-(tert-butoxycarbonyl)-L-lysine (246 mg 1.00 mmol) and Et₃N (278 μL, 2.00 mmol) in anhydrous DMF (4 mL) were stirred while solid NHS-ester 23 (270 mg, 1.2 mmol) was added. The reaction was stirred at rt and progress monitored by LC/MS. Upon consumption of starting material, EDC.HCl (287 mg, 1.50 mmol) and NHS (173 mg, 1.50 mmol) were added at rt. The mixture was stirred at rt overnight, after which chilled Et₂O was flowed in and resultant precipitate isolated by vacuum filtration. The NHS ester (319 mg, 0.704 mmol) and Et₃N (293 μL, 2.11 mmol) were combined in fresh anhydrous DMF (4 mL) with stirring while amine 22 (242 mg, 0.845 mmol) was added. Upon completion, diethyl ether was flowed in and the reaction stored at −20° C. overnight. The resultant precipitate was isolated by vacuum filtration and dried under suction to afford carbamate 26 (323 mg, 73%) as a beige solid. ¹H NMR (500 MHz, CD₃OD) δ 4.51 (dd, J=7.9, 4.8 Hz, 1H), 4.38-4.30 (m, 1H), 4.25-4.17 (m, 1H), 3.32-3.18 (m, 2H), 3.09-2.98 (m, J=6.8 Hz, 3H), 2.93 (dd, J=12.8, 4.9 Hz, 1H), 2.72 (d, J=12.7 Hz, 1H), 2.26-2.09 (m, 4H), 1.89-1.56 (m, 7H), 1.52-1.41 (m, 18H), 1.02 (d, J=2.2 Hz, 3H). ¹³C NMR (125 MHz, CD₃OD) δ 176.29, 175.43, 174.83, 174.76, 174.63, 174.59, 174.51, 166.03, 158.42, 79.77, 63.22, 63.15, 61.61, 56.89, 55.05, 55.02, 53.50, 41.03, 40.17, 39.91, 39.85, 36.78, 36.73, 32.56, 32.20, 31.38, 31.22, 31.19, 30.99, 30.95, 29.68, 29.57, 29.42, 28.80, 28.78, 26.74, 26.38, 26.29, 24.21, 24.13, 19.77, 19.69.

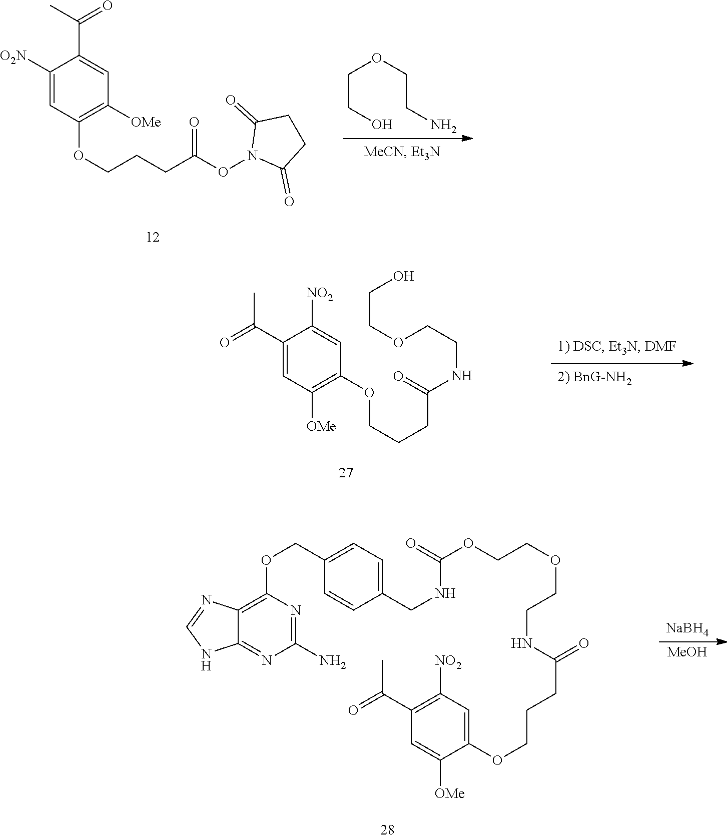

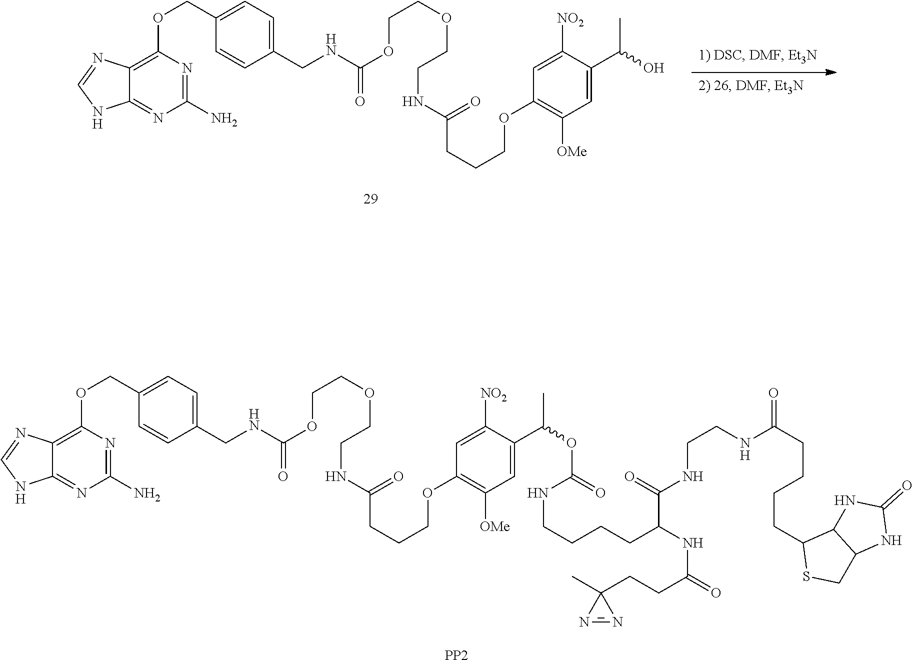 [SMILES: COc1cc(C(C)O)c([N+](=O)[O-])cc1OCCCC(=O)NCCOCCOC(=O)NCc1ccc(COc2nc(N)nc3[nH]cnc23)cc1 ; COc1cc(C(C)OC(=O)NCCCCC(NC(=O)CCC2(C)N=N2)C(=O)NCCNC(=O)CCCCC2SCC3NC(=O)NC32)c([N+](=O)[O-])cc1OCCCC(=O)NCCOCCOC(=O)NCc1ccc(COc2nc(N)nc3[nH]cnc23)cc1 ; "PP2"]

### 4-(4-acetyl-2-methoxy-5-nitrophenoxy)-N-[2-(2-hydroxyethoxy)ethyl]-butanamide

Neat 2-(2-aminoethoxy)ethanol (158 μL, 1.50 mmol) was added to a suspension of NHS-ester 12 (394 mg, 1.00 mmol) and Et₃N (150 μL, 1.00 mmol) in anhydrous MeCN (5 mL). The reaction was stirred at rt for 5 hours. The volatiles were removed under vacuo and the residue purified by reverse-phase HPLC, eluting with a gradient of MeOH in H₂O (40-95%). Pure amide 27 (248 mg, 64%) was isolated as a yellow oil. ¹H NMR (500 MHz, CDCl₃) δ 7.57 (s, 1H), 6.73 (s, 1H), 4.11 (t, J=6.1 Hz, 2H), 3.93 (s, 3H), 3.71 (t, J=4.4 Hz, 2H), 3.53 (t, J=5.1 Hz, 4H), 3.45 (q, J=5.1 Hz, 2H), 2.46 (s, 3H), 2.44 (t, J=7.3 Hz, 2H), 2.18 (p, J=6.7 Hz, 2H). ¹³C NMR (125 MHz, CDCl₃) δ 200.48, 154.27, 148.83, 138.35, 132.84, 108.79, 108.16, 77.36, 72.15, 69.69, 68.63, 56.69, 39.63, 32.56, 30.46, 24.93.

### Alcohol (29)

In an oven dried flask, DSC (316 mg, 1.23 mmol) was added to an anhydrous DMF (3 mL) solution of amide 27 (434 mg, 1.13 mmol) and Et₃N (428 μL, 3.08 mmol). The reaction was stirred overnight at room temperature before adding solid amine 7 (278 mg, 1.03 mmol). Stirring was continued at rt for an additional 3 hours. Upon completion, the reaction was diluted with H₂O and extracted with EtOAc. The combined organics were washed with brine, dried over Na₂SO₄, filtered, and solvent removed under vacuum to provide the methyl ketone (467 mg, 61%). ¹H NMR (500 MHz, CD₃OD) δ 7.80 (s, 1H), 7.45 (s, 1H), 7.38 (d, J=7.8 Hz, 2H), 7.31 (s, 1H), 7.22 (d, J=7.8 Hz, 2H), 5.41 (s, 2H), 4.24 (s, 2H), 4.16-4.10 (m, 2H), 3.95 (t, J=6.3 Hz, 1H), 3.87 (s, 2H), 3.59 (t. J=4.8 Hz, 2H), 3.49 (t. J=5.4 Hz, 2H), 2.34 (t, J=7.5 Hz, 2H), 2.03 (h, J=6.6 Hz, 2H), 1.43 (d, J=6.3 Hz, 3H). ¹³C NMR (125 MHz, CD₃OD) δ 175.39, 161.50, 158.97, 155.23, 148.02, 140.48, 138.93, 136.65, 129.54, 128.25, 109.79, 70.39, 70.35, 69.49, 68.64, 66.21, 65.02, 56.66, 45.14, 40.36, 36.94, 33.36, 31.63, 26.35, 25.16.

Subsequently, the ketone was taken up in MeOH (10 mL) and solid NaBH₄ added in portions (77.9 mg, 2.06 mmol) and vigorously stirred at rt. Once the intervening ketone was consumed the reaction was quenched with 10 mL H₂O and stirred for an additional 30 min. The mixture was then extracted with EtOAc, organics dried over Na₂CO₃, filtered, and concentrated to provide Alcohol 29 that was taken on without further purification.

### Photoproximity Probe 2 (PP2)

A solution of carbamate 26 (115 mg, 205 μmol) in MeOH (1 mL) was added to 4 M HCl.dioxane (2 mL) at 0° C. The temperature was allowed to come to rt while stirring for 2 hrs. Upon disappearance of starting material the volatiles were evaporated and the residue left to dry under vacuum overnight. The crude hydrochloride salt was used without further purification.

In a separate flask, alcohol 29 (70.1 mg, 102 μmol) and iPr₂NEt (68.0 μL, 411 μmol) were stirred at rt in anhydrous DMF (1 mL) while solid DSC (52.5 mg, 205 μmol) was added. The mixture was stirred overnight. A solution of the amine in DMSO (1 mL) was added and the mixture stirred until consumption of starting material. The volatiles were removed under vacuum and the residue purified by reverse phase HPLC eluting with a gradient of MeOH in H₂O (50-95%). The proximity probe PP2 (12.8 mg, 10%) was isolated as a yellow solid and characterized as mixture of diastereomers. ¹H NMR (500 MHz, DMSO-d⁶) δ 8.04 (s, 1H), 7.96 (t, J=5.6 Hz, 2H), 7.80 (s, 1H), 7.56 (s, 1H), 7.45 (d, J=7.9 Hz, 2H), 7.27 (d, J=7.7 Hz, 2H), 7.11 (s, 1H), 6.46 (d, J=9.1 Hz, 1H), 6.38 (s, 1H), 6.21 (s, 2H), 6.10 (q, J=6.5 Hz, 1H), 5.45 (s, 2H), 4.35-4.28 (m, 1H), 4.18 (d, J=6.3 Hz, 2H), 4.15-4.02 (m, 5H), 3.90 (s, 3H), 3.58-3.54 (m, 2H), 3.44-3.40 (m, 2H), 3.26-3.17 (m, 5H), 3.09 (d, J=15.4 Hz, 8H), 2.99-2.79 (m, 3H), 2.58 (d, J=12.5 Hz, 2H), 2.25 (t, J=7.5 Hz, 3H), 2.09-2.01 (m, 7H), 1.95 (q, J=6.9 Hz, 2H), 1.67-1.41 (m, 7H), 1.41-1.15 (m, 3H), 1.00-0.96 (m, 4H). ¹³C NMR (125 MHz, DMSO-d) δ 178.40, 178.33, 172.32, 171.87, 171.57, 170.97, 170.86, 162.76, 161.14, 160.91, 159.44, 156.44, 155.13, 153.56, 153.40, 146.76, 139.62, 139.24, 135.34, 133.54, 133.42, 128.52, 127.81, 127.08, 108.39, 69.05, 68.65, 68.32, 67.02, 66.51, 63.23, 61.03, 59.22, 56.19, 55.42, 55.35, 52.59, 43.57, 38.49, 38.18, 35.23, 31.61, 31.50, 29.78, 29.59, 29.08, 28.22, 28.06, 25.85, 25.20, 24.64, 22.70, 21.94, 19.29. HRMS (ESI⁺) calculated for C₅₅H₇₆N₁₆O₁₅SH [M+H]⁺ 1233.5475, found 1233.5480.

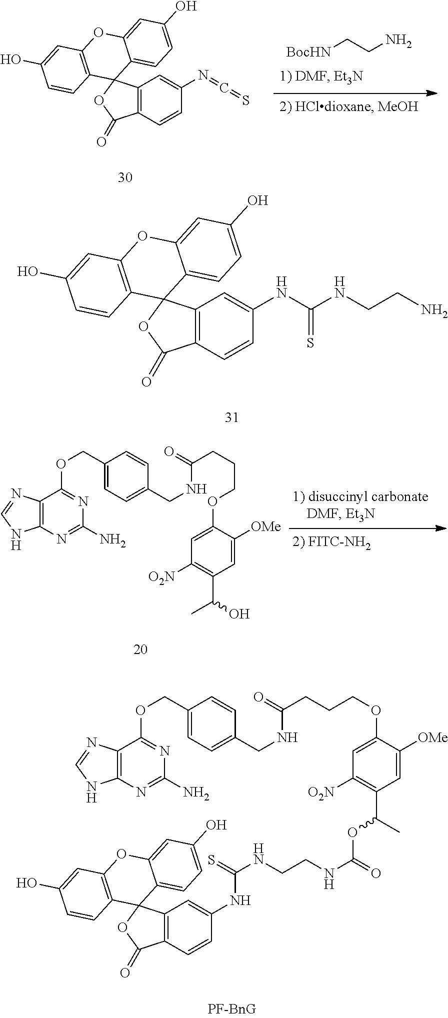 [SMILES: O=C1OC2(c3ccc(O)cc3Oc3cc(O)ccc32)c2cc(N=C=S)ccc21 ; CC(C)(C)OC(=O)NCCN ; NCCNC(=S)Nc1ccc2c(c1)C1(OC2=O)c2ccc(O)cc2Oc2cc(O)ccc21 ; COc1cc(C(C)O)c([N+](=O)[O-])cc1OCCCC(=O)NCc1ccc(COc2nc(N)nc3[nH]cnc23)cc1 ; COc1cc(C(C)OC(=O)NCCNC(=S)Nc2ccc3c(c2)C2(OC3=O)c3ccc(O)cc3Oc3cc(O)ccc32)c([N+](=O)[O-])cc1OCCCC(=O)NCc1ccc(COc2nc(N)nc3[nH]cnc23)cc1]

### Photocleavable FITC-Benzyl Guanine (PF-BnG)

Fluorescein isothiocyanate (FITC, 100 mg, 0.257 mmol) was added to a DMF solution (1 mL) of N-Boc-ethylenediamine (41.3 μL, 0.262 mmol) and Et₃N (7.2 μL, 51.4 μmol) then stirred at room temperature overnight. Upon completion the volatiles were remove under vacuum and the residue purified by reverse-phase HPLC eluting with a gradient of MeOH in H₂O. The purified carbamate (102 mg, 72%) was isolated as an orange solid and used directly.

The intervening carbamate (73.4 mg, 133 μmol) was stirred for 1 hour at room temperature in MeOH (2 mL), 4M HCl.dioxane (0.5 mL), and triisopropylsilane (47 μL). Once fully deprotected the volatiles were removed under vacuum and the residue used without further purification.

In a separate flask, N,N′-disuccinimidyl carbonate (42 mg, 163 μmol) was added to a DMF (1 mL) solution of Alcohol 20 (60.4 mg, 109 μmol) and iPr₂NEt (96 μL, 582 μmol). The mixture was left stir at room temperature overnight. The mixture was transferred to the above amine hydrochloride and stirred at room temperature. Upon completion, the solvent was removed, and the residue purified by reverse-phase HPLC eluting with a gradient of MeOH in H₂O to provide PF-BnG (36 mg, 22%1) as an orange-solid. ¹H NMR (500 MHz, CD₃OD) δ 8.03 (s, 1H), 7.95 (s, 1H), 7.67 (dd, J=8.4, 2.0 Hz, 1H), 7.49 (s, 1H), 7.41 (d, J=8.1 Hz, 2H), 7.25 (d, J=8.0 Hz, 2H), 7.14 (s, 1H), 7.08 (d, J=8.3 Hz, 1H), 6.73-6.62 (m, 4H), 6.53 (ddd, J=8.7, 4.0, 2.4 Hz, 2H), 6.28 (q, J=6.3 Hz, 1H), 5.53 (s, 2H), 4.35 (s, 2H), 4.03-3.91 (m, 2H), 3.89 (s, 3H), 3.64-3.55 (m, 1H), 3.45-3.33 (m, 1H), 3.30-3.24 (m, 2H), 2.41 (t, J=7.3 Hz, 2H), 2.07 (p, J=6.8 Hz, 2H), 1.55 (d, =6.4 Hz, 3H). ¹³C NMR (125 MHz, CD₃OD) δ 183.20, 175.18, 171.08, 161.38, 161.23, 160.21, 158.32, 155.63, 155.53, 154.13, 148.57, 140.85, 140.38, 136.20, 135.22, 130.42, 130.30, 129.88, 128.92, 128.66, 113.60, 111.43, 110.11, 109.38, 103.51, 69.87, 69.56, 69.50, 57.01, 43.83, 33.38, 26.34, 22.54. HRMS (ESI⁺) calculated for C₅₀H₄₆N₁₀O₁₃SH [M+H]⁺ 1027.3044, found 1027.3024.

### Example 2

### General Biological Methods

Cell Culture: Human embryonic kidney (HEK293T) cell lines were purchased from American Type Culture Collection (ATCC, Manassas, Va., United States of America) and all HEK293T lines were propagated in Delbecco's modified Eagle media (DMEM; Corning Inc., Corning, N.Y., United States of America) supplemented with 10% fetal bovine serum (FBS; Corning Inc., Corning, N.Y., United States of America) and 1% penicillin/streptomycin (Gibco Laboratories, Gaithersburg, Md., United States of America). All cell lines were grown at 37° C. in a 5% CO₂ humidified incubator.

SDS-PAGE and Western Blot: Cells were harvested by scraping, pelleted by centrifugation, washed twice with PBS and lysed in 8 M urea, 50 mM NH₄HCO₃ and EDTA-free complete protease inhibitor (Roche Holding AG, Basel, Switzerland). pH 8.0, at 4° C. Cells were sonicated (Fisher Scientific FB-505, Fischer Scientific, Hampton, N.H., United States of America), insoluble debris cleared by centrifugation, and the supernatant was diluted into 4× Laemmli buffer containing 50 mM dithiothreitol (DTT) or 6% beta-mercaptoethanol (OME) as reducing agents. Samples were prepared for SDS-PAGE by heating to 95° C. for 5 minutes, cooled to room temperature, resolved on NuPAGE Novex 4-12% Bis-Tris Protein Gels (Invitrogen, Carlsbad, Calif. United States of America) or 10% SDS-PAGE gel, and transferred to nitrocellulose membranes by standard western blotting methods. Membranes were blocked in 2% BSA in TBS containing 0.1% tween-20 (TBST) and probed with primary and secondary antibodies. Primary antibodies used in this study include: anti-FLAG-M2 (1:1000, F1804, Sigma Aldrich, St. Louis, Mo., United States of America), Streptavidin-IRdye800 (1:10,000, 92632230, Li-cor Biosciences, Lincoln, Nebr., United States of America). Secondary donkey anti-rabbit, donkey anti-goat, and donkey anti-mouse (Li-cor Biosciences, Lincoln, Nebr., United States of America), were used at 1:10,000 dilution in 2% BSA-containing TBST and incubated for 1 hour prior to washing and imaging on a Li-cor infrared scanner (Li-cor Biosciences, Lincoln, Nebr., United States of America. Densitometry measurements were performed with ImageJ software.

Circular polymerase extension cloning (CPEC) construction of mammalian plasmids: All PCR reactions were performed using NEB Q5 high-fidelity polymerase (M0491S; New England Biolabs, Ipswich, Mass., United States of America) and Promega dNTP mix (U1515; Promega Corporation, Madison, Wis., United States of America). Initial constructs were generated using the pSnapf vector from NEB (#N9183S; New England Biolabs, Ipswich, Mass., United States of America), the pFlag-Keap1 vector from Addgene (#28023; Addgene, Watertown, Mass., United States of America) along with the following CPEC primers purchased from IDT (Integrated DNA Technologies, Inc., Coralville, Iowa, United States of America):

pDC-002 (Keap1-SNAP-Flag3x):
(SEQ ID NO: 1)
F1 =
ATGGACAAAGACTGCGAAATGAAGCGCACCACCC
(SEQ ID NO: 2)
R1 =
ACCCAGCCCAGGCTTGCCCA
(SEQ ID NO: 3)
F2 =
GGCAAGCCTGGGCTGGGTgactacaaagaccatgacggtgattata
aagatcatgacat
(SEQ ID NO: 4)
R2 =
GTGCGCTTCATTTCGCAGTCTTTGTCCATGCTTCCGCCGCCgcggc
cgccacaggtaca
pDC-003 (SNAP-Keap1-Flag3x):
(SEQ ID NO: 5)
F1 =
atgcagccagatcccaggcctagc
(SEQ ID NO: 6)
R1 =
gatatctgcagaattccaccacactggactagtggatcc
(SEQ ID NO: 7)
F2 =
ccagtgtggtggaattctgcagatatcATGGACAAAGACTGCGAAA
TGAAGCGCACCAC
(SEQ ID NO: 8)
R2 =
gcctgggatctggctgcatGCTCCCTCCGCCGCCACCCAGCCCAGG
CTTGCCC

The constructs above were subcloned into the pLenti6N5-p53_R273H Addgene (#22934; Addgene, Watertown, Mass., United States of America) vector for lentiviral transduction using the following CPEC primers purchased from IDT (Integrated DNA Technologies. Inc, Coralville, Iowa, United States of America):

pDC-006 (Keap1-SNAP-Flag3x):
F1 =
(SEQ ID NO: 9)
tagtaatgagtttggaattaattctgtggaatgtgtgtcagttaggg
R1 =
(SEQ ID NO: 10)
ggtgaagggatcaattccaccacactgg
F2 =
(SEQ ID NO: 11)
GGTGGAATTGATCCCTTCACCatgcagccagatcccagg
R2 =
(SEQ ID NO: 12)
cacattccacagaattaattccaaactcattactacttgtcatcgtc
atccttgtagtcg
pDC-007 (SNAP-Keap1-Flag3x):
F1 =
(SEQ ID NO: 9)
tagtaatgagtttggaattaattctgtggaatgtgtgtcagttaggg
R1 =
(SEQ ID NO: 10)
ggtgaagggatcaattccaccacactgg
F2 =
(SEQ ID NO: 13)
ggtggaattgatcccttcaccATGGACAAAGACTGCGAAATGAAGC
R2 =
(SEQ ID NO: 12)
cacattccacagaattaattccaaactcattactacttgtcatcgtc
atccttgtagtcg

Transient and Stable Protein Expression in Cells: Mammalian cells stably expressing the KEAP1 SNAP-Tag fusions were obtained by co-transforming a 6 cm plate of HEK293T cells with 0.1 μg pCMV-VSV-G (Addgene #8454; Addgene, Watertown, Mass. United States of America), 0.9 μg pCMV delta R8.2 (Addgene #12263; Addgene, Watertown, Mass., United States of America), and 1.0 μg of either pDC-006 or pDC-007. The resultant viral media was collected at 24 and 48 hours, passed through a 0.45-micron filter, and diluted with serum-free DMEM containing 8 μg/mL polybrene (Sigma-Aldrich, St. Louis, Mo., United States of America), final concentration. Viral transduction was achieved by culturing a separate population of HEK293T in the diluted viral media for 24 hours. Afterward, the viral media was removed and the transduced cells grown in full DMEM containing 8 μg/mL blasticidin (Gibco Laboratories, Gaithersburg, Md., United States of America). Stable incorporation of the transgene was confirmed by western blotting using monoclonal anti-FLAG M2 antibody (Sigma-Aldrich, St. Louis, Mo., United States of America). Stable expression of SnapFlag, derived from pSnapf vector (NEB #N9183S, New England Biolabs, Ipswich, Mass., United States of America), was achieved through chemical selection of transiently transfect HEK293T cells with 500 μM G418 (Gibco Laboratories, Gaithersburg, Md., United States of America). Transient SNAP-FLAG protein expression was accomplished using transfection with lipofectamine 2000 according to manufacturer protocol.

In vitro photocleavage assay: Recombinant purified His₆-SNAP-Tag (20 μg) was incubated in 200 μL DPBS containing 5 μM PP1 for 1 hour at 37° C. Afterward, the reaction was divided into six 30 μL aliquots, placed on ice, and irradiated with 365 nm light using a SPECTROLINKER™ XL-1500a UV crosslinker (Spectroline, Spectronics, Corporation, Westbury, N.Y., United States of America). Samples were successively removed from irradiation after 0, 0.5, 1, 2.5, 5, 10 minutes of irradiation, diluted with loading buffer, and run on SDS-PAGE gel. Photocleavage was judged by in-gel fluorescence visualized using a ChemiDoc imaging system (Bio-Rad Laboratories, Hercules, Calif., United States of America) scanning for fluorescein.

PP1 close response and viability assay: Dose response—Each well of a 6-well plate was seeded with 300,000 HEK293T cells stably expressing either SNAP-FLAG or KEAP-SNAP. After reaching ˜90% confluency the growth media was removed, cells washed with DPBS, and treated with varying concentrations (0, 0.5, 1, 5, 15, 45 μM) of photoproximity probe PP1 in 500 μL serum-free DMEM for 2 hours at 37° C. Post treatment, media was aspirated and non-reacted probe washed out with 3 mL full DMEM over 40 minutes, changing the media twice. Cells were harvested, washed, and lysed in DPBS containing protease inhibitor using a tip sonicator. Lysate was normalized to 1 mg/mL then diluted with loading buffer and run on SDS-PAGE gel that was transferred by western blot to nitrocellulose. The nitrocellulose membrane was stained for biotin and Flag-Tag using streptavidin IR dye (Li-cor #926-32230; Li-cor Biosciences, Lincoln, Nebr., United States of America) and anti-Flag (Sigma #F3165, Sigma-Aldrich, St. Louis, Mo., United States of America; Li-cor #926-68072, Li-cor Biosciences, Lincoln, Nebr., United States of America). Fluorescence images were collected using an Odyssey infrared imager (Li-cor Biosciences, Lincoln, Nebr., United States of America).

Viability assay: HEK293T cells stably expressing SNAP-FLAG were seeded with at 5,000 cells per well in 50 μL DMEM in a 96-well plate. Twenty-four hours later, cells were treated with a varying concentration (0, 0.5, 1, 5, 15, 45 μM in sextuplicate) of photoproximity probe PP1 in a total volume of 100 μL DMEM. After 2 hours at 37° C., a 100 μL of CellTiter-Glo® (Promega Corporation, Madison, Wis., United States of America; 5-fold DPBS dilution) was added and the plate imaged using a SYNERGY™ Neo HST plate reader (BioTek, Winooski, Vt., United States of America).

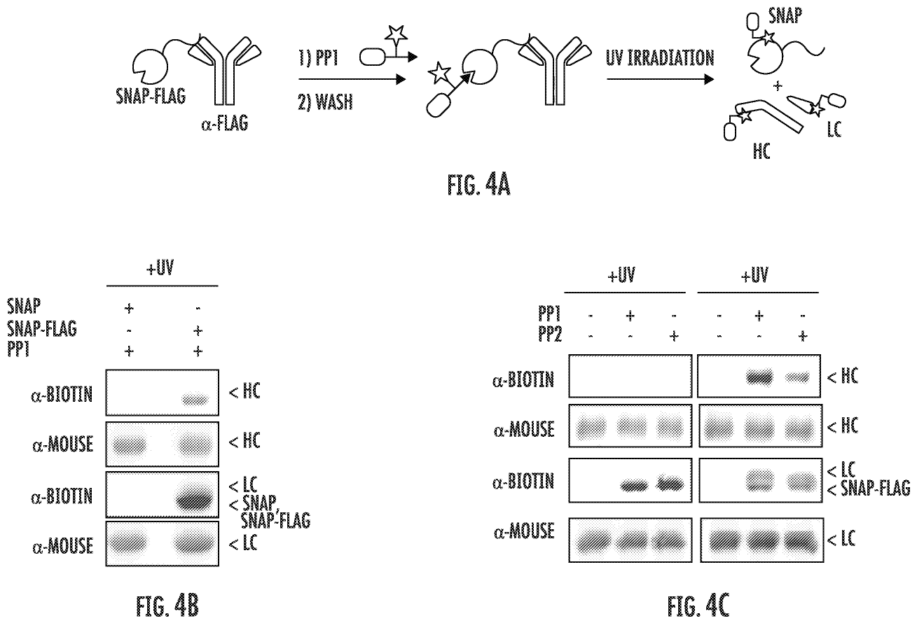

Proximity-labeling assays: Clarified cell lysate from HEK293T cells either transiently (see FIG. 4B) or stably (see FIG. 4C) expressing SNAP-Tag (NEB #N9183S; New England Biolabs, Ipswich, Mass., United States of America) or SnapFlag was normalized to 1 mg/mL and 250 μL aliquots incubated in a microcentrifuge tube with either 500 nM of PP1. PP2, or DMSO for 1 hour at 37° C. Washed anti-FLAG M2 affinity gel, 40 μL of a 50% slurry, was transferred to each reaction in 750 μL of DPBS and the resultant suspension left to rotate overnight at 4° C. (Sigma #A2220; Sigma-Aldrich, St. Louis, Mo., United States of America). Samples were then spun down at 4000×g, supernatant aspirated, and the resin washed with 1M urea in DPBS (1 mL×8) followed by DPBS (1 mL). After being resuspended in 100 μL of DPBS, the samples were placed on ice where they were irradiated with 365 nm light for 10 minutes (SPECTROLINKER™ XL-1500a UV crosslinker, Spectronics Corporation, Westbury, N.Y., United States of America). Once irradiated, the beads were washed with 100 mM pH 3.5 glycine buffer (100 μL×2) and DPBS (1 mL). To elute the remaining SNAP-FLAG and resin-bound FLAG-antibody the beads were boiled for 5 minutes at 95° C. in 20 μL 4×-loading buffer containing 8% SDS and 400 mM DTT. After which, 60 μL of DPBS was added and the suspension boiled at 95° C. for an additional 5 minutes. The samples were then run on SDS-PAGE gel and transferred to nitrocellulose where they were stained with anti-mouse IR dye (Li-cor #926-68072; Li-cor Biosciences, Lincoln, Nebr., United States of America) and streptavidin IR dye (Li-cor #926-32230; Li-cor Biosciences, Lincoln, Nebr., United States of America). Bands corresponding to biotinylated proteins and the FLAG antibody were visualized using an Odyssey infrared imager (Li-cor Biosciences, Lincoln, Nebr., United States of America).

SILAC cell culture methods and proteomic sample preparation: SILAC labeling was performed by growing cells for at least five passages in lysine- and arginine-free SILAC medium (RPMI; Invitrogen, Carlsbad, Calif. United States of America) supplemented with 10% dialyzed fetal calf serum, 2 mM L-glutamine and 1% Pen/Strep. “Light” and “heavy” media were supplemented with natural lysine and arginine (0.1 mg/mL), and ¹³C-, ¹⁵N-labeled lysine and arginine (0.1 mg/mL), respectively.

General protein digestion for LC-MS/MS analysis was performed by diluting protein (e.g. whole lysate or enriched proteins) in digestion buffer (8 M urea, 50 mM NH₄HCO₃, pH 8.0), followed by disulfide reduction with DTT (10 mM, 40 minutes, 50° C.), alkylation (iodoacetamide, 15 mM, 30 min, room temperature, protected from light) and quenching (DTT, 5 mM, 10 minutes, room temperature). The proteome solution was diluted 4-fold with ammonium bicarbonate solution (50 mM, pH 8.0). CaCl₂ added (1 mM) and digested with sequencing grade trypsin (˜1:100 enzyme/protein ratio; Promega Corporation, Madison, Wis., United States of America) at 37° C. while rotating overnight. Peptide digestion reactions were stopped by acidification to pH 2-3 with 1% formic acid, and peptides were then desalted on ZipTip C18 tips (100 μL, MilliporeSigma, Burlington, Mass., United States of America), dried under vacuum, resuspended with LC-MS grade water (Sigma Aldrich, St. Louis Mo., United States of America), and then lyophilized. Lyophilized peptides were dissolved in LC-MS/MS Buffer A (H₂O with 0.1% formic acid, LC-MS grade, Sigma Aldrich, St. Louis, Mo., United States of America) for proteomic analysis.

Sample preparation and streptavidin enrichment of/proteins biotinylated by P3 profiling: Quantitative proximity labeling study with SILAC quantitative proteomics was performed with “heavy” and “light” labeled HEK293T cells expressing KEAP SNAP fusion constructs. SILAC-labeled cells, grown to 80-90% confluency in 10 cm cell-culture treated plates (Denville) each, were incubated with DMSO alone (light cells) or PP1 probe (15 μM, heavy cells) for 2 hours in serum-free SILAC RPMI. After incubation, excess probes in “heavy” cells were pulled out by a replacement to “heavy” growth media in every 10 minutes for 2 times. All cells were then incubated in 2 mL cold PBS, UV irradiated using a Spectroline XL-1500A instrument (Spectronics Corporation. Westbury, N.Y., United States of America) for 15 minutes, scraped, washed with cold PBS (2×), and tiny aliquots of cells (20 μL out of 500 μL resuspended cells in PBS) from each cell plate were taken for the analysis of bulk proteome. These “heavy” and “light” cell aliquots were combined and digested by general protein digestion protocol described above.

The rest of cells (480 μL out of 500 μL resuspended cells in PBS) were pelleted and then lysed in RIPA lysis buffer (50 mM Tris, 150 mM NaCl, 1% Triton X-100, 0.5% deoxycholate, pH 7.4) supplemented with EDTA-free complete protease inhibitor (Roche Holding AG, Basel, Switzerland) and 1 mM DTT, at 4° C. After sonication, insoluble debris was cleared by centrifugation (17,000 g, 10 min). Streptavidin C1 magnetic beads (30 μL slurry, 65001, Invitrogen, Carlsbad, Calif., United States of America) were washed twice with RIPA buffer, and each cell lysate was separately incubated with the magnetic beads with rotation overnight at 4° C. The beads were subsequently washed five times with 0.5 mL of RIPA lysis buffer containing 1 mM DTT, combined together, then washed once with 1 mL of 1 M KCl, four times with 0.5 mL PBS, and two times with 2 M Urea in 25 mM ammonium bicarbonate. 500 μL of 6 M Urea in 50 mM ammonium bicarbonate was then added to the beads, and samples were reduced on resin by TCEP (10 mM final), with orbital shaking, for 20 minutes at 65° C. Samples were then alkylated by adding iodoacetamide (20 mM final), covered from the light and with orbital shaking, for 40 minutes at 37° C. The streptavidin magnetic beads were collected, washed once with 2 M Urea in 25 mM ammonium bicarbonate, and the buffer exchanged to 2 M Urea in 25 mM ammonium bicarbonate supplemented with 1 mM CaCl₂. Enriched proteins were digested on bead by the incubation of 2 μg sequencing grade trypsin overnight at 37° C. Following trypsinization, supernatant was collected, acidified with HPLC grade formic acid (2% final, pH 2-3), and peptides were desalted as indicated above.

Proteomic LC-MS/MS and data analysis: LC-MS/MS experiments were performed with an EASY-NLC™ 1000 ultra-high-pressure LC system (ThermoFisher Scientific, Waltham, Mass., United States of America) using a PEPMAP™ RSLC C18 column (ThermoFisher Scientific, Waltham, Mass., United States of America) heated to 45° C. (column: 75 μm×50 cm; 2 μm, 100 Å) coupled to a Q EXTRACTIVE™ HF orbitrap and EASY-SPRAY™ nanosource (ThermoFisher Scientific, Waltham, Mass., United States of America). Digested peptides (500 ng-1 μg) in MS/MS Buffer A were injected onto the column and separated using the following gradient of buffer B (0.1% Formic acid acetonitrile) at 300 nL/min; 2-2% buffer B over 5 minutes, 2-25% buffer B over 170 minutes, 25-40% buffer B over 40 minutes, 40-90% buffer B over 10 minutes, 90-90% buffer B over 5 minutes, 90-2% buffer B over 5 minutes, 2-2% buffer B over 5 minutes, 2-90% buffer B over 5 minutes, 90-90% buffer B over 3 minutes, 90-2% buffer B over 5 minutes, 2-2% buffer B over 3 minutes, 2-90% buffer B over 5 minutes, 90-90% buffer B over 5 minutes, 90-2% buffer B over 5 minutes, and 2-2% buffer B over 3 minutes. MS/MS spectra were collected from 0 to 240 minutes using a data-dependent, top 10 ion setting with the following settings: full MS scans were acquired at a resolution of 120,000, scan range of 375-1500 m/z, maximum IT of 60 ms, AGC target of 1e6, and data collection in profile mode. MS2 scans was performed by HCD fragmentation with a resolution of 30,000, AGC target of 1e5, maximum IT of 60 ms, NCE of 27, MSX count of 1, and data type in centroid mode. Isolation window for precursor ions was set to 2.0 m/z with isolation offset of 0.0 m/z. Peptides with charge state 1 and undefined were excluded and dynamic exclusion was set to twenty seconds. Furthermore, S-lens RF level was set to 60 with a spray voltage value of 2.20 kV and ionization chamber temperature of 275° C.

MS2 files were generated and searched using the ProLuCID algorithm (ProLuCID, Mississauga, Canada) in the Integrated Proteomics Pipeline (IP2) software platform. Human proteome data were searched using a concatenated target/decoy UniProt database (UniProt_Human_reviewed_04-10-2017.fasta). Basic searches were performed with the following search parameters: HCD fragmentation method; monoisotopic precursor ions; high resolution mode (3 isotopic peaks); precursor mass range 600-6,000 and initial fragment tolerance at 600 p.p.m.; enzyme cleavage specificity at C-terminal lysine and arginine residues with 3 missed cleavage sites permitted; static modification of +57.02146 on cysteine (carboxyamidomethylation); two total differential modification sites per peptide, including oxidized methionine (+15.9949); primary scoring type by XCorr and secondary by Zscore: minimum peptide length of six residues with a candidate peptide threshold of 500. A minimum of one peptide per protein and half-tryptic peptide specificity were required. Starting statistics were performed with a Δmass cutoff=15 p.p.m. with modstat, and trypstat settings. False-discovery rates of peptide (sfp) were set to 1%, peptide modification requirement (-m) was set to 1, and spectra display mode (-t) was set to 1. SILAC searchers were performed as above with “light” and “heavy” database searches of MS1 and MS2 files by including static modification of +8.014168 for lysine and +10.0083 for arginine in a parallel heavy search. SILAC quantification was performed using the QuantCompare algorithm, with a mass tolerance of 10 p.p.m. or less in cases where co-eluting peptide interfere. In general all quantified peptides have mass error within 3 p.p.m.

Quantitative proteomic data analyses of enriched proteomic samples: The SILAC ratios of the proteins from enriched proteomic samples were normalized by the median SILAC ratio of the corresponding bulk proteomic sample. The overall normalized SILAC data, from three biologically independent batches and three technical replication LC-MS/MS runs of each batch, were combined. The mean SILAC ratios of each protein were converted to Log₂ values, and P values were calculated by univariate two-sided t-test with a group of unnormalized SILAC ratios of the protein from enriched samples and a group of median SILAC ratios of the bulk samples. P values were further adjusted for Benjamini-Hochberg FDR correction and then converted to −Log₁₀ values. The volcano plots were plotted with x-axis of Log₂ SILAC values and γ-axis of −Log₁₀ P values (adj.), and the proteins which passed the filter (Probe-to-DMSO ratio >2. P adj.<0.005) were considered as actual enriched proteins with the P3 profiling platform.

### Example 3

### Design of the P3 Platform

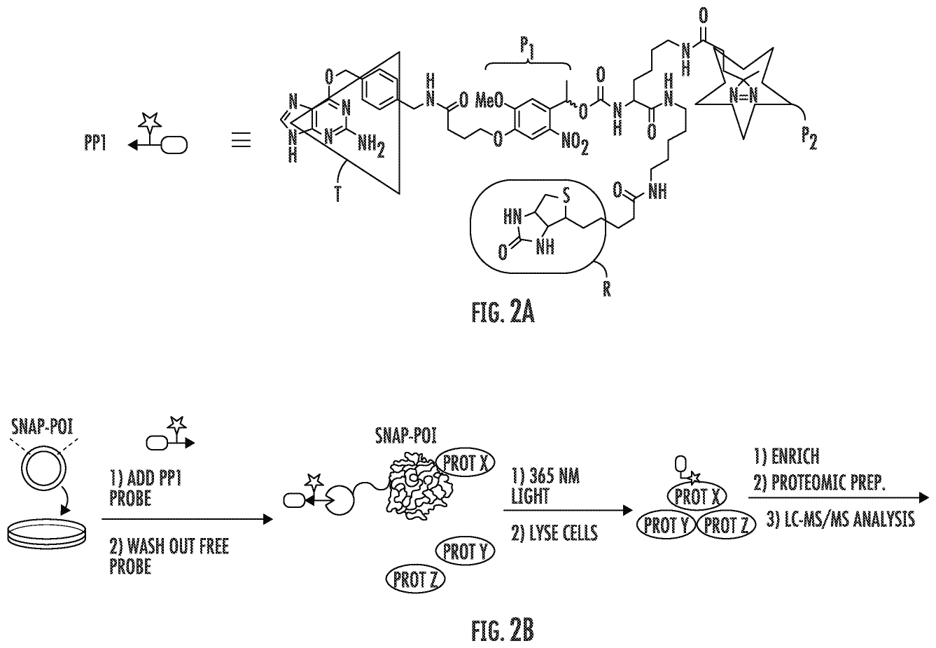

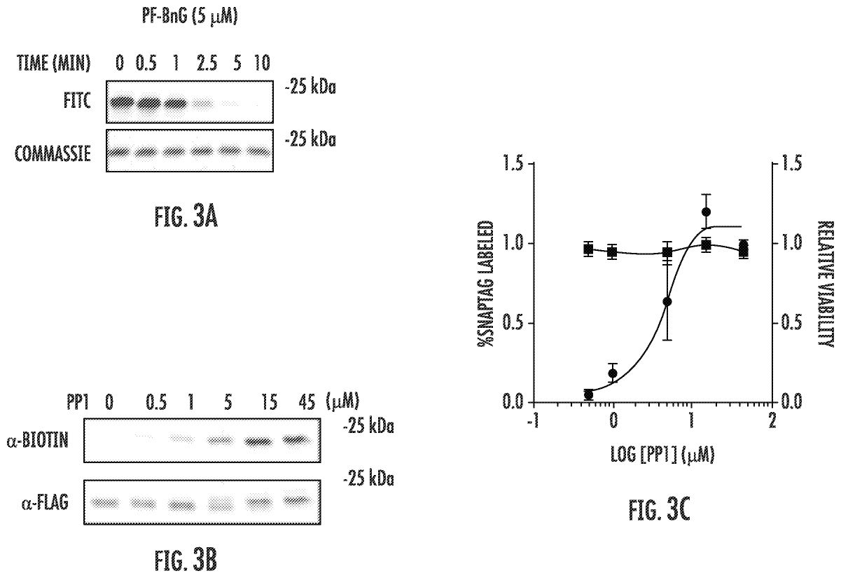

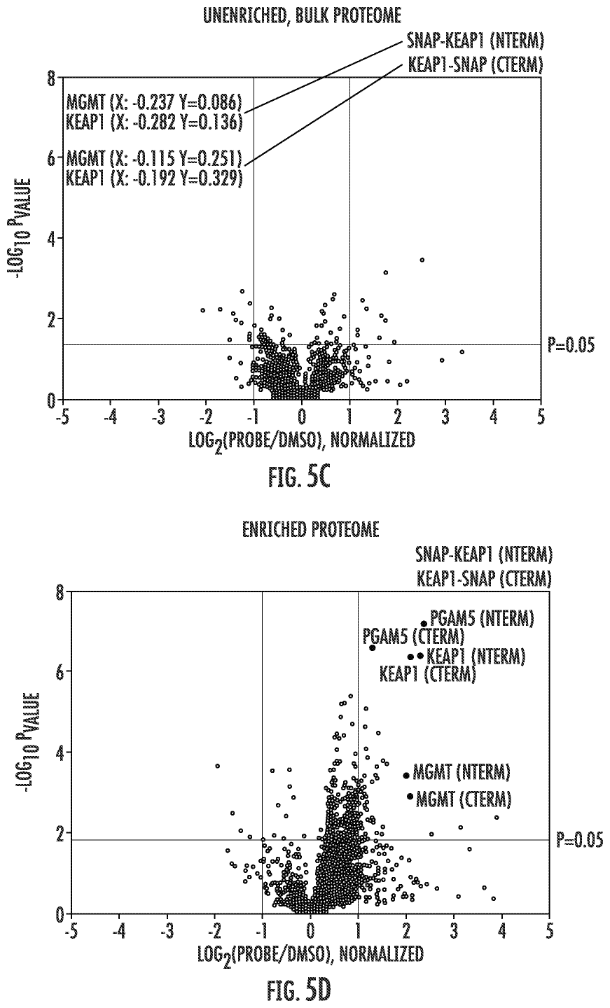

A first generation P3 chemical proteomic method was designed to facilitate several activities, including: 1) specific probe labeling of POIs in live cells; 2) spatial and temporal control of probe photoactivation and proximal labeling; 3) subsequent enrichment and identification of labeled proteins after cell lysis. See FIGS. 2A and 2B. To realize these activities, a modular photoproximity chemical probe, PP1, targeting a POI through a benzylguanine (BnG) recognition element, which has been shown to specifically label a “SNAP-Tag” protein (an engineered O⁶-methylguanine DNA methyltransferase, MGMT) that can be theoretically expressed as a genetically-encoded fusion on any POI, was prepared. See FIG. 2A. The BnG targeting element is connected to dual photoreactive elements, a central substituted nitroveratryl carbamate, and a tethered diazirine, which upon irradiation with 365 nm light will simultaneously trigger cleavage and diffusion of the probe away from the SNAP-Tag-POI, as well as unmasking of a highly reactive carbene, respectively. See FIG. 2A. Finally, the diazirine portion of PP1 is connected to a retrieval tag—in this case biotin—for recognition and enrichment of proteins that were covalently labeled in live cells. See FIG. 2A. The labeling and photocleavage capacity of the central nitrobenzyl carbamate linker was first verified using a cleavable BnG-FITC model probe (PF-BnG; see FIG. 6), which fluorescently labeled recombinant SNAP-Tag protein. See FIG. 3A. In line with other reports using photocleavable linkers for selective delivery of bioactive molecules on or in cells^{19,20}, it was found that irradiation of the labeled protein at 365 nm resulted in complete loss of fluorescence within minutes, validating the capacity to label and cleave BnG-nitrobenzyl probes. To determine whether the multifunctional PP1 probe could label SNAP-Tag protein in cells, HEK293T cells stably expressing FLAG-tagged SNAP-Tag protein (SNAP-FLAG) were treated with increasing doses of PP1 for two hours, which resulted in robust covalent labeling at low micromolar concentrations (EC₅₀=4.5 μM). See FIG. 3B. Cell viability was not affected at saturating doses of PP1 (see FIG. 3C), suggesting that cells expressing a SNAP-Tag-POI fusion could be pulse-labeled with PP1 with minimal perturbation to cell physiology.

### Example 4

### In Vitro Protein-Protein Interaction Profiling

To study if proximal protein interactors could be labeled in response to the dual photoactivation of PP1 in vitro, the interaction between SNAP-FLAG protein and an α-FLAG monoclonal antibody (mAb) was examined in cell lysate. See FIG. 4A. In this assay, proximal biotinylation of both heavy and light chains of the antibody was expected, as well as self-labeling of the SNAP-FLAG protein, under conditions where the protein complex is formed and the PP1 probe is photoactivated. See FIG. 4A. To test this hypothesis in the presence and absence of the protein-protein complex, SNAP protein that contained or lacked the FLAG epitope was treated with PP1 probe, followed by incubation with α-FLAG mAb, bead-based pulldown of the complexes, and subsequent photoactivation. Anti-mouse (recognizing both heavy and light chain α-FLAG) and streptavidin-IR western blot revealed robust biotinylation of both heavy and light chain fragments with SNAP-FLAG protein, but no protein-biotinylation in reactions using SNAP protein that lacked the FLAG sequence, validating the required protein-complex for proximal labeling by PP1. See FIG. 4B. The requirement for photoactivation of PP1, as well as a related probe PP2 (see FIG. 6) was tested by forming the SNAP-FLAG/α-FLAG complexes, and then comparing protein biotinylation with or without irradiation. Complexes labeled with PP1 or PP2, but not exposed to 365 nm light exhibited biotinylation of a protein at ˜20 kDa, consistent with SNAP protein by molecular mass, due to the retention of the active-site labeled probe. No proximal labeling of the heavy chain protein, or a closely migrating light chain protein was observed without irradiation. See FIG. 4C. By contrast, a discrete biotinylated band was formed on both heavy and light chains upon treatment with and irradiation of both PP1 and PP2. See FIG. 4C. Together, these data confirmed the light- and proximity-dependent labeling of protein complex partners with the P3 system.

### Example 5

### Photoproximity Profiling in Live Cells

The P3 platform was next used to determine whether proximal binding partners for a POI could be interrogated in live cells. In particular, a protein that would be challenging to study by existing proximity profiling methods was selected as the POI, i.e., KEAP1, which acts as the critical sensor protein at the center of the antioxidant response signaling network.

KEAP1 harbors numerous reactive cysteines that collectively sense alterations in the redox, metabolic and xenobiotic environments of the cell; these interactions ultimately control the sequestration and turnover of the NFE2L2 (also known as NRF2) transcription factor and the downstream antioxidant gene expression program²¹. Given the central role of KEAP1 protein in sensing and controlling the cellular environment, a global assessment of its binding partners in specific cells and under discrete conditions could provide novel insights into core cellular wiring and physical interactions with other pathways.

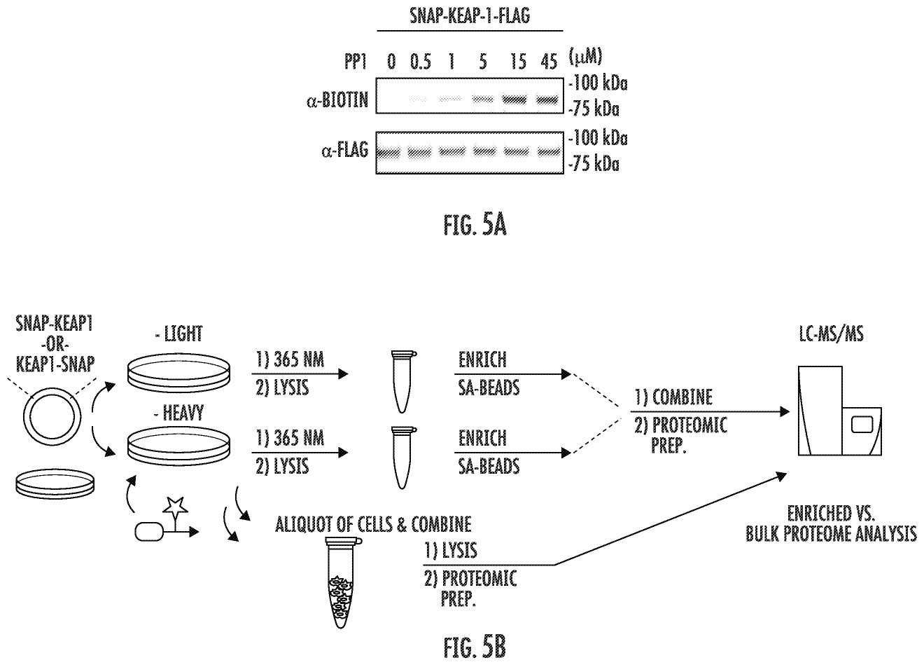

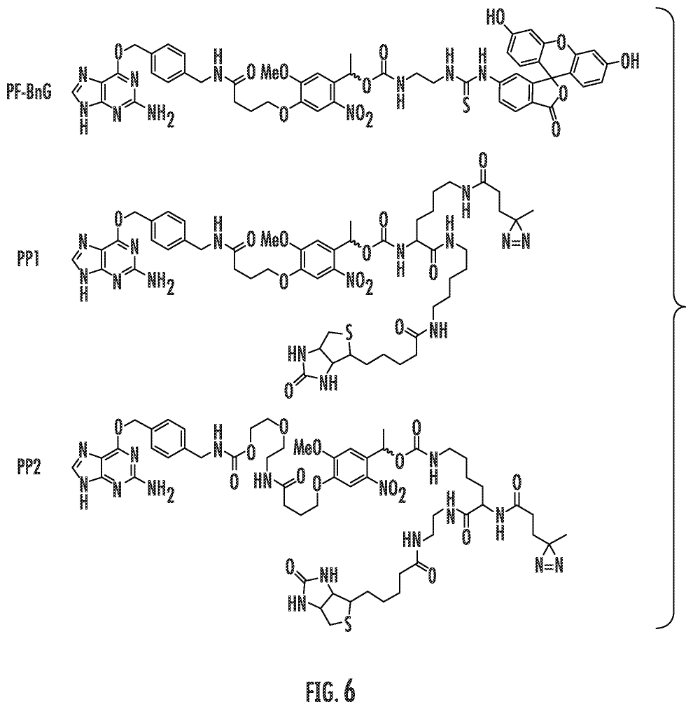

Both C- and N-terminal SNAP-Tag fusions of full-length, human KEAP1, in each case separated by a short linker were cloned. See FIG. 7. Stable expression in HEK293T cells resulted in the appearance of the KEAP1-SNAP protein monomer at the expected mass of 90 kDa using Western blot analysis. See FIG. 5A. Both C- and N-terminal SNAP fusions were functional, as indicated by dose-dependent labeling with PP1 in live cells. See FIG. 5A. The low micromolar EC₅₀ indicated that cellular studies with 15 μM PP1 would be suitable to label all fusion proteins prior to irradiation. Finally, it was expected that identification of probe-labeled binding partners, and therefore proteins proximal to KEAP1 in cells, would be improved by quantitative proteomic differentiation of enriched vs. background proteins. Therefore, SILAC-labeled cultures expressing both N-terminal (referred to as SNAP-KEAP1) and C-terminal (referred to as KEAP1-SNAP) fusion proteins with light and heavy arginine and lysine were prepared. See FIG. 5B. Matched cultures of heavy and light cells, each expressing the same SNAP-KEAP1 or KEAP1-SNAP construct, were then treated with PP1 probe or vehicle for 2 hr. Following compound washout, cells were irradiated, lysed in denaturing RIPA buffer, and exposed to a streptavidin-bead pulldown of biotinylated proteins from each sample. Separate LC-MS/MS analysis of the combined bulk proteome, and separately enriched proteomes, was performed to selectively identify proteins that are enriched in the heavy, PP1 proximity labeled proteome, relative to any background effects in the bulk proteome. Together these profiles were integrated to identify the proximal “social network” of KEAP1 in live cells. See FIG. 5B.

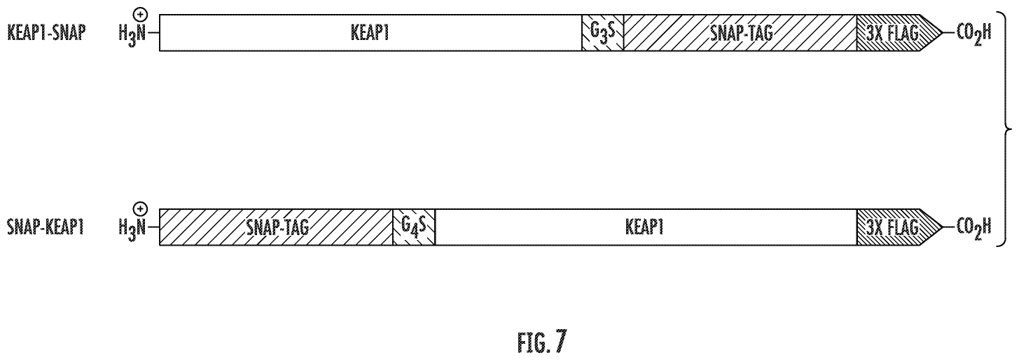

PP1 treatment had no significant effect on global protein abundance relative to vehicle treatment. See FIG. 5C. Both KEAP1 and SNAP-Tag (a modified MGMT sequence) proteins were detected in these profiles, and neither showed any enrichment in light or heavy proteome under P3 profiling conditions. In stark contrast, the biotin-enriched proteome profile was heavily skewed toward the “heavy”, PP1 probe-treated condition. See FIG. 5D. A conservative enrichment cutoff was applied that required a fold-change >2 and a multiple hypothesis test corrected P-value <0.05 from replicate technical and biological runs to identify proteins that were significantly enriched by PP1 photoproximity profiling. Both KEAP1 and MGMT proteins were at the top of the enriched profile from cells expressing either the C- and N-terminal KEAP1 fusions, confirming the proximal labeling and enrichment of the bait fusion protein. This labeling likely results from both within-sphere self-labeling, as well as labeling of adjacent KEAP1 bound in the non-covalent²² and covalent²³ homodimers that are known to form in cells. Indeed, higher enrichment ratios were observed for KEAP1 compared to MGMT, consistent with significant labeling of endogenous KEAP1 bound to the PP1-labeled SNAP-KEAP1 fusions in cells. See FIG. 5D. Conspicuously, the next most enriched protein in both profiles was phosphoglycerate mutase 5 (PGAM5), which is a validated KEAP1-interacting protein that harbors a consensus ‘ESGE’ KEAP1 binding site and has been implicated in tethering KEAP1 to the mitochondrial membrane^{24,25}. The global P3 profile also included significant enrichment of proteins involved in vesicle and membrane trafficking, ribosomal biogenesis, mitochondrial membrane transport, splicing, redox regulation, and other functional categories. See FIGS. 8A and 8B. Intriguingly, neither NRF2 nor Cul3, known KEAP1-binding partners, were detected in this steady state experiment. Likewise, the only published AP-MS study of KEAP1 binding proteins under basal conditions failed to detect NRF2 or Cul3²⁶. Despite completely different proteomic workflows, however, this AP-MS profile had significant overlap with that identified here, with PGAM5 being identified as one of the most enriched proteins in both profiles, as well as similar network-level enrichment of proteins involved in ribosome biogenesis, membrane trafficking, mRNA splicing and detoxification pathways. These data confirm that the P3 method can identify known binding interactors in live cells. The data further suggest that the detection of ubiquitin ligase substrates and complex members, which are subject to rapid turnover and degradation, can be performed by adjustment of experimental conditions.

### Example 6

### Discussion of Examples 3-5

A novel photoproximity protein profiling platform is described herein, which relics on complimentary photo-responsive chemical probes and genetically encoded SNAP-POI targeting in cells for light-triggered proximity labeling. In vitro experiments validated protein-protein interaction-dependent, and light-mediated covalent tagging of proteins bound to a PP1-labeled SNAP-Tag protein. Furthermore, intracellular labeling, light-triggered activation and proteome-wide proximity profiling of KEAP1 in live cells was demonstrated. These studies identified known, high-confidence interactors of KEAP1, and unveiled a steady state binding profile for this network. Future studies can determine, for example, how the KEAP1 network responds to oxidative or electrophilic stress. The modular nature of the SNAP-Tag fusion expression can provide for rapid redesign and proximity tagging of “prey” proteins detected in screens, which can themselves be profiled and integrated to grow larger, multi-component interaction maps.

This capacity to load a POI-fusion construct (e.g., a SNAP-Tag fusion construct) with a masked proximity labeling molecule in live cells without significant perturbation to cellular physiology can provide significant advantages relative to other live-cell proximity labeling technologies. First, the facile labeling and washout of free PP1, followed by light-triggered proximity labeling can permit very high temporal and contextual control of the cellular conditions that can be interrogated using the P3 platform relative to other approaches that have either prolonged incubation periods or no on/off-trigger. While not explored here, the light activated control of this system can also provide for selective spatial activation, and therefore proximity profiling, of a unique cellular field. Facile compound treatment and subsequent light activation can also be ideal for exploring signaling events, spatial compartments and proteins that could involve differential redox regulation, which could be perturbed by other profiling technologies. Additionally, one key design element for a high-fidelity spatial profiling platform is the reactivity of the tagging group, which sets the practical labeling radius as well as the reactivity profile with target biomolecules. In this regard, the masked carbene nucleophile in the first generation P3 probe can provide a highly restricted labeling radius, and broader chemical targeting capacity on proximal proteins relative to the acyl phosphate and phenoxyl radicals. Guided alteration of the structure of the central photocleavage group coupled with tuning of the photoreactive element can provide for the development of probes with differential labeling radii and altered target compatibility to provide higher resolution interactome maps. The P3 platform can also be used alongside existing proximity profiling methods to provide complimentary, layered datasets within the same biological contexts. Thus, it is anticipated that the presently disclosed platform can be broadly useful in elucidating molecular interaction networks inside living systems.

### Example 7

### Additional Studies

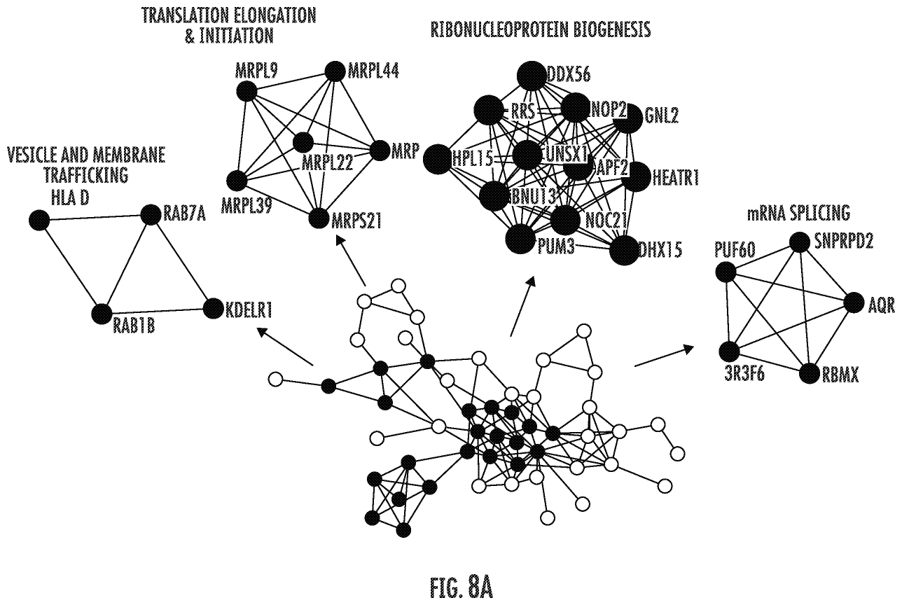

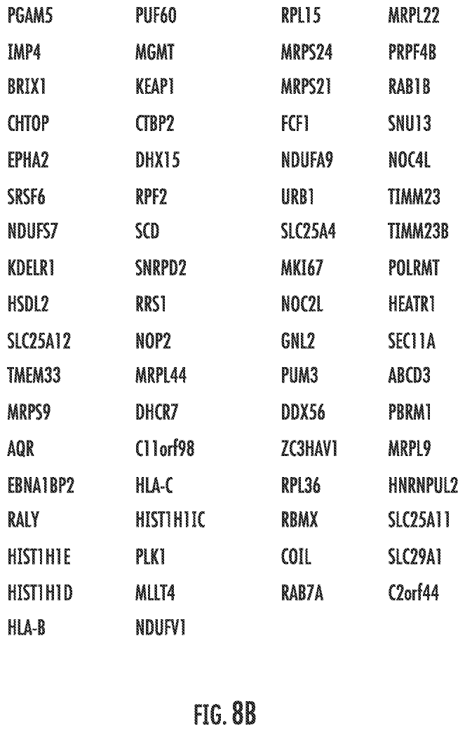

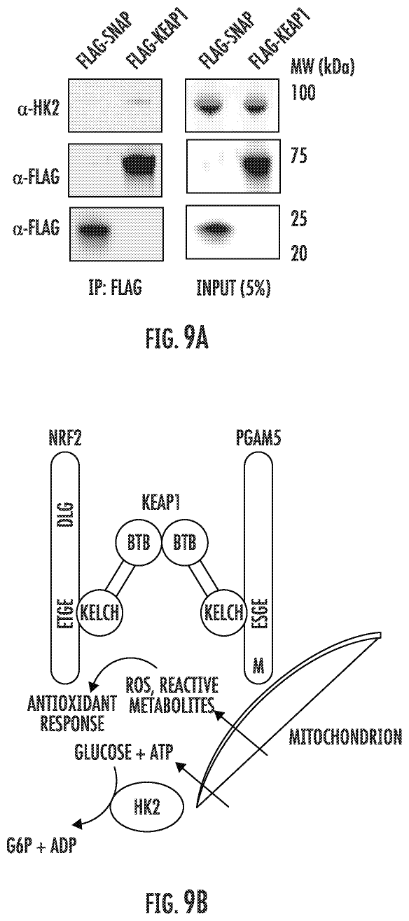

FIGS. 9A and 9B show the validation of hexokinase 2 interaction with KEAP1 determined according to the presently disclosed subject matter and a related model for KEAP1 localization to the mitochondrial membrane. FIG. 10 shows the detection of altered protein interactions in response to dynamic cellular stimuli detected according to the presently disclosed subject matter. FIGS. 11A and 11B show the relative photo reactivity response of diazirine and nitroveratryl, exemplary photoreactive and photocleavable moieties of the presently disclosed probes.

### Example 8

### Synthesis of Additional Photocleavable Photoproximity Probes

General Synthetic Methods: Reagents purchased from commercial suppliers were analytical grade and used without further purification. All reactions were carried out in oven dried flasks using anhydrous solvents (Acros Organics, Thermo Fisher Scientific, Waltham, Mass., United States of America) unless otherwise specified. Reaction progress was monitored by thin-layer chromatography (TLC) on MACHEREY-NAGEL™ SIL G-25 UV254 TLC plates (Macherey, Nagel GmbH & Co., KG, Düren, Germany), visualized with UV light, ceric ammonium molybdate (CAM), p-anisidine, bromophenol blue, 2,4-dinitrophenyl hydrazine (DNP), or KMnO₄ TLC stains. Nuclear magnetic resonance spectra were acquired using either a Bruker AVANCE II+ 500; 11.7 Tesla NMR or Bruker DRX 400; 9.3 Tesla NMR instrument (Bruker, Billerica, Mass., United States of America). Accurate mass measurements were obtained using an Agilent 6224 TOF-MS instrument (Agilent Technologies, Santa Clara, Calif., United States of America). When necessary, compounds were purified via flash column chromatography using Siliaflash F60 60 Å, 230-400 mesh silica gel (Silicycle Inc., Quebec City, Canada)

BG Alcohol-1. BG Alcohol-2, Biotin-C2-Amine, and Diazirine NHS Ester were prepared as described in Example 1, above, where BG Alcohol-1 corresponds to compound 29, BG-Alcohol-2 corresponds to compound 20, Biotin-C2-Amine corresponds to compound 22, and Diazirine NHS Ester corresponds to compound 23.

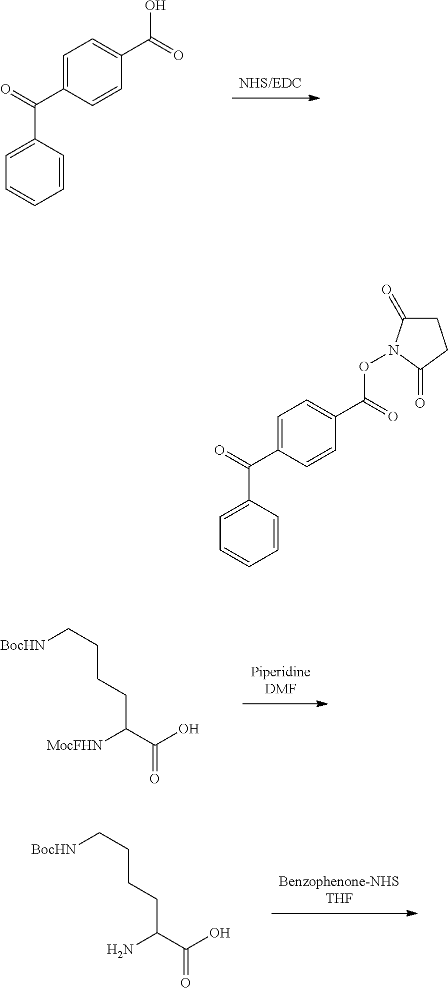

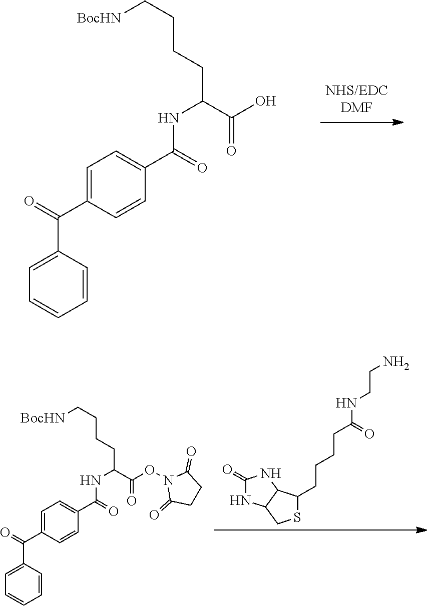 [SMILES: CC(C)(C)OC(=O)NCCCCC(NC(=O)c1ccc(C(=O)c2ccccc2)cc1)C(=O)O ; CC(C)(C)OC(=O)NCCCCC(NC(=O)c1ccc(C(=O)c2ccccc2)cc1)C(=O)ON1C(=O)CCC1=O ; NCCNC(=O)CCCCC1SCC2NC(=O)NC21]

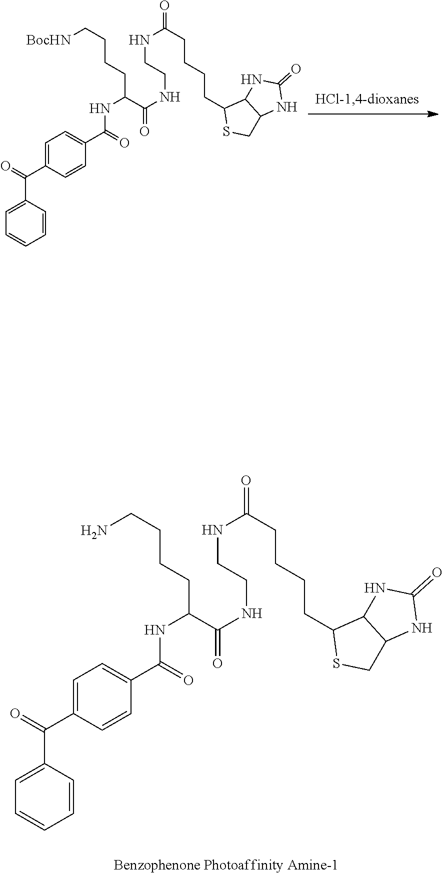 [SMILES: CC(C)(C)OC(=O)NCCCCC(NC(=O)c1ccc(C(=O)c2ccccc2)cc1)C(=O)NCCNC(=O)CCCCC1SCC2NC(=O)NC21 ; NCCCCC(NC(=O)c1ccc(C(=O)c2ccccc2)cc1)C(=O)NCCNC(=O)CCCCC1SCC2NC(=O)NC21]

### Benzophenone Photoaffinity Amine-1

4-(Phenyl-carbonyl) Benzoic Acid (5.0 mmol), EDC HCl (7.5 mmol), and NHS (7.5 mmol) were added to a round bottom flask and dissolved in DMF (0.5 M). After stirring at room temperature for 3 hours, LC/MS analysis revealed complete conversion to product with visible precipitation. The white solid was filtered and rinsed with dI and Et₂O. Benzophenone-NHS Ester was thoroughly dried via vacuum and used without further purification. To a dried round bottom flask, FMoc-Lys(Boc)-OH (10 mmol) was added and dissolved in DMF (0.5 M). Piperidine (100 mmol) was added and the reaction was allowed to stir at room temperature overnight. The next morning, the reaction was diluted with DMF and the resultant residue was filtered. The residue was then collected, sonicated in a small amount of methanol and filtered to yield H-Lys(Boc)-OH. The resulting white powder was dried thoroughly via vacuum and used in the next step without further purification. Next, H-Lys(Boc)-OH (4.292 mmol), benzophenone-NHS (4.635 mmol), NaHCO₃ (8.584 mmol), were suspended in THF/H₂O (2/1, 0.15 M) and allowed to stir overnight at room temperature. The next morning, the reaction was quenched with 10 mL of 1 M NaHSO₄ and extracted with EtOAc. The residue was then purified via column chromatography (DCM/MeOH, 50/1 to 25:1) to yield pure photo-Lys(Boc)-OH, photo-Lys(Boc)-OH (2.53 mmol), EDC.HCl (3.29 mmol), and NHS (3.29 mmol) were then added to a dry round bottom flask and dissolved in MeCN (0.08 M). The resulting solution was allowed to stir for 24 hr at room temperature before being added to freshly deprotected Biotin-C2-Amine (2.6 mmol) and TEA (5 mmol). The reaction was allowed to stir overnight at room temperature. Next, the solvents were removed and the residue was thoroughly rinsed with dI and Et₂O before being purified via HPLC to yield benzophenone-photoaffinity-(Boc)amine. This material (0.247 mmol) was then dissolved in 4M HCl in 1,4-dioxane and allowed to stir for 1 hr. at room temperature before removing all solvent to yield Benzophenone Photoaffinity Amine 1.

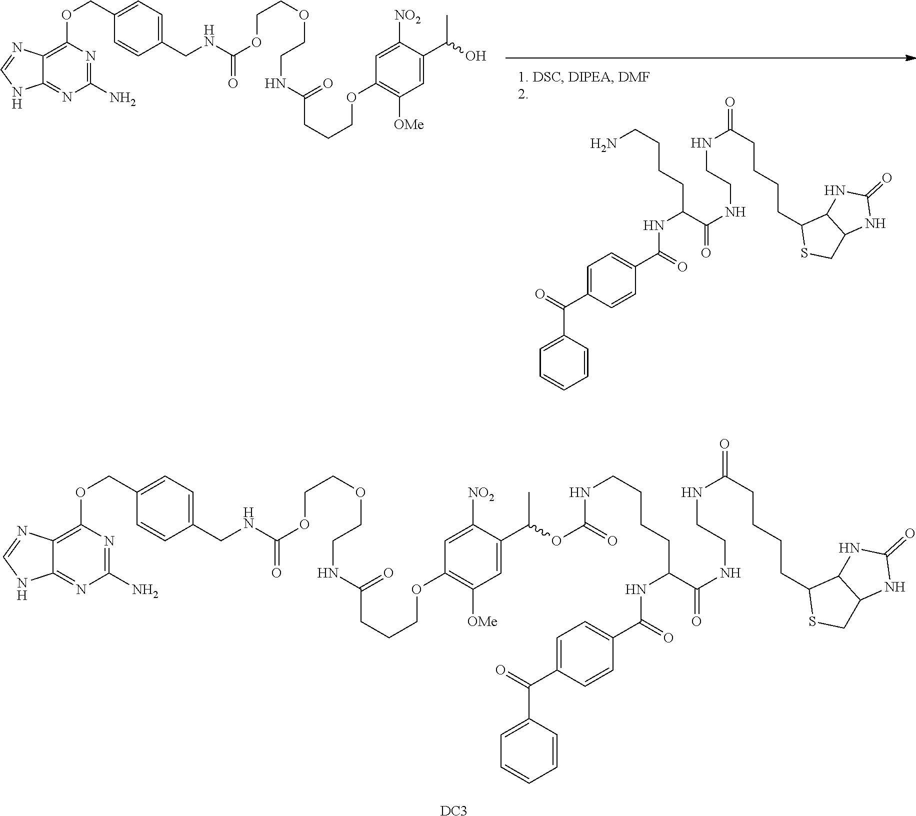 [SMILES: COc1cc(C(C)O)c([N+](=O)[O-])cc1OCCCC(=O)NCCOCCOC(=O)NCc1ccc(COc2nc(N)nc3[nH]cnc23)cc1 ; NCCCCC(NC(=O)c1ccc(C(=O)c2ccccc2)cc1)C(=O)NCCNC(=O)CCCCC1SCC2NC(=O)NC21 ; COc1cc(C(C)OC(=O)NCCCCC(NC(=O)c2ccc(C(=O)c3ccccc3)cc2)C(=O)NCCNC(=O)CCCCC2SCC3NC(=O)NC32)c([N+](=O)[O-])cc1OCCCC(=O)NCCOCCOC(=O)NCc1ccc(COc2nc(N)nc3[nH]cnc23)cc1 ; "DC3"]

BG Alcohol-1 (0.231 mmol) was charged to a dried round bottom flask and dissolved in DMF (0.2 M) and DIPEA (0.741 mmol). DSC (0.233 mmol) was then added and the reaction was allowed to stir for 3 hr. at mom temperature. After this time, the reaction was added to Benzophenone-Photoaffinity Amine 1 (0.247 mmol) and allowed to stir overnight. Solvents were then removed and the residue was rinsed with Et₂O/EtOAc and thoroughly dried. The material was then purified via HPLC to yield DC3 (27 mg).

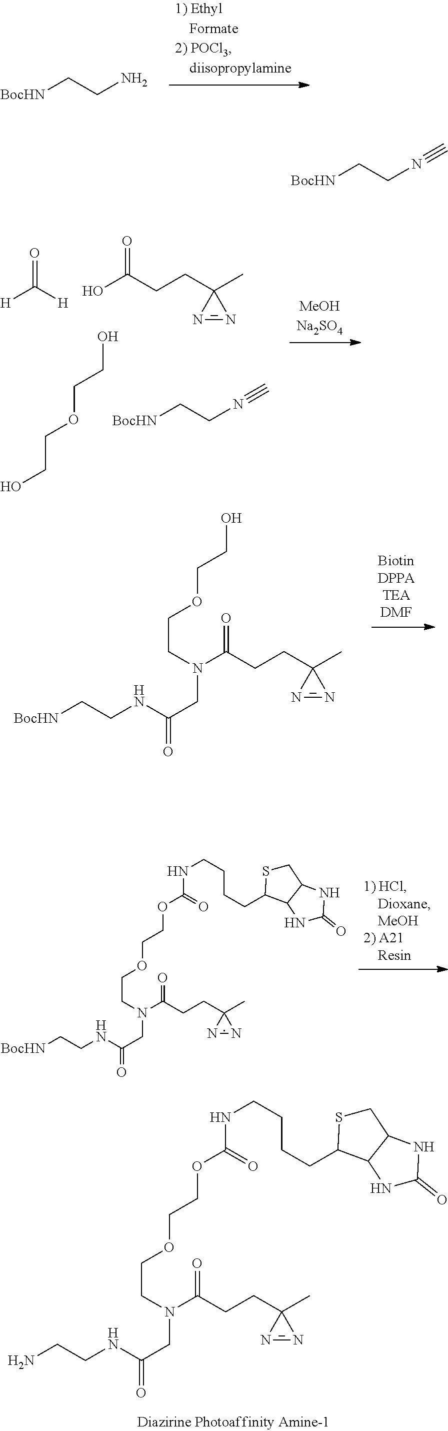

### Diazirine Photoaffinity Amine-1

Diazirine Photoaffinity Amine-1 t-Butyl-N-(2-Aminoethyl) Carbamate (50 mmol) was dissolved in neat ethyl formate (0.1 M) and left to stir at 50° C. for 5 hours after which solvent was removed. This material was dissolved in DCM (0.2 M) and diisopropyl amine (135 mmol) was added. POCl₃ (55 mmol) was then added dropwise at 0° C. The reaction was then quenched with 10% Na₂CO₃ in dI and extracted with DCM. The organic layer was then washed with dI, brine and dried with Na₂SO₄. The material was then purified via column chromatography (1:1 Hexanes:EtOAc) to isonitrile-1 as a thick brown syrup. 2-(2-amino-ethoxy)-ethane (2.4 mmol) and paraformaldehyde (2.4 mmol) were added to a dried round bottom flask, dissolved in MeOH (1.2 M) and allowed to stir overnight at room temperature. The next day, isonitrile-1 (2.0 mmol), and diazirine carboxylic acid (2.0 mmol) were added and the reaction was allowed to stir for an additional day. Solvents were then evaporated and the residue was absorbed onto silica before being subjected to column chromatography [DCM/MeOH, 50/1 to 25/1] to yield a pale yellow oil. This material (1.444 mmol) was then charged to a dry round bottom flask with DMF [0.24 M] and d-biotin (1.7328 mmol). DPPA (1.7328 mmol) was then added dropwise followed by TEA (2.455 mmol). The reaction was then heated to 65° C. and allowed to stir for 6 hours. The rection was brought to room temperature before being quenched with 1 M NaOH for 30 min at room temperature. After the usual extraction with EtOAc, the organics were dried with N₂SO₄ and filtered. Column chromatography (DCM/MeOH, 25/1 to 511 gradient) yielded Diazirine-Photoaffinity-(Boc)amine-1 a thick waxy syrup (179.7 mg). Diazirine-Photoaffinity-(Boc)amine 1 was then dissolved in MeOH (0.1 M) and 4M HCl in 1,4-dioxane (5 mmol of HCl) was added and allowed to stir for 1.5 hr. at room temperature. After removing all solvents, the residue was suspended in Amberlyst-A21 resin and filtered to yield Diazirine Photoaffinity Amine-1 (138.1 mg) as a brown syrup.

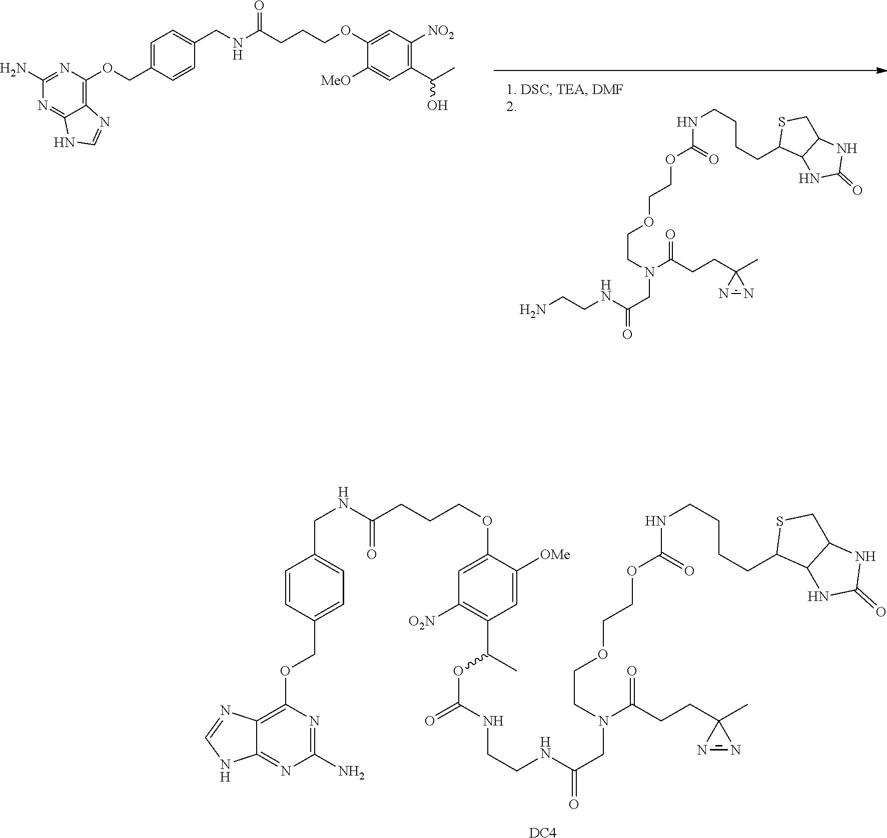 [SMILES: COc1cc(C(C)O)c([N+](=O)[O-])cc1OCCCC(=O)NCc1ccc(COc2nc(N)nc3[nH]cnc23)cc1 ; CC1(CCC(=O)N(CCOCCOC(=O)NCCCCC2SCC3NC(=O)NC32)CC(=O)NCCN)N=N1 ; COc1cc(C(C)OC(=O)NCCNC(=O)CN(CCOCCOC(=O)NCCCCC2SCC3NC(=O)NC32)C(=O)CCC2(C)N=N2)c([N+](=O)[O-])cc1OCCCC(=O)NCc1ccc(COc2nc(N)nc3[nH]cnc23)cc1 ; "DC4"]

BG Alcohol-2 (0.225 mmol) was charged to a dried round bottom flask and dissolved in DMF (0.1 M) and TEA (0.4725 mmol) was added. DSC (0.2367 mmol) was then added and the reaction was allowed to stir overnight at room temperature. After this time, the reaction was added to Diazirine Photoaffinity Amine-1 (0.248 mmol) and allowed to stir for 24 hr. Solvents were then removed and the residue was purified via column chromatography [DCM/MeOH, 50/1 to 5/1] to yield DC4.

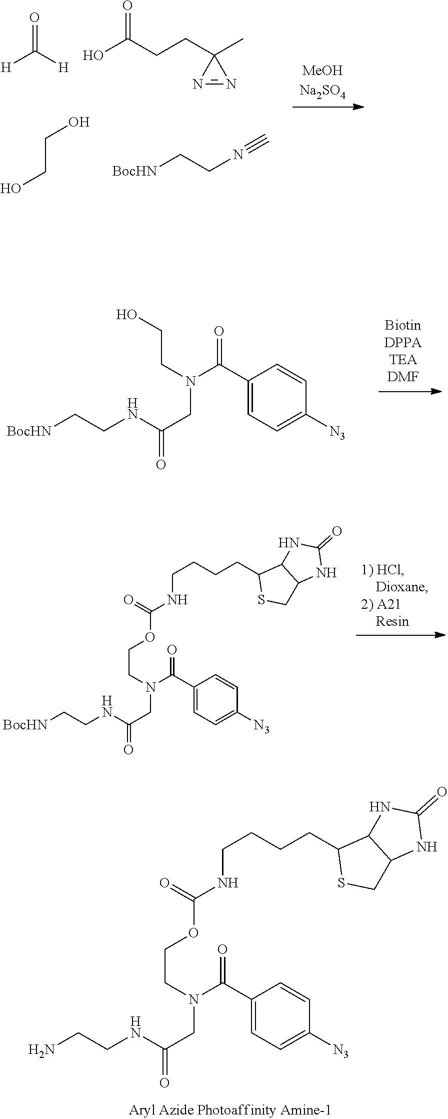

### Aryl Azide Photoaffinity Amine-1

p-amino Benzoic Acid (50 mmol) was added to a dried round bottom flask. Concentrated HCl (0.8 M) was then added on ice. NaNO₂ (50.5 mmol) was then added to the solution. Next, NaN₃ (150 mmol) was dissolved in dI (0.2 M) and this was added to the reaction solution. The usual workup and extraction with EtOAc yielded pure aryl azide carboxylic acid which was used without further purification. Ethanolamine (6 mmol) and paraformaldehyde (6 mmol) were then dissolved in MeOH (1.2 M) and allowed to stir overnight at room temperature. The next day, isonitrile 1 (2.0 mmol), and Aryl azide-carboxylic acid (2.0 mmol) were added and the reaction was allowed to stir for an additional day. The reaction was then diluted with EtOAc and washed with 1 M NaOH and 1 M NaHSO₄. The combined organics were then dried with Na₂SO₄ and filtered. This alcohol was used in the next step without further purification. This material (2.9699 mmol) was then charged to a dry round bottom flask with DMF [0.24 M] and d-biotin (3.2669 mmol). DPPA (4.4548 mmol) was then added dropwise followed by TEA (5.939 mmol). The reaction was then heated to 80° C. and allowed to stir for 6 hours. The reaction was brought to room temperature before being quenched with 1 M NaOH for 1 hr. at room temperature. After the usual extraction with EtOAc, the organics were washed with 1 M NaHSO₄ and dried over Na₂SO₄. The residue was then sonicated in Et₂O/EtOAc and filtered. Column chromatography was then performed [DCM/MeOH, 50/1 to 10/1] to yield Aryl azide-photoaffinity-(Boc)amine as a tacky brown solid (343.7 mg). Aryl azide-photoaffinity-(Boc)amine-1 (0.53 mmol) was then dissolved in MeOH (0.5 M) and 4 M HCl in dioxane (5.3 mmol) was added dropwise. The solution was allowed to stir at room temperature for 1 hr. before removing all solvents. The residue was suspended in MeOH and Amberlyst A21 resin was added. The mixture was filtered and solvents evaporated from flow through to yield Aryl Azide Photoaffinity Amine-1 to be used in the next reaction without further purification.

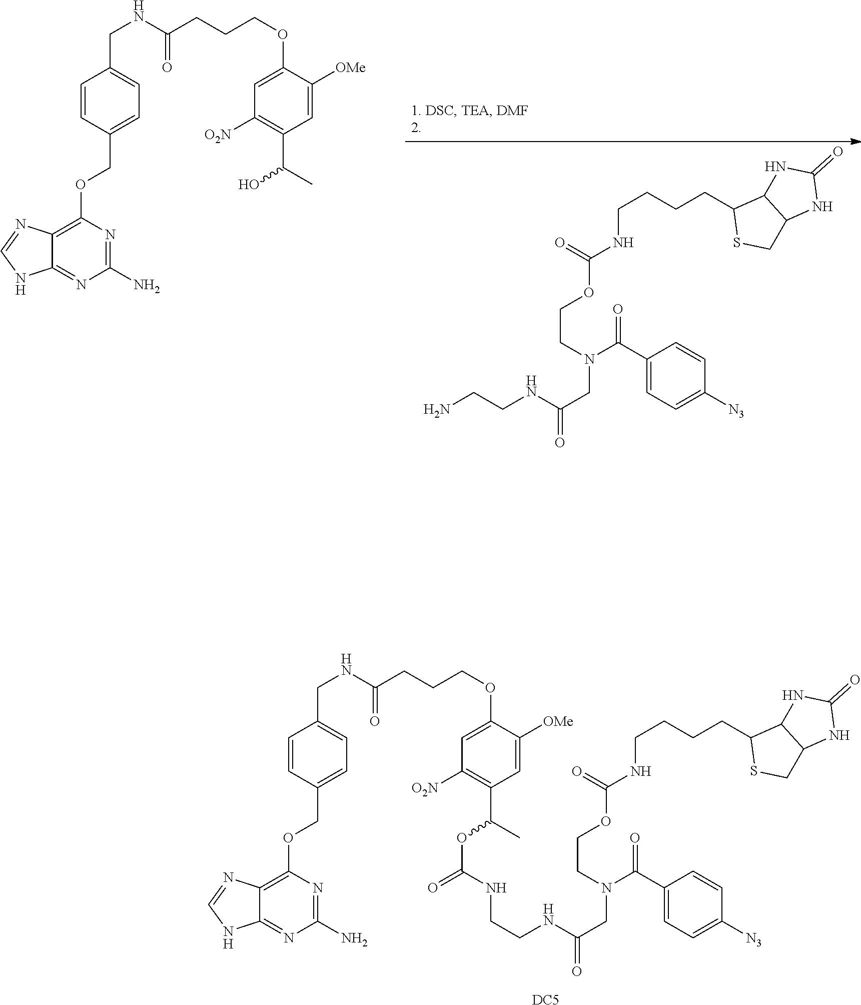 [SMILES: COc1cc(C(C)O)c([N+](=O)[O-])cc1OCCCC(=O)NCc1ccc(COc2nc(N)nc3[nH]cnc23)cc1 ; [N-]=[N+]=Nc1ccc(C(=O)N(CCOC(=O)NCCCCC2SCC3NC(=O)NC32)CC(=O)NCCN)cc1 ; COc1cc(C(C)OC(=O)NCCNC(=O)CN(CCOC(=O)NCCCCC2SCC3NC(=O)NC32)C(=O)c2ccc(N=[N+]=[N-])cc2)c([N+](=O)[O-])cc1OCCCC(=O)NCc1ccc(COc2nc(N)nc3[nH]cnc23)cc1 ; "DC5"]

### DC5

BG Alcohol-2 (0.5841 mmol) was charged to a dried round bottom flask and dissolved in DMF (0.1 M) and TEA (1.062 mmol) was added. DSC (0.6106 mmol) was then added and the reaction was allowed to stir for 4 hours at room temperature. After this time, Aryl-azide-photoaffinity amine-1 (0.531 mmol) and was allowed to stir overnight at room temperature. The next day, solvents were evaporated and the residue was sonicated in EtOAc:Et₂O (3:1) and supernatant was removed. The residue was then sonicated in MeOH:Et₂O (1:1) and supernatant was removed. The residue was them dissolved in DMF, precipitated with dI and filtered to yield pure DC5.

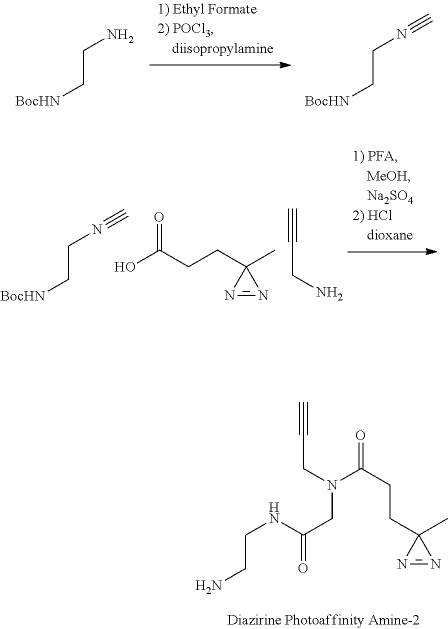

### Diazirine Photoaffinity Amine-2

Propargylamine (2 mmol) and paraformaldehyde (2 mmol) were charged to a dried round bottom flask, dissolved in MeOH (2 M) and allowed to stir overnight at room temperature. The next day, isonitrile 1 (1.0 mmol), and diazirine-carboxylic acid (1.0 mmol) were added and the reaction was allowed to stir for an additional day. The reaction was then diluted with EtOAc and washed with 1 M NaOH and 1 M NaHSO₄. The combined organics were then subjected to a silica plug using EtOAc. The final residue was dissolved in MeOH (0.1 M) and 4 M HCl in dioxane (10 mmol) was added. Removal of all the solvents afforded pure Diazirine Photoaffinity Amine-2 to be used without further purification.

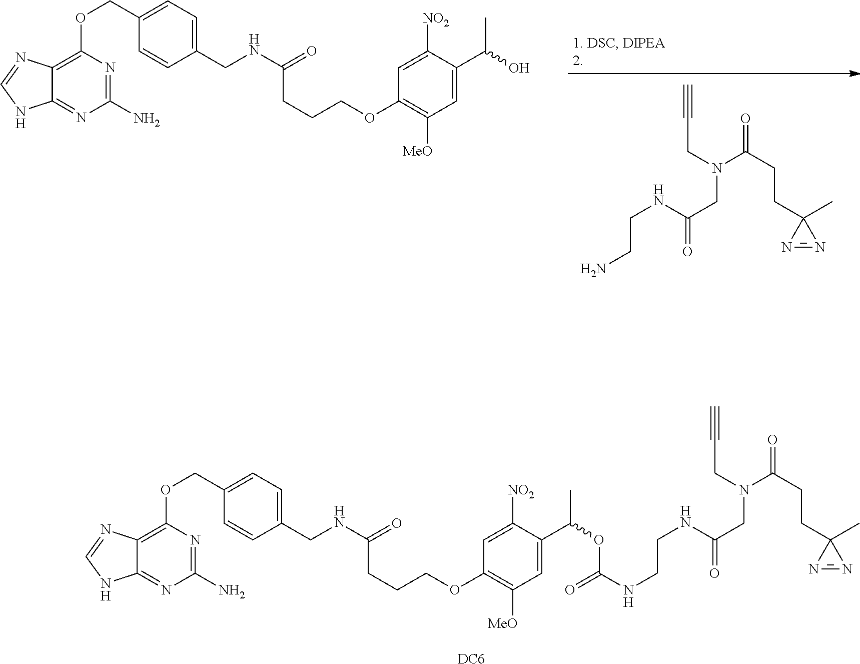 [SMILES: COc1cc(C(C)O)c([N+](=O)[O-])cc1OCCCC(=O)NCc1ccc(COc2nc(N)nc3[nH]cnc23)cc1 ; C#CCN(CC(=O)NCCN)C(=O)CCC1(C)N=N1 ; C#CCN(CC(=O)NCCNC(=O)OC(C)c1cc(OC)c(OCCCC(=O)NCc2ccc(COc3nc(N)nc4[nH]cnc34)cc2)cc1[N+](=O)[O-])C(=O)CCC1(C)N=N1 ; "DC6"]

### DC6

BG Alcohol-2 (0.4133 mmol) was charged to a dried round bottom flask and dissolved in DMF (0.2 M). DSC (0.4174 mmol) was then added, followed by DIPEA (1.24 mmol) and the reaction was allowed to stir for 4 hr. at room temperature. After this time, Diazirine Photoaffinity Amine-2 (0.62 mmol) and was allowed to stir overnight at room temperature. The next day the reaction was diluted with EtOAc and washed with water and brine. Solvents were removed from the combined organics and the resulting residue was purified via HPLC to afford DC6.

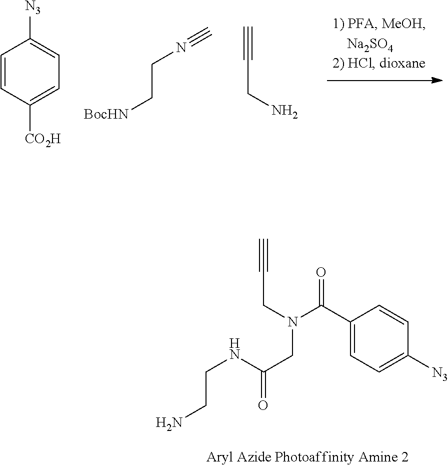

### Aryl Azide Photoaffinity Amine 2

Propargylamine (2 mmol) and paraformaldehyde (2 mmol) were dissolved in MeOH (2 M) and allowed to stir overnight at room temperature. After 5 hr., isonitrile-1 (1.0 mmol), and aryl azide-carboxylic acid (1.0 mmol) were added and the reaction was allowed to stir overnight at room temperature. The reaction was then diluted with EtOAc and washed with 1 M NaOH and 1 M NaHSO₄. The combined organics were then recrystallized from MeOH and Et₂O. The final residue was dissolved in MeOH (0.1 M) and 4 M HCl in dioxane (10 mmol) was added. Removal of all the solvents afforded pure Aryl Azide Photoaffinity Amine-2 (126.2 mg) to be used without further purification.

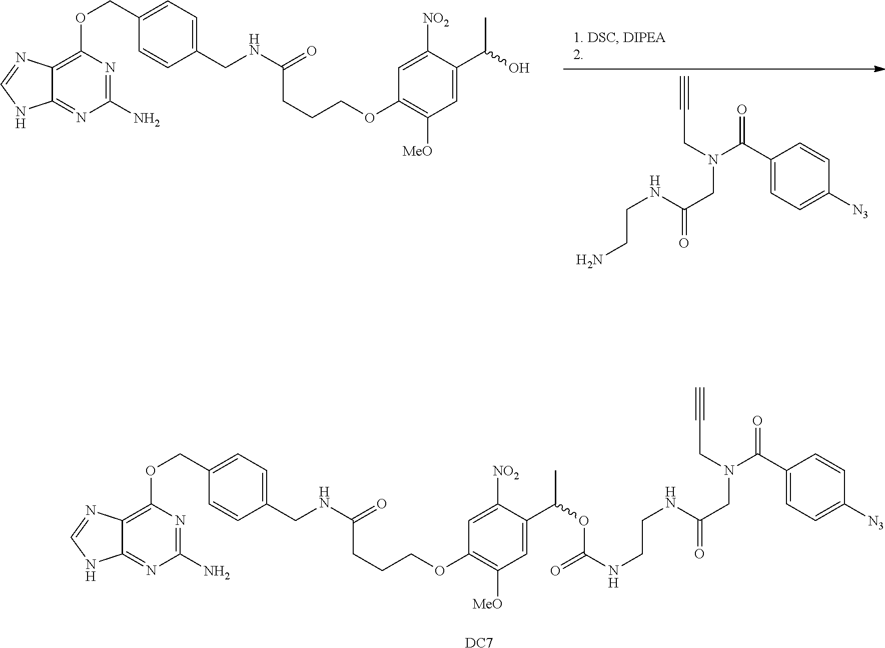 [SMILES: COc1cc(C(C)O)c([N+](=O)[O-])cc1OCCCC(=O)NCc1ccc(COc2nc(N)nc3[nH]cnc23)cc1 ; C#CCN(CC(=O)NCCN)C(=O)c1ccc(N=[N+]=[N-])cc1 ; C#CCN(CC(=O)NCCNC(=O)OC(C)c1cc(OC)c(OCCCC(=O)NCc2ccc(COc3nc(N)nc4[nH]cnc34)cc2)cc1[N+](=O)[O-])C(=O)c1ccc(N=[N+]=[N-])cc1 ; "DC7"]

### DC7

BG Alcohol-2 (0.1813 mmol) was charged to a dried round bottom flask and dissolved in DMF (0.2 M). DSC (0.1831 mmol) was then added, followed by DIPEA (0.54 mmol) and the reaction was allowed to stir for 5 hr. at room temperature. After this time, Aryl azide-photoaffinity amine-2 (0.36 mmol) was added and allowed to stir overnight at room temperature. The next day the reaction was diluted with EtOAc and washed with water and brine. Solvents were removed from the combined organics and the resulting residue was purified via HPLC to afford DC7 (17.3 mg).

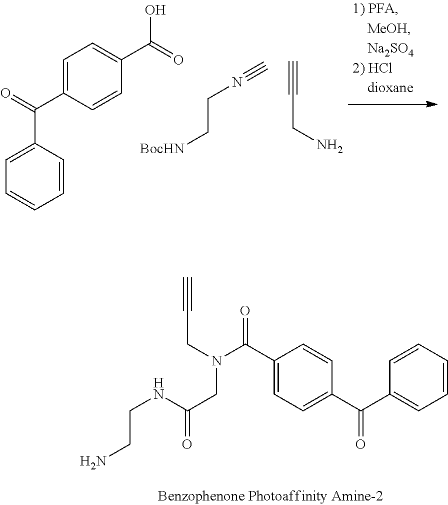

### Benzophenone Photoaffinity Amine-2

Propargylamine (2 mmol) and paraformaldehyde (2 mmol) were dissolved in MeOH (2 M) and allowed to stir at room temperature. After 5 hr., isonitrile-1 (1.0 mmol), and benzophenone-carboxylic acid (1.0 mmol) were added and the reaction was allowed to stir overnight at room temperature. The reaction was then diluted with EtOAc and washed with 1 M NaOH and 1 M NaHSO₄. The combined organics were then recrystallized from MeOH and Et₂O The final residue was dissolved in MeOH (0.1 M) and 4 M HCl in dioxane (10 mmol) was added. Removal of all the solvents afforded pure Benzophenone Photoaffinity Amine-2 (yellow syrup, 232 mg) to be used without further purification

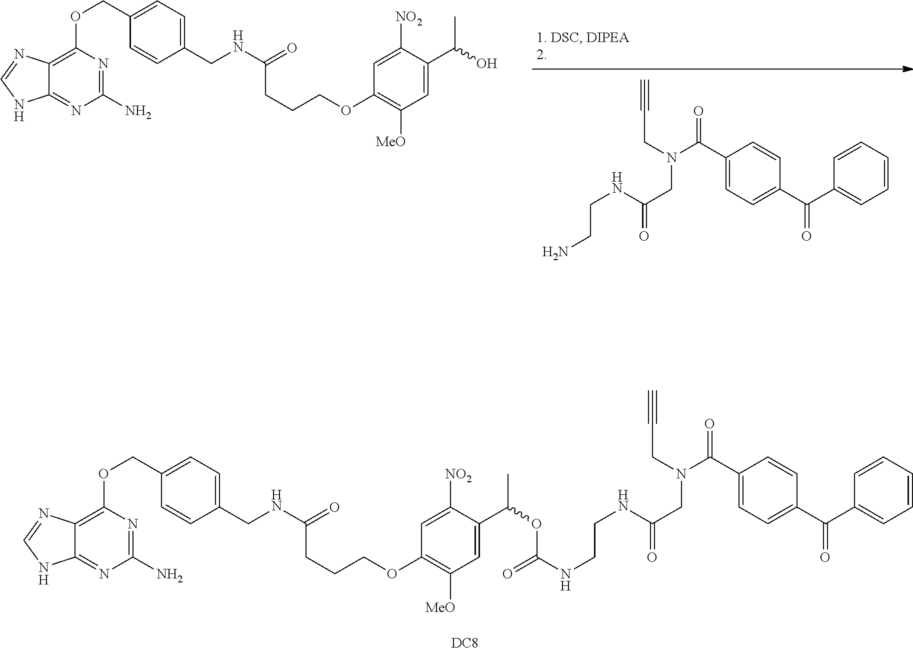 [SMILES: COc1cc(C(C)O)c([N+](=O)[O-])cc1OCCCC(=O)NCc1ccc(COc2nc(N)nc3[nH]cnc23)cc1 ; C#CCN(CC(=O)NCCN)C(=O)c1ccc(C(=O)c2ccccc2)cc1 ; C#CCN(CC(=O)NCCNC(=O)OC(C)c1cc(OC)c(OCCCC(=O)NCc2ccc(COc3nc(N)nc4[nH]cnc34)cc2)cc1[N+](=O)[O-])C(=O)c1ccc(C(=O)c2ccccc2)cc1 ; "DC8"]

### DC8

BG Alcohol-2 (0.1813 mmol) was charged to a dried round bottom flask and dissolved in DMF (0.09 M). DSC (0.1831 mmol) was then added, followed by DIPEA (0.5439 mmol) and the reaction was allowed to stir for 4 hr. at room temperature. After this time, Benzophenone-photoaffinity amine-2 (0.36 mmol) was added and allowed to stir overnight at room temperature. The next day, the reaction was diluted with EtOAc and washed with water and brine. Solvents were removed from the combined organics and the resulting residue was purified via HPLC to afford DC8 (37.3 mg).

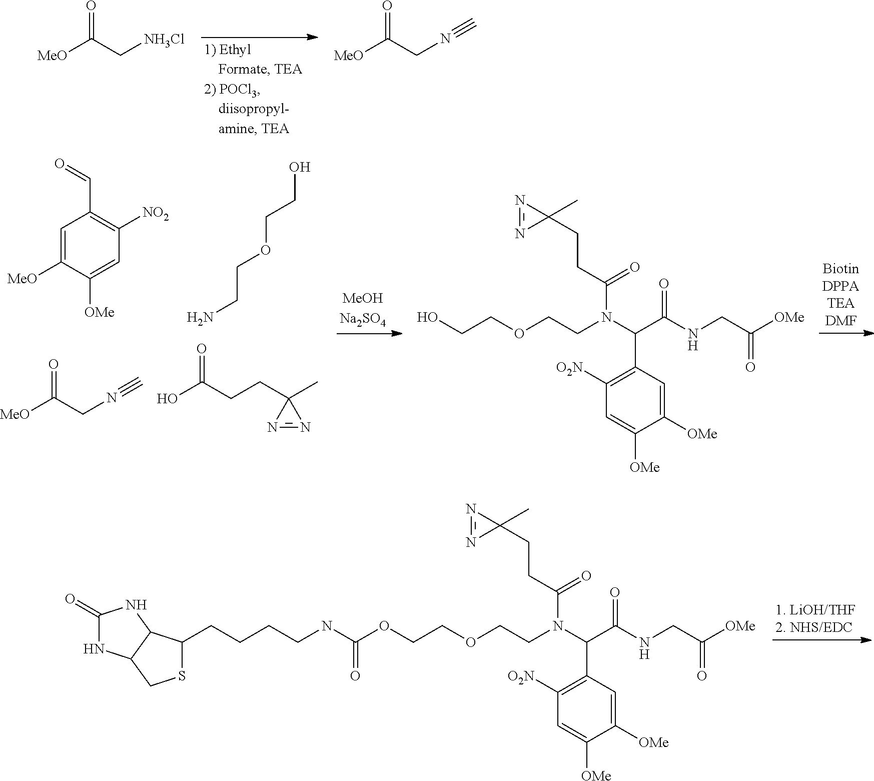

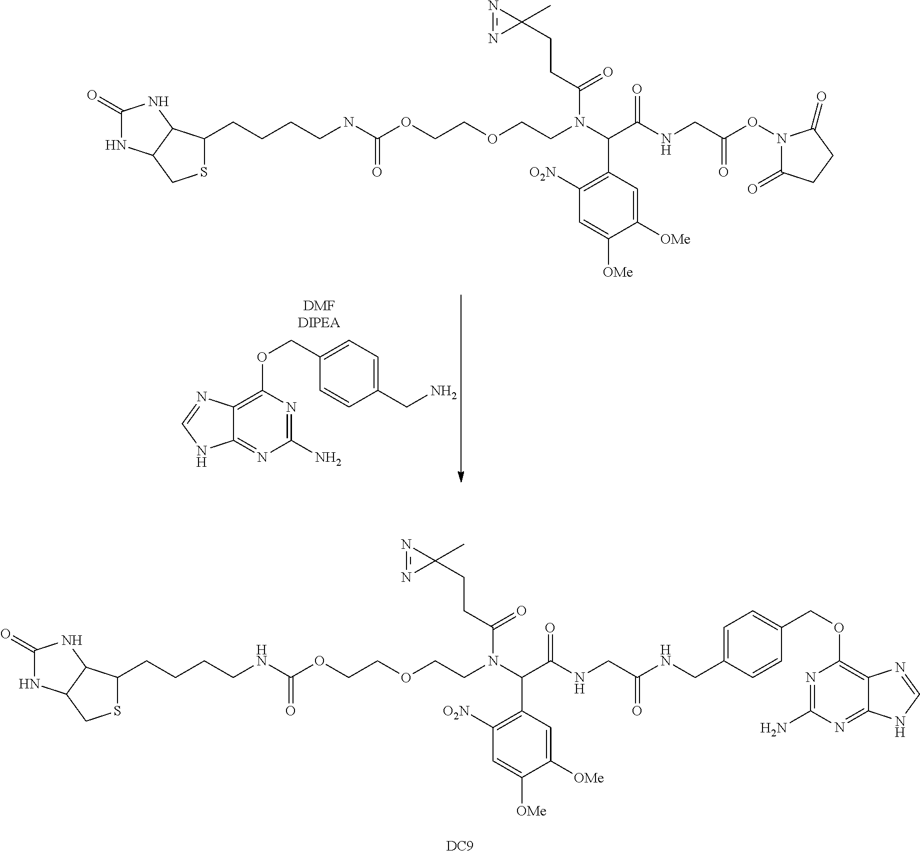 [SMILES: COc1cc(C(C(=O)NCC(=O)ON2C(=O)CCC2=O)N(CCOCCOC(=O)NCCCCC2SCC3NC(=O)NC32)C(=O)CCC2(C)N=N2)c([N+](=O)[O-])cc1OC ; NCc1ccc(COc2nc(N)nc3[nH]cnc23)cc1 ; COc1cc(C(C(=O)NCC(=O)NCc2ccc(COc3nc(N)nc4[nH]cnc34)cc2)N(CCOCCOC(=O)NCCCCC2SCC3NC(=O)NC32)C(=O)CCC2(C)N=N2)c([N+](=O)[O-])cc1OC ; "DC9"]

### DC9

Glycine methyl ester hydrochloride (40 mmol) was dissolved in neat ethyl formate (0.2 M), TEA (60 mmol) and left to stir at 50° C. for 5 hr. after which solvent was removed. This material was then dissolved in DCM (0.2 M) and diisopropyl amine (128 mmol) was added. POCl₃ (48 mmol) was then added dropwise at 0° C. The reaction was then quenched with 10% Na₂CO₃ in dI and extracted with DCM. The organic layer was then washed with dI, brine and dried over Na₂SO₄ to afford isonitrile-2 (1.0979 g) as a thin red-brown oil. 3,4-dimethoxybenzaldehyde (20 mmol) was added to a dried round bottom flask. TFA was then added and the solution was cooled to 0° C. NaNO₃ (60 mmol) was added and the reaction was allowed to stir for 4 hr. at 0° C. The reaction was then quenched with 100 mL dI. The resulting precipitate was then filtered, rinsed with dI, and dried to afford a yellow solid. The aldehyde (1.1 mmol) was then dissolved in MeOH (1 M) and 2-(2-amino ethoxy)-ethanol (1.1 mmol). The reaction was allowed to stir for 5 hr. and then diazirine-NHS ester (1.0 mmol), and isonitrile-2 (1.0 mmol) were added and the reaction was allowed to stir overnight at room temperature. The reaction was then diluted with EtOAc and washed with 1 M NaOH and 1 M NaHSO₄. The residue was then subjected to column chromatography [DCM/MeOH, 50/1 to 10/1 gradient] to yield pure alcohol.

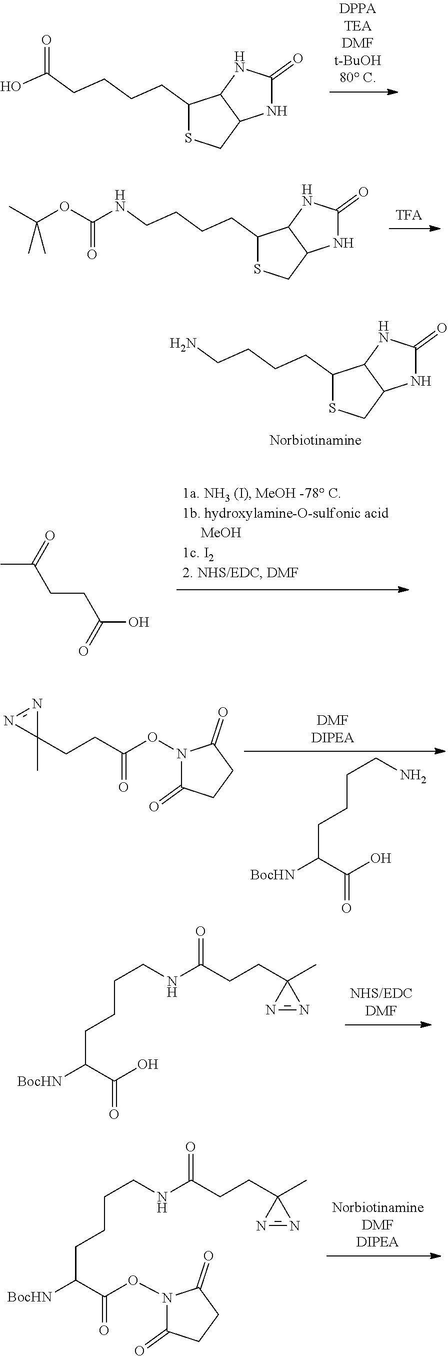

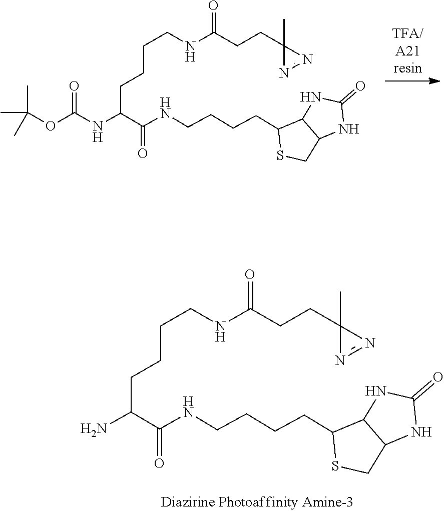 [SMILES: CC1(CCC(=O)NCCCCC(NC(=O)OC(C)(C)C)C(=O)NCCCCC2SCC3NC(=O)NC32)N=N1 ; CC1(CCC(=O)NCCCCC(N)C(=O)NCCCCC2SCC3NC(=O)NC32)N=N1]

### Diazirine Photoaffinity Amine-3

To a dry round bottom flask, Diazirine-NHS ester (2.22 mmol), Boc-Lysine-OH (3.33 mmol) were added and dissolved in DMF (0.25 M). DIPEA (5.55 mmol) was added and the reaction was allowed to stir overnight. The next day, dI was added and the reaction was extracted thoroughly with Et₂O and washed sparingly with water and brine. After evaporating, the resulting residue was purged with N2 to yield the carboxylic acid as a yellow oil (0.735 g). The carboxylic acid (2.06 mmol) was then taken up in DMF (0.3 M). NHS (3.09 mmol) and EDC (3.09 mmol) were added and the reaction was allowed to stir overnight at room temperature. The next morning, dI was added and the reaction was extracted thoroughly with Et₂O. Combined organics were then washed with water, brine, and then dried over with Na₂SO₄. The residue was put on high vacuum and stored overnight at −20° C. to yield diazirine-Lys-NHS-Ester as an off-white wax (0.6415 g). Next, diazirine-Lys-NHS-Ester (0.655 mmol) and norbiotinamine (1.96 mmol) were added to a dried round bottom flask. The contents were then dissolved in DMF (0.1 M) and DIPEA (1.31 mmol) was added. The contents were allowed to stir at room temperature overnight. The next day, a mixture of dI/NH₄C was added to the reaction and the contents were extracted thoroughly with EtOAc. The combined organics were then washed with dI and dried over Na₂SO₄. N₂ was then used to remove all solvents overnight. The crude oil was then treated with dI, which caused precipitation of a white solid. After a thorough rinse with dI and subsequent vacuum evaporation, diazirine-photoaffinity-(Boc)amine-3 was then collected (0.316 g) and used without further purification. Diazirine-photoaffinity-(Boc)-amine-3 (0.57 mmol) was taken up in a mixture of TFA/DCM (2:1, 0.1M) and allowed to stir for 3 hr. at room temperature. N₂ was then used to remove all solvents overnight. The residue was then dissolved in MeOH and added to Amberlyst A21 resin to stir for 30 min. The resin was then filtered, and solvents were evaporated to yield Diazirine Photoaffinity Amine-3 as a white solid (0.238 g).

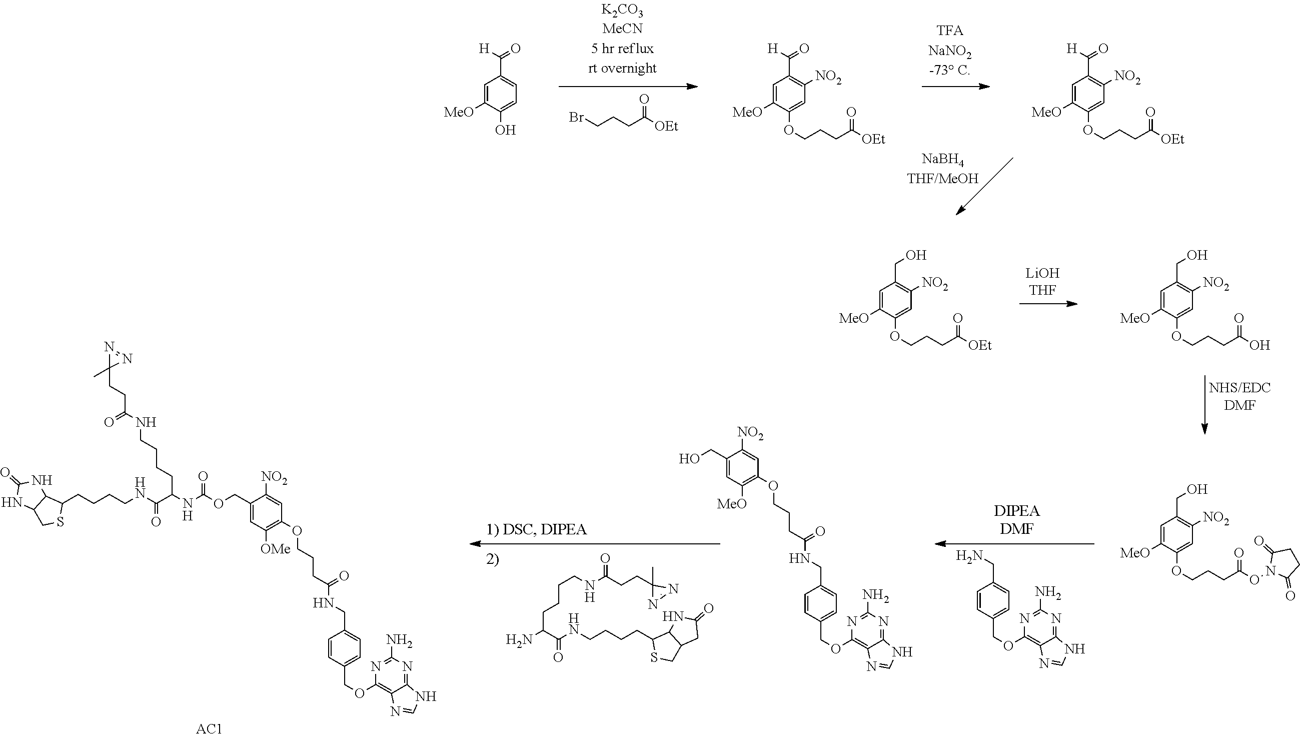

### AC1

BG Alcohol-3 (0.0465 mmol) was charged to a dried round bottom flask and dissolved in DMF (0.2 M). DSC (0.05115 mmol) was then added, followed by DIPEA (0.1395 mmol) and the reaction was allowed to stir overnight at room temperature. After this time, diazirine-photoaffinity amine-3 (0.0558 mmol) was added and allowed to stir for 24 hr. at room temperature. The next day the solvents were evaporated and the resulting residue was treated with dI. The resulting yellow precipitate was filtered. The solid was taken up in MeOH and the suspension was sonicated thoroughly. After filtration, the solid was then purified via HPLC to yield AC1 (3.2 mg) as a yellow solid.

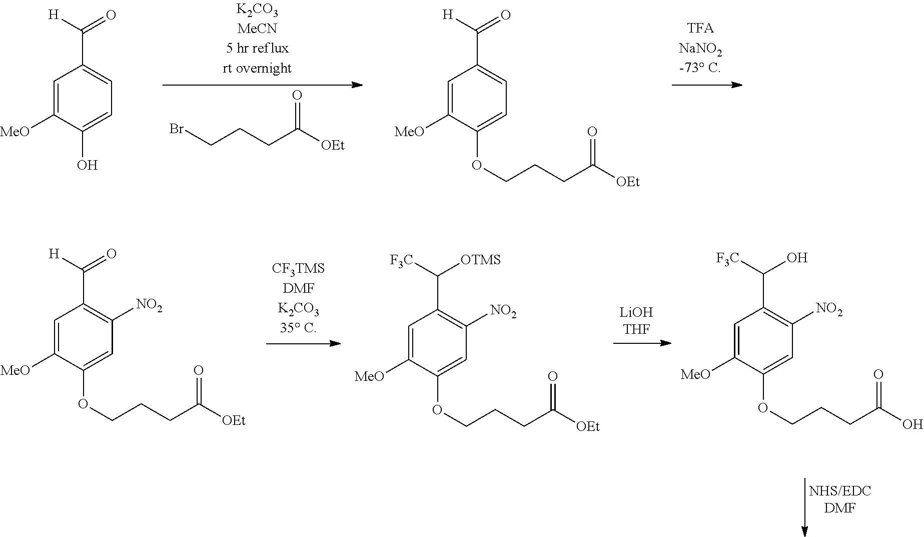

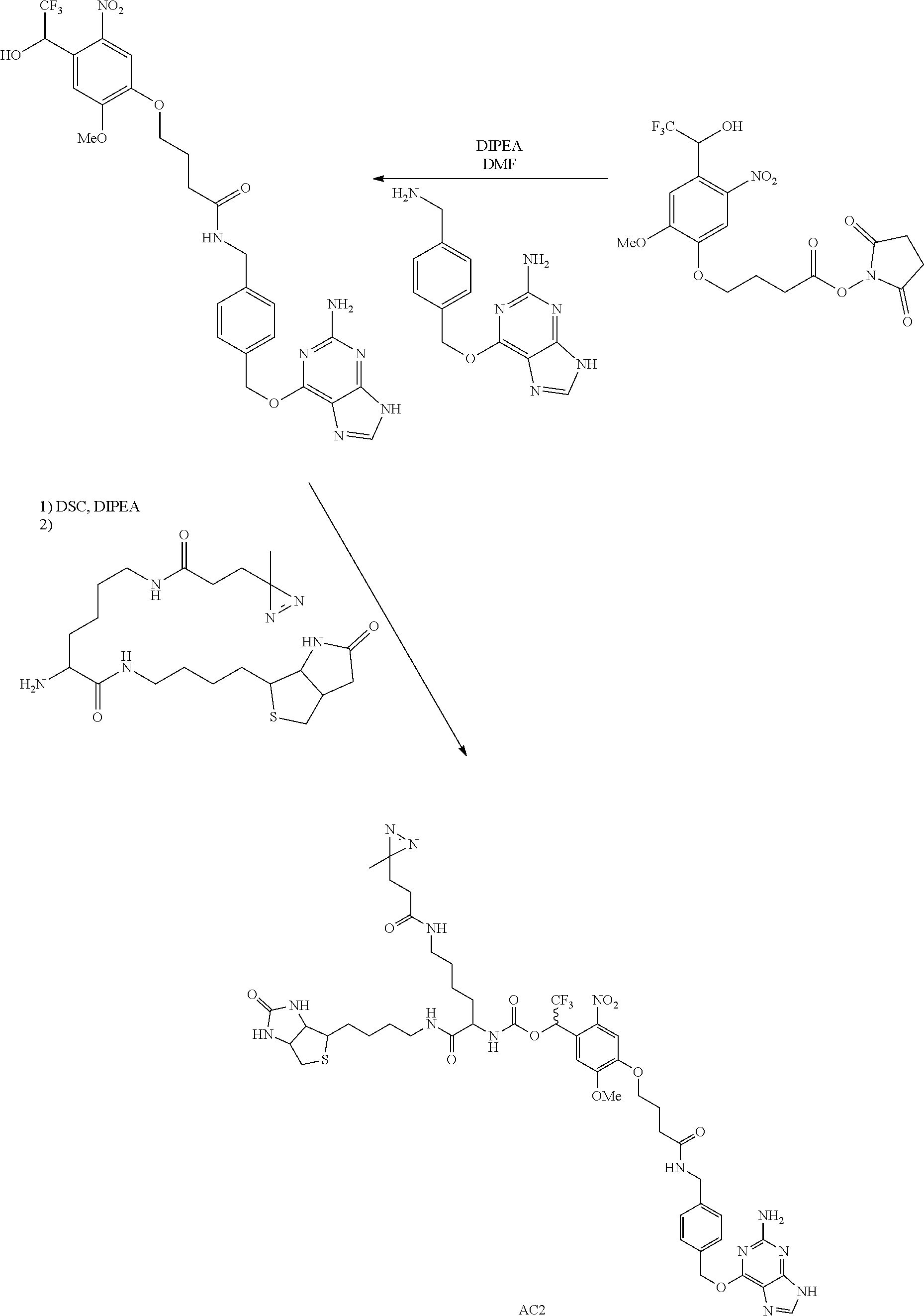 [SMILES: COc1cc(C(O)C(F)(F)F)c([N+](=O)[O-])cc1OCCCC(=O)NCc1ccc(COc2nc(N)nc3[nH]cnc23)cc1 ; NCc1ccc(COc2nc(N)nc3[nH]cnc23)cc1 ; COc1cc(C(O)C(F)(F)F)c([N+](=O)[O-])cc1OCCCC(=O)ON1C(=O)CCC1=O ; CC1(CCC(=O)NCCCCC(N)C(=O)NCCCCC2SCC3CC(=O)NC32)N=N1 ; COc1cc(C(OC(=O)NC(CCCCNC(=O)CCC2(C)N=N2)C(=O)NCCCCC2SCC3NC(=O)NC32)C(F)(F)F)c([N+](=O)[O-])cc1OCCCC(=O)NCc1ccc(COc2nc(N)nc3[nH]cnc23)cc1 ; "AC2"]

### AC2

BG Alcohol-4 (0.073 mmol) was charged to a dried round bottom flask and dissolved in DMF (0.2 M). DSC (0.81 mmol) was then added, followed by DIPEA (0.146 mmol) and the reaction was allowed to stir overnight at room temperature. After this time, diazirine-photoaffinity amine-3 (0.0558 mmol) was added and allowed to stir for 24 hr. at room temperature. The next day, solvents were removed from the reaction and dI was added. The resulting yellow precipitate was filtered and rinsed with dI and Et₂O. The solid was then purified via HPLC to yield AC2 (2.7 mg) as a yellow solid.

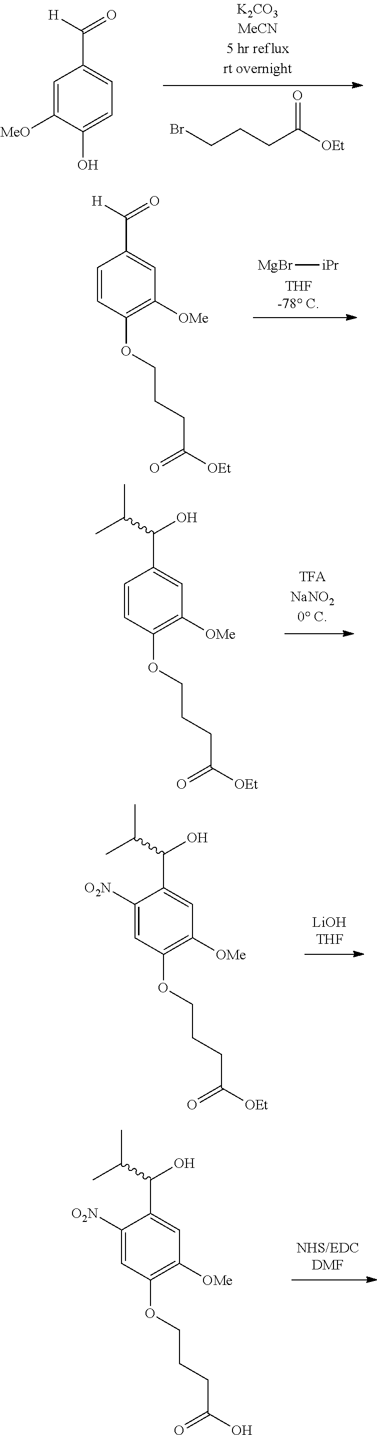

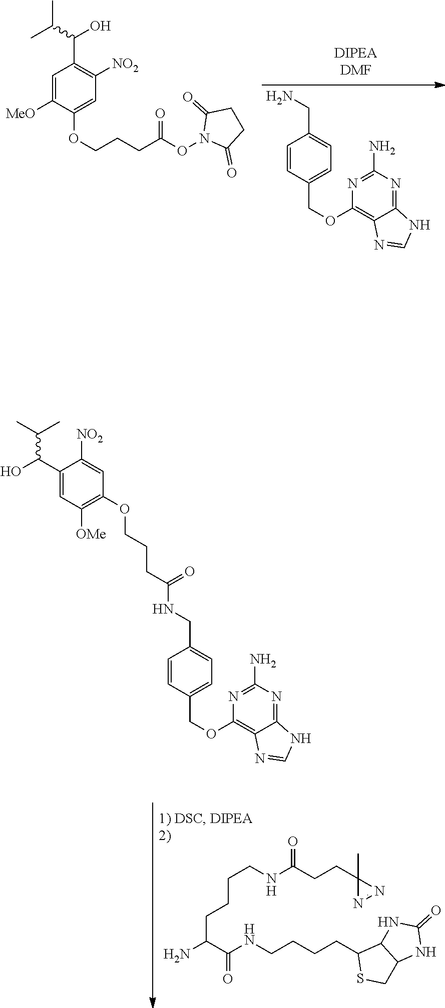 [SMILES: COc1cc(C(O)C(C)C)c([N+](=O)[O-])cc1OCCCC(=O)ON1C(=O)CCC1=O ; NCc1ccc(COc2nc(N)nc3[nH]cnc23)cc1 ; COc1cc(C(O)C(C)C)c([N+](=O)[O-])cc1OCCCC(=O)NCc1ccc(COc2nc(N)nc3[nH]cnc23)cc1 ; CC1(CCC(=O)NCCCCC(N)C(=O)NCCCCC2SCC3NC(=O)NC32)N=N1]

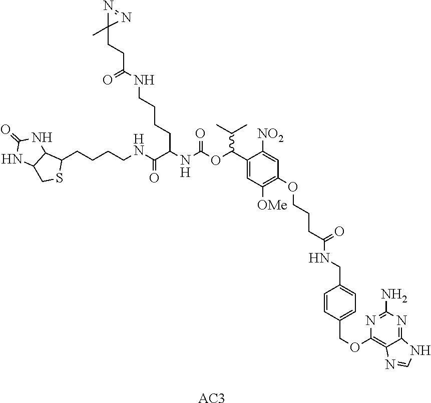 [SMILES: COc1cc(C(OC(=O)NC(CCCCNC(=O)CCC2(C)N=N2)C(=O)NCCCCC2SCC3NC(=O)NC32)C(C)C)c([N+](=O)[O-])cc1OCCCC(=O)NCc1ccc(COc2nc(N)nc3[nH]cnc23)cc1 ; "AC3"]

### AC3

BG Alcohol-5 was charged to a dried round bottom flask and dissolved in DMF. DSC was then added, followed by DIPEA (0.5439 mmol) and the reaction was allowed to stir at room temperature for 5 hr. After this time, diazirine-photoaffinity amine-3 was added and allowed to stir for 24 hr. at room temperature. Solvents were then removed from the reaction and the residue was dissolved in EtOAc. The organic layer was then washed with water, brine and then dried over Na₂SO₄. Solvents were removed and the solid was then purified via column chromatography to yield AC3 as a yellow solid.

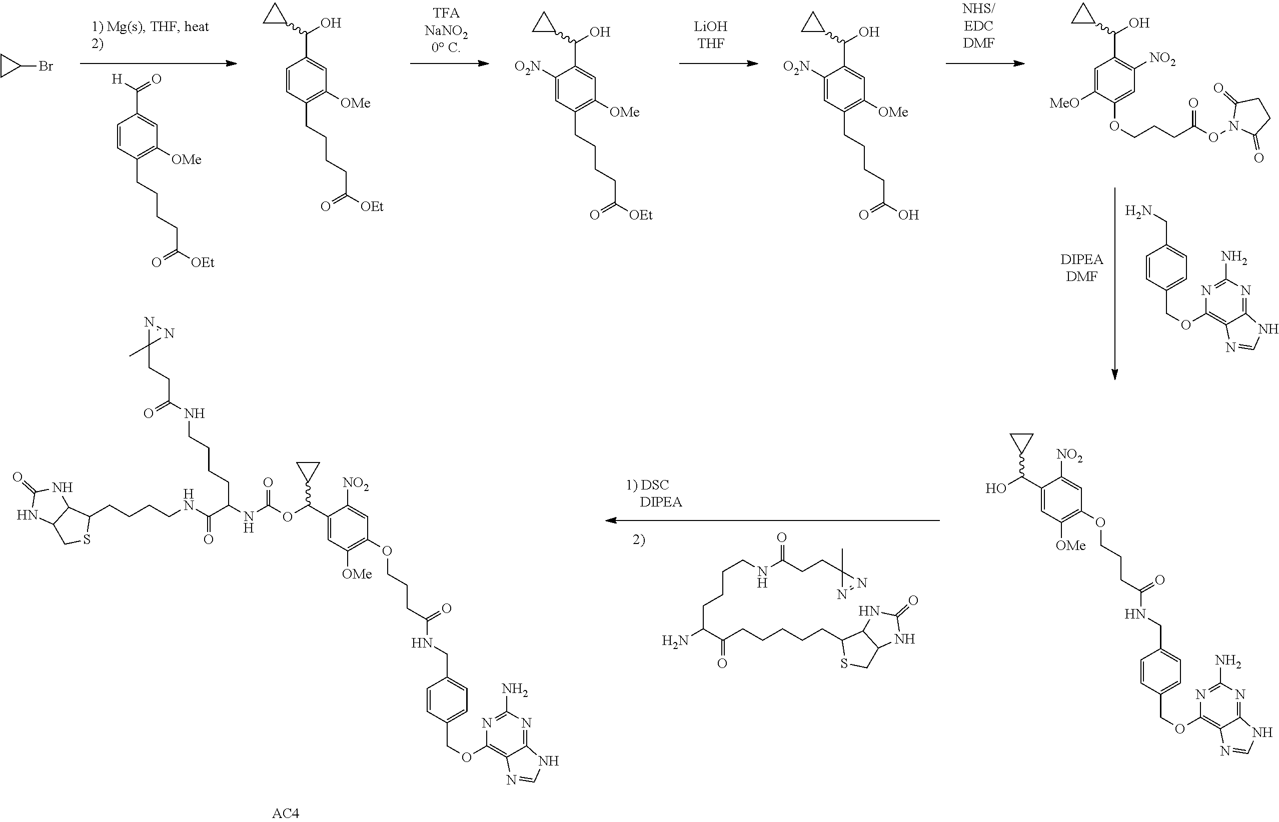

AC4 is prepared as shown in the scheme above, in an analogous manner as the synthesis of AC3, only using cyclopropylbromide to prepare cyclopropyl magnesium bromide in place of the isopropyl magnesium bromide used in the synthesis of AC3.

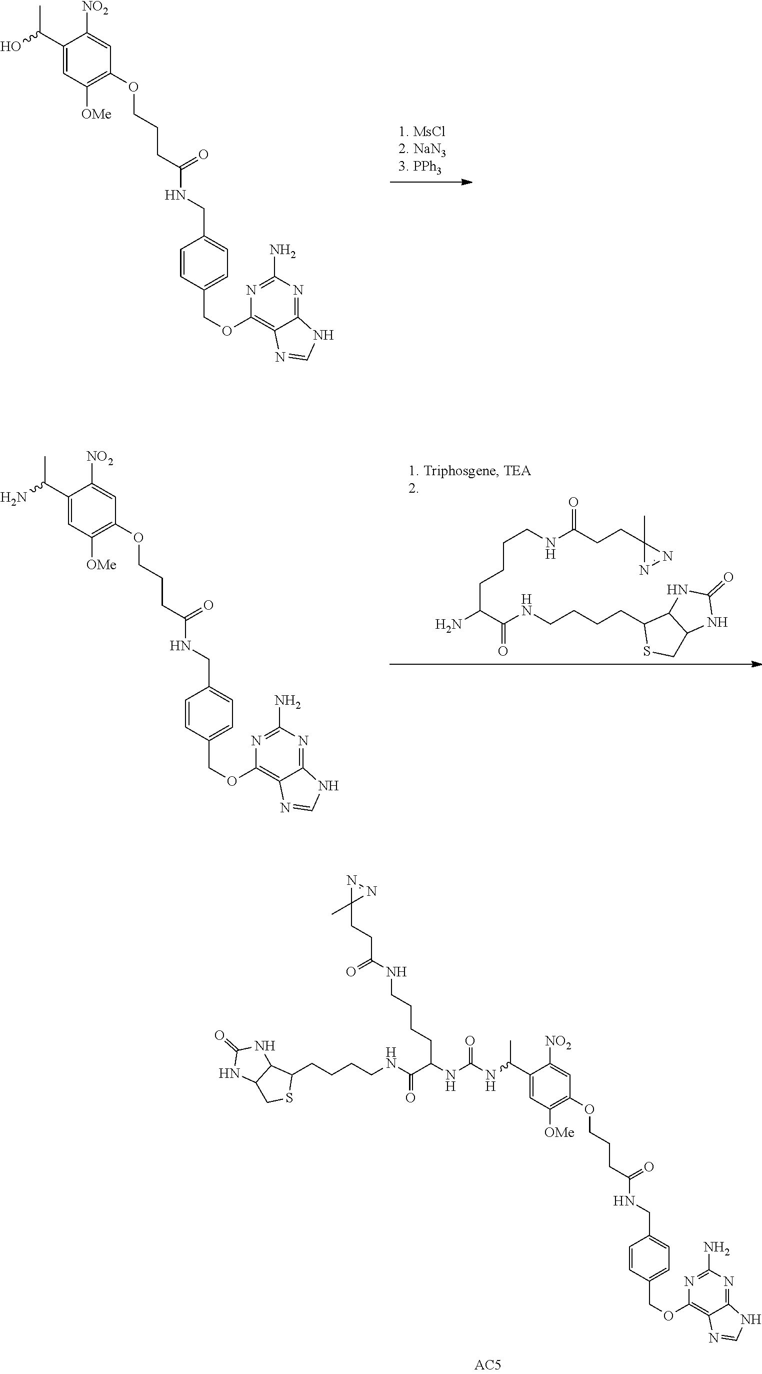 [SMILES: COc1cc(C(C)O)c([N+](=O)[O-])cc1OCCCC(=O)NCc1ccc(COc2nc(N)nc3[nH]cnc23)cc1 ; COc1cc(C(C)N)c([N+](=O)[O-])cc1OCCCC(=O)NCc1ccc(COc2nc(N)nc3[nH]cnc23)cc1 ; CC1(CCC(=O)NCCCCC(N)C(=O)NCCCCC2SCC3NC(=O)NC32)N=N1 ; COc1cc(C(C)NC(=O)NC(CCCCNC(=O)CCC2(C)N=N2)C(=O)NCCCCC2SCC3NC(=O)NC32)c([N+](=O)[O-])cc1OCCCC(=O)NCc1ccc(COc2nc(N)nc3[nH]cnc23)cc1]

### AC5

AC5, which includes a urea bond in place of the carbamate bond in AC1-AC4, is prepared from an alcohol intermediate from the synthesis of AC3, as shown in the scheme above.

### Example 9

### Photoproximity Labelling with AC1

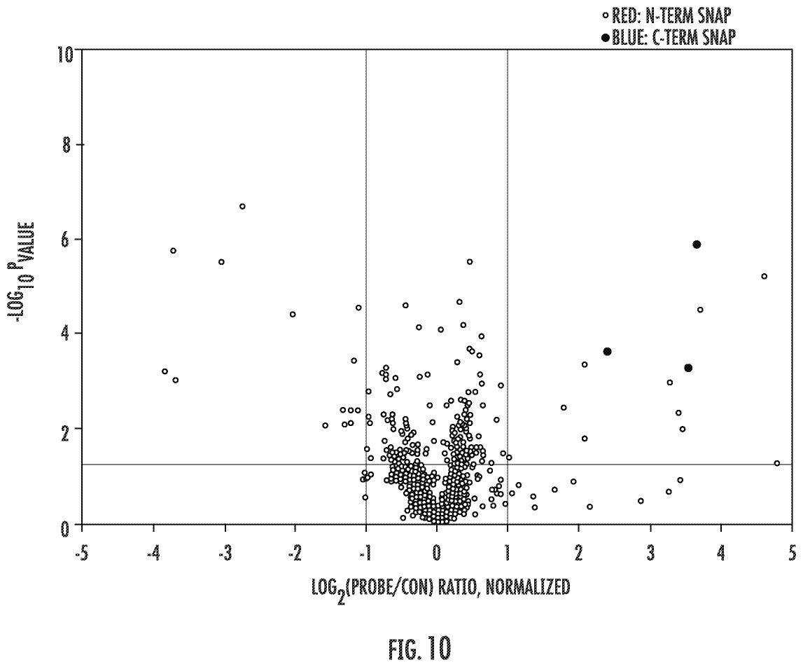

To study if proximal protein interactors could be labeled in response to the dual photoactivation of AC1 (see FIG. 12A) in vitro, the interaction between SNAP-FLAG protein and an α-FLAG monoclonal antibody (mAb) was examined in cell lysate. See FIG. 4A. Briefly, 250 μL aliquots of cell lysate (HEK293T, SNAP-Flag) was incubated in a microcentrifuge tube with either 500 nM of photoprobe, or DMSO for 1 hour at 37° C. Washed anti-FLAG M2 affinity gel, 40 μL of a 50% slurry, was transferred to each reaction in 750 μL of DPBS and the resultant suspension left to rotate overnight at 4° C. (Sigma #A2220; Sigma-Aldrich, St. Louis, Mo., United States of America). Samples were then spun down at 4000×g, supernatant aspirated, and the resin washed with 1M urea in DPBS (1 mL×8) followed by DPBS (1 mL). After being resuspended in 250 μL of DPBS, the +UV samples were placed on ice where they were irradiated with 365 nm light for 10 minutes (SPECTROLINKER™ XL-1500a UV crosslinker, Spectronics Corporation, Westbury, N.Y., United States of America). Once irradiated, the beads were washed with 1 M Urea (1 mL×2) and DPBS (1 mL×2). To elute the remaining SNAP-FLAG and resin-bound FLAG-antibody the beads were boiled for 5 minutes at 95° C. in 20 μL 4×-loading buffer containing 8% SDS and 400 mM DTT. After which, 60 μL of DPBS was added and the suspension boiled at 95° C. for an additional 5 minutes. The samples were then run on SDS-PAGE gel and analyzed via Western Blot. As shown in FIG. 12B, AC1 covalently labels SNAP-tag protein, and subsequently labels a proximal protein binding partner (anti-FLAG IgG in this case) when activated by light.

To determine if AC1 affects cell viability, a viability assay was performed. Briefly, HEK293T cells stably expressing Snap-Flag were seeded at 5,000 cells per well in 100 μL DMEM in a 96-well plate. Upon >90% confluency, media was gently removed and cells were treated with a varying concentration (0, 1, 5, 10, 15, 20, 50 uM) in sextuplicate of AC-1 in a total volume of 75 μL DMEM. After 2 hours at 37° C., media was aspirated and cells were gently washed with 75 ml of serum free DMEM. After final aspiration, media was replaced with 75 ul of DMEM and 75 μL of Cell Titer-Glo™ (Promega Corporation, Madison, Wis., United States of America) was added. The cells were placed on a shaker for 2 minutes and allowed to equilibrate at room temperature for 10 minutes. Luminescence was recorded using a SYNERGY™ Neo HST plate reader (BioTek, Winooski, Vt., United States of America). As shown in FIG. 12C, AC1 does not impair cell growth or viability at doses and times used for PhotoPPI experiments in cells.

AC1 labeling was further studied in live cells (HEK293T expressing Snap-FLAG-KEAP1 protein fusion), each well of a 6-well plate was seeded with 300,000 HEK293T cells stably expressing KEAP1-SF. After reaching ˜90% confluency the growth media was removed, cells washed with DPBS, and treated with varying concentrations (0, 5, 15, 50 μM) of AC1 in 750 μL serum-free DMEM for 1 or 2 hours at 37° C. Post treatment, media was aspirated and non-reacted probe washed out with 1 ml of warm PBS twice. Cold RIPA buffer was added to each well (RIPA+DTT+PI). This study was repeated using 0, 0.5, 1, 5, 10, 20, 30, and 50 μM concentrations of AC1. Biotin-labeling of SNAP-FLAG-KEAP1 shows that AC1 is cell permeable and labels protein at low micromolar concentrations. See FIGS. 12D-12G.

### Example 10

### Photo Reactivity of Nitroveratryl Photocleavable Group Variation

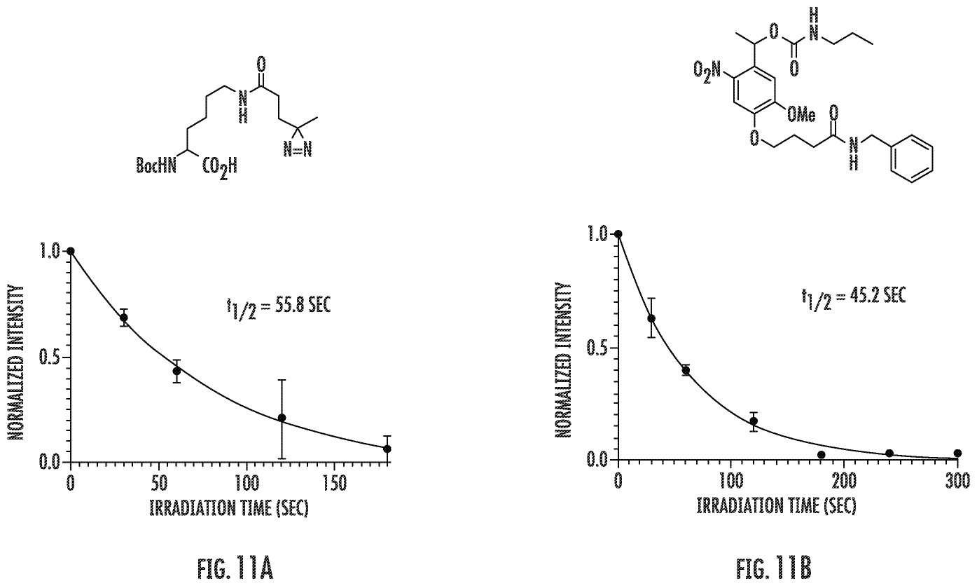

To study the photo-reactivity of the isopropyl-substituted variation of the nitroveratryl photocleavable group of the AC photoPPI probes, AC-M2 (FIG. 13A), a model compound of the AC3 photoprobe was prepared as described in Example 8. The model probe was irradiated and the photocleavage reaction was followed by measuring the signal intensity of the starting material over time. See FIG. 13B. The iso-propyl variation of the photocleavage group was still functional and, as hypothesized, more reactive than previously tested photocleavage groups.

### Example 11

### Synthesis of Catalytic Photo PPI Probe System Components

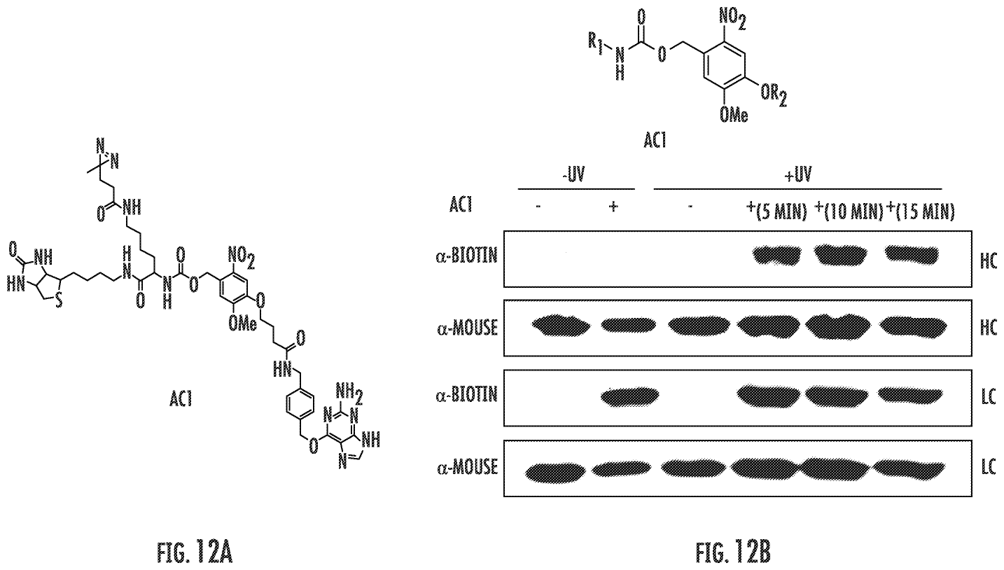

As shown in FIG. 14, the presently disclosed catalytic photoPPI platform operates under the same general workflow as the photoPPI platform shown in FIG. 2B, where a photoactive small molecule is delivered to and covalently labels a fusion protein of interest (POI, shown here as a SNAP-POI fusion). In the catalytic version, however, this molecule is a photosensitizer, which will activate another labeling molecule in the presence of light. So this version can result in catalytic activation of many ‘tagging’ molecules in the proximity of theoretically any fusion protein of interest inside or outside of cells. The catalytic photoPPI system includes a combination of photocatalytic probe and probe substrate that retain photoactive properties, have cell membrane permeability, and are free of interference with normal cellular physiology. In addition, the probe substrate ideally has high target protein labeling capacity.

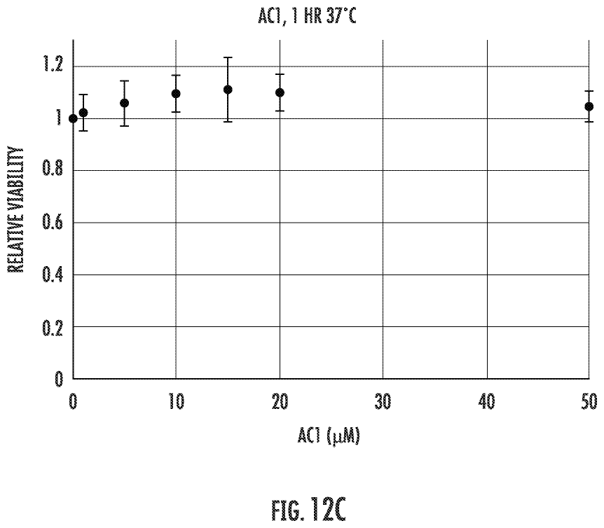

An initial probe system was prepared including a photocatalytic probe comprising a modified flavin scaffold as a catalyst. The modified flavin scaffold has suitable properties required for intracellular delivery, fusion-POI labeling and catalytic photoactivation. The flavin scaffold is linked to a binding moiety, such as benzylguanine. The benzylguanine can localize the catalyst to the SNAP-labeled POI within or on a cell. One exemplary catalytic probe incorporating the flavin-benzylguanine combination is FBG. See FIG. 15A. The probe linker can displace the photocatalytic portion at a desired distance from the SNAP-labeled POI to minimize self-labeling and profile the molecules interacting with the POI in a desired target radius. The initial probe system further included an alkyne- or biotin-derivatized phenol. See FIG. 15B. The phenol group can be converted to a phenoxy radical following complex formation with the excited triplet state of the flavin scaffold of the catalyst upon photon excitation. Many derivatives of the phenol can be used. In addition, other groups, such as anilines and diazirines, can also be used in place of the phenol.

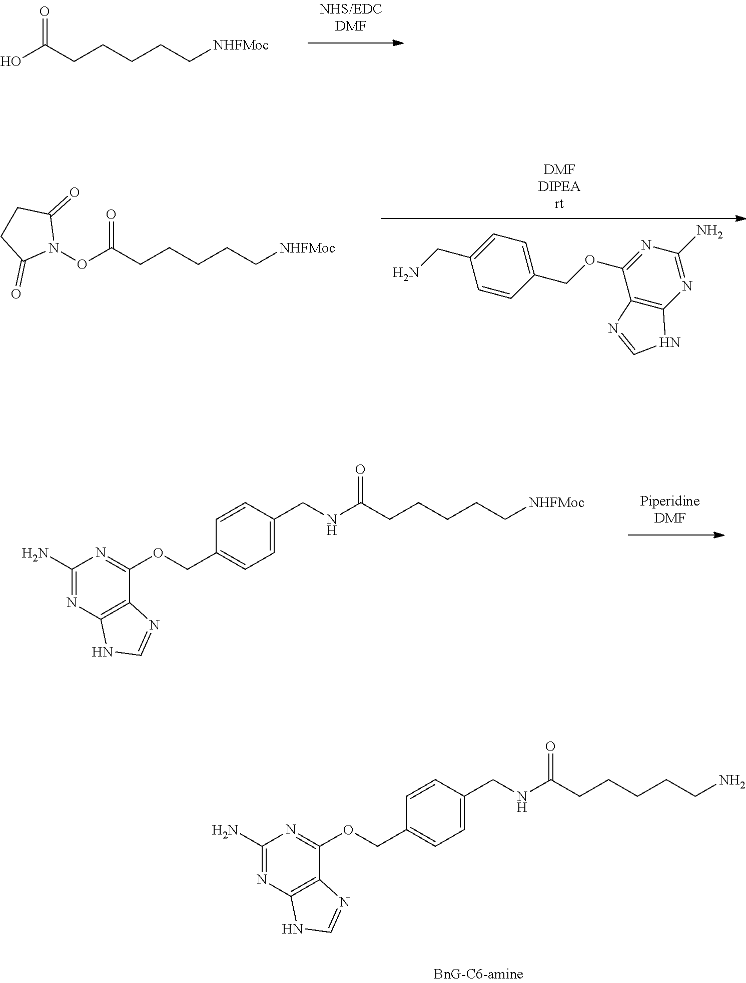

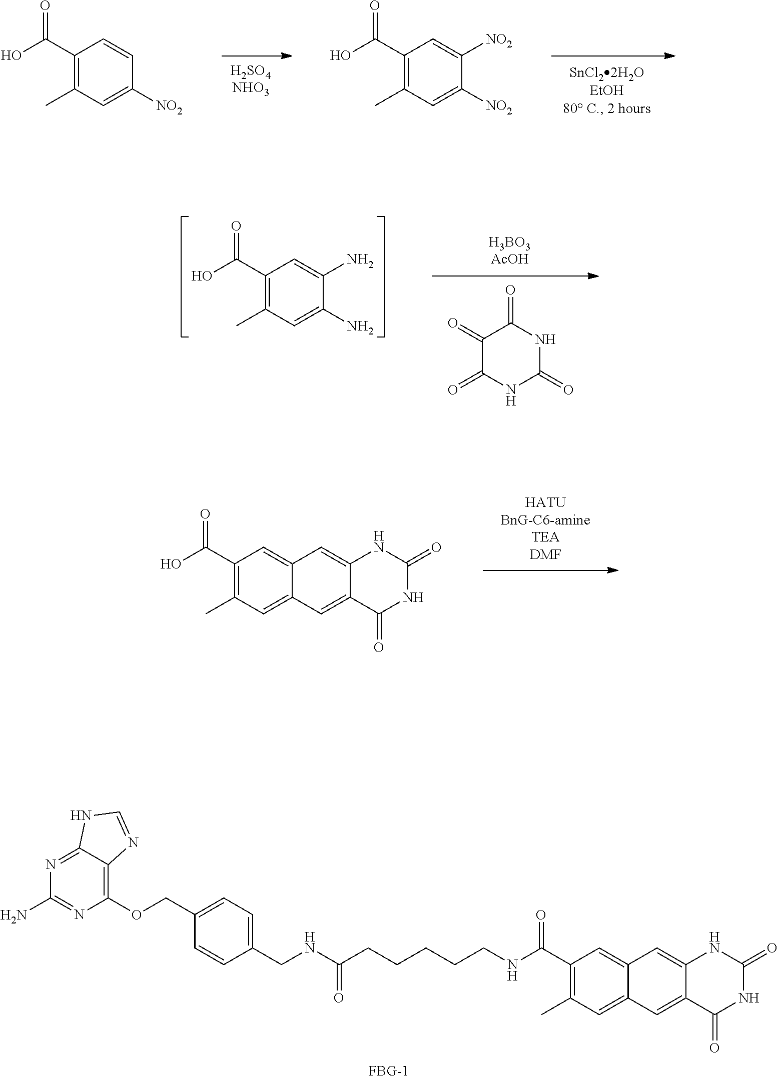

### General Synthetic Method for Flavin-Benzylguanine (FBG) Probes

To a dried round bottom flask, 2-Methyl-4-nitrobenzoic acid (40 mmol) was added. Concentrated sulfuric acid (40 mL) was then added and cooled to 0° C. Next, concentrated nitric acid (40 mL) added dropwise and the reaction was allowed to gradually reach room temperature over the course of an hour. The reaction was allowed to stir for 24 hr. before cooling to 0° C. and adding dI. The mixture was allowed to stir thoroughly before filtering and rinsing with cold dI and hexanes. After drying via vacuum, 3-Methyl-4,5-dinitrobenzoic acid remained as a bright yellow-orange solid. 3-Methyl-4,5-dinitrobenzoic (1 Eq.) was then dissolved in neat ethanol (0.5 M). SnCl₂.2H₂O (6 Eq.) and the reaction was heated to 80° C. The reaction was stirred for 2 hr. before being cooled to room temperature and added to cold dI. The suspension was then basified with 1 M NaOH to pH 7 at 0° C. and allowed to stir for 1 hr. Acetic acid was then added dropwise to attain a pH of 5. The resulting orange precipitate was then filtered and the flow through was put on vacuum in order to remove excess alcohol. Next, the solution was extracted thoroughly with EtOAc. Combined organics were then washed with brine and filtered. Solvents were removed and this material was put through a silica plug using EtOAc as an eluent. Following removal of the solvent, the resulting orange solid was used in the next step without further purification. This solid (1 Eq.) was added to a dried round bottom flask and dissolved in acetic acid (0.15 M). Next, boric acid (1 Eq.) and alloxan monohydrate (1 Eq.) were added and allowed to stir for 2 hr. at room temperature. After this time, a solid precipitated from solution and was filtered. The yellow solid was washed thoroughly with cold acetic acid and then ether. Removal of solvents via vacuum afforded Flavin-Carboxylic acid as a yellow solid. The acid was then added to a dried round bottom flask and dissolved in DMF and DIPEA was added. HATU was added at room temperature before adding BnG-C6-amine and the reaction was allowed to stir overnight. Removal of solvents afforded a residue which was purified using HPLC to afford FBG1 as a yellow solid [LCMS, 638 (M+1)].

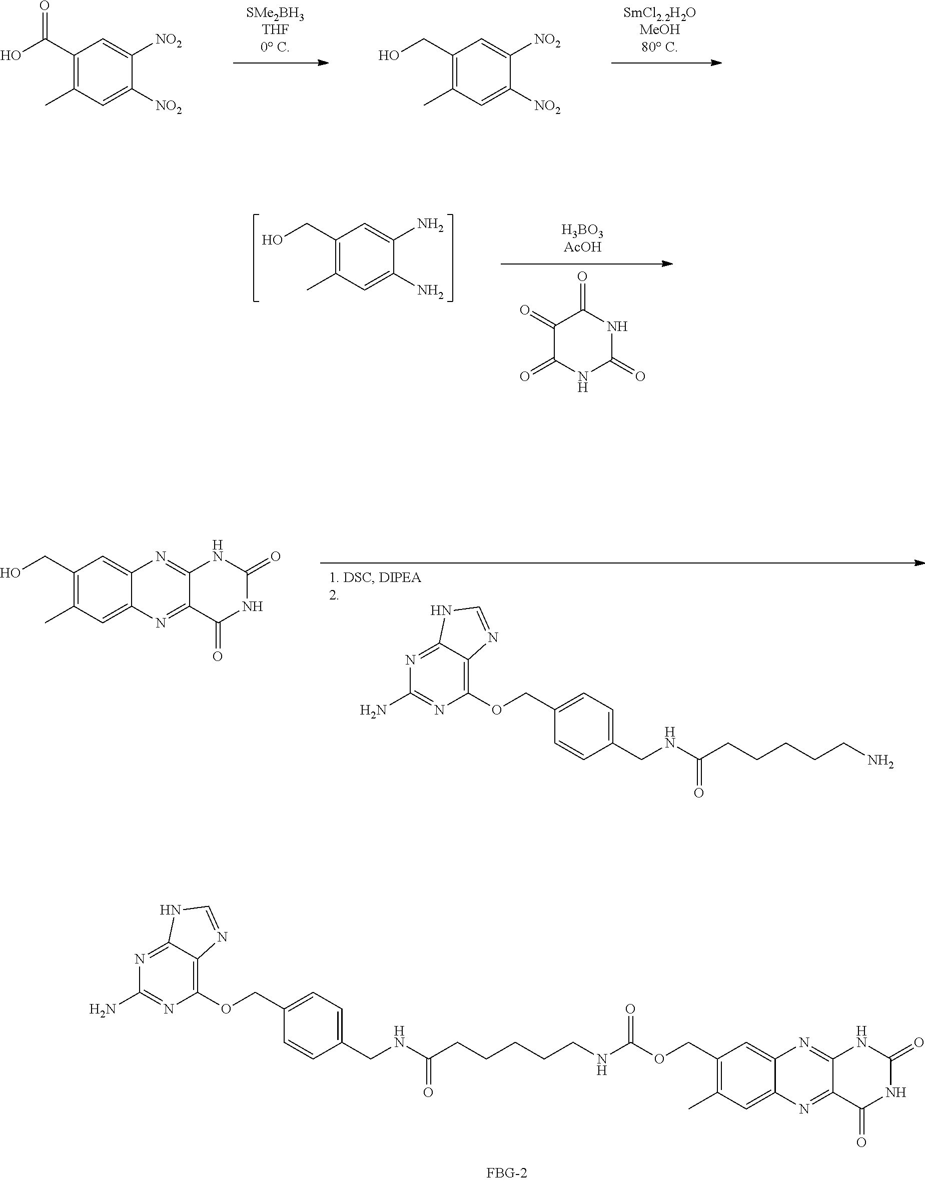

FBG-2, where a direct bond between the flavin scaffold and the linker is replaced by a oxymethylene group, is prepared as shown in the scheme above, by reducing the carboxylic acid group of 3-Methyl-4,5-dinitrobenzoic acid to form a benzyl alcohol prior to forming the flavin scaffold.

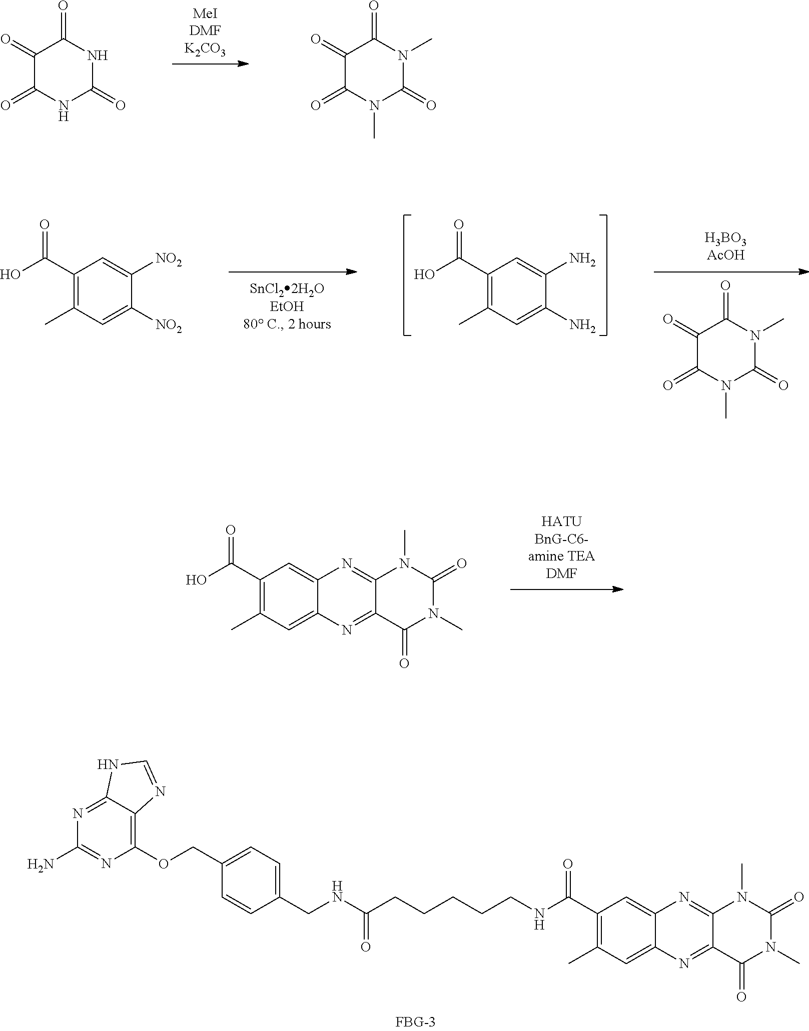

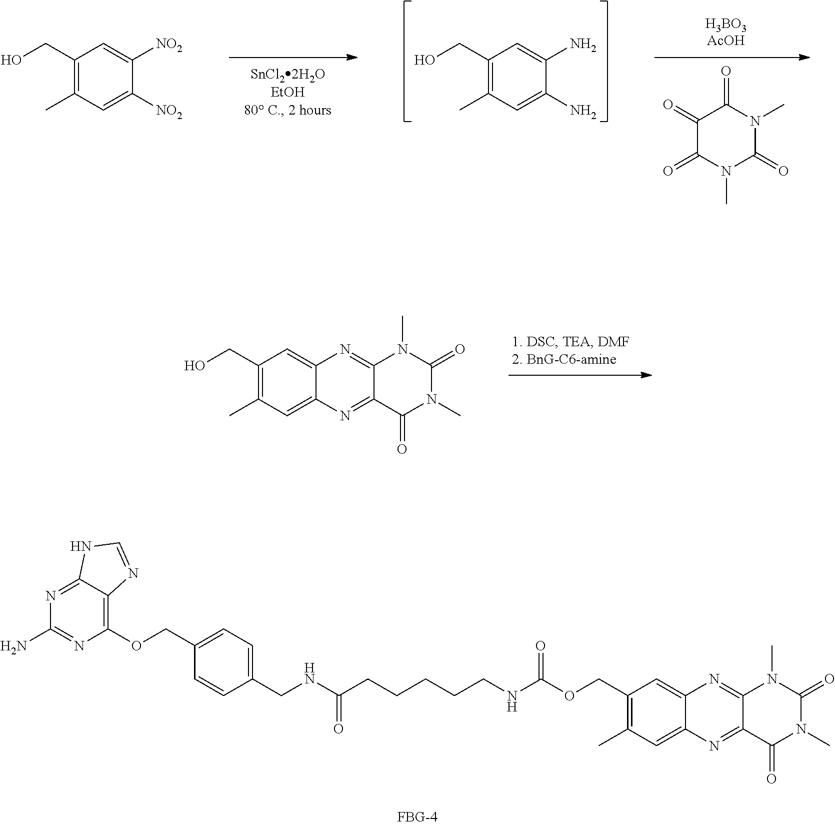

As shown in the scheme above, FBG-3 and FBG-4 are prepared in an analogous manner as FBG-1 and FBG-2, only using N-methylated alloxan in place of the alloxan monohydrate. The N-methylated alloxan is prepared by treating alloxan with methyl iodide in the presence of K₂CO₃.

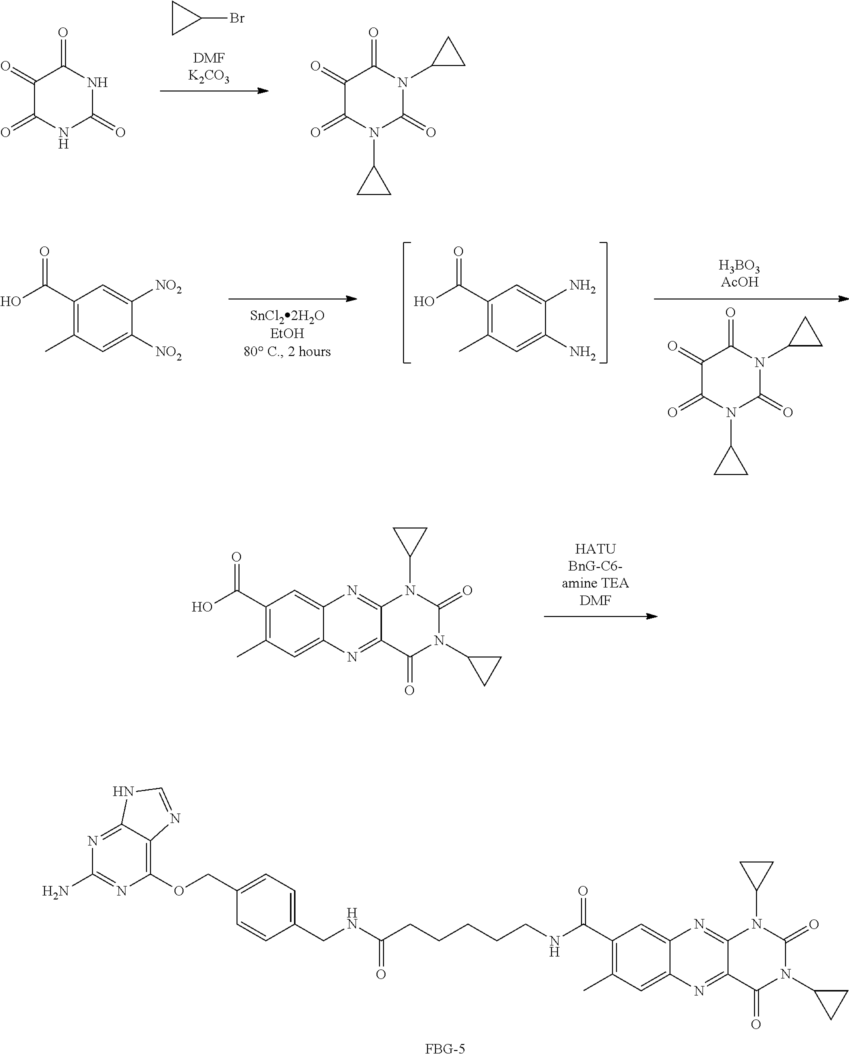

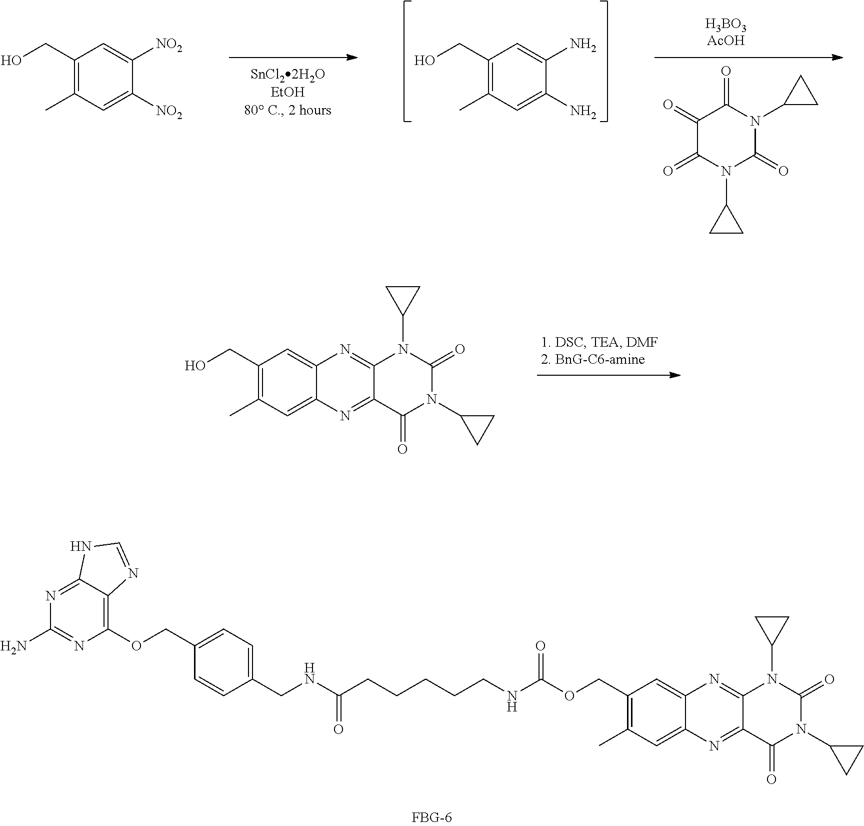

As shown in the scheme above, FBG-5 and FBG-6 are prepared in an analogous manner as FBG-1 and FBG-2, only using N-cyclopropyl alloxan in place of the alloxan monohydrate. The N-cyclopropyl alloxan is prepared by treating alloxan with cyclopropyl bromide in the presence of K₂CO₃.

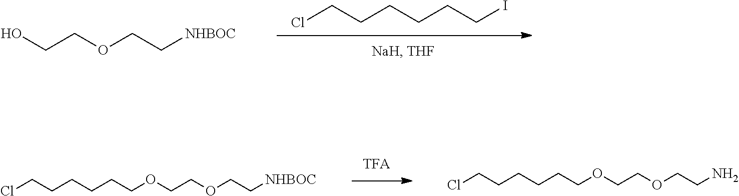

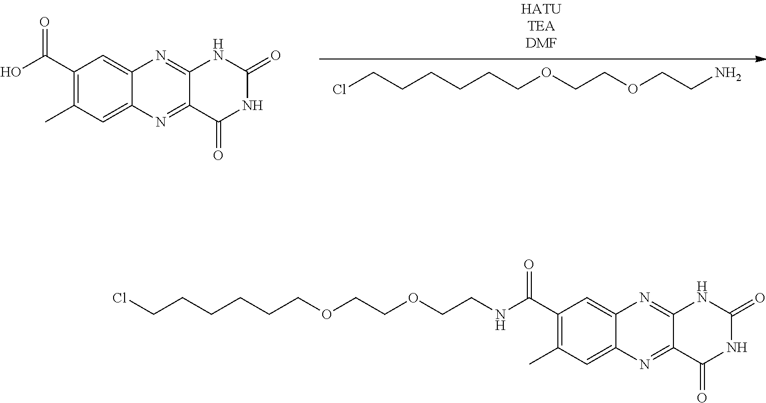 [SMILES: Cc1cc2nc3c(=O)[nH]c(=O)[nH]c3nc2cc1C(=O)O ; NCCOCCOCCCCCCCl ; Cc1cc2nc3c(=O)[nH]c(=O)[nH]c3nc2cc1C(=O)NCCOCCOCCCCCCCl]

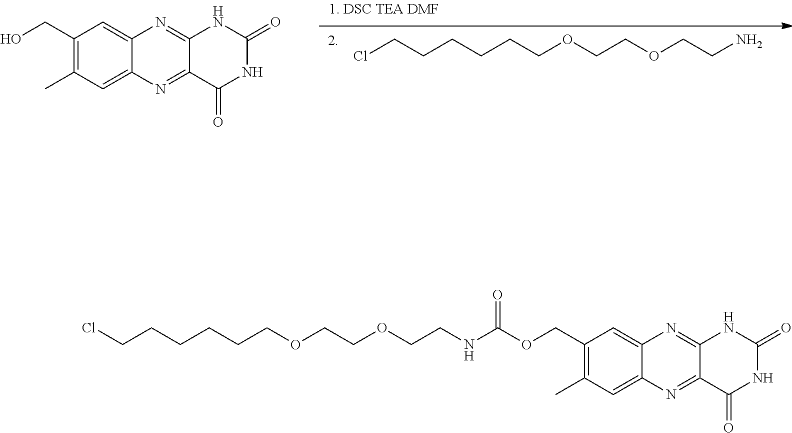 [SMILES: Cc1cc2nc3c(=O)[nH]c(=O)[nH]c3nc2cc1CO ; NCCOCCOCCCCCCCl ; Cc1cc2nc3c(=O)[nH]c(=O)[nH]c3nc2cc1COC(=O)NCCOCCOCCCCCCCl]

Halo flavin probes are prepared from the carboxylic acid or benzyl alcohol versions of the flavin scaffold as shown in the scheme above, by forming an amide or carbamate with an amine prepared from an ether synthesized from a dihalo alkane (e.g., 1-chloro-6-iodo-hexane) and an N-protected aminoalcohol (N-protected 2-(aminoethyl)ethanol).

### Probe Substrate Synthesis:

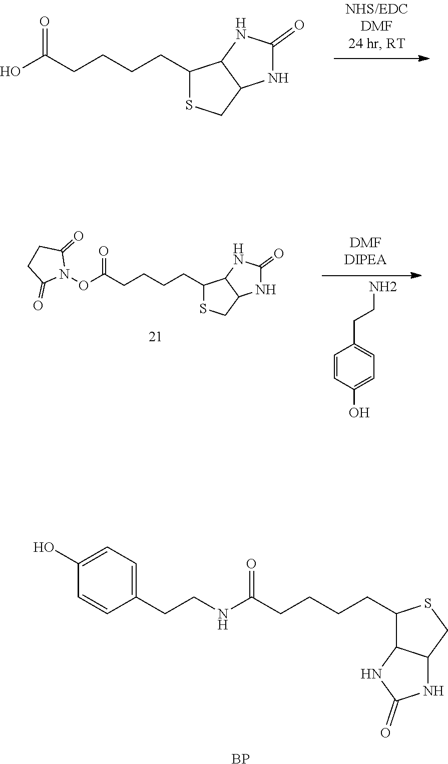 [SMILES: O=C(O)CCCCC1SCC2NC(=O)NC21 ; O=C1NC2CSC(CCCCC(=O)ON3C(=O)CCC3=O)C2N1 ; NCCc1ccc(O)cc1 ; O=C(CCCCC1SCC2NC(=O)NC21)NCCc1ccc(O)cc1 ; "BP"]

Probe substrate BP was prepared as shown above, starting by making the NHS ester of biotin carboxylic acid (i.e., compound 21 from Example 1). BP is formed by preparing the amide of the NHS ester (compound 21) by contacting the NHS ester with an amine (i.e., tyramine) in the presence of a non-nucleophilic based (DIPEA).

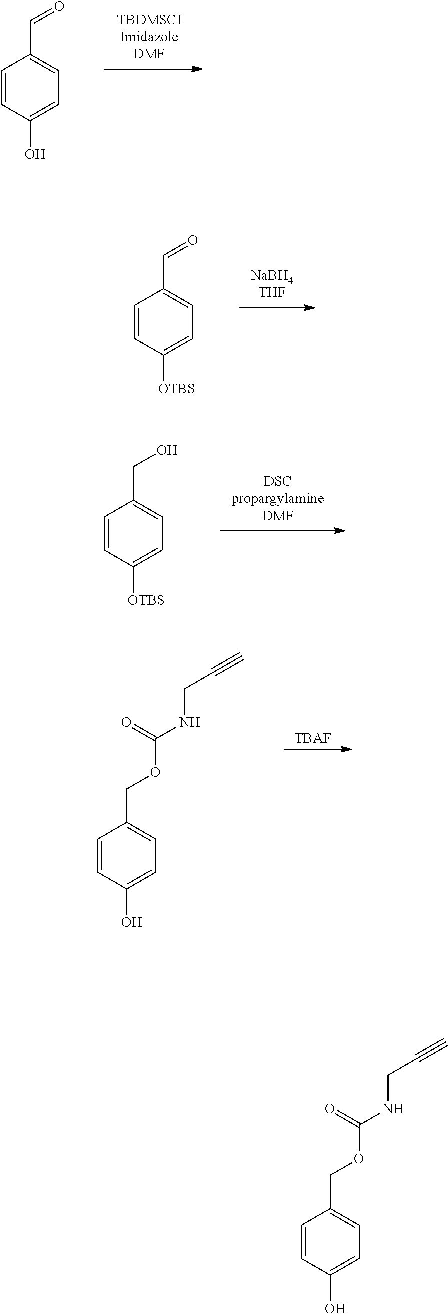 [SMILES: O=Cc1ccc(O)cc1 ; CC(C)(C)[Si](C)(C)Oc1ccc(C=O)cc1 ; CC(C)(C)[Si](C)(C)Oc1ccc(CO)cc1 ; C#CCNC(=O)OCc1ccc(O)cc1 ; "TBAF" ; C#CCNC(=O)OCc1ccc(O)cc1]

The scheme above shows a route to a phenol-alkyne probe substrate. The phenol group of 4-hydroxybenzaldehyde is first protected as a silyl ether using tert-butyldimethylsilyl chloride. Then the aldehyde is reduced to an alcohol using sodium borohydride. The benzyl alcohol is reacted with propargylamine and DSC to form a carbamate and the phenol is deprotected using TBAF.

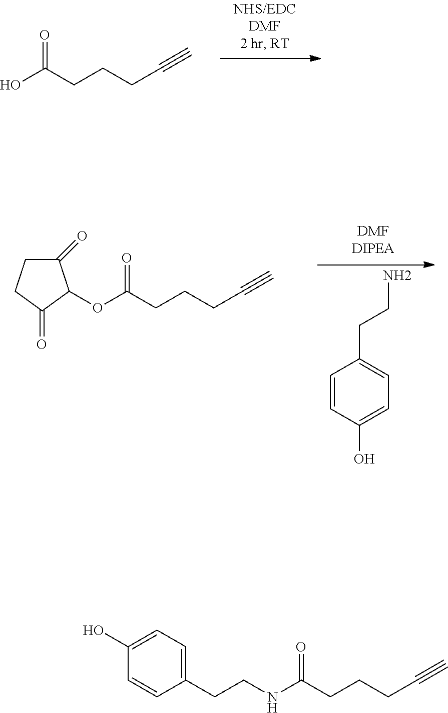

Another phenol-alkyne probe substrate is prepared as shown above, by preparing the NHS ester of 5-hexynoic acid. The NHS ester is then contacted with tyramine in the presence of DIPEA.

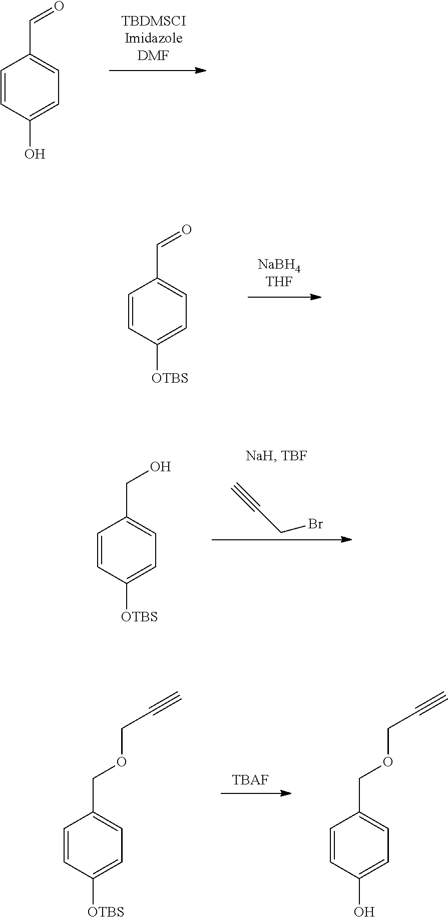

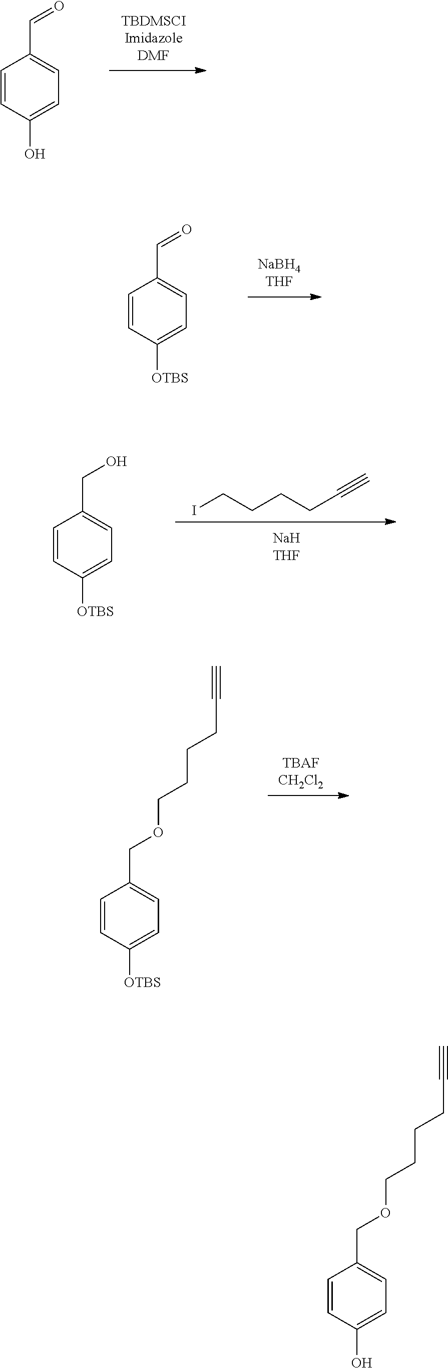

The scheme above shows a synthetic route to other phenol-alkyne probe substrates. The phenol group of 4-hydroxylbenzaldehyde is protected as a silyl ether and the aldehyde group is reduced. The resulting benzyl alcohol is reacted with a halo alkyne (1-bromo-2-propyne or 1-iodo-5-hexyne) to form an ether and the phenol group is deprotected.

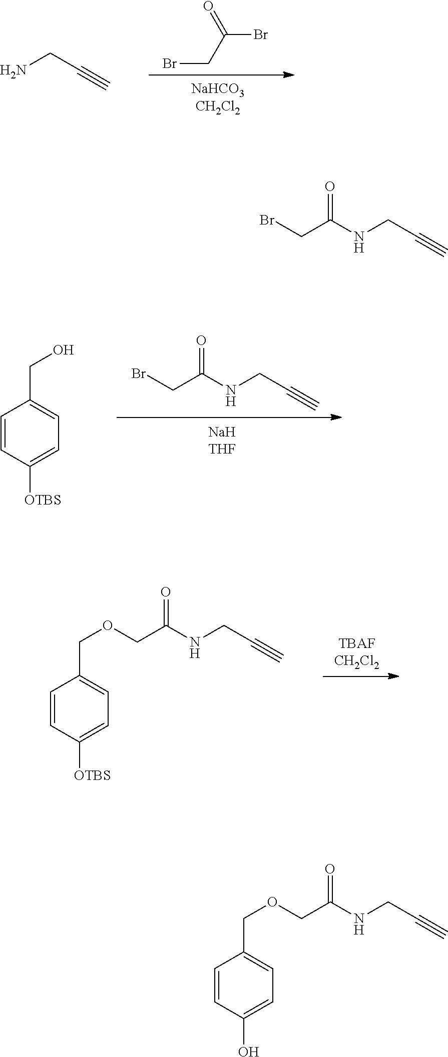

An additional phenol-alkyne probe substrate is prepared above by first synthesizing an amide from a 2-haloacetyl halide and an amine-substituted alkyne. This amide is then reacted with the benzyl alcohol prepared by reducing the aldehyde group of a silyl ether of 4-hydroxybenzaldehyde.

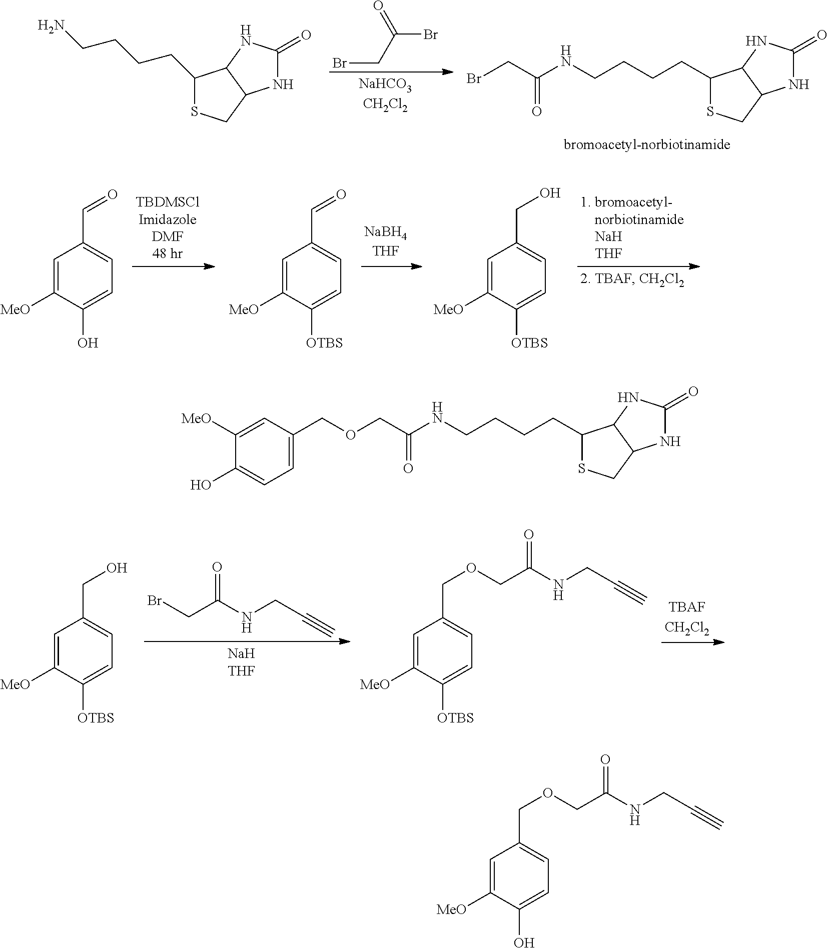

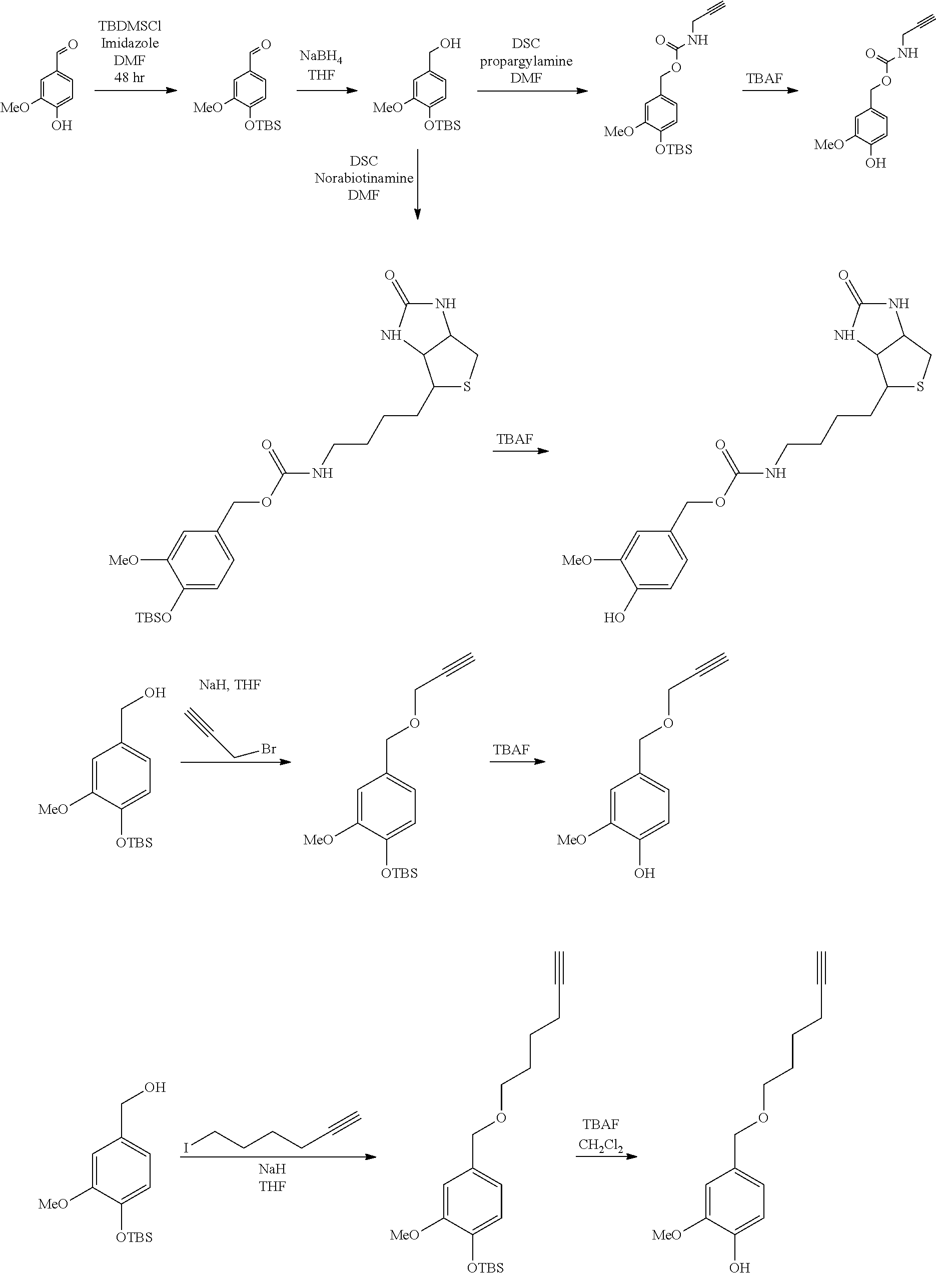

Substituted phenol-alkyne or substituted phenol-biotin probe substrates are prepared using similar routes to the syntheses of the phenol-alkyne and phenol-biotin probe substrates, as shown in the scheme above.

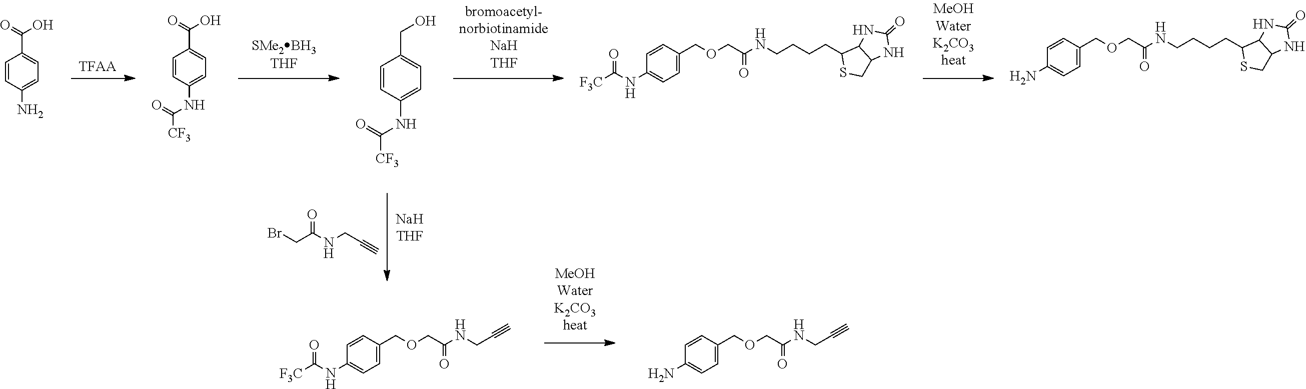

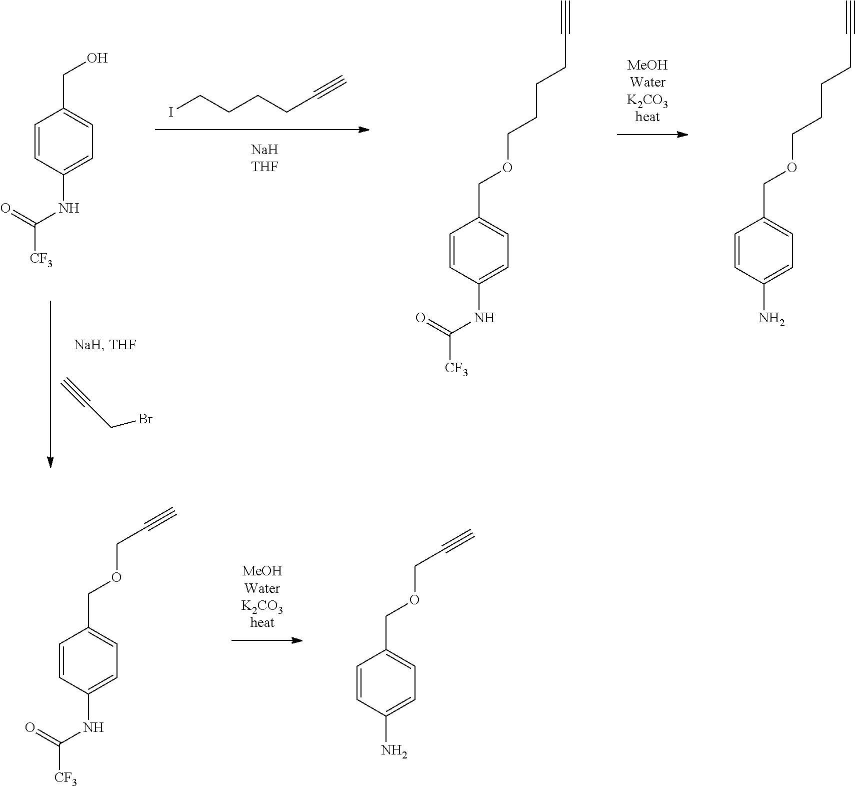

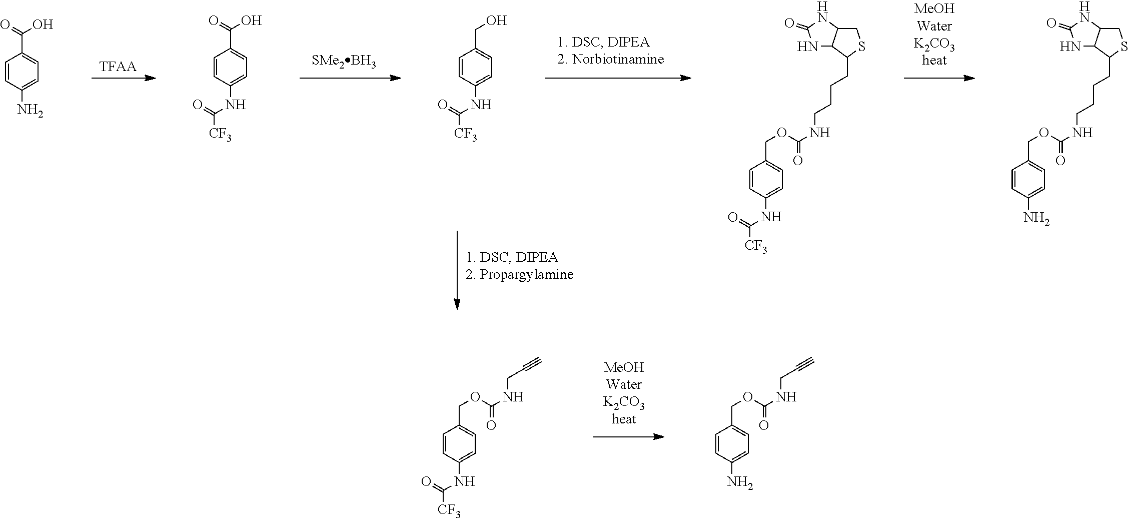

Exemplary aniline-containing probe substrates are prepared using methods analogous to those used to prepare the phenol- and substituted phenol-containing probe substrates, as shown above.

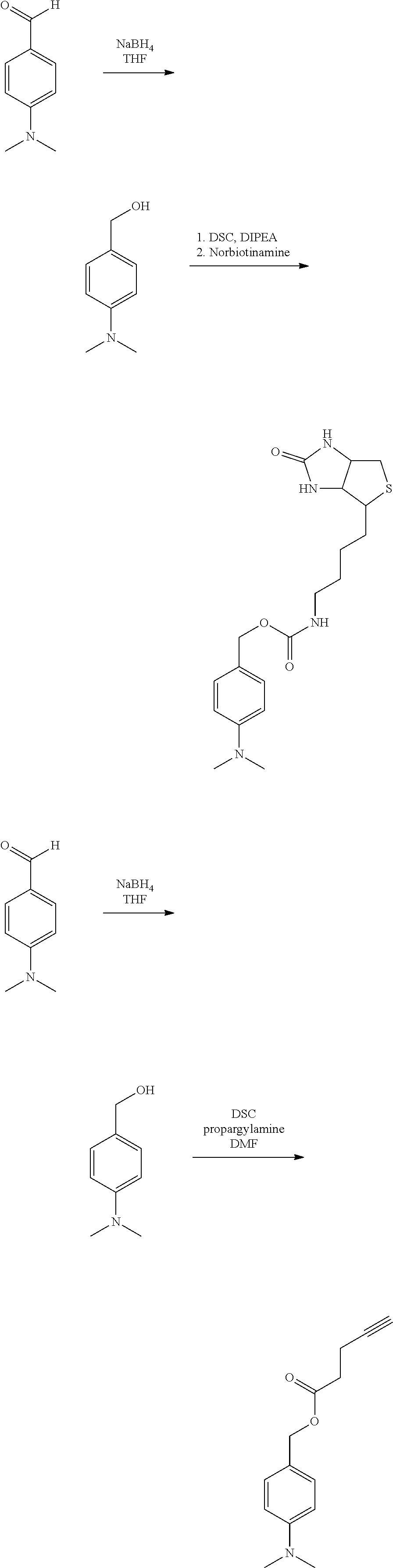 [SMILES: CN(C)c1ccc(C=O)cc1 ; CN(C)c1ccc(CO)cc1 ; CN(C)c1ccc(COC(=O)NCCCCC2SCC3NC(=O)NC32)cc1 ; CN(C)c1ccc(C=O)cc1 ; CN(C)c1ccc(CO)cc1 ; C#CCCC(=O)OCc1ccc(N(C)C)cc1]

Similarly. N-substituted aniline-containing probe substrates are prepared as shown above, again analogously to the methods of preparing the phenol-containing probe substrates.

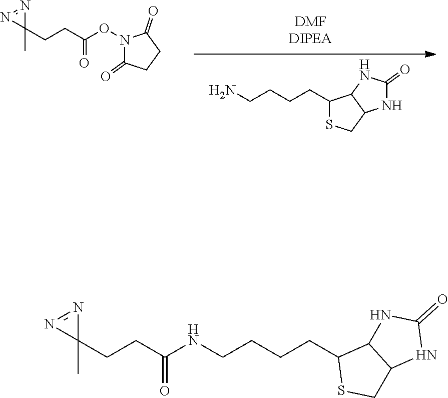 [SMILES: CC1(CCC(=O)ON2C(=O)CCC2=O)N=N1 ; NCCCCC1SCC2NC(=O)NC21 ; CC1(CCC(=O)NCCCCC2SCC3NC(=O)NC32)N=N1]

A diazirine-biotin probe substrate is prepared by reacting the diazirine NHS-ester (compound 23 from Example 1) with norbiotinamine. The diazirine-biotin probe substrate can be activated using the flavin probe catalysts at a higher wavelength than the phenol- and aniline-containing probe substrates (e.g., at about 495 nm).

### Example 12

### In Vitro Phenol Oxidation Via Flavin Photocatalyst

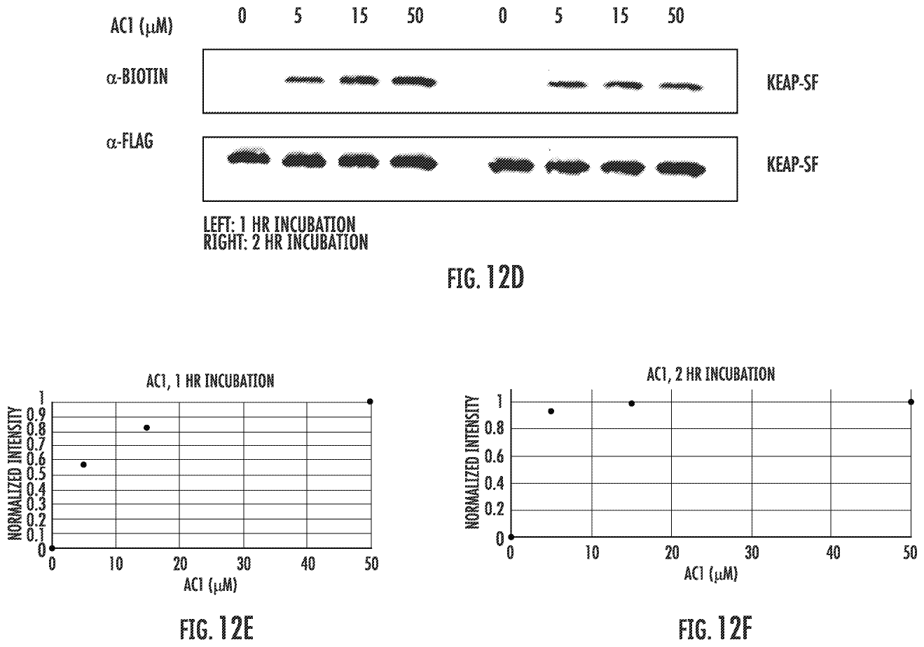

As a proof-of-concept of the catalytic photoproximity profiling system, the photo-catalysis of the oxidation of a model biotin-phenol (BP) probe substrate was studied using an LC-MS assay and flavin carboxylic acid as the photocatalyst. See FIG. 16A. Briefly, a 200 mM solution of the phenol-biotin probe substrate was prepared in water from a 20 mM stock solution. 500 ml of the solution was added to 3 Epp. Tubes. Flavin catalyst (20 mM) was added to +h/+FC samples (5 mM stock). The +UV samples were placed on ice and positioned approximately 4 cm from the source of irradiation. The +UV samples were then irradiated with 365 nm light for 15 minutes on ice and the resulting solutions analyzed via LC-MS. As shown in FIGS. 16B and 16C, the photoactivity of the flavin catalyst with the biotin-phenol (BP)-probe substrate was confirmed. Free flavin-catalyst showed robust conversion of the BP substrate probe to a crosslinked species (later eluting).

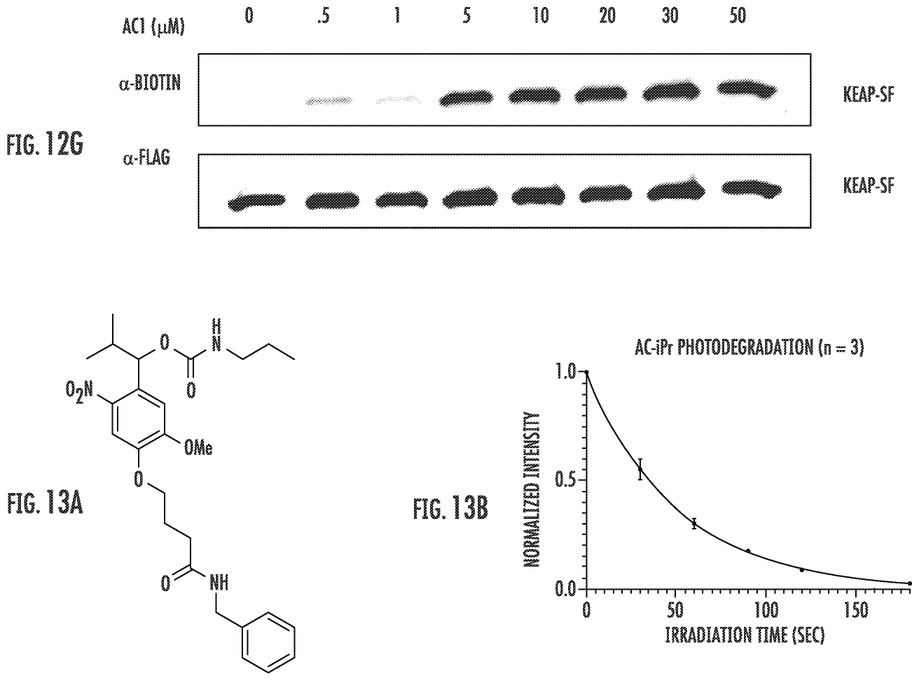

An analogous study was performed using the model benzylguanine-derivatives flavin catalyst (FBG-1) shown in FIG. 15A as the catalyst in place of the flavin carboxylic acid of FIG. 16A. As shown in FIG. 17A, the photoactivity of the model benzylguanine-derivatized flavin catalyst (FBG-1) with biotin-phenol (BP)-substrate was confirmed via LC-MS. Conversion of the substrate probe to a later eluting cross-linked species (see FIG. 17B) was observed.

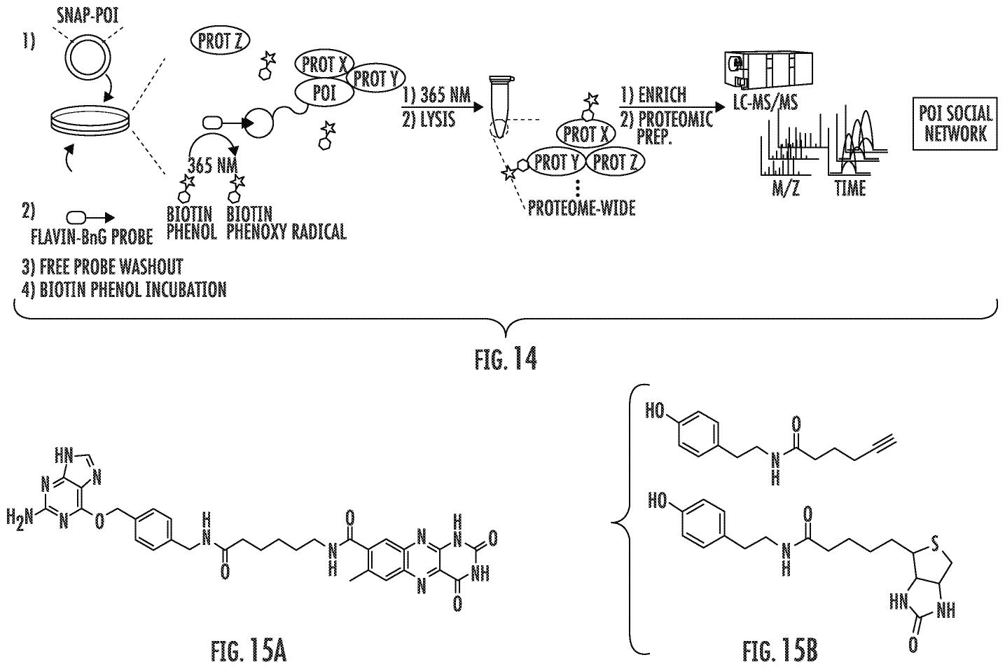

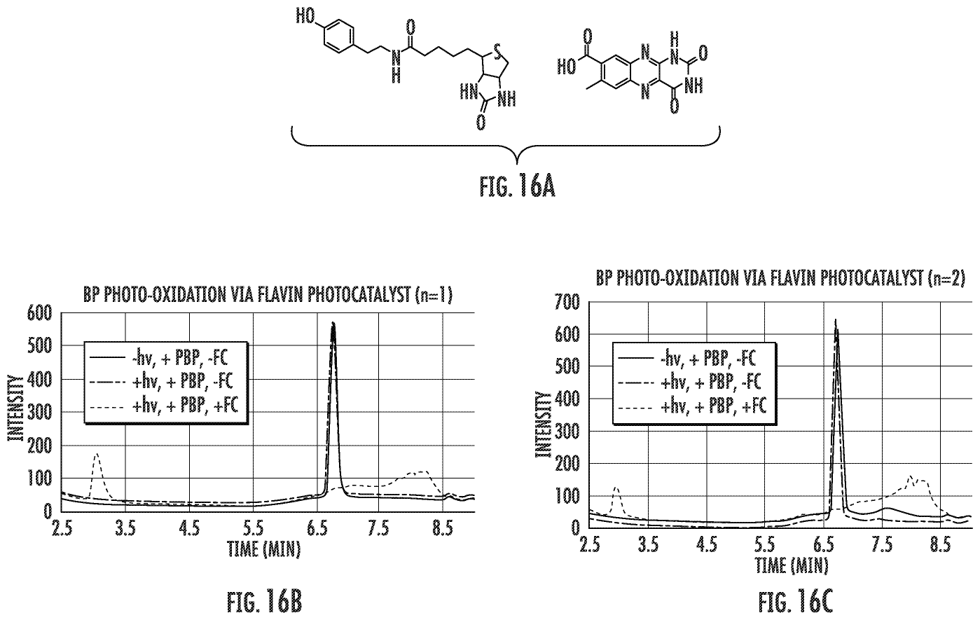

As a further proof-of-concept of the photocatalytic system, the in vitro labeling of BSA was studied using the same biotin-phenol (BP) substrate, using either flavin carboxylic acid of FBG-1. Briefly, a 200 mM solution of the BP substrate was prepared in water from a 20 mM stock solution. 500 ml of the solution was added to each of 3 Eppendorf tubes. Catalyst (20 mM) was added to +FC samples from 5 mM stock solutions. An equivalent volume of DMSO was added to −PC and −FBG samples. The +UV samples were placed on ice and positioned approximately 4 cm from the source of irradiation. The +UV samples were then irradiated with 365 nm light for 15 min on ice. 60 ml aliquots of each sample were removed. To each aliquot was added 20 ml 4× loading buffer, and the resulting solutions were vortexed and heated at 95° C. for 10 minutes. 10 ml of each sample was analyzed via western blot/Coomassie stain. As shown in FIG. 18, the study using flavin carboxylic acid as the catalyst confirmed the activity of the combination of the flavin catalyst and the BP substrate biochemically, demonstrating biotin-labeling of protein (bovine serum albumin, BSA) in bulk solution. Strong, covalent biotinylation of BSA was only observed when both the substrate and catalyst were present and light was used (+UV). Similarly, as shown in FIG. 19, the biochemical activity of the combination of the benzylguanine-derivative catalyst (FBG-1) and the BP substrate was confirmed by the demonstration of the biotin-labeling of BSA in bulk solution. Again, strong covalent biotinylation of BSA was only observed when both BP and FBG-1 were present and the solution was irritated (+UV).

With these results in hand, the in vitro anti-Flag photolabeling study was performed using the catalytic probe system with the FBG-1 catalyst and the BP probe substrate. Briefly, 250 μL aliquots of cell lysate (HEK293T. SNAP-Flag) were incubated in a microcentrifuge tube with either 500 nM of FBG1 or DMSO for 1 hour at 37° C. Washed anti-FLAG M2 affinity gel, 40 μL of a 50% slurry, was transferred to each reaction in 750 μL of DPBS and the resultant suspension left to rotate overnight at 4° C. Samples were then spun down at 4000×g, supernatant aspirated, and the resin washed with 1M urea in DPBS (1 mL×8) followed by DPBS (1 mL). After being resuspended in 250 μL of DPBS (+/−BP), +UV samples were placed on ice where they were irradiated with 365 nm light for 15 minutes. Beads were washed with 1M Urea (1 mL×2) and DPBS (1 mL×2) and boiled for 5 minutes at 95° C. in 20 μL 4×-loading buffer containing 8% SDS and 400 mM DTT. 60 μL of DPBS was added to the suspension and it was boiled at 95° C. for an additional 5 minutes. Analysis with gel/western blot was performed.

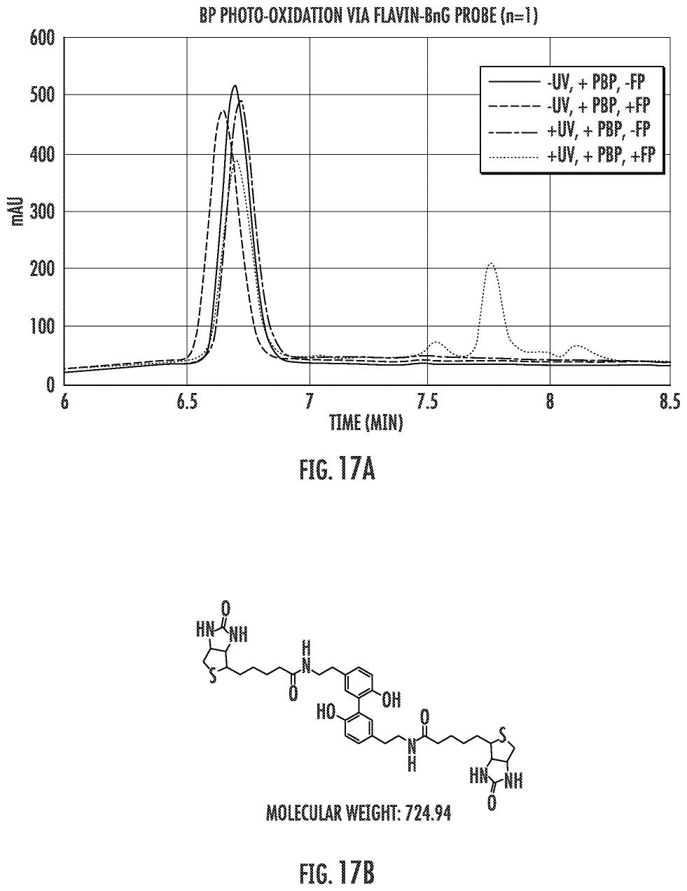

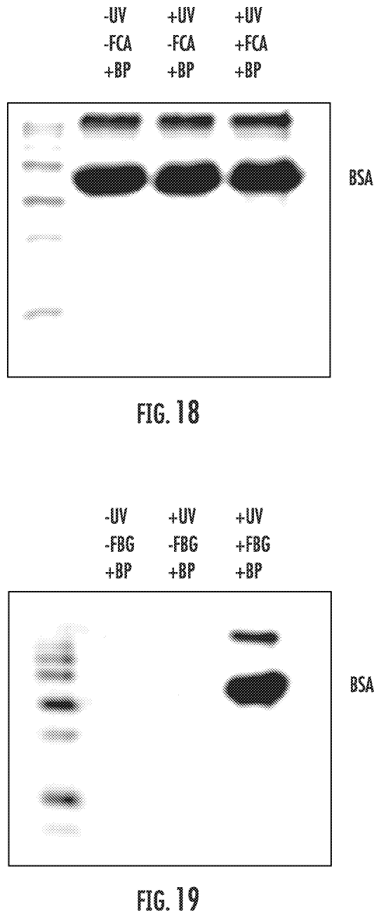

As shown in FIG. 20, the FBG1 photocatalyst covalently labels SNAP-tag protein, and subsequently labels a proximal protein binding partner (anti-FLAG IgG in this case) when substrate (BP) is present, and the system is activated by light. Proximal protein labeling is apparent by the appearance of covalent biotin on the IgG protein. Thus, the CatPhotoPPI system appears to be successful in the biotin labeling of proteins involved in protein-protein interactions in response to light. FIG. 21 shows a side-by-side comparison of the results of the anti-FLAG assay using the photocatalytic system with FBG-1 and the BP substrate and a noncatalytic probe comprising a photocleavable group (AC1). As observed in FIG. 21, catalytic PhotoPPI appears to result in higher biotin signal relative to the stoichiometric PhotoPPI labeling.

### Example 13

### In-Situ Snap Labeling/BG-FITC Competition Assay

The wells of a 12-well plate were seeded with HEK293T cells expressing KEAP1-SF (˜150,000 cells/well). Once at >90% confluency, the cells were washed with PBS (0.5 mL). The cells were then treated with varying doses of Flavin-BG (FBG-1) (0, 1, 2, 5, 10, 15, 25, 50, or 75 mM) in serum-free DMEM and incubated for 2 hours at 37° C. Media was aspirated and the cells washed over the course of 40 min with DMEM at 37° C. (1 mL/well for 20 min×2). The media was again aspirated and the cells washed with warm PBS (1 mL). Cells were lysed in cold RIPA+PI+DTT+20 mM FITC-BG. Then the cells were agitated for 20 min at 4° C. and transferred to Epp tubes. The samples were prepared for in gel fluorescence using 20 μl 4×LB+60 μl lysate and heated to 95° C. for 5 min. The gels were run and imaged.

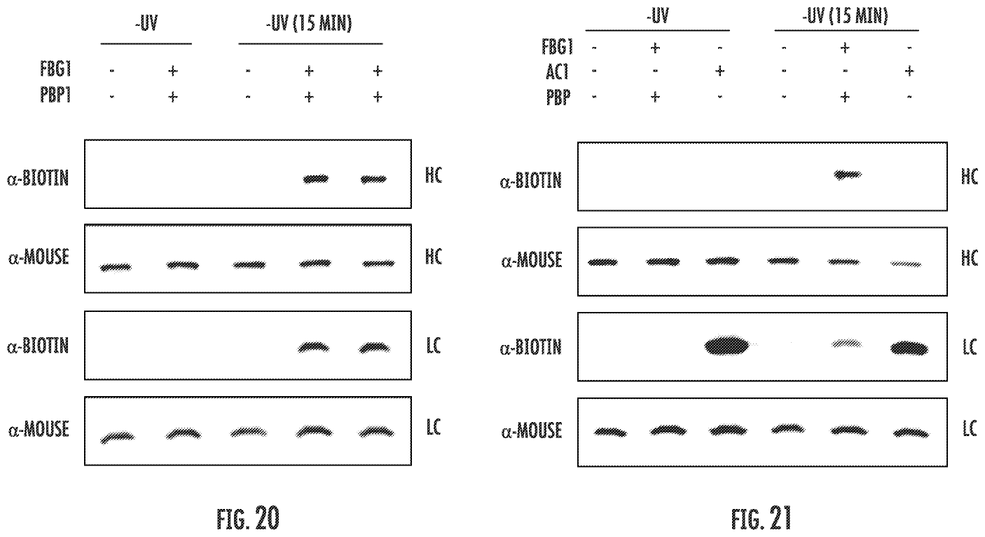

Results indicated that FBG-1 is cell permeable and labels SNAP protein in cells. The results also showed competition of increasing doses of FBG-1 with FITC-BnG labeling of intracellular SNAP protein. See FIG. 22A. FBG-1 shows robust labeling of the SNAP-POI in the low to mid micromolar range (EC₅₀=11 mM). See FIG. 22B.

### Example 14

### Cell Viability Assay for FBG-1

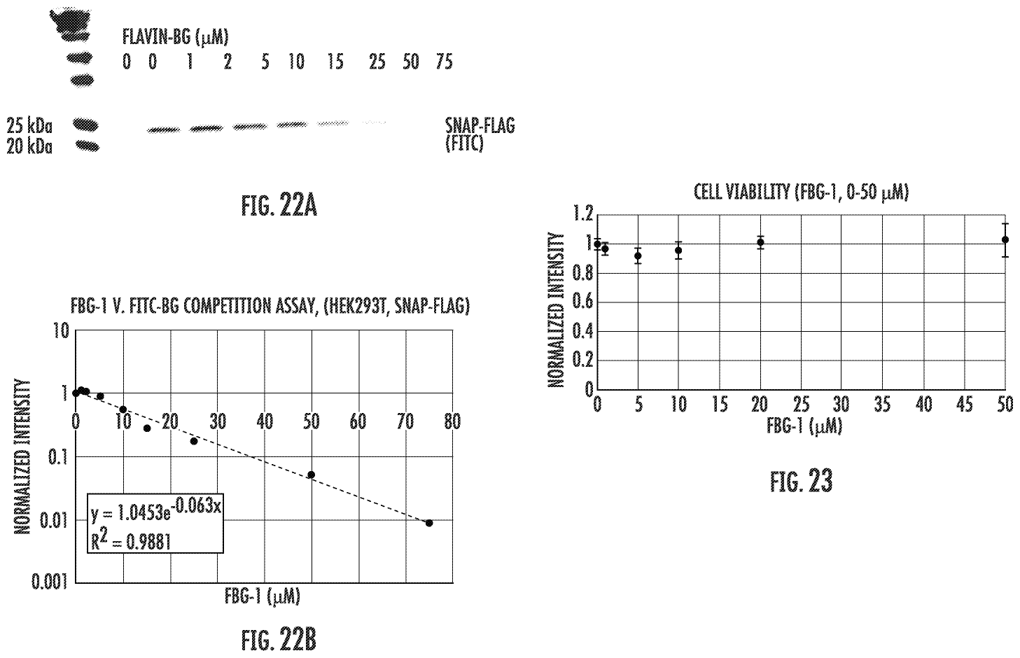

HEK293T cells stably expressing Snap-Flag were seeded at 5,000 cells per well in 100 μL DMEM in a 96-well plate. Upon >90% confluency, media was gently removed and cells were treated with a varying concentration (0, 1, 5, 10, 20, 50 uM) in sextuplicate of FBG-1 in a total volume of 75 μL DMEM. After 2 hours at 37° C., media was aspirated and cells were gently washed with 75 ul of serum free DMEM. After final aspiration, media was replaced with 75 ul of DMEM and 75 μL of Cell Titer-Glo™ (Promega Corporation, Madison, Wis., United States of America) was added. The cells were placed on a shaker for 2 minutes and allowed to equilibrate at room temperature for 10 minutes. Luminescence was recorded using a SYNERGY™ Neo HST plate reader (BioTek, Winooski, Vt., United States of America). As shown in FIG. 23, FBG-1 is not toxic in cells at time points and doses used for SNAP-POI labeling.

### Example 15

### In Situ Catalytic PhotoPPI with FBG-1

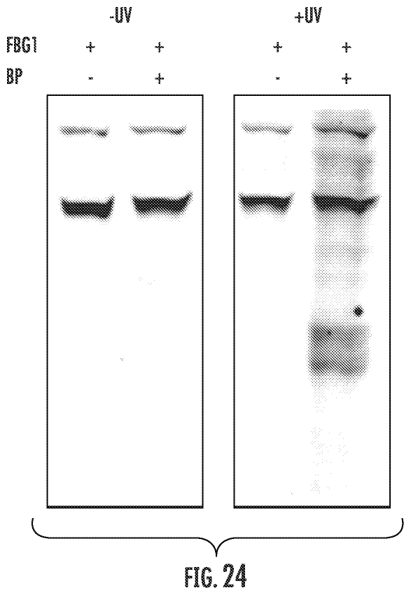

FBG1/BP labeling was further studied in live cells (HEK293T expressing Snap-FLAG-KEAP1 protein fusion). Each well of two 6-well plates was seeded with 300,000 HEK293T cells stably expressing KEAP1-SF. After reaching ˜90% confluency, the cells were washed with PBS (1 mL). Media (1 mL) was added according to conditions in serum free media ((1)+/−FBG; 30 uM, serum free or (2)+/−DMSO) and incubated at 37° C. for 2 hours. Washout (2 mL every 20 minutes) was performed twice. Media (2 ml) was added: (1)+/−BP; 250 uM from 500 mM stock, 30 min, 37° C. or (2)+/−DMSO. Samples were irradiated at 365 nm for 15 minutes on ice. Media was aspirated and the cells was with 1 mL PBS two times. The cells were lysed with RIPA with PI/DTT. Lysate was stored at −78° C. and aliquot samples for western blot analysis (20 ul 4×LB/60 ul lysate) removed. Samples were run on a gel and transferred for western blot analysis (primary antibody; a-flag, 1:2500), overnight at 4° C.; secondary antibody: streptavidin IR 800, mouse 680, 1:10,000). Bands corresponding to biotinylated proteins and the FLAG antibody were visualized using an Odyssey infrared imager (Li-cor Biosciences, Lincoln, Nebr., United States of America). As shown in FIG. 24, live cell labeling of SNAP-POI with FBG1, followed by incubation with substrate (BP) results in intracellular proximity labeling in the presence of light.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
1. Scott, J. D.; Pawson, T., Cell signaling in space and time: where proteins come together and when they're apart. Science 2009, 326 (5957), 1220-4.
2. Collins, B. C.; Gillet, L. C.; Rosenberger, G.; Rost, H. L.; Vichalkovski, A.; Gstaiger, M.; Aebersold, R., Quantifying protein interaction dynamics by SWATH mass spectrometry: application to the 14-3-3 system. Nature methods 2013, 10 (12), 1246-53.
3. Roux, K. J.; Kim, D. I.; Raida, M.; Burke, B., A promiscuous biotin ligase fusion protein identifies proximal and interacting proteins in mammalian cells. The Journal of cell biology 2012, 196 (6), 801-10.
4. Gupta, G. D.; Coyaud, E.; Goncalves, J.; Mojarad, B. A.; Liu, Y.; Wu, Q.; Gheiratmand, L.; Comartin, D.; Tkach, J. M.; Cheung, S. W.; Bashkurov, M.; Hasegan, M.; Knight, J. D.; Lin, Z. Y.; Schueler, M.; Hildebrandt, F.; Moffat, J.; Gingras, A. C.; Raught, B.; Pelletier, L., A Dynamic Protein Interaction Landscape of the Human Centrosome-Cilium Interface. Cell 2015, 163 (6), 1484-99.
5. Branon, T. C.; Bosch, J. A.; Sanchez. A. D.; Udeshi, N. D.; Svinkina, T.; Carr, S. A.; Feldman, J. L.; Perrimon, N.; Ting, A. Y., Efficient proximity labeling in living cells and organisms with TurboID. Nature biotechnology 2018, 36 (9), 880-887.
6. Hill, Z. B.; Pollock, S. B.; Zhuang, M.; Wells, J. A., Direct Proximity Tagging of Small Molecule Protein Targets Using an Engineered NEDD8 Ligase. Journal of the American Chemical Society 2016, 138 (40), 13123-13126.
7. Liu, Q.; Zheng, J.; Sun, W.; Huo, Y.; Zhang, L.; Hao, P.; Wang, H.; Zhuang, M., A proximity-tagging system to identify membrane protein-protein interactions. Nature methods 2018, 15 (9), 715-722.
8. Ge, Y.; Chen, L.; Liu, S.; Zhao, J.; Zhang, H.; Chen, P. R., Enzyme-Mediated Intercellular Proximity Labeling for Detecting Cell-Cell Interactions. Journal of the American Chemical Society 2019, 141 (5), 1833-1837.
9. Honke, K.; Kotani, N., The enzyme-mediated activation of radical source reaction: a new approach to identify partners of a given molecule in membrane microdomains. J Neurochem 2011,116 (5), 690-5.
10. Martell, J. D.; Decrinck, T. J.; Sancak, Y.; Poulos, T. L.; Mootha, V. K.; Sosinsky, G. E.; Ellisman, M. H.; Ting, A. Y., Engineered ascorbate peroxidase as a genetically encoded reporter for electron microscopy. Nature biotechnology 2012, 30 (11), 1143-8.
11. Rhee, H. W.; Zou, P.; Udeshi, N. D.; Martell, J. D.; Mootha, V. K.; Carr, S. A.; Ting, A. Y., Proteomic mapping of mitochondria in living cells via spatially restricted enzymatic tagging. Science 2013, 339 (6125), 1328-31.
12. Hung, V.; Zou, P.; Rhee, H. W.; Udeshi, N. D.; Cracan, V.; Svinkina, T.; Carr, S. A.; Mootha, V. K.; Ting, A. Y., Proteomic mapping of the human mitochondrial intermembrane space in live cells via ratiometric APEX tagging. Molecular cell 2014, 55 (2), 332-41.
13. Pack, J.; Kalocsay, M.; Staus, D. P.; Wingler, L.; Pascolutti, R.; Paulo, J. A.; Gygi, S. P.; Kruse, A. C., Multidimensional Tracking of GPCR Signaling via Peroxidase-Catalyzed Proximity Labeling. Cell 2017, 169 (2), 338-349 e11.
14. Weerapana, E.; Wang, C.; Simon, G. M.; Richter. F.; Khare, S.; Dillon, M. B.; Bachovchin, D. A.; Mowen, K.; Baker, D.; Cravatt, B. F., Quantitative reactivity profiling predicts functional cysteines in proteomes. Nature 2010, 468 (7325), 790-5.
15. Yang, J.; Carroll, K. S.; Liebler, D. C., The Expanding Landscape of the Thiol Redox Proteome. Molecular & cellular proteomics: MCP 2016, 15 (1), 1-11.
16. van der Reest, J.; Lilla, S.; Zheng, L.; Zanivan, S.; Gottlieb. E., Proteome-wide analysis of cysteine oxidation reveals metabolic sensitivity to redox stress. Nat Commun 2018, 9 (1), 1581.
17. Juillerat, A.; Gronemeyer, T.; Keppler, A.; Gendreizig, S.; Pick, H.; Vogel, H.; Johnsson, K., Directed evolution of O6-alkylguanine-DNA alkyltransferase for efficient labeling of fusion proteins with small molecules in vivo. Chemistry & biology 2003, 10 (4), 313-7.
18. Mollwitz, B.; Brunk, E.; Schmitt, S.; Pojer, F.; Bannwarth, M.; Schiltz, M.; Rothlisberger, U.; Johnsson, K., Directed evolution of the suicide protein O(6)-alkylguanine-DNA alkyltransferase for increased reactivity results in an alkylated protein with exceptional stability. Biochemistry 2012, 51 (5), 986-94.
19. Fang, X.; Fu, Y.; Long, M. J.; Haegele, J. A.; Ge, E. J.; Parvez, S.; Aye. Y., Temporally controlled targeting of 4-hydroxynonenal to specific proteins in living cells. Journal of the American Chemical Society 2013, 135 (39), 14496-9.
20. Chen, X.; Venkatachalapathy. M.; Kamps, D.; Weigel, S.; Kumar, R.; Orlich. M.; Garrecht, R.; Hirtz, M.; Niemeyer, C. M., Wu, Y. W.; Dehmelt, L., “Molecular Activity Painting”: Switch-like, Light-Controlled Perturbations inside Living Cells. Angew Chem Int Ed Engl 2017, 56 (21), 5916-5920.
21. Suzuki. T.; Yamamoto, M., Stress-sensing mechanisms and the physiological roles of the Keap1-Nrf2 system during cellular stress. The Journal of biological chemistry 2017, 292 (41), 16817-16824.
22. Ogura, T.; Tong, K. I.; Mio, K.; Maruyama, Y.; Kurokawa, H.; Sato, C.; Yamamoto, M., Keap1 is a forked-stem dimer structure with two large spheres enclosing the intervening, double glycine repeat, and C-terminal domains. Proceedings of the National Academy of Sciences of the United States of America 2010, 107 (7), 2842-7.
23. Bollong, M. J.; Lee, G.; Coukos, J. S.; Yun, H.; Zambaldo, C.; Chang, J. W.; Chin, E. N.; Ahmad, I.; Chatterjee, A. K.; Lairson. L. L.; Schultz, P. G.; Moellering, R. E., A metabolite-derived protein modification integrates glycolysis with KEAP1-NRF2 signalling. Nature 2018, 562 (7728), 600-604.
24. Lo, S. C.; Hannink, M., PGAM5 tethers a ternary complex containing Keap1 and Nrf2 to mitochondria. Exp Cell Res 2008, 314 (8), 1789-803.
25. Lo, S. C.; Hannink, M., PGAM5, a Bcl-XL-interacting protein, is a novel substrate for the redox-regulated Keap1-dependent ubiquitin ligase complex. The Journal of biological chemistry 2006, 281 (49), 37893-903.
26. Sowa, M. E.; Bennett, E. J.; Gygi, S. P.; Harper, J. W., Defining the human deubiquitinating enzyme interaction landscape. Cell 2009, 138 (2), 389-403.

It will be understood that various details of the presently disclosed subject matter may be changed without departing from the scope of the presently disclosed subject matter. Furthermore, the foregoing description is for the purpose of illustration only, and not for the purpose of limitation.

1. A photoactive chemical probe or probe system for proximity profiling of biological interactions, wherein the photoactive chemical probe or probe system comprises a target recognition moiety capable of specifically binding a first binding partner associated with a biological target of interest (BTOI), optionally wherein the first binding partner is a peptide or protein tag attached to the BTOI; a detectable moiety or precursor thereof; and at least two photoactive moieties, wherein one of said photoactive moieties is a photocleavable or photocatalytic moiety.

2. The photoactive chemical probe or probe system of claim 1, wherein the probe or probe system comprises a photoactive probe having a structure of Formula 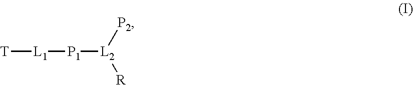
wherein:
T is a target recognition moiety capable of specifically binding a first binding partner, optionally wherein the first binding partner is a peptide or protein tag attached to a biological target of interest;
L₁ is a bivalent linker;
P₁ is a photocleavable moiety;
L₂ is a trivalent linker moiety;
P₂ is a photoreactive moiety; and
R is a detectable moiety or a precursor thereof capable of specifically binding a second binding partner, subject to the proviso that the first and second binding partners are different.

3. The photoactive probe or probe system of claim 1, wherein the photoactive probe or probe system comprises a probe system comprising:
a photocatalytic probe having a structure of Formula (VII):
T-L₁₀-P_{c}; and
a probe substrate having a structure of Formula (VIII):
P₃-L₁₁-R;
wherein:
T is a target recognition moiety capable of specifically binding a first binding partner, optionally wherein the first binding partner is a peptide or protein tag attached to a biological target of interest;
L₁₀ and L₁₁ are bivalent linkers;
P_{c} is a photocatalytic moiety;
P₃ is a photoreactive moiety that is capable of undergoing a reaction catalyzed by P_{c}; and
R is a detectable moiety or a precursor thereof capable of specifically binding a second binding partner, subject to the proviso that the first and second binding partners are different.

4. The photoactive probe or probe system of claim 2, wherein R comprises biotin, a biotin analog, or an alkyne.

5. (canceled)

6. The photoactive probe or probe system of claim 2, wherein T comprises a moiety selected from the group consisting of a benzylguanine group, a chloroalkane group, a benzylcytosine group, an azide, biotin, desthiobiotin, AP1867 or an orthogonal FK506 analog, and a methotrexate derivative.

7. (canceled)

8. The photoactive prove or probe system of claim 2, wherein P₂ comprises a diazirine derivative, a benzophenone derivative, or an aryl azide derivative.

9. (canceled)

10. The photoactive probe or probe system of claim 2, wherein L₁ is selected from —NH—C(═O)-alkylene-; —NH—C(═O)—O—CH₂CH₂—O—; and —NH—C(═O)—O—CH₂CH₂—NH—C(═O)-alkylene-, wherein said alkylene is substituted or unsubstituted, optionally wherein said alkylene is propylene.

11. The photoactive probe or probe system of claim 2, wherein L₂ is selected from the group consisting of: 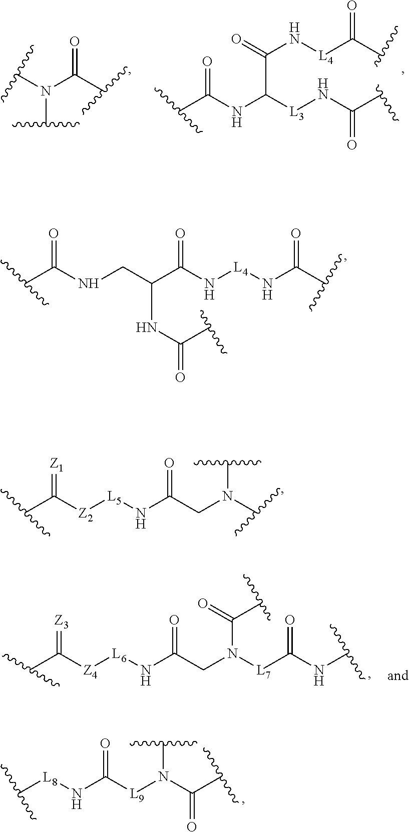
wherein each L₃, L₄, L₅, L₆, L₇, L₈, and L₉ is alkylene, wherein said alkylene is substituted or unsubstituted, optionally wherein said alkylene comprises one or more oxygen atoms inserted along the alkylene group; wherein Z₁ and Z₃ are selected from O and S; and wherein Z₂ and Z₄ are selected from O, S, and NH; optionally wherein L₂ is selected from: 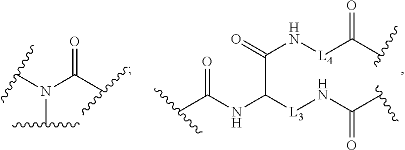
wherein L₃ is butylene and L₄ is pentylene; and 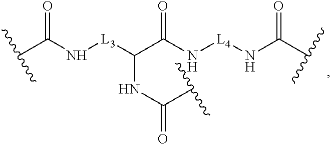
wherein L₃ is butylene and L₄ is ethylene.

12. The photoactive probe or probe system of claim 2, wherein P₁ comprises a divalent nitroaryl derivative, a divalent coumarin derivative, or a divalent hydroxyaryl derivative.

13. (canceled)

14. The photoactive probe or probe system of claim 2, wherein the compound of Formula (I) has a structure of Formula (II): 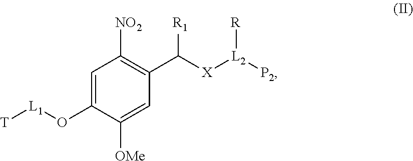
wherein:
T, L₁, L₂, R, and P₂ are as defined for the compound of Formula (I);
X is selected from O, NR′, and S, wherein R′ is selected from H and alkyl; and
R₁ is selected from H, alkyl, perhaloalkyl, and cyano.

15. (canceled)

16. The photoactive probe or probe system of claim 14, wherein the probe is selected from:

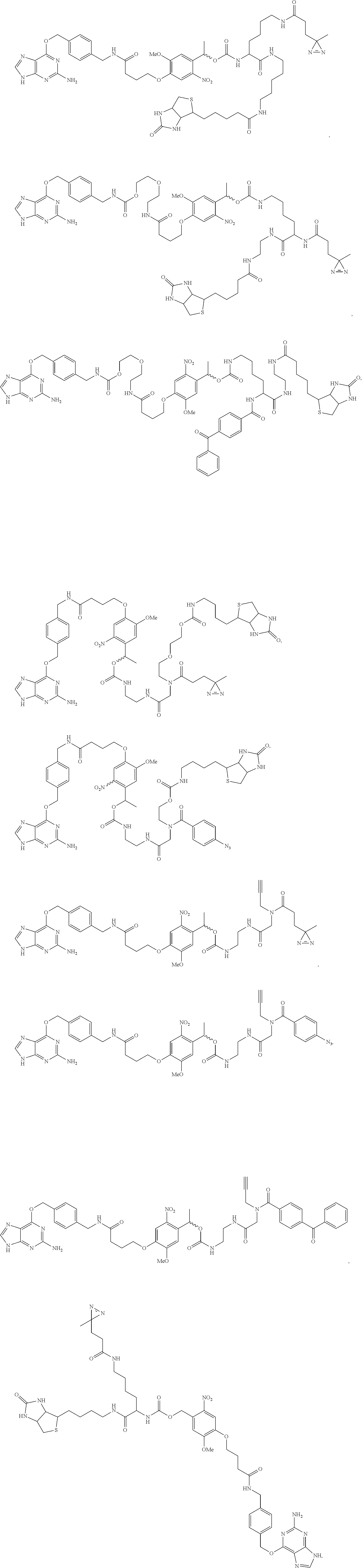

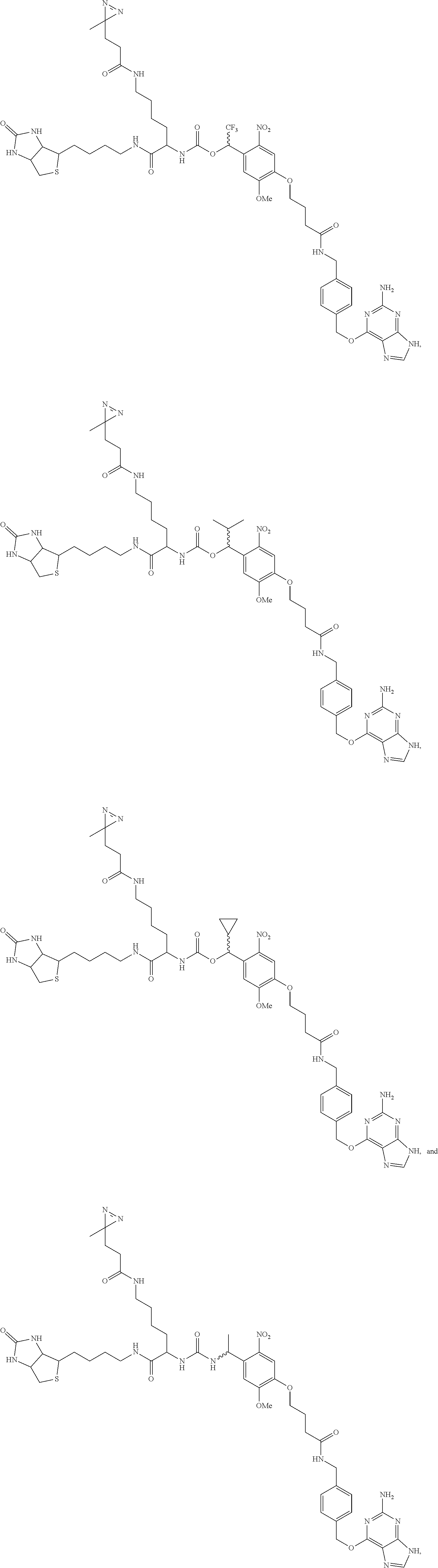

17. The photoactive probe or probe system of claim 2, wherein L₂ is —N—C(═O)—, and the compound of Formula (I) has a structure of Formula (IIIa) or Formula (IIIb): 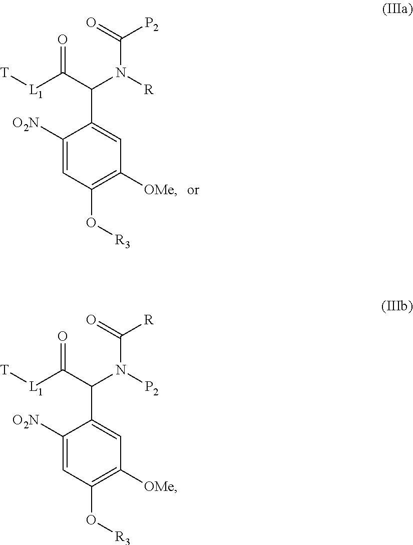
wherein:
T, L₁, R, and P₂ are as defined for the compound of Formula (I); and
R₃ is alkyl, optionally methyl.

18. (canceled)

19. The photoactive probe or probe system of any-ene-f claim 2, wherein the compound of Formula (I) has a structure of Formula (IVa) or (IVb): 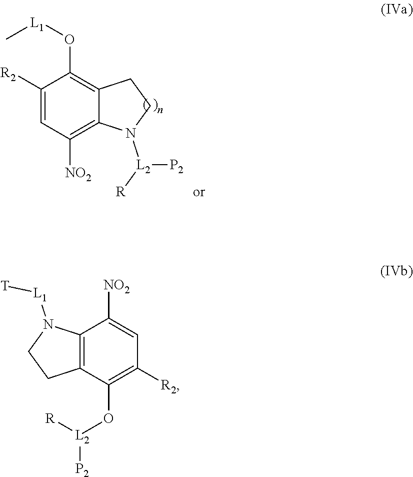
wherein:
T, L₁, L₂, R and P₂ are as defined for Formula (I);
n is 1 or 2; and
R₂ is selected from NO₂ and H.

20. (canceled)

21. The photoactive probe or probe system of claim 2, wherein the compound of Formula (I) has a structure of Formula (Va) or (Vb): 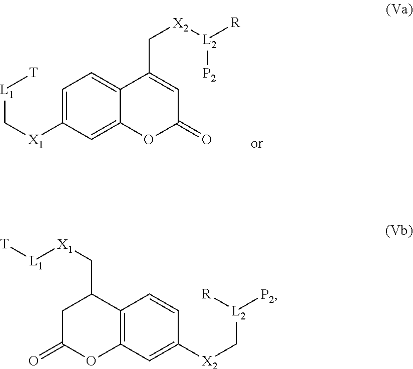
Wherein:
T, L₁, L₂, R, and P₂ are as defined for the compound of Formula (I); and
X₁ and X₂ are independently selected from O, NR′, and S, wherein R′ is H or alkyl.

22. (canceled)

23. The photoactive probe or probe system of claim 2, wherein the compound of Formula (I) has a structure of one of Formula (VIa) and (VIb): 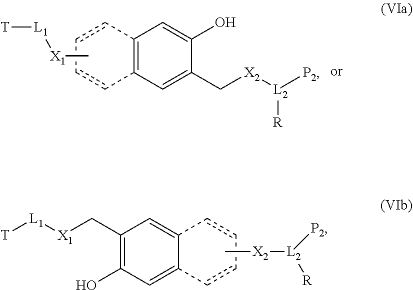
wherein:
T, L₁, L₂, P₂, and R are as defined for the compound of Formula (I);
the dotted lines can be present or absent, and when absent, X₁ or X₂ is substituted on the remaining aryl ring; and
X₁ and X₂ are independently selected from O, NR′, and S, wherein R′ is selected from H and alkyl.

24. (canceled)

25. The photoactive probe or probe system of claim 3, wherein P_{c} is a monovalent isoalloxazine moiety, optionally having the structure: 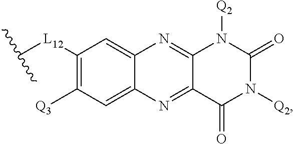
wherein:
L₁₂ is present or absent and when present is a bivalent moiety selected from the group consisting of —O-alkylene, —S-alkylene, —NQ₄-alkylene, and alkylene, wherein said alkylene is substituted or unsubstituted; and
each of Q₁, Q₂, Q₃ and Q₄ are independently selected from H, alkyl, and cycloalkyl.

26. (canceled)

27. (canceled)

28. The photoactive probe or probe system of claim 3, wherein the compound of Formula (VII) is selected from:

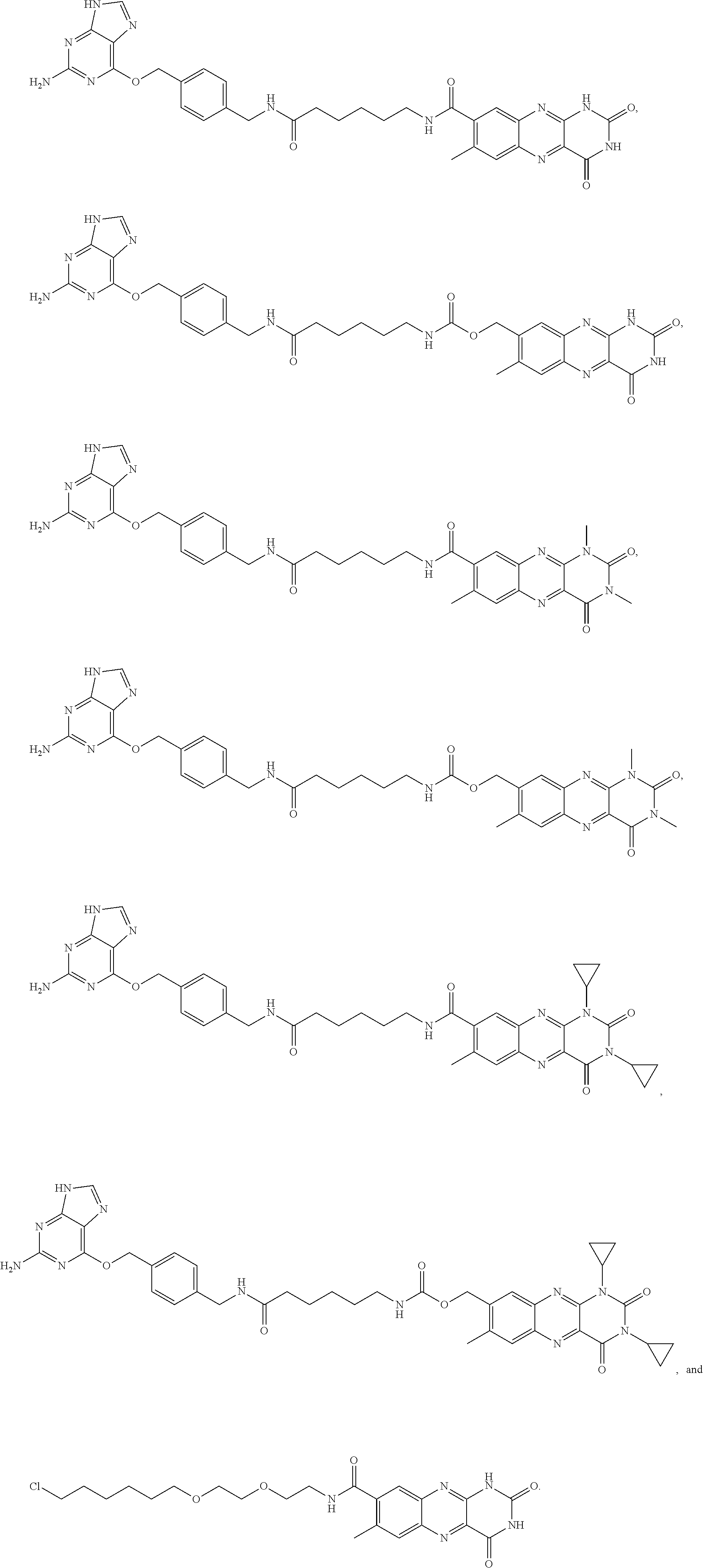

29. The photoactive probe or probe system of claim 3, wherein P₃ is selected from a phenol, an aniline, and a diazirine.

30. The photoactive probe or probe system of claim 3, wherein the probe substrate has a structure selected from the group consisting of:

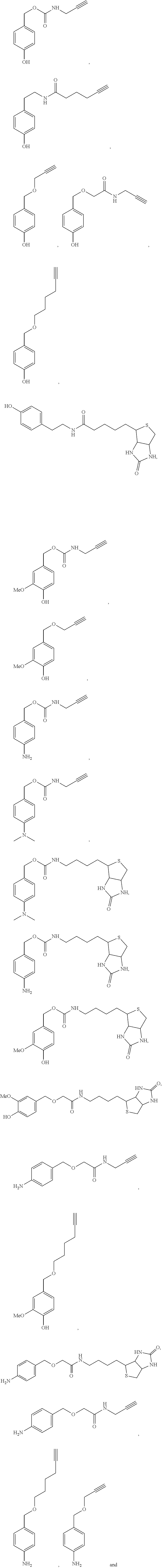

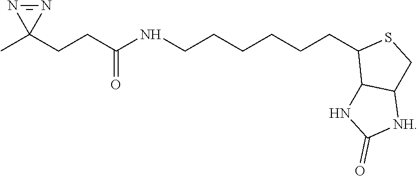 [SMILES: CC1(CCC(=O)NCCCCCCC2SCC3NC(=O)NC32)N=N1]

31-34. (canceled)

35. A method for detecting a spatiotemporal interaction of a biological target of interest (BTOI), optionally a cell or protein of interest, wherein the method comprises:
(a) labeling the BTOI with a moiety comprising a first binding partner;
(b) contacting the BTOI with a photoactive probe comprising: (i) a moiety that binds the first binding partner, (ii) a photoreactive moiety attached to a moiety that binds a second binding partner, and (iii) a photocleavable moiety attaching (i) and (ii); and
(c) exposing the probe to light, thereby cleaving the photocleavable moiety and causing the photoreactive moiety to diffuse from the BTOI and react covalently or non-covalently with one or more biological entities in proximity to the BTOI and within a diffusion radius associated with the chemical probe, thereby labeling said one or more biological entities with the moiety that binds a second binding partner.

36. The method of claim 35, wherein a diffusion radius of the photoactive probe and a radius of interrogation of spatiotemporal interactions of the BTOI is adjustable based on the reactivity of the photoreactive moiety and/or the reactivity of the photocleavable moiety.

37. The method of claim 36, wherein the method comprises contacting the BTOI with two or more chemical probes, wherein each of said two or more chemical probes has a different diffusion radius and the moiety that binds a second binding partner of each of said two or more chemical probes binds a different second binding partner.

38. The method of claim 35, wherein the contacting is performed in a live cell, a cell culture, a tissue sample, a bodily fluid sample, or an organ sample.

39. (canceled)

40. The method of claim 35, wherein the method comprises detecting one or more cell-cell interactions, one or more cell-protein interactions, and/or one or more cell-drug interactions.

41. The method of any one of claim 35, wherein the method comprises detecting one or more protein-protein interactions; one or more protein-metabolite interactions; one or more protein-nucleic acid interactions, optionally one or more protein-RNA or protein-DNA interactions; and/or one or more protein-drug interactions.

42. A method for detecting a spatiotemporal interaction of a biological target of interest (BTOI), optionally a cell or protein of interest, wherein the method comprises:
(a) providing a sample comprising a BTOI labeled with a moiety comprising a first binding partner;
(b) contacting the BTOI with a photocatalytic probe comprising: (i) a moiety that binds the first binding partner and (ii) a photocatalytic moiety;
(c) contacting the sample with one or more probe substrates, wherein each probe substrate comprises: (iii) a photoreactive moiety that is capable of undergoing a reaction catalyzed by the photocatalytic moiety and (iv) a detectable moiety or precursor thereof that is capable of specifically binding a second binding partner; and
(d) exposing the sample to light, thereby exciting said photocatalytic moiety and causing the photocatalytic moiety to catalyze a reaction where the photoreactive moiety is transformed into a moiety that can react covalently or non-covalently with one or more biological entities in proximity to the BTOI, thereby labeling said one or more biological entities with the moiety that binds a second binding partner.

43-46. (canceled)

47. A method of detecting interactions of a biological target of interest (BTOI), the method comprising:
(a) providing a sample comprising a labelled BTOI, wherein said labelled BTOI comprises the BTOI and a detectable tag; optionally wherein said BTOI is a cell or a protein, further optionally wherein the detectable tag is protein or peptide;
(b) contacting the sample with a photoactive probe or probe system of claim 2, wherein the target recognition moiety T specifically binds to the detectable tag of the labelled BTOI;
(c) exposing the sample to light, thereby
(i) triggering the cleavage of the photocleavable moiety P₁ and the activation of the photoreactive moiety P₂, wherein the photoreactive moiety P₂ reacts to form a covalent linkage with a second entity in proximity to the POI, thereby tagging said second entity with the detectable moiety R; or
(ii) activating the photocatalytic moiety Pc, thereby catalyzing a reaction of the photoreactive moiety P3, transforming said photoreactive moiety P3 into a moiety that can react to form a covalent linkage with a second entity in proximity to the POI, thereby tagging said second entity with the detectable moiety R; and
(d) detecting the detectable moiety R, thereby detecting the second entity interacting with or in proximity to the BTOI.

48. The method of claim 47, wherein the BTOI is a protein of interest (POI) and providing a sample comprising a labelled BTOI comprises providing a sample comprising a labelled POI, wherein said labelled POI comprises the POI and a detectable tag; optionally wherein the detectable tag is protein or peptide, further optionally wherein the detectable tag is selected from a SNAP-tag, a Halo-Tag, a Clip-Tag, a receptor engineered with strained cyclooctyne, monomeric streptavidin, neutravidin, avidin, FKBP12 or a mutant thereof, and DHFR; wherein the target recognition moiety T of the chemical probe specifically binds to the detectable tag of the labelled POI; and wherein detecting the detectable moiety R of the chemical probe, thereby detecting the protein in proximity to the POI.

49-56. (canceled)

57. A kit comprising:
(a) a photoactive probe or probe system of claim 1; and
(b) one or more of: a cell culture medium, optionally containing one or more heavy isotopes; a buffer; and a solid support material comprising a binding partner of the detectable moiety, optionally wherein said solid support material comprises streptavidin-coated beads.
